(19) United States
(12) Patent Application Publication
(10) Pub. No.: US 2026/0354988 A1
(43) Pub. Date: Nov. 12, 2026

(54) MODULAR ADJUSTABLE BLADE GUIDE BLOCK FOR JOINT REVISION SURGERY

(71) Applicant: MAP Medical Solutions, LLC, Twin Falls, ID (US)

(72) Inventor: Mark B. Wright, Rupert, ID (US)

(73) Assignee: MAP Medical Solutions, LLC, Twin Falls, ID (US)

(21) Appl. No.: 19/747,535

(22) Filed: Jul. 20, 2026

Related U.S. Application Data
(63) Continuation-in-part of application No. 18/083,215, filed on Dec. 16, 2022, now Pat. No. 12,685,545.

(51) Int. Cl. A61F 2/46; A61B 17/3211; A61B 17/00; A61B 17/32

(52) U.S. Cl. CPC A61F 2/4607; A61B 17/3211; A61B 2017/00477; A61B 2017/320052; A61F 2002/4619

(57) ABSTRACT

The present application is directed to a Modular Adjustable Blade Guide Block for Joint Revision Surgery for removal of an existing implant and includes a modular adjustable blade guide block can be configured in a one-piece, two-piece or three-piece configuration, and blade guide block modules can be added or removed as required. A removeable lateral adjustment piece and a removable medial piece are provided. A trunnion securing assembly is provided. A blade centering backpressure plate is provided. All disclosed configurations of the guide block accommodate one or more additional fixation points. All configurations of the guide block are structured to be used with rigid and flexible osteotome blades. All configurations of the guide blocks are structured to be used with robotics and navigation technologies. The one-piece, two-piece and three-piece guide blocks all facilitate implant size adjustments for rapid, efficient and complete removal of an existing prosthesis during joint revision surgery.

### CROSS-REFERENCE TO RELATED APPLICATIONS

The present non-provisional patent application is a continuation-in-part of Applicant’s patent application number 18/083,215 filed on Dec. 16, 2022, which application is a divisional of US patent application serial number 18/075,574 filed on Dec. 6, 2022 and issued as US Pat. No. 12,478,389 B2 on Nov. 25, 2025, which application is a continuation-in-part of Applicant’s patent application serial number 17/899,521 filed on Aug. 30, 2022 and issued as US Pat. No. 12,502,183 B2 on Dec. 23, 2025, which application is a continuation-in-part of Applicant’s patent application serial number 17/542,636 filed on Dec. 6, 2021 and issued as US Pat. No. 12,484,913 B2 on Dec. 2, 2025, which application is a continuation-in-part of Applicant’s patent application serial number 17/218,000 filed on Mar. 30, 2021 and issued as US Pat. No. 11,992,422 B2 on May 28, 2024, which application is a continuation-in-part of serial number 16/398,564 filed on Apr. 30, 2019 and issued as US Patent No. 11,116,525 on Sep. 14, 2021, which US Patent claims the benefit of US provisional patent application serial number 62/665,894 filed on May 2, 2018.

### FIELD OF THE INVENTION

This application relates to a surgical apparatus used on joint replacement revision surgery in the areas of the hip joint, shoulder joint and knee joint. More particularly, the present application is directed to a Modular Adjustable Blade Guide Block for Joint Revision Surgery for removal of an existing implant and includes a modular adjustable osteotome blade/surgical chisel guide block, and a plurality of rigid and flexible lateral shoulder release osteotome and guide blades, rigid and flexible medial calcar osteotome and guide blades, rigid anterior and posterior osteotome blades of differing sizes and curved medial calcar and lateral shoulder release osteotome and guide blades. The modular blade guide block has a plurality of rearward blade guide slots, a central cavity and a plurality of guide slots having a two-way adjustable L-shaped guide plate. The modular adjustable blade guide block central cavity is positioned over the trunnion end of the existing prosthesis to be removed and secured to the prothesis. The modular adjustable blade guide block can be configured in a 1-piece, 2-piece or 3-piece configuration, and blade guide block modules can be added or removed as required. The 2-piece and 3-piece modular adjustable blade guide blocks enable adjustment to the size of the implant being extracted or of the specific soft tissue envelope found in the patient. The varying sizes of the flexible and rigid blades also enable adjustment to the size of the implant being extracted. Straight, curved and compound curved osteotome blades are guided by the blade guide slots to cut the prothesis free. The guide block is secured to the implant and can be adjusted to guide the direction and angle of the blades inserted therein, then the flexible and rigid blades cut the implant away from the bone to allow the implant to be removed by the action of a slide hammer. The adjustable guide blocks as well as the varying sizes and configurations of straight, curved and compound curved osteotome blades/surgical chisels and related accessories may be sold as a complete surgeon’s hospital kit. The Modular Adjustable Blade Guide Block for Joint Revision Surgery disclosed herein facilitates implant size adjustments for rapid, efficient and complete removal of an existing prosthesis during joint revision surgery.

### BACKGROUND OF THE INVENTION

There is growing need to provide a new and refined apparatus and method for performing delicate surgical operations including hip, shoulder and knee revisions. The similarity in these operations is that the implants have to be inserted into a major bone in the area and when there is a problem with them the prosthesis has to be removed.

Extraction of an existing implant can be a challenging task. When the implant is set it is cemented or uncemented into the bone to be secured to the bone. Implants are of varying size, shape and configuration and chosen by the orthopedic surgeon based on the requirements of the patient. Femoral hip stems are one of the most challenging implants to be removed during revision surgery.

MAP Medical Solutions, LLC and inventor Mark B. Wright MD have been at the forefront of developing technology in the field of joint revision surgery. Apparatus and methods development started in 2017 and initially culminated in the filing of US provisional patent application Serial No. 62/665,894, filed on May 2, 2018 covering the first of its kind joint revision surgery apparatus which included a blade guide block having a plurality of blade guide slots and a central cavity.

As a follow-up to the aforementioned US provisional patent application, non-provisional US patent application Serial No. 16/398,564 was filed on Apr. 30, 2019 and matured into US Pat. No. 11,116,525 B2 issued on Sep. 14, 2021. This patent disclosed, taught and claimed a Joint Revision Surgery Apparatus which includes a blade guide block which has a plurality of blade guide slots and a central cavity. The blade guide block central cavity is positioned over the trunnion end of the existing prosthesis to be removed and secured to the prothesis. Straight, curved and compound curved knife blades are guided by the blade guide slots to cut the prothesis free. The securing of the guide blade block support apparatus against the femur is accomplished by the means of a T handle screw or eyebolt threading into one of the threaded orifice in the blade guide block frame. The guide blocks in varying sizes and configurations, straight and curved knifes blades and related accessories may be sold as a complete kit. The Joint Revision Surgery Apparatus facilitates rapid, efficient and complete removal of an existing prosthesis during joint revision surgery.

As a follow-up to US Pat. No. 11,116,525 B2, a continuation-in-part (CIP) patent application was filed as Serial No. 17/218,000 on Mar. 30, 2021 and matured into US Pat. No. 11,992,422 B2 on May 28, 2024. This patent disclosed, taught and claimed a Joint Revision Surgery Apparatus which includes a blade guide block which has a plurality of blade guide slots, both straight and curved, and a central cavity. The blade guide block central cavity is positioned over the trunnion end of the existing prosthesis to be removed and secured to the prothesis. The guide blade block is secured to the trunnion of the prosthesis to be extracted using retaining rings housed within the guide block. Straight, curved and compound curved knife blades are guided by the blade guide slots to cut the prothesis free. The guide blocks, in varying sizes and configurations, straight and curved knifes blades and related accessories may be sold as a complete kit. The Joint Revision Surgery Apparatus facilitates rapid, efficient and complete removal of an existing prosthesis during joint revision surgery, and significantly increases positive medical outcomes for joint revision procedures.

As a follow-up to US Pat. No. 11,992,422 B2, a continuation-in-part (CIP) patent application was filed as Serial No. 17/542,636 on Dec. 6, 2021 and matured into US Pat. No. 12,484,913 B2 on Dec. 2, 2025. This patent disclosed, taught and claimed a Joint Revision Surgery Apparatus which includes a surgical knife blade guide block which has a plurality of rearward blade guide slots, a central cavity and a large forward slot having an adjustable knife blade guide pressure plate. The surgical knife blade guide block central cavity houses a stem trunnion securing member and is positioned over the trunnion end of the existing prosthesis to be removed and secured to the prothesis. The guide blade block is secured to the trunnion of the prosthesis to be extracted using a stem trunnion securing member housed within the guide block. Straight, curved and compound curved knife blades are guided by the blade guide slots to cut the prothesis free. The Joint Revision Surgery Apparatus facilitates rapid, efficient and complete removal of an existing prosthesis during joint revision surgery, and significantly increases positive medical outcomes for joint revision procedures.

As a follow-up to US Pat. No. 12,484,913 B2, a continuation-in-part (CIP) patent application was filed as Serial No. 17/899,521 on Aug. 30, 2022 and matured into US Pat. No. 12,502,183 B2 on Dec. 23, 2025. This patent disclosed, taught and claimed a Joint Revision Surgery Apparatus which includes a surgical knife blade guide block which has a plurality of rearward blade guide slots, a central cavity and a plurality of guide slots having a two-way adjustable L-shaped guide plate. The surgical knife blade guide block central cavity houses a stem trunnion securing member and is positioned over the trunnion end of the existing prosthesis to be removed and secured to the prothesis. The guide blade block is secured to the trunnion of the prosthesis to be extracted using a stem trunnion securing member housed within the guide block. Straight, curved and compound curved knife blades are guided by the blade guide slots to cut the prothesis free. The Joint Revision Surgery Apparatus facilitates rapid, efficient and complete removal of an existing prosthesis during joint revision surgery, and significantly increases positive medical outcomes for joint revision procedures.

As a follow-up to US Pat. No. 12,502,183 B2, a continuation-in-part (CIP) patent application was filed as Serial No. 18/075,574 on Dec. 6, 2022 and matured into US Pat. No. 12,478,389 B2 on Nov. 25, 2025. This patent disclosed, taught and claimed a Joint Revision Surgery System and Method which includes a surgical osteotome blade guide block which has a plurality of rearward blade guide slots, a central cavity and a plurality of forward guide slots having a two-way adjustable L-shaped guide plate. The surgical osteotome blade guide block central cavity houses a stem trunnion securing and is secured to the prothesis to be extracted. The guide blade block is secured to the trunnion of the prosthesis to be extracted using a stem trunnion securing member housed within the guide block. Straight, curved, flexible and rigid and compound curved osteotome blades are guided by the blade guide slots to cut the implant free from the bone. The Joint Revision Surgery System and Method facilitates rapid, efficient and complete removal of an existing prosthesis during joint revision surgery, and significantly increases positive medical outcomes for joint revision procedures.

More particularly, US Pat. No. 12,478,389 was directed to four primary components, namely:
(1) the osteotome blade/surgical chisel guide block assembly;
(2) the osteotome blade/surgical chisel locking handle assembly;
(3) the surgical slide hammer; and
(4) the various flexible medial calcar osteotome blades/surgical chisels, the lateral shoulder release blade, the anterior and posterior blades and the general purpose release chisel of the Joint Revision Surgery System and Method all work synergistically together to make extraction of an implant more efficient, less time consuming and with significantly less blood loss by the patient, all of which result in improved patient outcomes. Moreover, this system can be adjusted for any size and shape of implant to be extracted, including collared and non-collared prostheses. The guide block assembly embodiment of the joint revision surgery system and method wherein an assembled osteotome blade/surgical chisel guide block having an adjustable L-shaped osteotome blade/surgical chisel guide plate having a lower portion and an upper portion, can be adjusted in two separate ways. First, the adjustment plate retaining screw can be threaded outwardly (lifted) or threaded inwardly (lowered) to retract or extend the lower adjustment plate towards or away from the stem. Second, the adjustment plate retaining screw can be removed altogether and the guide plate lower section shifted to a different guide slot within the plurality of blade guide slots, with that guide slot being closer or farther away from the stem to be extracted. One or both of the aforementioned guide plate adjustments can be made to successfully guide a cutting blade down to a stem of varying size, and in this way the guide block assembly can accommodate varying sized stems to be removed during revision surgery. Moreover, both a collared and non-collared stem can be extracted by making the appropriate adjustments to the length and distance of the adjustable guide plate.

As a follow-up to US Pat. No. 12,478,389 B2, a continuation (CON) patent application was filed as Serial No. 18/083,215 on Dec. 16, 2022 and is currently pending. This patent disclosed, taught and claimed a Joint Revision Surgery Osteotome Blades and Surgical Chisel Blades which includes a surgical osteotome blade guide block which has a plurality of rearward blade guide slots, a central cavity and a plurality of forward guide slots having a two-way adjustable L-shaped guide plate. The surgical osteotome blade guide block central cavity houses a stem trunnion securing and is secured to the prothesis to be extracted. The guide blade block is secured to the trunnion of the prosthesis to be extracted using a stem trunnion securing member housed within the guide block. Straight, curved, flexible and rigid and compound curved osteotome blades are guided by the blade guide slots to cut the implant free from the bone. The Joint Revision Surgery Osteotome Blades and Surgical Chisel Blades facilitates rapid, efficient and complete removal of an existing prosthesis during joint revision surgery, and significantly increases positive medical outcomes for joint revision procedures.

More particularly, US Serial No. 18/083,215 was directed to the four primary components, namely:
(1) the osteotome blade/surgical chisel guide block assembly;
(2) the osteotome blade/surgical chisel locking handle assembly;
(3) the surgical slide hammer; and
(4) the various flexible medial calcar osteotome blades/surgical chisels, the lateral shoulder release blade, the anterior and posterior blades and the general purpose release chisel blades of the Joint Revision Surgery Osteotome Blades and Surgical Chisel Blades with an emphasis on the blades cutting edge configurations, sizes and uses. Currently, some femoral stems are being designed and manufactured with a metaphyseal/diaphyseal joint (MDJ) and this presents a problem when cutting the femoral implant from the cement/bone interface (see FIG. 12 below). Moreover, differing sized stems are chosen to be implanted into different size patients anatomy. This presents specific challenges for joint revision surgeons not yet overcome by existing technology in this area.

Even with all of these numerous innovations for various joint revision surgery apparatuses previously provided by MAP Medical Solutions, LLC and inventor Mark B. Wright MD, development of improvements were ongoing. This application is directed to improving the way femoral stems, especially those with an MDJ, are extracted. In this regard, through continuing research and development, it was determined that:
(a) having multiple points of fixation to the stem to be extracted, over and above the single trunnion fixation points;
(b) having the capacity to adjustably guide a flexible cutting blade down the lateral shoulder using a rigid guide blade;
(c) having a multiple modular adjustable guide block capable of being configured for varying stem extraction purposes;
(d) having an improved backpressure blade structured to achieve better centering of the cutting and guide blades;
(e) having multiple sized flexible cutting blades, anterior/posterior cutting blades and rigid guide blades; and
(f) having a multiple modular adjustable guide block capable of being employed using medical robotics instrumentation, navigation and tracking, would all be improvements to the existing technology which would propel the innovations into the next phase of joint revision surgery moving forward into the future. These innovations in femoral stem extraction are the subject matter of this patent application.

Therefore, even though these previously described innovations in revision stem removal may be suitable for the specific individual purposes to which they address, they differ significantly from the present Modular Adjustable Blade Guide Block for Joint Revision Surgery, capable of being adjusted for size and MDJ configuration (see FIG. 12 below), and deployed by robotics and navigation systems, as hereinafter contrasted. None of the foregoing prior art teaches or suggests the particular unique features and components of the Modular Adjustable Blade Guide Block for Joint Revision Surgery and thus clarifies the need for further improvements in the types of surgical device systems which can be used for these purposes, namely, making extraction of existing protheses and implants easier and revision surgery more efficient and more effective, leading to improved patient outcomes.

In this respect, before explaining at least one embodiment of the Modular Adjustable Blade Guide Block for Joint Revision Surgery in detail, it is to be understood that the design is not limited in its application to the details of construction and to the arrangement of the components set forth in the following description or illustrated in the drawings. The Modular Adjustable Blade Guide Block for Joint Revision Surgery disclosed herein is capable of other embodiments and of being practiced and carried out in various ways. Also, it is to be understood that the phraseology and terminology employed herein are for the purpose of description and should not be regarded as limiting.

### SUMMARY OF THE INVENTION

The principle advantage of the of the Modular Adjustable Blade Guide Block for Joint Revision Surgery is to enable the performance of a significantly quicker, safer and more successful joint revision surgery procedure, resulting in significantly less blood loss during the joint revision surgery procedure and much shorter operating room use times during the joint revision surgery procedure.

Another advantage of using the Modular Adjustable Blade Guide Block for Joint Revision Surgery is to perform joint revision surgery with less instrumentation and tools which are more efficient and effective in removing femoral implants.

Another advantage of using the Modular Adjustable Blade Guide Block for Joint Revision Surgery is to have quicker times to have full weight bearing capability after joint revision surgery, fewer complications during and after joint revision surgery, resulting in less morbidity and mortality following joint revision surgery.

Another advantage of using the Modular Adjustable Blade Guide Block for Joint Revision Surgery is that it will accommodate the removal of varying sizes of femoral implants which femoral stem are manufactured in varying sizes and configurations.

An advantage of the Modular Adjustable Blade Guide Block for Joint Revision Surgery is to provide a blade guide block having three primary configurations, including (1) a monolithic one-piece osteotome/surgical chisel blade guide block assembly; (2) a two-piece osteotome/surgical chisel blade guide block assembly; and (3) a three-piece osteotome/surgical chisel blade guide block assembly all of which are capable of accurately guiding a variety of both flexible and rigid osteotome/surgical chisel cutting blades.

An advantage of the Modular Adjustable Blade Guide Block for Joint Revision Surgery is that it provides a central trunnion securing member assembly as a fixation point, as well as being structured to accommodate additional fixation points as required.

Another advantage of using the Modular Adjustable Blade Guide Block for Joint Revision Surgery is that it provides a laterally adjustable removeable lateral piece which can be adjusted for differing sizes of femoral implants as required.

Another advantage of the Modular Adjustable Blade Guide Block for Joint Revision Surgery is to both provide and accommodate rigid and flexible uniquely shaped and compound curved lateral guide blades, flexible uniquely shaped medial calcar osteotome blades and surgical chisel blades, as well as curved lateral shoulder release osteotome blades and surgical chisel blades, general purpose osteotome blades and surgical chisel blades and anterior and posterior osteotome blades and surgical chisel blades.

And yet another advantage of the Modular Adjustable Blade Guide Block for Joint Revision Surgery is to provide one or more threaded orifices within the guide block which enable the connection of one or more additional fixation points.

Another advantage of the Modular Adjustable Blade Guide Block for Joint Revision Surgery is to provide one or more threaded orifices within the guide block which enable the connection of one or more robotics arrays and trackers.

And a further advantage of the Modular Adjustable Blade Guide Block for Joint Revision Surgery is to provide a modular adjustable blade guide block capable of being used with robotics and navigation technologies.

In summary, a one-piece modular adjustable blade guide block is disclosed, comprising
(a) a one piece body including a central portion, a medial portion and a lateral portion and having a centrally located cavity;
(b) a trunnion securing member assembly mounted within the centrally located cavity;
(c) a plurality of blade guide slots located in the one piece body medial portion; and
(d) a plurality of blade guide slots located in the one piece body lateral portion. The one-piece modular adjustable blade guide block wherein said one piece body includes a threaded orifice located in the central portion. The one-piece modular adjustable blade guide block further including a fixation screw having a screw knob and screw threads wherein said screw threads mate with said threaded orifice located in the central portion of said one piece body. The one-piece modular adjustable blade guide block wherein said plurality of blade guide slots located in the one piece body medial portion are configured to accept an upwardly and downwardly adjustable backpressure plate. And the one-piece modular adjustable blade guide block wherein said upwardly and downwardly adjustable backpressure plate further includes blade centering tabs.

Additionally, a two-piece modular adjustable blade guide block is disclosed, comprising:
(a) a two piece body including a central piece having a central portion and a medial portion and a removable adjustable lateral piece and having a centrally located cavity within said central portion;
(b) a trunnion securing member assembly mounted within the centrally located cavity;
(c) a plurality of blade guide slots located in the removable adjustable lateral piece; and
(d) a plurality of blade guide slots located in the one piece body medial portion. The two-piece modular adjustable blade guide block wherein said removable adjustable lateral piece is slidably secured to the central portion using an adjustment screw. The two-piece modular adjustable blade guide block further wherein said central piece includes a threaded orifice located in the central portion. The two-piece modular adjustable blade guide block further including a fixation screw having a screw knob and screw threads wherein said screw threads mate with said threaded orifice located in the central portion. The two-piece modular adjustable blade guide block further including a robotics navigation tracker array having screw threads wherein said screw threads mate with said threaded orifice located in the central portion. The two-piece modular adjustable blade guide block wherein said plurality of blade guide slots located in the medial portion are configured to accept an upwardly and downwardly adjustable backpressure plate. And the two-piece modular adjustable blade guide block wherein said upwardly and downwardly adjustable backpressure plate further includes blade centering tabs.

Furthermore, a three-piece modular adjustable blade guide block, comprising:
(a) a three piece body including a central piece having a central portion and having a centrally located cavity within said central portion, a removeable medial piece and a removable adjustable lateral;
(b) a trunnion securing member assembly mounted within the centrally located cavity;
(c) a plurality of blade guide slots located in the removable adjustable lateral piece; and
(d) a plurality of blade guide slots located in the medial piece. The three-piece modular adjustable blade guide block wherein said removable adjustable lateral piece is slidably secured to the central portion using an adjustment screw. The three-piece modular adjustable blade guide block further wherein said central piece includes a threaded orifice located in the central portion. The three-piece modular adjustable blade guide block further including a fixation screw having a screw knob and screw threads wherein said screw threads mate with said threaded orifice located in the central portion. The three-piece modular adjustable blade guide block further including a robotics navigation tracker array having screw threads wherein said screw threads mate with said threaded orifice located in the central portion. The three-piece modular adjustable blade guide block wherein said plurality of blade guide slots located in the medial portion are configured to accept an upwardly and downwardly adjustable backpressure plate. The three-piece modular adjustable blade guide block wherein said upwardly and downwardly adjustable backpressure plate further includes blade centering tabs. The three-piece modular adjustable blade guide block according to claim 12, wherein said medial piece further includes a threaded orifice configured to accept a fixation point screw. The three-piece modular adjustable blade guide block further including a fixation point screw having a screw knob and screw threads and configured to thread into said threaded orifice located in said medial piece. The three-piece modular adjustable blade guide block wherein said central piece includes one or more threaded orifices. The three-piece modular adjustable blade guide block further including one or more fixation point screws having a screw knob and screw threads and wherein said one or more fixation point screws are configured to be threaded into said threaded orifices located in said central piece.

Moreover, the one-piece, two-piece and three-piece modular adjustable blade guide blocks all include a plurality of blade guide slots located in the lateral portion or piece which are configured to accept rigid lateral guide blades and flexible lateral cutting blades. And the one-piece, two-piece and three-piece modular adjustable blade guide blocks all include a plurality of blade guide slots located in the medial portion or piece which are configured to accept rigid compound curved medial calcar guide blades and flexible medial calcar cutting blades.

With respect to the above description then, it is to be realized that the optimum dimensional relationships for the parts of the Modular Adjustable Blade Guide Block for Joint Revision Surgery, to include variations in size, materials, shape, form, function and manner of operation, assembly and use, are deemed readily apparent and obvious to one skilled in the art, and all equivalent relationships to those illustrated in the drawings and described in the specification are intended to be encompassed by the present design. Therefore, the foregoing is considered as illustrative only of the principles of the Modular Adjustable Blade Guide Block for Joint Revision Surgery. Further, since numerous modifications and changes will readily occur to those skilled in the art, it is not desired to limit the Modular Adjustable Blade Guide Block for Joint Revision Surgery to the exact construction and operation shown and described, and accordingly, all suitable modifications and equivalents may be resorted to, falling within the scope of this application. While the apparatus and method has or will be described for the sake of grammatical fluidity with functional explanations, it is to be expressly understood that the claims, unless expressly formulated under 35 USC 112, or similar applicable law, are not to be construed as necessarily limited in any way by the construction of “means” or “steps” limitations, but are to be accorded the full scope of the meaning and equivalents of the definition provided by the claims under the judicial doctrine of equivalents, and in the case where the claims are expressly formulated under 35 USC 112 are to be accorded full statutory equivalents under 35 USC 112, or similar applicable law. The Modular Adjustable Blade Guide Block for Joint Revision Surgery can be better visualized by turning now to the following drawings wherein like elements are referenced by like numerals.

### BRIEF DESCRIPTION OF THE DRAWINGS

The accompanying drawings, which are incorporated in and form a part of this specification, illustrate embodiments of the Modular Adjustable Blade Guide Block for Joint Revision Surgery and together with the description, serve to explain the principles of this application.

FIG. 1 depicts a top, side elevational and perspective view of a previously disclosed 2-piece embodiment of the joint revision surgery apparatus illustrating an assembled osteotome blade guide block having a previously disclosed adjustable L- shaped medial osteotome blade guide backpressure plate fully extended downwardly.

FIG. 2 depicts a side elevational view of the previously disclosed 2-piece embodiment of the joint revision surgery apparatus illustrating an assembled osteotome blade guide block having a previously disclosed adjustable L- shaped osteotome blade guide backpressure plate fully extended downwardly.

FIG. 3 depicts an enlarged side elevational view of the previously disclosed upper section backpressure guide plate and previously disclosed backpressure guide plate adjustment screw having an integral retaining washer welded to the underside of the upper section backpressure guide plate.

FIG. 4 depicts a front view of the previously disclosed previously disclosed 2-piece embodiment of the joint revision surgery apparatus illustrating a previously disclosed assembled osteotome blade guide block having an adjustable L- shaped osteotome blade guide backpressure plate fully extended downwardly.

FIG. 5 depicts a cross-sectional view of the previously disclosed 2-piece embodiment of the joint revision surgery apparatus illustrating an assembled osteotome blade guide block having a previously disclosed adjustable L- shaped osteotome blade guide backpressure plate fully extended downwardly of FIG. 4.

FIG. 6 depicts a side view of a previously disclosed stem trunnion securing piece.

FIG. 7 depicts a cross-sectional view of the previously disclosed stem trunnion securing piece, as shown in FIG. 6.

FIG. 8 depicts a bottom view of the previously disclosed stem trunnion securing piece.

FIG. 9 depicts a side perspective view of the previously disclosed stem trunnion securing piece.

FIG. 10 depicts a cross-sectional view of the previously disclosed embodiment of the joint revision surgery apparatus illustrating a 2-piece assembled osteotome blade guide block having an adjustable L- shaped osteotome blade guide backpressure plate, showing the osteotome blades inserted into the forward and rearward osteotome blade guide slots within the guide block, for removal of a collared implanted femoral stem.

FIG. 11A depicts a cross-sectional side elevational view of a previously disclosed surgical blade locking osteotome handle assembly having a surgical osteotome blade inserted therein, here shown in place attached to a surgical osteotome blade guide block assembly.

FIG. 11B depicts a rear view of the previously disclosed surgical blade locking osteotome handle assembly having a surgical osteotome blade inserted therein, here shown in place attached to a surgical osteotome blade guide block assembly, as shown in FIG. 11A.

FIG. 12 depicts a typical femoral implant configuration and describes the various parts and portions thereof, illustrating in particular the metaphyseal/diaphyseal junction (MDJ) which will be discussed in detail in relation to the apparatus system improvements described below.

### 1-PIECE

FIG. 13 depicts a top, side elevational and perspective view of a monolithic 1-piece blade guide block having an additional fixation point feature.

FIG. 14 depicts a side elevational view of the monolithic 1-piece blade guide block having an additional fixation point feature as shown in FIG. 13.

FIG. 15 depicts a top plan view of the monolithic 1-piece blade guide block having an additional fixation point feature as shown in FIG. 13.

FIG. 16 depicts a cross-sectional view of the monolithic 1-piece blade guide block having an additional fixation point feature as shown in FIG. 15.

FIG. 17 depicts a top, side elevational and perspective view of a monolithic 1-piece blade guide block having a fixation point screw illustrating a rigid lateral guide blade guiding a flexible lateral cutting blade toward the lateral shoulder surface of the femoral stem to be extracted.

FIG. 18 depicts a side elevational view of the monolithic 1-piece blade guide block having a fixation point screw shown in FIG. 17, illustrating a rigid lateral blade guiding a flexible lateral cutting blade toward the lateral shoulder surface of the femoral stem to be extracted.

FIG. 19 depicts a top plan view of the monolithic 1-piece blade guide block having a fixation point screw shown in FIG. 17, illustrating a rigid lateral blade guiding a flexible lateral cutting blade toward the lateral shoulder surface of the femoral stem to be extracted.

FIG. 20 depicts a cross-sectional view of the monolithic 1-piece blade guide block having a fixation point screw shown in FIG. 19, illustrating a rigid lateral blade guiding a flexible lateral cutting blade toward the lateral shoulder surface of the femoral stem to be extracted.

FIG. 21 depicts a top, side elevational and perspective view of a monolithic 1-piece blade guide block having a fixation point screw illustrating a rigid lateral blade guiding a flexible lateral cutting blade, here shown advanced all the way down the lateral shoulder surface of the femoral stem to be extracted.

FIG. 22 depicts a side elevational view of the monolithic 1-piece blade guide block having a fixation point screw shown in FIG. 21, illustrating a rigid lateral blade guiding a flexible lateral cutting blade, here shown advanced all the way down the lateral shoulder surface of the femoral stem to be extracted.

FIG. 23 depicts a top plan view of the monolithic 1-piece blade guide block having a fixation point screw shown in FIG. 21, illustrating a rigid lateral blade guiding a flexible lateral cutting blade, here shown advanced all the way down the lateral shoulder surface of the femoral stem to be extracted.

FIG. 24 depicts a cross-sectional view of the monolithic 1-piece blade guide block having a fixation point screw shown in FIG. 23, illustrating a rigid lateral blade guiding a flexible lateral cutting blade advanced all the way down the lateral shoulder surface of the femoral stem to be extracted.

### 2-PIECE

FIG. 25 depicts a top, side elevational and perspective view of a 2-piece modular blade guide block illustrating a laterally movably adjustable piece housing the lateral blade guide slots.

FIG. 26 depicts a side elevational view of the 2-piece modular blade guide block illustrating a laterally movably adjustable piece housing the lateral blade guide slots as shown in FIG. 25.

FIG. 27 depicts a top plan view of the 2-piece modular blade guide block illustrating a laterally movably adjustable piece housing the lateral blade guide slots as shown in FIG. 25.

FIG. 28 depicts a cross-sectional view of the 2-piece modular blade guide block illustrating a laterally movably adjustable piece housing the lateral blade guide slots as shown in FIG. 27.

FIG. 29 depicts a side elevational view of the 2-piece modular blade guide block illustrating a laterally movably adjustable piece housing the lateral blade guide slots, illustrating the laterally movably adjustable piece moving outwardly laterally.

FIG. 30 depicts a side elevational view of the 2-piece modular blade guide block illustrating a laterally movably adjustable piece housing the lateral blade guide slots, illustrating the laterally movably adjustable piece moving further outwardly laterally than shown in FIG. 29.

FIG. 31 depicts a top plan view of the 2-piece modular blade guide block illustrating a laterally movably adjustable piece housing the lateral blade guide slots, illustrating the laterally movably adjustable piece moving further outwardly laterally as shown in FIG. 30.

FIG. 32 depicts a cross-sectional view of the 2-piece modular blade guide block illustrating a laterally movably adjustable piece housing the lateral blade guide slots, illustrating the laterally movably adjustable piece moving further outwardly laterally as shown in FIG. 31.

FIG. 33 depicts an exploded perspective view of the 2-piece modular blade guide block illustrating a laterally movably adjustable piece housing the lateral blade guide slots.

FIG. 34 depicts an exploded side elevational view of the 2-piece modular blade guide block illustrating a laterally movably adjustable piece housing the lateral blade guide slots.

FIG. 35 depicts a partially exploded top plan view of the 2-piece modular blade guide block illustrating a laterally movably adjustable piece housing the lateral blade guide slots.

FIG. 36 depicts a fully exploded cross-sectional view of the 2-piece modular blade guide block illustrating a laterally movably adjustable piece housing the lateral blade guide slots as shown in FIG. 35.

FIG. 37 depicts a top, side elevational and perspective view of a 2-piece modular blade guide block illustrating a laterally movably adjustable piece housing the lateral blade guide slots, here shown with the rigid lateral blade guiding the flexible lateral cutting blade down the lateral surface of the femoral stem to be extracted.

FIG. 38 depicts a side elevational view of the 2-piece modular blade guide block illustrating a laterally movably adjustable piece housing the lateral blade guide slots, here shown with the rigid lateral blade guiding the flexible lateral cutting blade down the lateral surface of the femoral stem to be extracted as shown in FIG. 37.

FIG. 39 depicts a top plan view of the 2-piece modular blade guide block illustrating a laterally movably adjustable piece housing the lateral blade guide slots, here shown with the rigid lateral blade guiding the flexible lateral cutting blade down the lateral surface of the femoral stem to be extracted as shown in FIG. 37.

FIG. 40 depicts a cross-sectional view of the 2-piece modular blade guide block illustrating a laterally movably adjustable piece housing the lateral blade guide slots, here shown with the rigid lateral blade guiding the flexible lateral cutting blade down the lateral surface of the femoral stem to be extracted as shown in FIG. 39.

FIG. 41 depicts a partially exploded top, side elevational and perspective of a 2-piece modular blade guide block illustrating a laterally movably adjustable piece housing the lateral blade guide slots, here shown with an additional fixation point feature.

FIG. 42 depicts a partially exploded side elevational view of the 2-piece modular blade guide block illustrating a laterally movably adjustable piece housing the lateral blade guide slots, with an additional fixation point screw as shown in FIG. 41.

FIG. 43 depicts a partially exploded side elevational view of the 2-piece modular blade guide block illustrating a laterally movably adjustable piece housing the lateral blade guide slots, with an additional fixation point screw as shown in FIG. 41.

FIG. 44 depicts a partially exploded cross-sectional view of the 2-piece modular blade guide block illustrating a laterally movably adjustable piece housing the lateral blade guide slots, with an additional fixation point screw as shown in FIG. 43.

FIG. 45 depicts a top, side elevational and perspective of a 2-piece modular blade guide block illustrating a laterally movably adjustable piece housing the lateral blade guide slots, here shown with an additional fixation point screw threaded all the way down and affixed to the femoral stem to be extracted.

FIG. 46 depicts a side elevational view of the 2-piece modular blade guide block illustrating a laterally movably adjustable piece housing the lateral blade guide slots, with an additional fixation point screw threaded all the way down and affixed to the femoral stem to be extracted as shown in FIG. 45.

FIG. 47 depicts a top plan view of the 2-piece modular blade guide block illustrating a laterally movably adjustable piece housing the lateral blade guide slots, with an additional fixation point screw threaded all the way down and affixed to the femoral stem to be extracted as shown in FIG. 45.

FIG. 48 depicts a cross-sectional view of the 2-piece modular blade guide block illustrating a laterally movably adjustable piece housing the lateral blade guide slots, with an additional fixation point screw threaded all the way down and affixed to the femoral stem to be extracted as shown in FIG. 47.

FIG. 49 depicts a partially exploded top, side elevational and perspective of a 2-piece modular blade guide block illustrating a removed laterally movably adjustable piece, here shown with an additional fixation point screw.

FIG. 50 depicts a partially exploded side elevational view of the 2-piece modular blade guide block illustrating a removed laterally movably adjustable piece, here shown with an additional fixation point screw, as shown in FIG. 49.

FIG. 51 depicts a partially exploded top plan view of the 2-piece modular blade guide block illustrating a removed laterally movably adjustable piece, here shown with an additional fixation point screw, as shown in FIG. 49.

FIG. 52 depicts a partially exploded cross-sectional view of the 2-piece modular blade guide block illustrating a removed laterally movably adjustable piece, here shown with an additional fixation point screw, as shown in FIG. 51.

FIG. 53 depicts a top, side elevational and perspective of a 2-piece modular blade guide block illustrating a removed laterally movably adjustable piece, here shown with an additional fixation point screw having the screw all the way down and affixed to the femoral stem to be extracted.

FIG. 54 depicts a side elevational view of the 2-piece modular blade guide block illustrating a removed laterally movably adjustable piece, with an additional fixation point screw having the screw all the way down and affixed to the femoral stem to be extracted as shown in FIG. 53.

FIG. 55 depicts a top plan view of the 2-piece modular blade guide block illustrating a removed laterally movably adjustable piece, with an additional fixation point showing the screw all the way down and affixed to the femoral stem to be extracted as shown in FIG. 53.

FIG. 56 depicts a cross-sectional view of the 2-piece modular blade guide block illustrating a removed laterally movably adjustable piece, with an additional fixation point screw having the screw all the way down and affixed to the femoral stem to be extracted as shown in FIG. 55.

### 3-PIECE

FIG. 57 depicts a top, side elevational and perspective view of a 3-piece modular blade guide block illustrating a laterally movably adjustable modular piece and a medial modular piece attached to the central universal piece, also showing a medial modular piece retaining pin feature.

FIG. 58 depicts a side elevational view of the 3-piece modular blade guide block illustrating a laterally movably adjustable modular piece and the medial modular piece attached to the central universal piece, also showing a medial piece retaining pin as shown in FIG. 57.

FIG. 59 depicts a top plan view of the 3-piece modular blade guide block illustrating a laterally movably adjustable modular piece and the medial modular piece attached to the central universal piece, also showing a medial piece retaining pin as shown in FIG. 57.

FIG. 60 depicts a cross-sectional view of the 3-piece modular blade guide block illustrating a laterally movably adjustable modular piece and the medial modular piece attached to the central universal piece, also showing a medial piece retaining pin as shown in FIG. 59.

FIG. 61 depicts an exploded top, side elevational and perspective view of a 3-piece modular blade guide block illustrating the laterally movably adjustable modular piece and the medial modular piece detached from the central universal piece, and also showing a medial piece retaining pin.

FIG. 62 depicts an exploded side elevational view of the 3-piece modular blade guide block illustrating the laterally movably adjustable modular piece and the medial modular piece detached from the central universal piece, also showing a medial piece retaining pin as shown in FIG. 61.

FIG. 63 depicts an exploded top plan view of the 3-piece modular blade guide block illustrating the laterally movably adjustable modular piece and the medial modular piece detached from the central universal piece, also showing a medial piece retaining pin as shown in FIG. 62.

FIG. 64 depicts an exploded cross-sectional view of the 3-piece modular blade guide block illustrating the laterally movably adjustable modular piece and the medial modular piece detached from the central universal piece, also showing a medial piece retaining pin as shown in FIG. 63.

FIG. 65 depicts a top, side elevational and perspective view of a 3-piece modular blade guide block illustrating a laterally movably adjustable modular piece removed and the medial modular piece attached to the central universal piece, also showing a medial piece retaining pin.

FIG. 66 depicts a side elevational view of the 3-piece modular blade guide block illustrating a laterally movably adjustable modular piece removed and the medial modular piece attached to the central universal piece, also showing a medial piece retaining pin as shown in FIG. 65.

FIG. 67 depicts a top plan view of the 3-piece modular blade guide block illustrating a laterally movably adjustable piece removed and the medial piece attached to the central universal piece, also showing a medial piece retaining pin as shown in FIG. 65.

FIG. 68 depicts a cross-sectional view of the 3-piece modular blade guide block illustrating a laterally movably adjustable modular piece removed and the medial modular piece attached to the central universal piece, also showing a medial piece retaining pin as shown in FIG. 67.

FIG. 69 depicts a top, side elevational and perspective view of a 3-piece modular blade guide block illustrating the laterally movably adjustable modular piece and the medial modular piece attached to the central universal piece, also showing a medial piece retaining pin, and showing a backpressure plate guiding a flexible medial calcar cutting blade down the medial calcar of a femoral stem to be removed, and also showing a rigid lateral guide blade guiding a flexible lateral cutting blade down the lateral surface of a femoral stem to be extracted.

FIG. 70 depicts a side elevational view of the 3-piece modular blade guide block illustrating the laterally movably adjustable modular piece and the medial modular piece attached to the central universal piece, also showing a medial piece retaining pin, and showing a backpressure plate guiding a cutting blade down the medial calcar of a femoral stem to be removed, and also showing a rigid lateral guide blade guiding a flexible lateral cutting blade down the lateral surface of a femoral stem to be extracted as shown in FIG. 69.

FIG. 71 depicts a top plan view of the 3-piece modular blade guide block illustrating the laterally movably adjustable modular piece and the medial modular piece attached to the central universal piece, also showing a medial piece retaining pin, and showing a backpressure plate guiding a flexible medial calcar cutting blade down the medial calcar of a femoral stem to be removed, and also showing a rigid lateral guide blade guiding a flexible lateral cutting blade down the lateral surface of a femoral stem to be extracted as shown in FIG. 69.

FIG. 72 depicts a cross-sectional view of the 3-piece modular blade guide block illustrating the laterally movably adjustable modular piece and the medial modular piece attached to the central universal piece, also showing a medial piece retaining pin, and showing a backpressure plate guiding a flexible medial calcar cutting blade down the medial calcar of a femoral stem to be removed, and also showing a rigid lateral guide blade guiding a flexible lateral cutting blade down the lateral surface of a femoral stem to be extracted as shown in FIG. 71.

FIG. 73 depicts a top, side elevational and perspective view of a 3-piece modular blade guide block illustrating a laterally movably adjustable modular piece attached to the central universal piece and a medial modular piece removed, also showing the laterally movably adjustable piece extended laterally outward from the central universal piece.

FIG. 74 depicts a side elevational view of the 3-piece modular blade guide block illustrating a laterally movably adjustable modular piece attached to the central universal piece and the medial modular piece removed, also showing the laterally movably adjustable modular piece extended laterally outward from the central universal piece as shown in FIG. 73.

FIG. 75 depicts a top plan view of the 3-piece modular blade guide block illustrating a laterally movably adjustable modular piece attached to the central universal piece and the medial modular piece removed, also showing the laterally movably adjustable piece extended laterally outward from the central universal piece as shown in FIG. 73.

FIG. 76 depicts a cross-sectional view of the 3-piece modular blade guide block illustrating a laterally movably adjustable modular piece attached to the central universal piece and the medial modular piece removed, also showing the laterally movably adjustable piece extended laterally outward from the central universal piece as shown in FIG. 75.

FIG. 77 depicts a partially exploded top, side elevational and perspective view of a 3-piece modular blade guide block illustrating a laterally movably adjustable modular piece and the medial modular piece attached to the central universal piece, also showing an additional fixation point feature.

FIG. 78 depicts a partially exploded side elevational view of the 3-piece modular blade guide block illustrating a laterally movably adjustable modular piece and the medial modular piece attached to the central universal piece, also showing an additional fixation point screw as shown in FIG. 77.

FIG. 79 depicts a partially exploded top plan view of the 3-piece modular blade guide block illustrating a laterally movably adjustable modular piece and the medial modular piece attached to the central universal piece, also showing an additional fixation point screw as shown in FIG. 77.

FIG. 80 depicts a partially exploded cross-sectional view of the 3-piece modular blade guide block illustrating a laterally movably adjustable modular piece and the medial modular piece attached to the central universal piece, also showing an additional fixation point screw as shown in FIG. 79.

FIG. 81 depicts a partially exploded top, side elevational and perspective view of a 3-piece modular blade guide block illustrating a laterally movably adjustable modular piece attached to the central universal piece and the medial modular piece removed, also showing an additional fixation point screw.

FIG. 82 depicts a partially exploded side elevational view of a 3-piece modular blade guide block illustrating a laterally movably adjustable modular piece attached to the central universal piece and the medial modular piece removed, also showing an additional fixation point screw as shown in FIG. 81.

FIG. 83 depicts a top plan view of a 3-piece modular blade guide block illustrating a laterally movably adjustable modular piece attached to the central universal piece and the medial modular piece removed, also showing an additional fixation point screw as shown in FIG. 81.

FIG. 84 depicts a cross-sectional view of a 3-piece modular blade guide block illustrating a laterally movably adjustable modular piece attached to the central universal piece and the medial modular piece removed, also showing an additional fixation point screw as shown in FIG. 83.

FIG. 85 depicts a top, side elevational and perspective view of a 3-piece modular blade guide block illustrating the laterally movably adjustable modular piece attached to the central universal piece and the medial modular piece removed, also showing an additional fixation point screw extended all the way down to make contact with the femoral stem to be extracted.

FIG. 86 depicts a side elevational view of the 3-piece modular blade guide block illustrating the laterally movably adjustable modular piece attached to the central universal piece and the medial modular piece removed, also showing an additional fixation point screw extended all the way down to make contact with the femoral stem to be extracted as shown in FIG. 85.

FIG. 87 depicts a top, side elevational and perspective view of a 3-piece modular blade guide block illustrating the laterally movably adjustable modular piece and the medial modular piece attached to the central universal piece, also showing a medial piece retaining pin, and showing two additional fixation point features, one extending downward from the laterally movably adjustable modular piece and one extending inward from the medial modular piece.

FIG. 88 depicts a left side elevational view of the 3-piece modular blade guide block illustrating the laterally movably adjustable modular piece and the medial modular piece attached to the central universal piece, also showing a medial piece retaining pin, and showing two additional fixation point screws, one extending downward from the laterally movably adjustable modular piece and one extending inward from the medial modular piece as shown in FIG. 87.

FIG. 89 depicts a right side elevational view of the 3-piece modular blade guide block illustrating the laterally movably adjustable modular piece and the medial modular piece attached to the central universal piece, also showing a medial modular piece retaining pin, and showing two additional fixation point screws, one extending downward from the laterally movably adjustable modular piece and one extending inward from the medial modular piece as shown in FIG. 87.

FIG. 90 depicts a top plan view of the 3-piece modular blade guide block illustrating the laterally movably adjustable modular piece and the medial modular piece attached to the central universal piece, also showing a medial piece retaining pin, and showing two additional fixation point screws, one extending downward from the laterally movably adjustable modular piece and one extending inward from the medial modular piece as shown in FIG. 89.

FIG. 91 depicts a cross-sectional view of the 3-piece modular blade guide block illustrating the laterally movably adjustable modular piece and the medial modular piece attached to the central universal piece, also showing a medial piece retaining pin, and showing two additional fixation point screws, one extending downward from the laterally movably adjustable modular piece and one extending inward from the medial modular piece as shown in FIG. 90.

FIG. 92 depicts a top, side elevational and perspective view of a 3-piece modular blade guide block illustrating the laterally movably adjustable modular piece and the medial modular piece attached to the central universal piece, also showing a medial piece retaining pin, and showing four additional fixation point features, one extending downward from the laterally movably adjustable modular piece, one extending inward from the medial modular piece, one extending inward from the right side of the central universal piece and one extending inward from the right side of the central universal piece.

FIG. 93 depicts a side elevational view of the 3-piece modular blade guide block illustrating the laterally movably adjustable modular piece and the medial modular piece attached to the central universal piece, also showing a medial piece retaining pin, and showing four additional fixation point screws, one extending downward from the laterally movably adjustable modular piece, one extending inward from the medial modular piece, one extending inward from the right side of the central universal piece and one extending inward from the right side of the central universal piece as shown in FIG. 92.

FIG. 94 depicts a bottom, side elevational and perspective view of the 3-piece modular blade guide block illustrating the laterally movably adjustable modular piece and the medial modular piece attached to the central universal piece, also showing a medial piece retaining pin, and showing four additional fixation point screws, one extending downward from the laterally movably adjustable modular piece, one extending inward from the medial modular piece, one extending inward from the right side of the central universal piece and one extending inward from the right side of the central universal piece as shown in FIG. 92.

FIG. 95 depicts a top plan view of the 3-piece modular blade guide block illustrating the laterally movably adjustable modular piece and the medial modular piece attached to the central universal piece, also showing a medial piece retaining pin, and showing four additional fixation point screws, one extending downward from the laterally movably adjustable modular piece, one extending inward from the medial modular piece, one extending inward from the right side of the central universal piece and one extending inward from the right side of the central universal piece as shown in FIG. 92.

FIG. 96 depicts a cross-sectional view of the 3-piece modular blade guide block illustrating the laterally movably adjustable modular piece and the medial modular piece attached to the central universal piece, also showing a medial piece retaining pin, and showing four additional fixation point screws, one extending downward from the laterally movably adjustable modular piece, one extending inward from the medial modular piece, one extending inward from the right side of the central universal piece and one extending inward from the right side of the central universal piece as shown in FIG. 95.

FIG. 97 depicts a bottom and right side perspective view of the 3-piece modular blade guide block illustrating the laterally movably adjustable modular piece and the medial modular piece attached to the central universal piece, also showing a medial piece retaining pin, and showing four additional fixation point screws, one extending downward from the laterally movably adjustable modular piece, one extending inward from the medial modular piece, one extending inward from the right side of the central universal piece and one extending inward from the right side of the central universal piece as shown in FIG. 92.

FIG. 98 depicts a bottom and left side perspective view of the 3-piece modular blade guide block illustrating the laterally movably adjustable modular piece and the medial modular piece attached to the central universal piece, also showing a medial piece retaining pin, and showing four additional fixation point screws, one extending downward from the laterally movably adjustable modular piece, one extending inward from the medial modular piece, one extending inward from the right side of the central universal piece and one extending inward from the right side of the central universal piece as shown in FIG. 97.

FIG. 99 depicts a top, side elevational and perspective view of a 3-piece modular blade guide block illustrating the laterally movably adjustable modular piece and the medial modular piece attached to the central universal piece, also showing a medial piece retaining pin, and showing four additional fixation point screws, one extending downward from the laterally movably adjustable modular piece, one extending inward from the medial modular piece, one extending inward from the right side of the central universal piece and one extending inward from the right side of the central universal piece, and also showing a rigid guide blade extending downward to contact the lateral shoulder of a femoral stem to be extracted.

FIG. 100 depicts a side elevational view of the 3-piece modular blade guide block illustrating the laterally movably adjustable modular piece and the medial modular piece attached to the central universal piece, also showing a medial piece retaining pin, and showing four additional fixation point screws, one extending downward from the laterally movably adjustable modular piece, one extending inward from the medial modular piece, one extending inward from the right side of the central universal piece and one extending inward from the right side of the central universal piece, and also showing a rigid lateral guide blade extending downward to contact the lateral shoulder of a femoral stem to be extracted as shown in FIG. 99.

FIG. 101 depicts a rear elevational view of the 3-piece modular blade guide block illustrating the laterally movably adjustable modular piece and the medial modular piece attached to the central universal piece, also showing a medial piece retaining pin, and showing four additional fixation point screws, one extending downward from the laterally movably adjustable modular piece, one extending inward from the medial modular piece, one extending inward from the right side of the central universal piece and one extending inward from the right side of the central universal piece, and also showing a rigid lateral guide blade extending downward to contact the lateral shoulder of a femoral stem to be extracted as shown in FIG. 99.

FIG. 102 depicts a top plan view of the 3-piece modular blade guide block illustrating the laterally movably adjustable modular piece and the medial modular piece attached to the central universal piece, also showing a medial piece retaining pin, and showing four additional fixation point screws, one extending downward from the laterally movably adjustable modular piece, one extending inward from the medial modular piece, one extending inward from the right side of the central universal piece and one extending inward from the right side of the central universal piece, and also showing a rigid lateral guide blade extending downward to contact the lateral shoulder of a femoral stem to be extracted as shown in FIG. 99.

FIG. 103 depicts a cross-sectional view of the 3-piece modular blade guide block illustrating the laterally movably adjustable modular piece and the medial modular piece attached to the central universal piece, also showing a medial piece retaining pin, and showing four additional fixation point screws, one extending downward from the laterally movably adjustable modular piece, one extending inward from the medial modular piece, one extending inward from the right side of the central universal piece and one extending inward from the right side of the central universal piece, and also showing a rigid lateral guide blade extending downward to contact the lateral shoulder of a femoral stem to be extracted as shown in FIG. 102.

FIG. 104A depicts a top, side elevational and perspective view of an alternate embodiment of a 2-piece modular blade guide block illustrating a laterally movably adjustable modular piece having a screw adjustment feature capable of very fine lateral extension adjustment.

FIG. 104B depicts a side elevational view of an alternate embodiment of a 2-piece modular blade guide block illustrating a laterally movably adjustable modular piece having a screw adjustment feature capable of very fine lateral extension adjustment as shown in FIG. 104A.

FIG. 104C depicts a bottom view of an alternate embodiment of a 2-piece modular blade guide block illustrating a laterally movably adjustable modular piece having a screw adjustment feature capable of very fine lateral extension adjustment as shown in FIG. 104B.

FIG. 105A depicts a rear view of an alternate embodiment of a 2-piece modular blade guide block illustrating a laterally movably adjustable modular piece having a screw adjustment feature capable of very fine lateral extension adjustment as shown in FIG. 104C.

FIG. 105B depicts a top plan view of an alternate embodiment of a 2-piece modular blade guide block illustrating a laterally movably adjustable modular piece having a screw adjustment feature capable of very fine lateral extension adjustment as shown in FIG. 105A.

FIG. 105C depicts a cross-sectional view of an alternate embodiment of a 2-piece modular blade guide block illustrating a laterally movably adjustable modular piece having a screw adjustment feature capable of very fine lateral extension adjustment as shown in FIG. 105B.

FIG. 106A depicts a bottom, side and perspective view of an alternate embodiment of a 2-piece modular blade guide block illustrating a laterally movably adjustable modular piece having a screw adjustment feature capable of very fine lateral extension adjustment as shown in FIG. 104.

FIG. 106B depicts a bottom, side and perspective view of an alternate embodiment of a 2-piece modular blade guide block illustrating a laterally movably adjustable modular piece having a screw adjustment feature capable of very fine lateral extension adjustment as shown in FIG. 106A.

FIG. 107A depicts a partially exploded top, side elevational and perspective view of an alternate embodiment of a 2-piece modular blade guide block illustrating a laterally movably adjustable modular piece having a screw adjustment feature capable of very fine lateral extension adjustment, here shown with the laterally movable modular piece extended fully outward.

FIG. 107B depicts a partially exploded rear view of an alternate embodiment of a 2-piece modular blade guide block illustrating a laterally movably adjustable modular piece having a screw adjustment feature capable of very fine lateral extension adjustment as shown in FIG. 107A.

FIG. 107C depicts a partially exploded rear view of an alternate embodiment of a 2-piece modular blade guide block illustrating a laterally movably adjustable modular piece having a screw adjustment feature capable of very fine lateral extension adjustment as shown in FIG. 107B.

FIG. 108 depicts an exploded side elevational view of an alternate embodiment 2-piece modular blade guide block illustrating a laterally movably adjustable modular piece having a screw adjustment feature capable of very fine lateral extension adjustment, here shown with the lateral piece fully extended outward, as shown in FIG. 107A.

FIG. 109 depicts an exploded top plan view of an alternate embodiment 2-piece modular blade guide block illustrating a laterally movably adjustable modular piece having a screw adjustment feature capable of very fine lateral extension adjustment, here shown with the lateral piece fully extended outward, as shown in FIG. 108.

FIG. 110 depicts an exploded cross-sectional view of an alternate embodiment 2-piece modular blade guide block illustrating a laterally movably adjustable modular piece having a screw adjustment feature capable of very fine lateral extension adjustment, here shown with the lateral piece fully extended outward, as shown in FIG. 109.

FIG. 111 depicts a bottom, side and perspective view of an alternate embodiment of a 2-piece modular blade guide block illustrating a laterally movably adjustable modular piece having a screw adjustment feature capable of very fine lateral extension adjustment as shown in FIG. 109.

FIG. 112 depicts a bottom, side and perspective view of an alternate embodiment of a 2-piece modular blade guide block illustrating a laterally movably adjustable modular piece having a screw adjustment feature capable of very fine lateral extension adjustment as shown in FIG. 111.

FIG. 113A depicts a top, side elevation and perspective view of an alternate embodiment 2-piece modular blade guide block illustrating a laterally movably adjustable modular piece having a screw adjustment feature capable of very fine lateral extension adjustment, here shown with the lateral piece partially disengaged and with the lateral piece fully contracted inward, as shown in FIG. 104.

FIG. 113B depicts a rear view of an alternate embodiment 2-piece modular blade guide block illustrating a laterally movably adjustable modular piece having a screw adjustment feature capable of very fine lateral extension adjustment, here shown with the lateral piece partially disengaged as shown in FIG. 113A.

FIG. 113C depicts a top, side elevation and perspective view of an alternate embodiment 2-piece modular blade guide block illustrating a laterally movably adjustable modular piece having a screw adjustment feature capable of very fine lateral extension adjustment, here shown with the lateral piece fully engaged and with the lateral piece fully contracted inward, as shown in FIG. 104.

FIG. 114A depicts a top, side elevation and perspective view of an alternate embodiment 2-piece modular blade guide block illustrating a laterally movably adjustable modular piece having a screw adjustment feature capable of very fine lateral extension adjustment, here shown with the lateral piece partially engaged and with the lateral piece fully extended outward.

FIG. 114B depicts a rear of an alternate embodiment 2-piece modular blade guide block illustrating a laterally movably adjustable modular piece having a screw adjustment feature capable of very fine lateral extension adjustment, here shown with the lateral piece partially engaged and with the lateral piece fully extended outward.

FIG. 114C depicts a top, side elevation and perspective view of an alternate embodiment 2-piece modular blade guide block illustrating a laterally movably adjustable modular piece having a screw adjustment feature capable of very fine lateral extension adjustment, here shown with the lateral piece partially engaged and with the lateral piece partially extended outward.

FIG. 115A depicts a top, side elevation and perspective view of an alternate embodiment 2-piece modular blade guide block illustrating a laterally movably adjustable modular piece having a screw adjustment feature capable of very fine lateral extension adjustment, here shown with the lateral piece fully engaged and with the lateral piece partially extended outward.

FIG. 115B depicts a top, side elevation and perspective view of the alternate embodiment 2-piece modular blade guide block illustrating a laterally movably adjustable modular piece having a screw adjustment feature capable of very fine lateral extension adjustment, here shown with the lateral piece fully engaged and with the lateral piece partially extended outward as shown in FIG. 115A.

FIG. 116A depicts a top plan view of the alternate embodiment 2-piece modular blade guide block illustrating a laterally movably adjustable modular piece having a screw adjustment feature capable of very fine lateral extension adjustment, here shown with the lateral piece fully engaged and with the lateral piece partially extended outward as shown in FIG. 115B.

FIG. 116B depicts a cross-sectional view of the alternate embodiment 2-piece modular blade guide block illustrating a laterally movably adjustable modular piece having a screw adjustment feature capable of very fine lateral extension adjustment, here shown with the lateral piece fully engaged and with the lateral piece partially extended outward as shown in FIG. 116A.

FIG. 117 depicts a front view of an improved backpressure plate having blade centering guide tabs thereon.

FIG. 118 depicts a rear, top and side elevational perspective view of the improved backpressure plate having blade centering guide tabs thereon.

FIG. 119 depicts a top plan view of the improved backpressure plate having blade centering guide tabs thereon.

FIG. 120 depicts a bottom view of the improved backpressure plate having blade centering guide tabs thereon.

FIG. 121 depicts a side elevational view of the improved backpressure plate having blade centering guide tabs thereon.

FIG. 122 depicts a top, side elevational and perspective view of a monolithic 1-piece modular blade guide block illustrating an improved backpressure plate having blade centering guide tabs adjustably mounted within the monolithic 1-piece modular blade guide block.

FIG. 123 depicts a side elevational view of the monolithic 1-piece modular blade guide block illustrating an improved backpressure plate having blade centering guide tabs adjustably mounted within the monolithic 1-piece modular blade guide block as shown in FIG. 122.

FIG. 124 depicts a bottom, side elevational and perspective view of the monolithic 1-piece modular blade guide block illustrating an improved backpressure plate having blade centering guide tabs adjustably mounted within the monolithic 1-piece modular blade guide block as shown in FIG. 122.

FIG. 125 depicts a top plan view of the monolithic 1-piece modular blade guide block illustrating an improved backpressure plate having blade centering guide tabs adjustably mounted within the monolithic 1-piece modular blade guide block as shown in FIG. 122.

FIG. 126 depicts a cross-sectional view of the monolithic 1-piece modular blade guide block illustrating an improved backpressure plate having blade centering guide tabs adjustably mounted within the monolithic 1-piece modular blade guide block as shown in FIG. 125.

FIG. 127 depicts a bottom, rear, right side elevational and perspective view of the monolithic 1-piece modular blade guide block illustrating an improved backpressure plate having blade centering guide tabs adjustably mounted within the monolithic 1-piece modular blade guide block as shown in FIG. 126.

FIG. 128 depicts a bottom, rear, left side elevational and perspective view of the 1-piece modular blade guide block illustrating an improved backpressure plate having blade centering guide tabs adjustably mounted within the 1-piece modular blade guide block as shown in FIG. 127.

FIG. 129 depicts a top, side elevational and perspective view of a 2-piece modular blade guide block having a laterally adjustable modular lateral piece illustrating an improved backpressure plate having blade centering guide tabs adjustably mounted within the 2-piece modular blade guide block.

FIG. 130 depicts a side elevational view of the 2-piece modular blade guide block having a laterally adjustable modular lateral piece illustrating an improved backpressure plate having blade centering guide tabs adjustably mounted within the 2-piece modular blade guide block as shown in FIG. 129.

FIG. 131 depicts a bottom, right side elevational and perspective view of the 2-piece modular blade guide block having a laterally adjustable modular lateral piece illustrating an improved backpressure plate having blade centering guide tabs adjustably mounted within the 2-piece modular blade guide block as shown in FIG. 130.

FIG. 132 depicts a top plan view of the 2-piece modular blade guide block having a laterally adjustable modular lateral piece illustrating an improved backpressure plate having blade centering guide tabs adjustably mounted within the 2-piece modular blade guide block as shown in FIG. 130.

FIG. 133 depicts a cross-sectional view of the 2-piece modular blade guide block having a laterally adjustable modular lateral piece illustrating an improved backpressure plate having blade centering guide tabs adjustably mounted within the 2-piece modular blade guide block as shown in FIG. 132.

FIG. 134 depicts a bottom, rear, right side elevational and perspective view of the 2-piece modular blade guide block having a laterally adjustable modular lateral piece illustrating an improved backpressure plate having blade centering guide tabs adjustably mounted within the 2-piece modular blade guide block as shown in FIG. 133.

FIG. 135 depicts a bottom, rear, left side elevational and perspective view of the 2-piece modular blade guide block having a laterally adjustable modular lateral piece illustrating an improved backpressure plate having blade centering guide tabs adjustably mounted within the 2-piece modular blade guide block as shown in FIG. 133.

FIG. 136 depicts a top, side elevational and perspective view of a 3-piece modular blade guide block having a laterally adjustable modular lateral piece and a medial modular piece illustrating an improved backpressure plate having blade centering guide tabs adjustably mounted within the 3-piece modular blade guide block.

FIG. 137 depicts a top, side elevational and perspective view of the 3-piece modular blade guide block having a laterally adjustable modular lateral piece and a medial modular piece illustrating an improved backpressure plate having blade centering guide tabs adjustably mounted within the 3-piece modular blade guide block as shown in FIG. 136.

FIG. 138 depicts a bottom, right side elevational and perspective view of the 3-piece modular blade guide block having a laterally adjustable modular lateral piece and a medial modular piece illustrating an improved backpressure plate having blade centering guide tabs adjustably mounted within the 3-piece modular blade guide block as shown in FIG. 136.

FIG. 139 depicts a top plan view of the 3-piece modular blade guide block having a laterally adjustable modular lateral piece and a medial modular piece illustrating an improved backpressure plate having blade centering guide tabs adjustably mounted within the 3-piece modular blade guide block as shown in FIG. 136.

FIG. 140 depicts a bottom, right side elevational and perspective view of the 3-piece modular blade guide block having a laterally adjustable modular lateral piece and a medial modular piece illustrating an improved backpressure plate having blade centering guide tabs adjustably mounted within the 3-piece modular blade guide block as shown in FIG. 136.

FIG. 141 depicts a cross-sectional view of the 3-piece modular blade guide block having a laterally adjustable modular lateral piece and a medial modular piece illustrating an improved backpressure plate having blade centering guide tabs adjustably mounted within the 3-piece modular blade guide block as shown in FIG. 139.

FIG. 142 depicts a bottom, left side elevational view of the 3-piece modular blade guide block having a laterally adjustable modular lateral piece and a medial modular piece illustrating an improved backpressure plate having blade centering guide tabs adjustably mounted within the 3-piece modular blade guide block as shown in FIG. 140.

FIG. 143A depicts a top plan view of a small width sized curved rigid lateral guide blade having a blade stop thereon.

FIG. 143B depicts a front view of the small width sized curved rigid lateral guide blade having a blade stop shown in FIG. 143A.

FIG. 144A depicts a top plan view of a medium width sized curved rigid lateral guide blade having a blade stop.

FIG. 144B depicts a front view of the medium width sized curved rigid lateral guide blade having a blade stop shown in FIG. 144A.

FIG. 145A depicts a top plan view of a large width sized curved rigid lateral guide blade having a blade stop.

FIG. 145B depicts a front view of the large width sized curved rigid lateral guide blade having a blade stop shown in FIG. 145A.

FIG. 146 depicts a rear view of the large width sized curved rigid lateral guide blade having a blade stop shown in FIG. 145B, illustrating the beveled edge.

FIG. 147 depicts a rear view of the medium width sized curved rigid lateral guide blade having a blade stop shown in FIG. 144B, illustrating the beveled edge.

FIG. 148 depicts a rear view of the small width sized curved rigid lateral guide blade having a blade stop shown in FIG. 143B, illustrating the beveled edge.

FIG. 149A depicts a top plan view of a small width sized curved flexible lateral cutting blade having a cutting edge and an elongated blade shaft.

FIG. 149B depicts a front view of the small width sized curved flexible lateral cutting blade having a cutting edge and an elongated blade shaft as shown in FIG. 149A.

FIG. 150A depicts a top plan view of a medium width sized curved flexible lateral cutting blade having a cutting edge and an elongated blade shaft.

FIG. 150B depicts a front view of the medium width sized curved flexible lateral cutting blade having a cutting edge and an elongated blade shaft as shown in FIG. 150A.

FIG. 151A depicts a top plan view of a large width sized curved flexible lateral cutting blade having a cutting edge and an elongated blade shaft.

FIG. 151B depicts a front view of the large width sized curved flexible lateral cutting blade having a cutting edge and an elongated blade shaft as shown in FIG. 151A.

FIG. 152 depicts a rear view of the large width sized curved flexible lateral cutting blade having a cutting edge and an elongated blade shaft as shown in FIG. 151B, illustrating the beveled cutting edge.

FIG. 153 depicts a rear view of the medium width sized curved flexible lateral cutting blade having a cutting edge and an elongated blade shaft as shown in FIG. 150B, illustrating the beveled cutting edge.

FIG. 154 depicts a rear view of the small width sized curved flexible lateral cutting blade having a cutting edge and an elongated blade shaft as shown in FIG. 149B, illustrating the beveled cutting edge.

FIG. 155A depicts a top plan view of a small width sized curved flexible medial calcar cutting blade having a cutting edge and a short blade shaft.

FIG. 155B depicts a front view of the small width sized curved flexible medial calcar cutting blade having a cutting edge and a short blade shaft as shown in FIG. 155A.

FIG. 156A depicts a top plan view of a medium width sized curved flexible medial calcar cutting blade having a cutting edge and a short blade shaft.

FIG. 156B depicts a front view of the medium width sized curved flexible medial calcar cutting blade having a cutting edge and a short blade shaft as shown in FIG. 156A.

FIG. 157A depicts a top plan view of a large width sized curved flexible medial calcar cutting blade having a cutting edge and a short blade shaft.

FIG. 157B depicts a front view of the large width sized curved flexible medial calcar cutting blade having a cutting edge and a short blade shaft as shown in FIG. 157A.

FIG. 158 depicts a rear view of the large width sized curved flexible medial calcar cutting blade having a cutting edge and a short blade shaft as shown in FIG. 157B, illustrating the beveled cutting edge.

FIG. 159 depicts a rear view of the medium width sized curved flexible medial calcar cutting blade having a cutting edge and a short blade shaft as shown in FIG. 156B, illustrating the beveled cutting edge.

FIG. 160 depicts a rear view of the small width sized curved flexible medial calcar cutting blade having a cutting edge and a short blade shaft as shown in FIG. 155B, illustrating the beveled cutting edge.

FIG. 161 depicts a front view of a large sized anterior/posterior (A/P) osteotome, which blade is right-left (R-L) oriented and illustrates the beveled cutting edge.

FIG. 162 depicts a front view of a medium sized anterior/posterior (A/P) osteotome, which blade is right-left (R-L) oriented and illustrates the beveled cutting edge.

FIG. 163 depicts a front view of a small sized anterior/posterior (A/P) osteotome, which blade is right-left (R-L) oriented and illustrates the beveled cutting edge.

FIG. 164 depicts a rear view of a small sized anterior/posterior (A/P) osteotome, which blade is right-left (R-L) oriented as shown in FIG. 163.

FIG. 165 depicts a rear view of a medium sized anterior/posterior (A/P) osteotome, which blade is right-left (R-L) oriented as shown in FIG. 162.

FIG. 166 depicts a rear view of a large sized anterior/posterior (A/P) osteotome, which blade is right-left (R-L) oriented as shown in FIG. 161.

FIG. 167 depicts a front view of a large sized anterior/posterior (A/P) osteotome, which blade is left-right (L-R) oriented and illustrates the beveled cutting edge.

FIG. 168 depicts a front view of a medium sized anterior/posterior (A/P) osteotome, which blade is left-right (L-R) oriented and illustrates the beveled cutting edge.

FIG. 169 depicts a front view of a small sized anterior/posterior (A/P) osteotome, which blade is left-right (L-R) oriented and illustrates the beveled cutting edge.

FIG. 170 depicts a rear view of a small sized anterior/posterior (A/P) osteotome, which blade is left-right (L-R) oriented as shown in FIG. 169.

FIG. 171 depicts a rear view of a medium sized anterior/posterior (A/P) osteotome, which blade is left-right (L-R) oriented as shown in FIG. 168.

FIG. 172 depicts a rear view of a large sized anterior/posterior (A/P) osteotome, which blade is left-right (L-R) oriented as shown in FIG. 167.

FIG. 173A depicts a top plan view of a small sized compound curved rigid medial calcar blade, illustrating that the cutting blade is curved in two planes.

FIG. 173B depicts a rear view of a small sized compound curved rigid medial calcar blade, illustrating that the cutting blade is curved in two planes as shown in FIG. 173A.

FIG. 174A depicts a top plan view of a medium sized compound curved rigid medial calcar blade, illustrating that the cutting blade is curved in two planes.

FIG. 174B depicts a rear view of a medium sized compound curved rigid medial calcar blade, illustrating that the cutting blade is curved in two planes as shown in FIG. 174A.

FIG. 175A depicts a top plan view of a large width sized compound curved rigid medial calcar blade, illustrating that the cutting blade is curved in two planes.

FIG. 175B depicts a rear view of a large width sized compound curved rigid medial calcar blade, illustrating that the cutting blade is curved in two planes as shown in FIG. 175A.

FIG. 176 depicts a side elevational view of the large width sized compound curved rigid medial calcar blade, illustrating that the cutting blade is curved in two planes, the latitudinal plane and the longitudinal plane, as shown in FIG. 175A and 175B.

FIG. 177 depicts a top, side and perspective view of the small width sized compound curved rigid medial calcar blade, illustrating that the cutting blade is curved in two planes as shown in FIG. 173B.

FIG. 178 depicts a top, side and perspective view of the medium width sized compound curved rigid medial calcar blade, illustrating that the cutting blade is curved in two planes as shown in FIG. 174B.

FIG. 179 depicts a top, side and perspective view of the large width sized compound curved rigid medial calcar blade, illustrating that the cutting blade is curved in two planes as shown in FIG. 175B.

FIG. 180 depicts a front view of a large width sized compound curved rigid medial calcar blade, illustrating that the cutting blade is curved in two planes as shown in FIG. 175B, here showing the beveled cutting edge.

FIG. 181 depicts a front view of a medium width sized compound curved rigid medial calcar blade, illustrating that the cutting blade is curved in two planes as shown in FIG. 174B, here showing the beveled cutting edge.

FIG. 182 depicts a front view of a small width sized compound curved rigid medial calcar blade, illustrating that the cutting blade is curved in two planes as shown in FIG. 173B, here showing the beveled cutting edge.

FIG. 183 depicts a top, side elevational and perspective view of an embodiment of the joint revision surgery apparatus illustrating a rigid osteotome blade/surgical chisel attached to a powered impaction device that is part of/attached to a robotic arm.

FIG. 184 depicts a top, side elevational and perspective view of an embodiment of the joint revision surgery apparatus illustrating a flexible osteotome blade/surgical chisel attached to a powered impaction device that is part of/attached to a robotic arm.

FIG. 185, 186 and 187 depict a direct head on as well as both oblique views as an example of the navigation, computer navigation or robotic navigation array or tracker that may be attached to the universal central piece of the guide block and or the femoral stem.

FIG. 188, 189 and 190 depicts a direct head on as well as both oblique views of an array as used with navigation, computer navigation or robotic navigation The array or tracker may be attached to the universal central piece of the guide block and or the femoral stem or both.

FIG. 191 and 192 depicts a perspective and side elevational view of an embodiment of the joint revision surgery apparatus illustrating a rigid osteotome blade/surgical chisel attached to a powered impaction device that is part of/attached to a robotic arm.

FIG. 193 depicts a top elevational view of an embodiment of the joint revision surgery apparatus illustrating a rigid osteotome blade/surgical chisel attached to a powered impaction device that is part of/attached to a robotic arm.

FIG. 194 depicts a side elevational view of an embodiment of the joint revision surgery apparatus illustrating a rigid or flexible osteotome blade/surgical chisel attached to a powered impaction device that is part of/attached to a robotic arm.

FIG. 195, 196 and 197 depicts a perspective, side and top view of an embodiment of the joint revision surgery apparatus illustrating the modular adjustable guide block attached to the femoral stem with an array or tracker attached to the modular adjustable guide block.

FIG. 198 depicts a side view of an embodiment of the joint revision surgery apparatus illustrating the modular adjustable guide block attached to the femoral stem with an array or tracker attached to the modular adjustable guide block.

FIG. 199, 200 and 201 depicts a perspective, side and top view of an embodiment of the joint revision surgery apparatus illustrating the modular adjustable guide block attached to the femoral stem with an array or tracker attached to the modular adjustable guide block.

FIG. 202 depicts a side view of an embodiment of the joint revision surgery apparatus illustrating the modular adjustable guide block attached to the femoral stem with an array or tracker attached to the modular adjustable guide block.

FIG. 203, 204 and 205 depicts a perspective, side and top view of an embodiment of the joint revision surgery apparatus illustrating the modular adjustable guide block attached to the femoral stem with an array or tracker attached to the modular adjustable guide block.

FIG. 206 depicts a side view of an embodiment of the joint revision surgery apparatus illustrating the modular adjustable guide block attached to the femoral stem with an array or tracker attached to the modular adjustable guide block.

FIG. 207 depicts a side view of an embodiment of the joint revision surgery apparatus illustrating a rigid osteotome blade/surgical chisel attached to a hand held powered impaction device that is guided by computer navigation.

FIG. 208 and 209 depicts a perspective and side elevational view of an embodiment of the joint revision surgery apparatus illustrating a rigid osteotome blade/surgical chisel attached to a hand held powered impaction device that is guided by computer navigation.

FIG. 210 depicts a perspective view of an embodiment of the joint revision surgery apparatus illustrating the modular adjustable guide block attached to the femoral stem with an array or tracker attached to the modular adjustable guide block.

FIG. 210 depicts a perspective view of an embodiment of the joint revision surgery apparatus illustrating the modular adjustable guide block attached to the femoral stem with an array or tracker attached to the modular adjustable guide block.

FIG. 211 depicts a side view of an embodiment of the joint revision surgery apparatus illustrating the modular adjustable guide block attached to the femoral stem with an array or tracker attached to the modular adjustable guide block.

FIG. 212 and 213 depicts a side view of an embodiment of the joint revision surgery apparatus illustrating the modular adjustable guide block attached to the femoral stem with an array or tracker attached to the modular adjustable guide block.

FIG. 214 depicts a side view of an embodiment of the joint revision surgery apparatus illustrating the modular adjustable guide block attached to the femoral stem with an array or tracker attached to the modular adjustable guide block.

FIG. 215 depicts a side elevational view of an embodiment of the joint revision surgery apparatus illustrating the modular adjustable guide block attached to the femoral stem with an array or tracker attached to the modular adjustable guide block.

### DETAILED DESCRIPTION OF THE PREFERRED EMBODIMENTS

As required, the detailed embodiments of the present Modular Adjustable Blade Guide Block for Joint Revision Surgery 10A, 100, 200, 300, 350, 400, 450, 500 and 600 are disclosed herein, however, it is to be understood that the disclosed embodiments are merely exemplary of the design that may be embodied in various forms. Therefore, specific functional and structural details disclosed herein are not to be interpreted as limiting, but merely as basic for the claims and as a representative basis for teaching one skilled in the art to variously employ the present design in virtually any appropriately detailed structure.

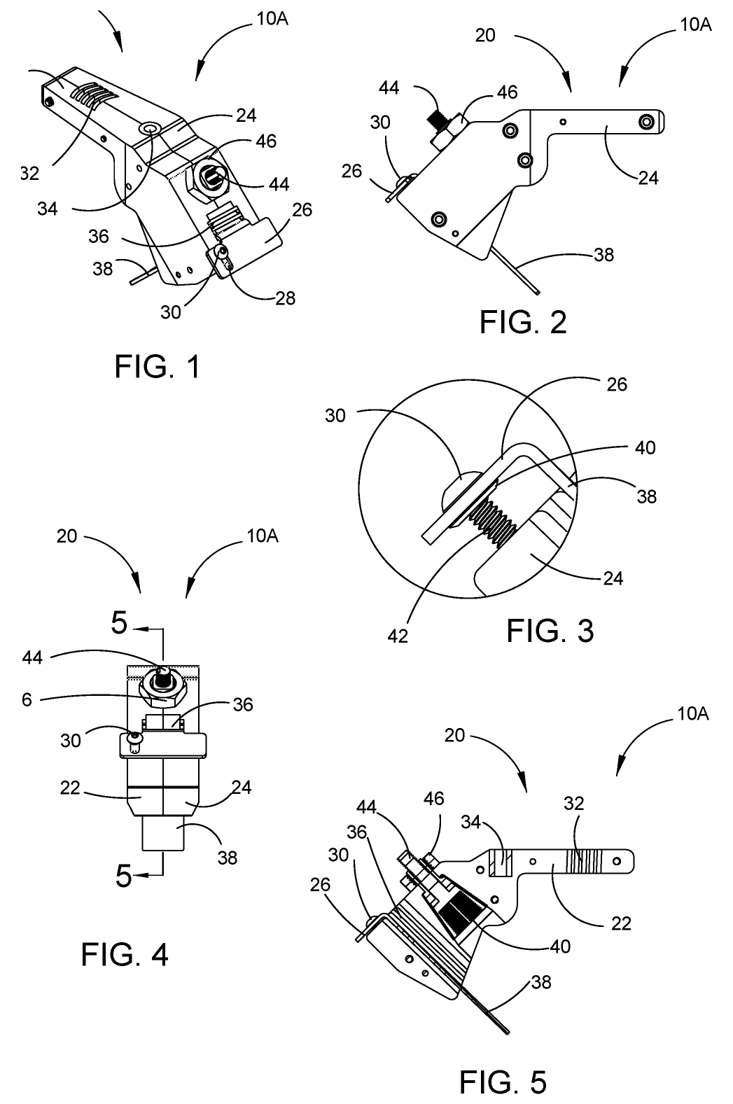

As previously described in US patent application serial No. 18/083,215 which application is incorporated in its entirety herein, FIG. 1 depicts a top, side elevational and perspective view of an embodiment of the joint revision surgery apparatus 10A illustrating an assembled osteotome blade/surgical chisel guide block 20 (hereinafter referred to as an osteotome blade guide block 20) having an adjustable L-shaped osteotome blade guide plate 38 fully extended downwardly and positioned in the frontmost blade guide slot within the plurality of blade guide slots 36. As previously described in US patent application serial No. 17/899,521 and US patent application serial No. 18/075,574 both of which US patent applications are hereby incorporated in their entirety herein, the guide block is made from two half sections 22 and 24 held together using Allen screws and alignment pins (see the exploded view in FIG. 23 below). The L-shaped osteotome blade guide plate has an upper portion 26 and a lower portion 38. The guide plate upper portion includes an adjustable guide plate retaining screw slot 28 in which the adjustable guide plate retaining screw 30 is threaded into a threaded orifice (not shown here) within the guide block assembly 20. Integral to the guide block assembly 20 is a plurality of forward blade guide slots 36 for accepting the adjustable guide plate 38 and the osteotome blades (not shown, see below), and a central cavity 40. The central cavity 40 is configured to accept a trunnion securing member 44 (best seen in FIG. 5, and the trunnion securing member is secured using securing nut 46. Additionally, the assembled guide block 20 defines a plurality of rearward blade guide slots. In this way, the guide block can be adjusted for any size femoral stem implant encountered for removal during revision surgery. The various adjustments are described in greater detail below. This view clearly illustrates the position of the guide plate adjustment screw 30 and the adjustment screw slot 28 within the upper section of the L-shaped guide plate 26. The adjustment screw 30 can be adjusted upwardly and downwardly by threading in or out of the assembled guide block 20, and it can be placed in any of the plurality of blade guide slots 36 within the guide block 20. To move the lower section guide plate 38 to another guide slot in the plurality of guide slots 36, one must unthread and remove the adjustment screw 30 and then replace it by sliding it to another position so the guide plate lower section 38 can be placed in a different guide slot 36

As previously described in US patent application serial No. 18/083,215 which application is incorporated in its entirety herein, FIG. 2 depicts a side elevational view of an embodiment of the joint revision surgery apparatus 10A illustrating an assembled osteotome blade guide block 20 having an adjustable L-shaped osteotome blade guide plate 38 fully extended downwardly. This view better shows the lower guide plate 38 extending downward below the guide block assembly 20.

As previously described in US patent application serial No. 18/083,215 which application is incorporated in its entirety herein, FIG. 3 depicts an enlarged side elevational view of the upper section guide plate 26 and guide plate adjustment screw 30 having an integral retaining washer 40 welded to the underside of the upper section guide plate 26. The guide plate retaining screw 30 here is partially threaded out exposing the threaded section 42. This retaining washer makes it possible for the guide plate upper section 26 to rise and be secured in an upward position when the adjustment plate retaining screw 30 is threaded upwardly or to be lowered when the retaining screw 30 is threaded in downwardly. It is anticipated that a free spinning retaining washer (see retaining washer 41 as shown in FIG. 14) can be used in place of this welded on retaining washer 40 to accomplish the same task of securing the adjustment plate 26 and 38.

As previously described in US patent application serial No. 18/083,215 which application is incorporated in its entirety herein, FIG. 4 depicts a front view of an alternate embodiment of the joint revision surgery apparatus 10A illustrating an assembled osteotome blade guide block 20 having an adjustable L-shaped osteotome blade guide plate 38 fully extended downwardly. The guide block is made from two half sections 22 and 24. This view better shows the lower guide plate 38 extending downward into the plurality of forward blade guide slots 36 within the guide block assembly 20.

As previously described in US patent application serial No. 18/083,215 which application is incorporated in its entirety herein, FIG. 5 depicts a cross-sectional view of an embodiment of the joint revision surgery apparatus 10A illustrating an assembled osteotome blade/surgical chisel guide block 20 having an adjustable L- shaped osteotome blade guide plate upper section 26 and lower section 38 fully extended downwardly of FIG. 4. This view illustrates the position of the central cavity 40 and the trunnion securing member 44 within the central cavity 40 secured by the securing nut 46. It also clearly shows the plurality of blade guide slots 36 and the lower guide plate 38 extending down into the foremost of the plurality of guide slots 36. The lower guide plate 38 can be adjusted by being positioned in any one of the plurality of guide slots 36 by removing the retaining screw 30 and shifting the lower guide plate 38 to a different slot within the plurality of guide slots 36.

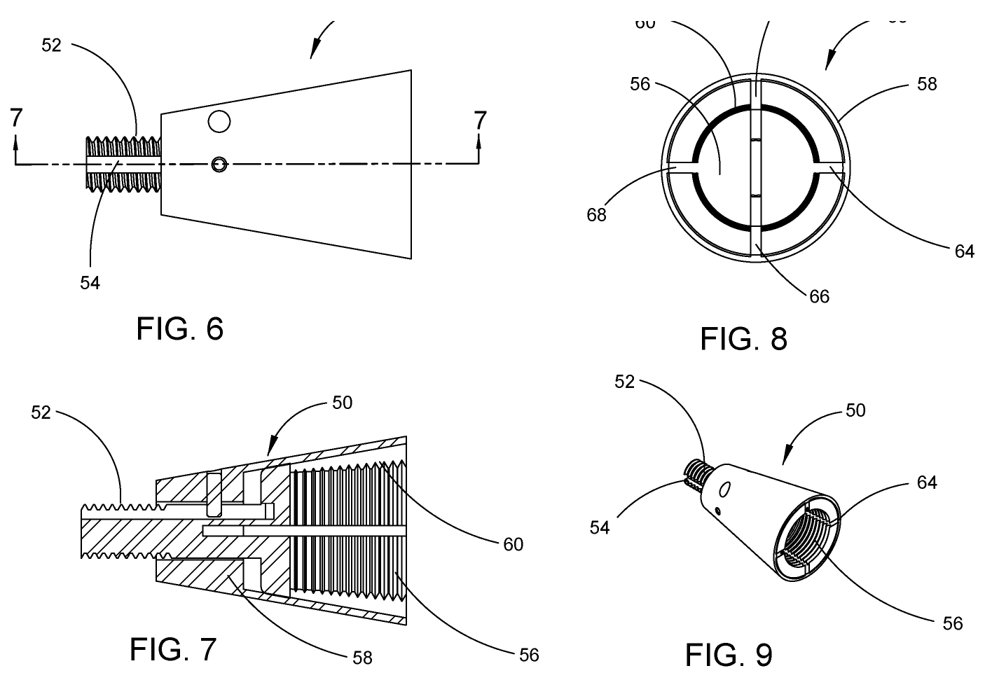

As previously described in US patent application serial No. 18/083,215 which application is incorporated in its entirety herein, FIG. 6 depicts a side elevational view of a stem trunnion securing piece 50 having a threaded portion 52 and a groove 54 running the length of the threaded portion 52. The stem trunnion securing piece 50 is conical in shape and tapers from a large diameter to a small diameter towards the threaded portion 52. The groove 54 is used to orient mounting the stem trunnion securing piece 50 within the central cavity 40 of the assembly 20 (not shown, see FIG. 5) and when mounted therein, to keep the stem trunnion securing piece 50 from rotating during a revision surgery operation.

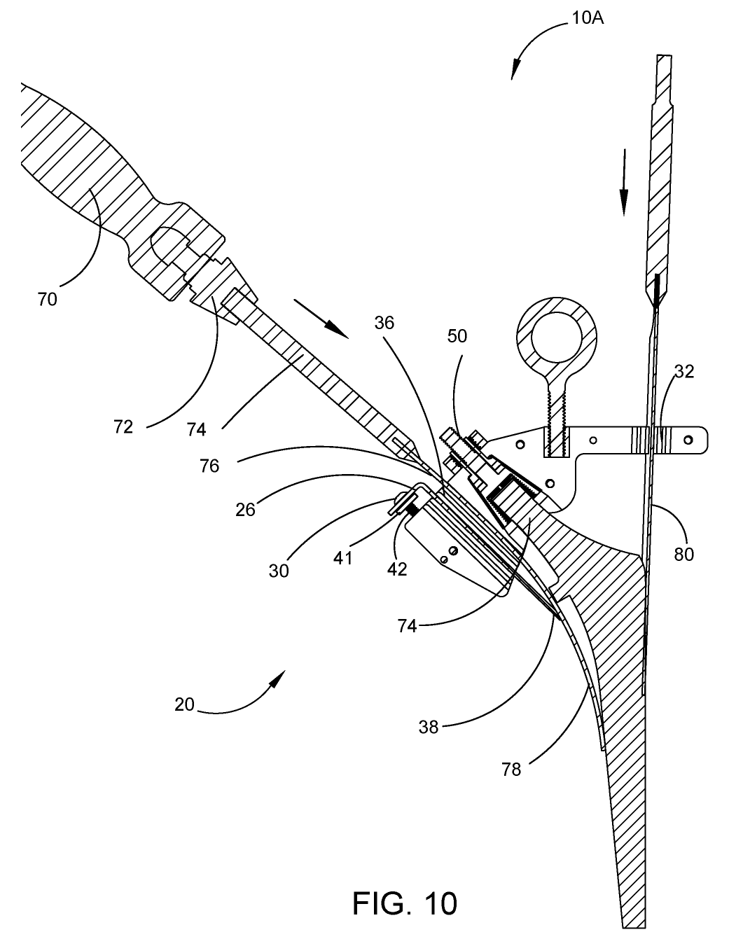

As previously described in US patent application serial No. 18/083,215 which application is incorporated in its entirety herein, FIG. 7 depicts a cross-sectional view of the stem trunnion securing piece 50, as shown in FIG. 6. The stem trunnion securing piece 50 has a partially hollow body 58, having a ribbed inner portion 56 running roughly halfway from the large diameter to the small diameter proximal to the threaded portion 52. Within the outer body 58 of the stem trunnion securing piece 50 is a ribbed inner portion 56 having a plurality of circular ribs 60 thereon. The ribbed inner portion 56 is slidably affixed to the outer body. The ribbed inner portion 60 within the hollow section 56 also has exterior slots (not shown). When the nut 46 (not shown, see FIG. 5) is placed on the threaded portion 52 and tightened, this moves the inner ribbed portion 56 upwardly the outer body 58 of the stem trunnion securing piece 50 and tightens the stem trunnion securing piece 50 around the stem trunnion 74 (see FIG. 10), by decreasing the overall diameter of the inner ribbed portion 56. See further description of this operation below.

As previously described in US patent application serial No. 18/083,215 which application is incorporated in its entirety herein, FIG. 8 depicts a bottom view of a stem trunnion securing piece 50 illustrating the outer body 58 and the inner ribbed portion 60 within the hollow section 56. Four slots 62, 64, 66 and 68 are located at 90 degrees from each other around the ribbed inner portion 56. In operation, the stem trunnion securing piece 50 is placed on the stem trunnion (see FIG. 10), and the plurality of circular ribs 60 make contact with the stem trunnion (see FIG. 10). When the nut 46 is placed on the threaded portion 52 and tightened, this moves the inner ribbed portion upwardly in the outer body 58 and tightens the stem trunnion securing piece 50 around the stem trunnion (see FIG. 10), by decreasing the overall diameter of the ribbed inner portion 56 around the stem trunnion. The four slots 62, 64, 66 and 68 pinch together and the slot gap distance decreases as the nut 46 is tightened and the inner ribbed portion 56 moves relative to the outer body 58. This effectively tightly and securely clamps the stem trunnion securing piece 50 to the stem trunnion. Once secured, the stem trunnion 50 cannot be released from the stem trunnion securing piece 50 until the nut 46 is loosened and the diameter of the ribbed inner portion 56 is increased to its original size. The nut 46 may be tightened and loosened using a standard wrench, such as a 10 mm box wrench.

As previously described in US patent application serial No. 18/083,215 which application is incorporated in its entirety herein, FIG. 9 depicts a side perspective view of a stem trunnion securing piece 50 illustrating the threaded portion 52 having a slot 54 therein, and the inner ribbed portion 56 having four slots therein, here with one slot 64 shown labeled by a reference character. It is anticipated that varying sizes of the stem trunnion securing piece 50 will be made available to accommodate the varying sizes of the stem trunnions to be extracted. Different sizes of the stem trunnion securing piece 50 will be readily mounted by placing the stem trunnion securing piece 50 into the central cavity 40 then securing it in place using the nut 46. In this way, the stem trunnion securing piece 50 will also be replaceable and reusable as when the nut 46 is loosened the stem trunnion 74 is released after extraction of the stem.

As previously described in US patent application serial No. 18/083,215 which application is incorporated in its entirety herein, FIG. 10 depicts a cross-sectional view of the alternate embodiment of the joint revision surgery apparatus 10A illustrating an assembled osteotome blade guide block 20 having an adjustable L-shaped osteotome blade guide plate upper portion 26 and lower portion 38, in use during revision surgery showing the surgical osteotome blades 76 and 80 inserted into the forward 36 and rearward 32 osteotome blade guide slots within the assembled guide block 20, for removal of a collared implanted femoral stem 82. The collared femoral stem trunnion section 74 has been secured to the trunnion securing member 50. This FIG. 10 again, represents Step 6 of the procedure for revision surgery to remove a collared femoral stem implant 82, namely, Step 6: continue downward pressure and motion of the medial calcar cutting blade 78 until it advances past the point of initial contact with the collared femoral stem implant 82 to be removed and begins cutting the collared femoral stem implant 76 away from the femur bone. In this FIG. 10 note that the L-shaped guide plate upper portion 26 and lower portion 38 are positioned within the guide slot closest to the collared femoral stem implant 82. Note that the guide plate lower portion section 38 is secured at a more raised position relative to its lowest point possible, a bit farther away from the collared femoral stem implant 82 to be removed during revision surgery. This allows the guide plate 38 to guide the cutting blade 76 down passed the collar on the collared stem 82. These two aforementioned two-way distance adjustments allow the guide block 20 to be used with varying sized collared and non-collared femoral stem implants. By inserting the cutting blade 80 into the appropriate slot for the implant size, lateral cutting blade 80 can cut down the lateral side of the implant 74 and free the implant from the bone. However, this technique only works for a limited number of sizes of femoral stem implants.

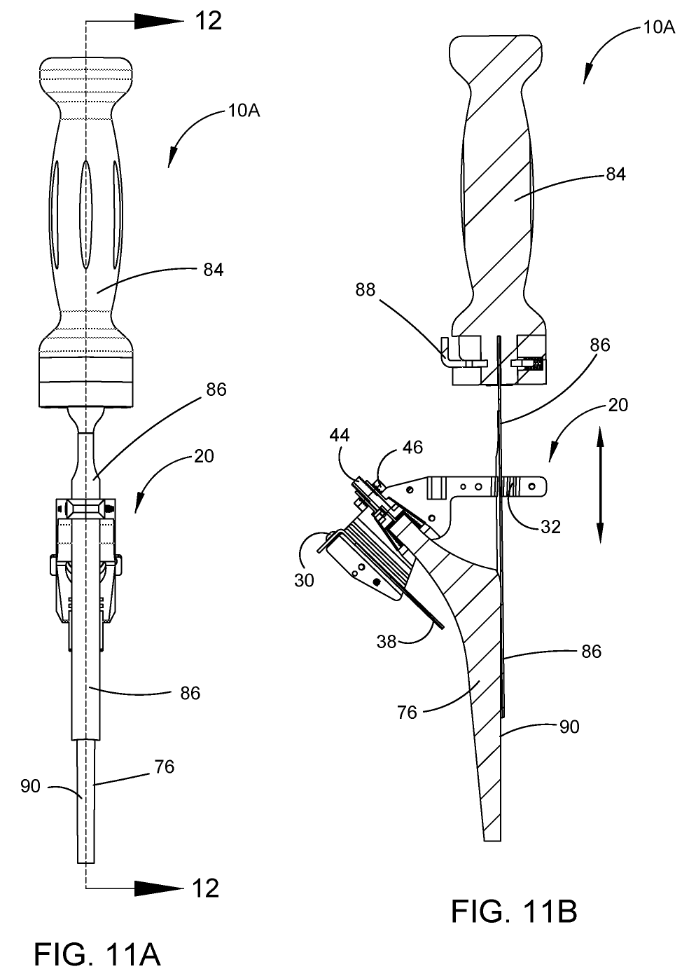

As previously described in US patent application serial No. 18/083,215 which application is incorporated in its entirety herein, FIG. 11A depicts a rear view of a surgical blade locking osteotome handle assembly 10A having a surgical osteotome blade 86 inserted therein, here shown on a handle assembly 84 in place attached to a surgical osteotome blade guide block assembly 20. In this view, the surgical osteotome blade guide block assembly 20 is attached to an implant 76 and is being used to guide the osteotome blade 86 down through the guide block rearward guide slots in order to cut the shoulder section of implant 76 away from the femur bone (not shown).

As previously described in US patent application serial No. 18/083,215 which application is incorporated in its entirety herein, FIG. 11B depicts a cross-sectional side elevational view of a surgical blade locking osteotome handle assembly 10A having a surgical osteotome blade 86 inserted therein, here shown on a handle assembly 84 in place attached to a surgical osteotome blade guide block assembly 20, as shown in FIG. 11. Recall that surgical osteotome blade guide block assembly 20 comprises a lower guide plate 38, a guide plate adjustment screw 30 and a trunnion securing member 44 securely held to the surgical osteotome blade guide block assembly 20 by nut 46. The plurality of rearward guide slots 32 are also clearly seen here. Based on the size of the implant 76, the surgeon chooses a single slot within the plurality of rearward guide slots 32 to extend the blade 86 downwardly, which blade 86 has been secured to the handle 84 with locking lever 88 in the outward or locked position. In this way, the lateral shoulder of the implant 90 is cut away from the bone (not shown) to be released in a revision surgery procedure. The combination of using the osteotome blade 86 secured within the handle assembly 84 and extending the blade 84 downwardly through the guide slot to cut the makes for a very efficient and time saving revision surgery resulting in significantly improved patient outcomes. Again, however, this technique only works for a limited number of sizes of femoral stem implants.

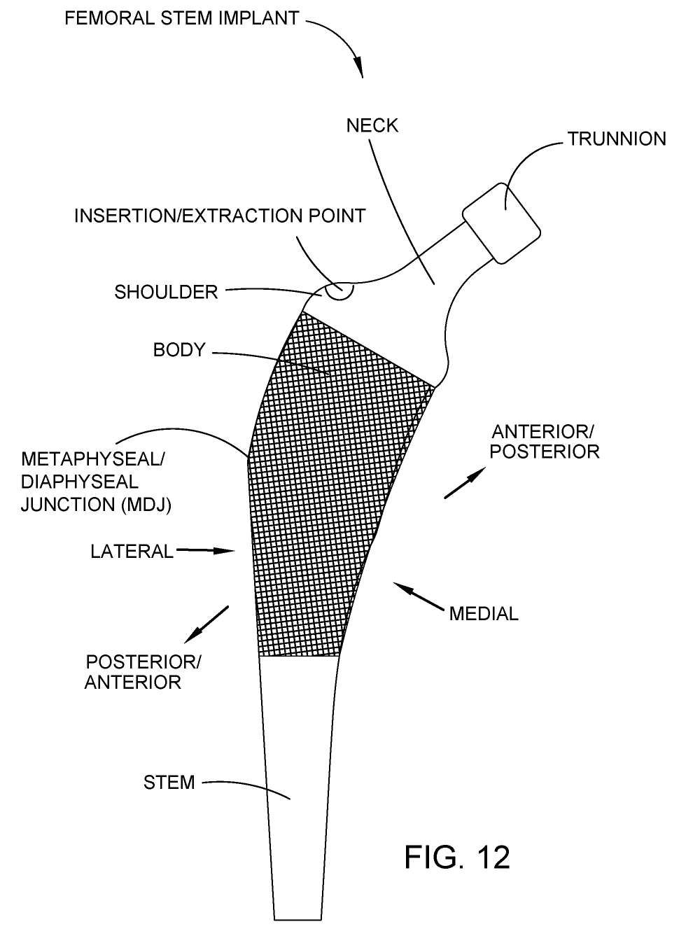

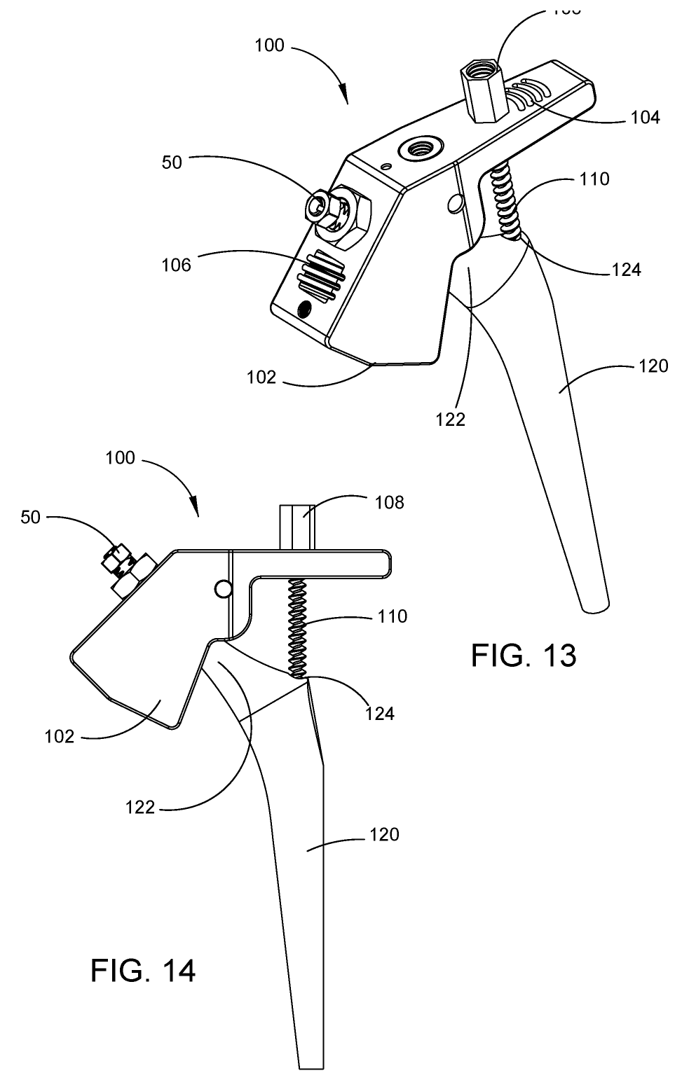

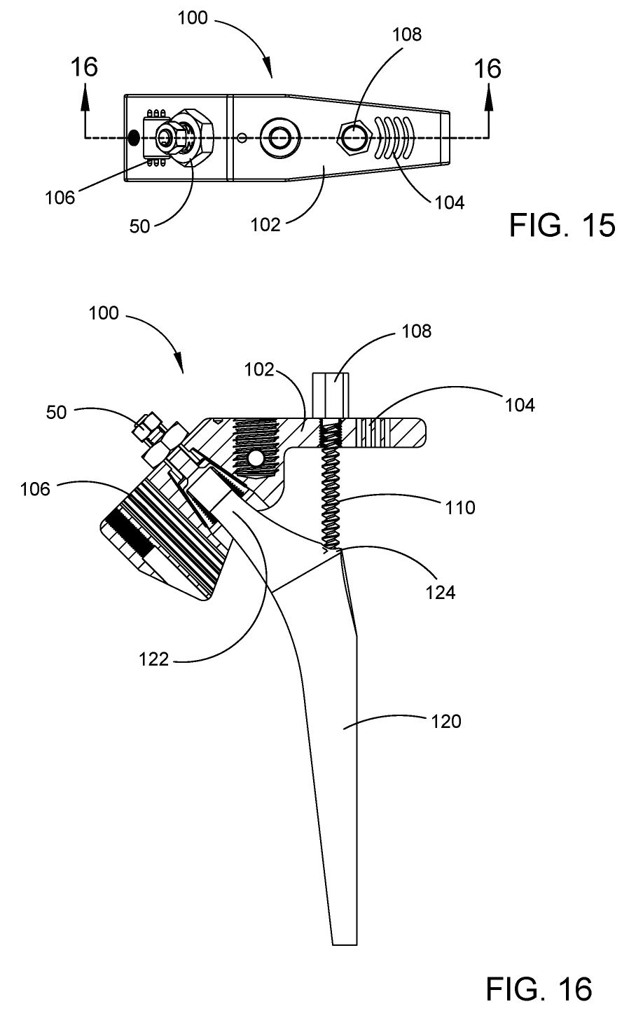

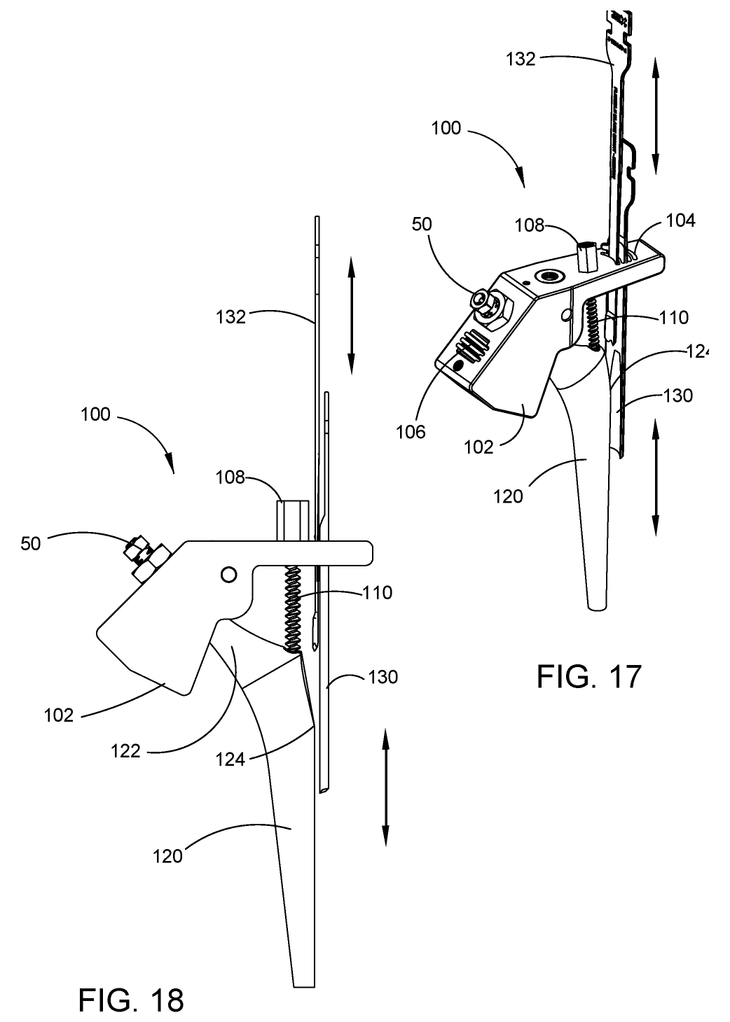

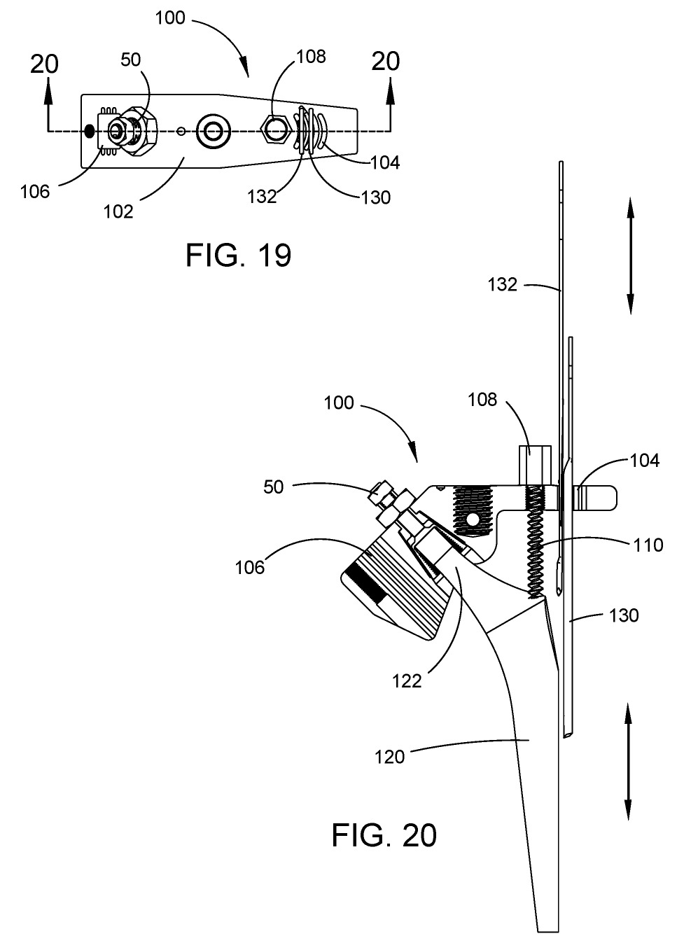

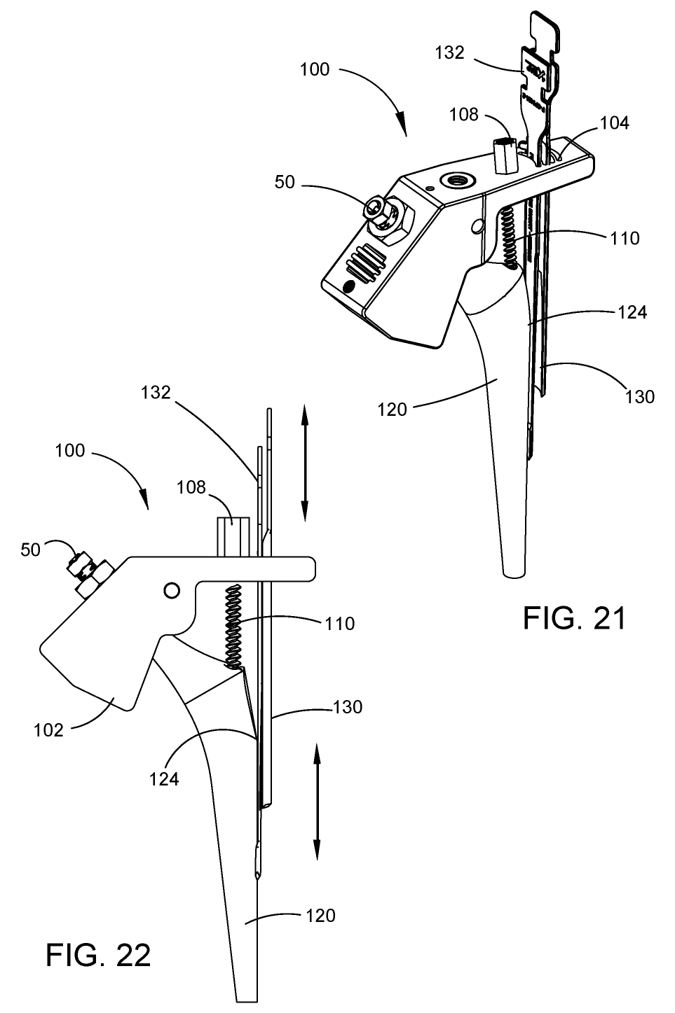

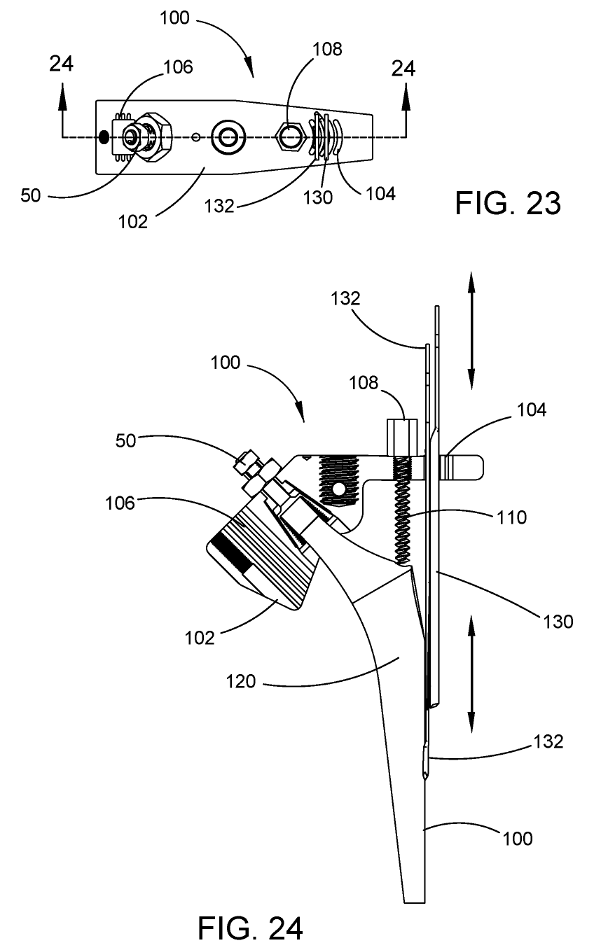

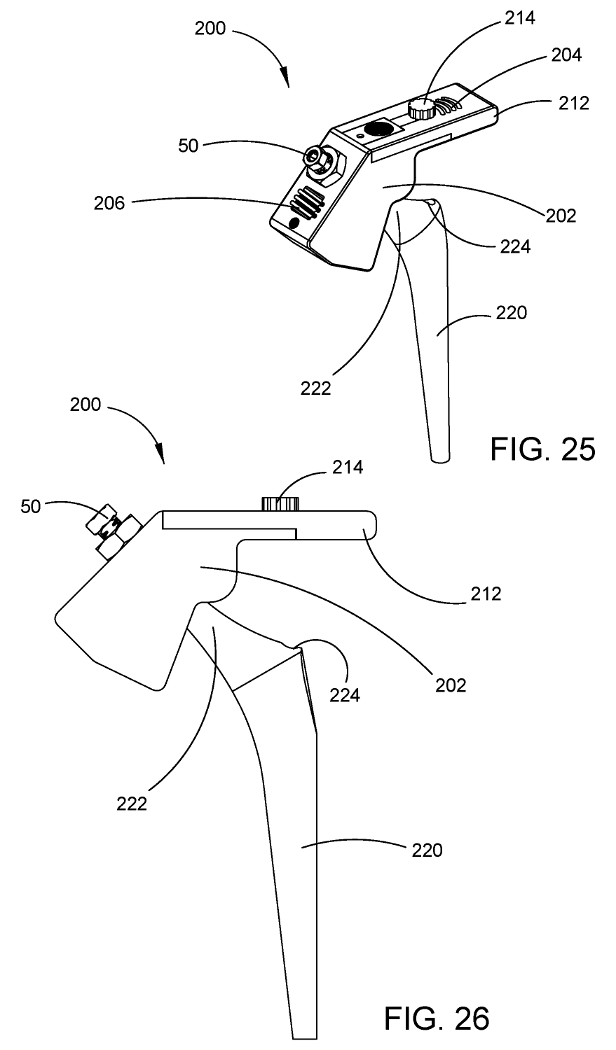

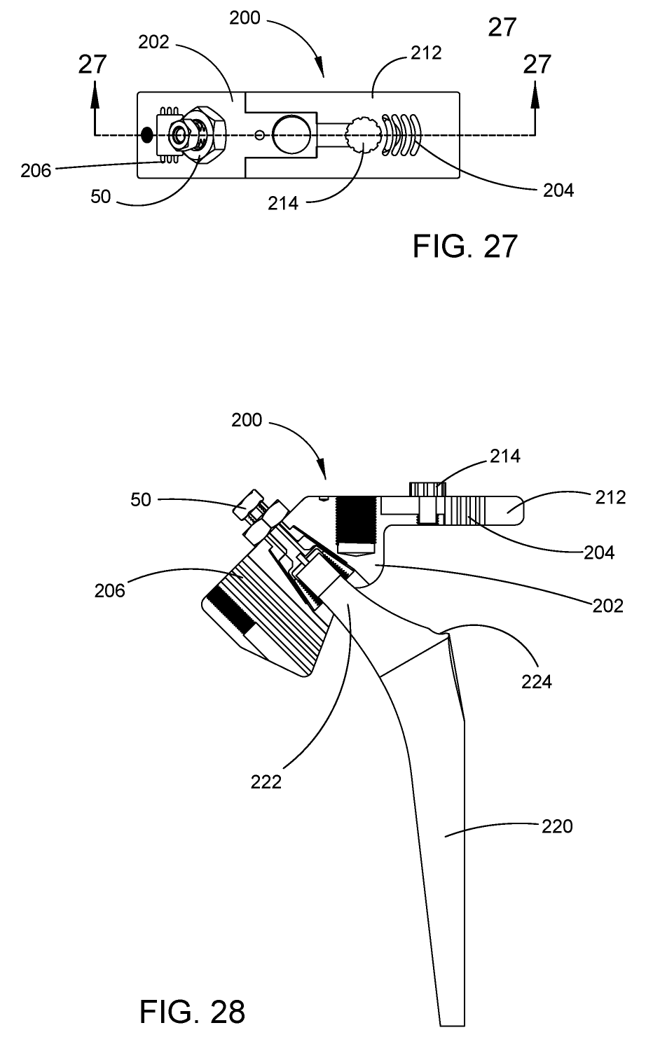

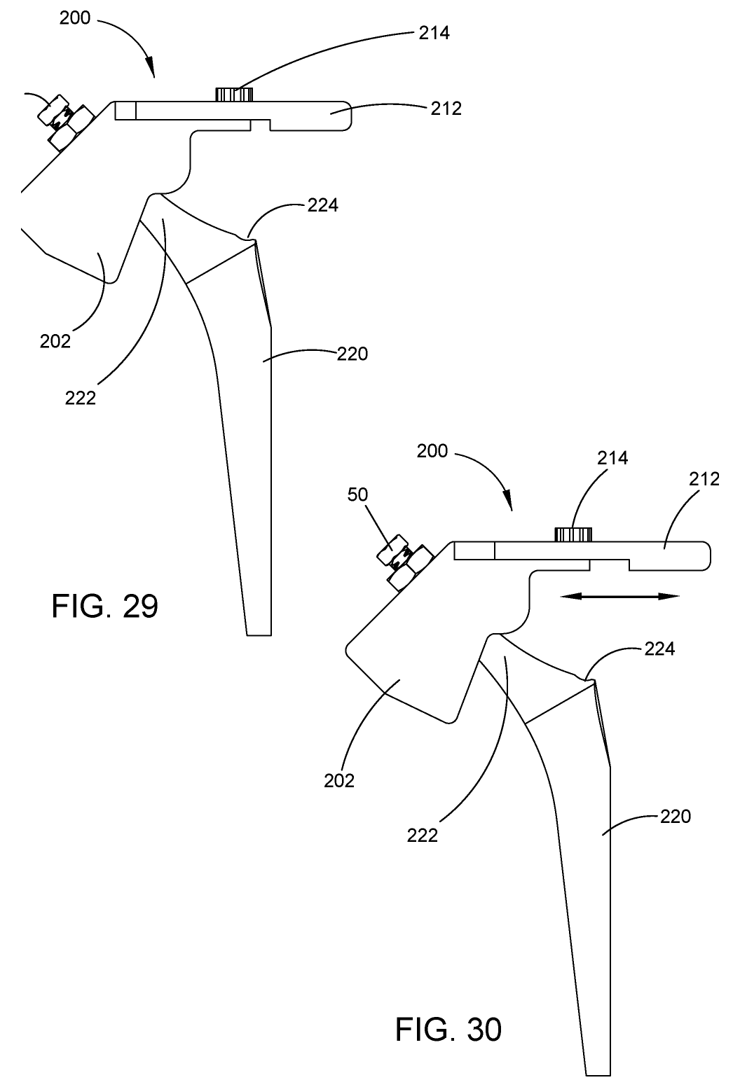

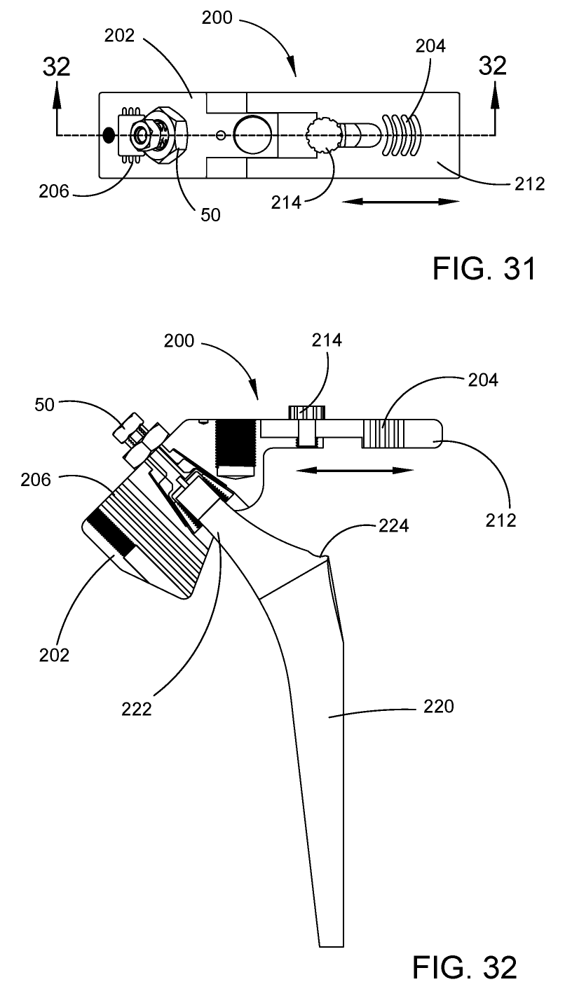

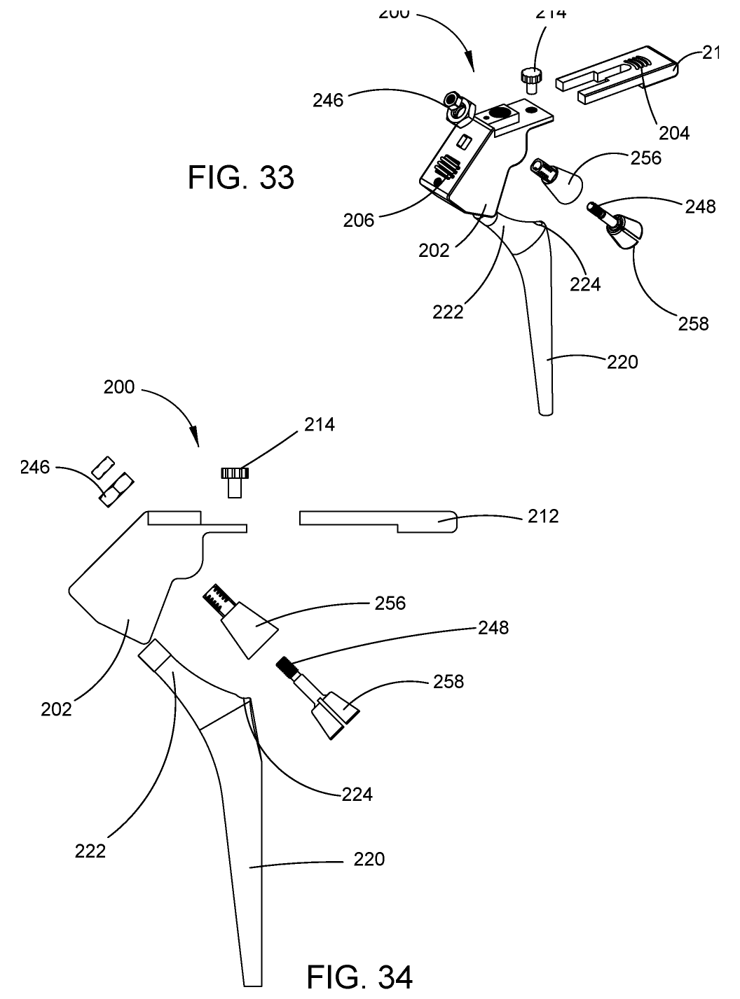

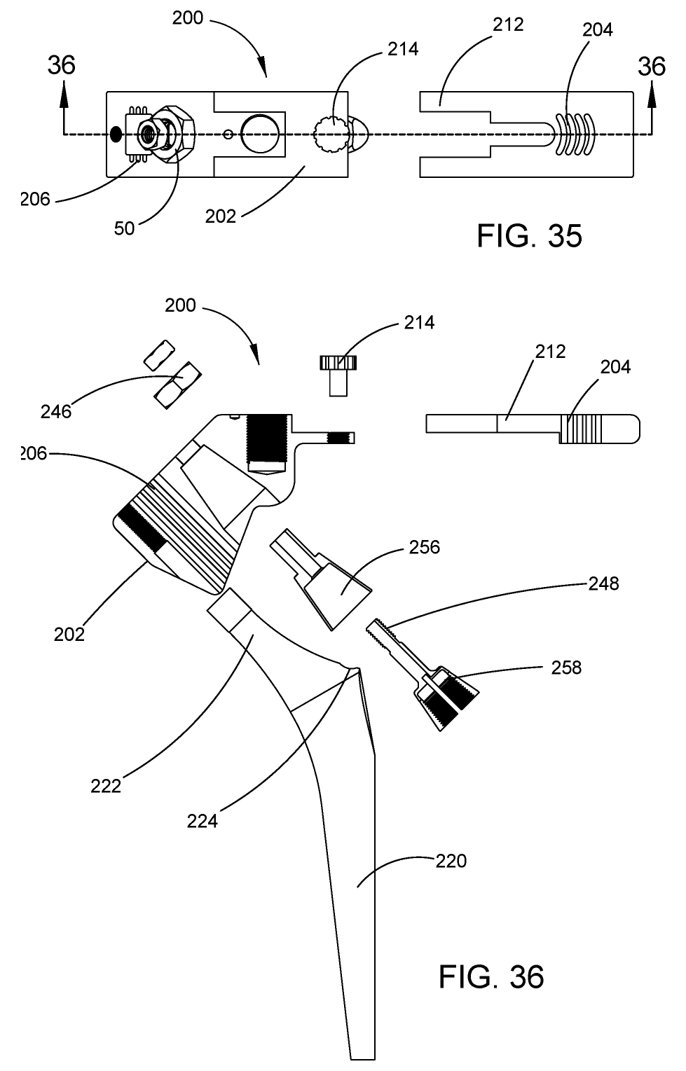

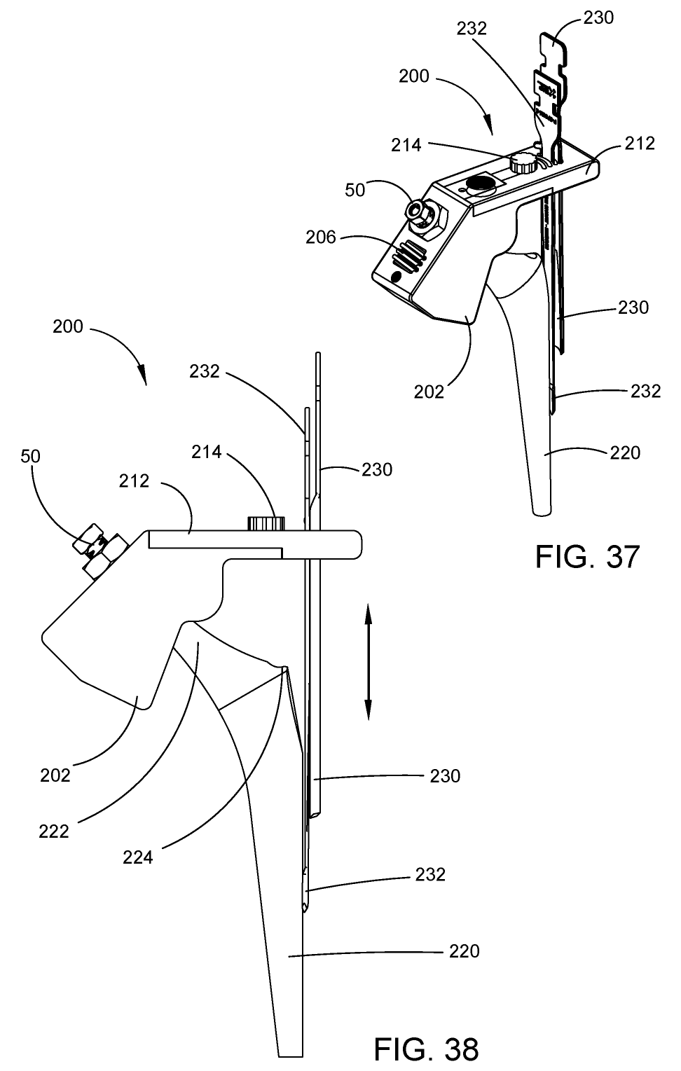

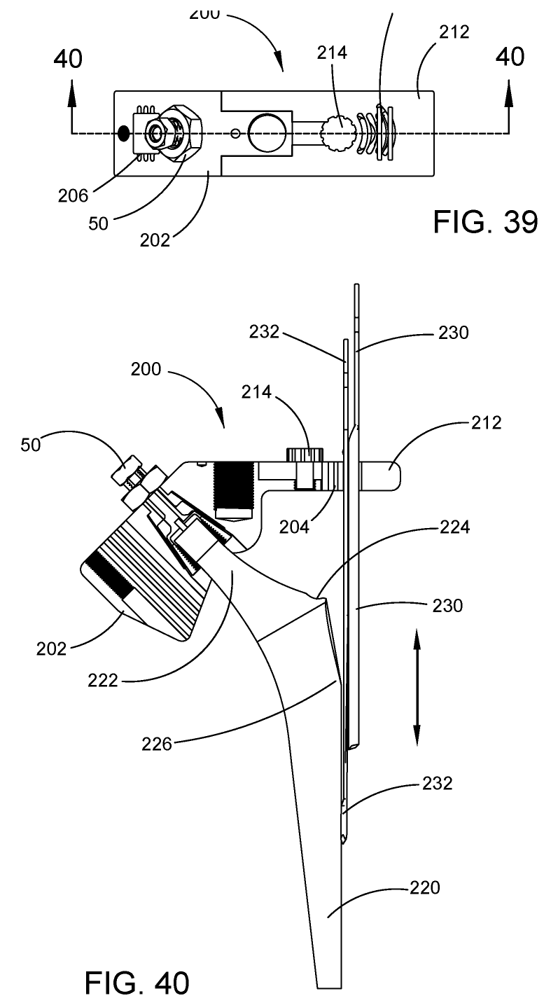

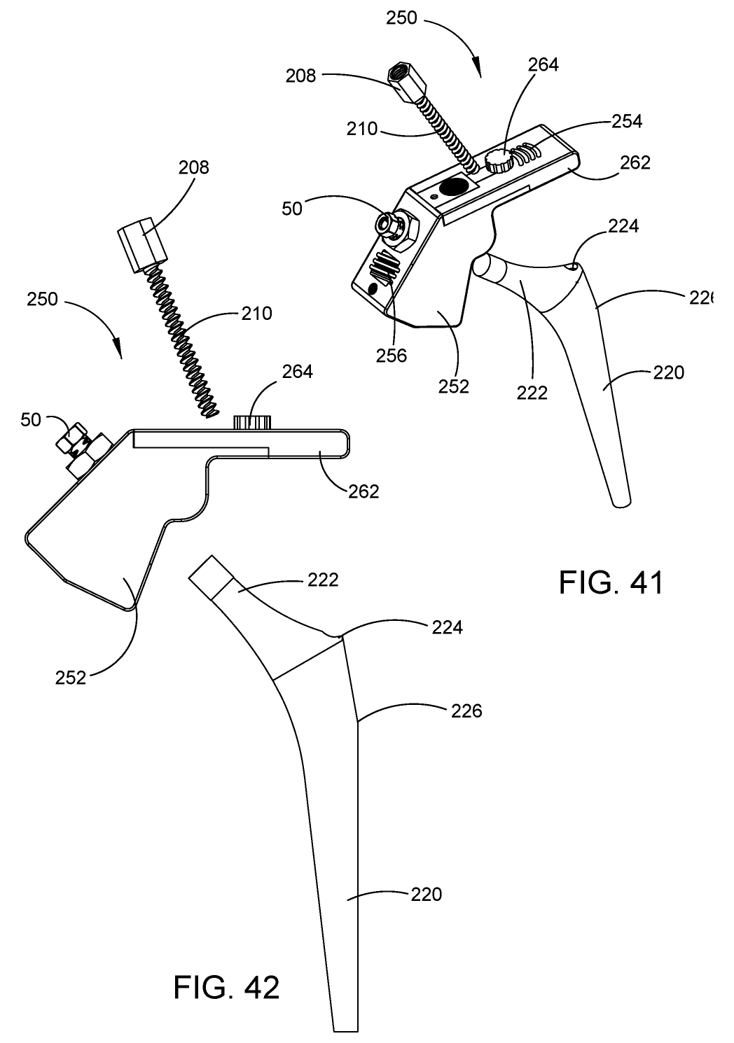

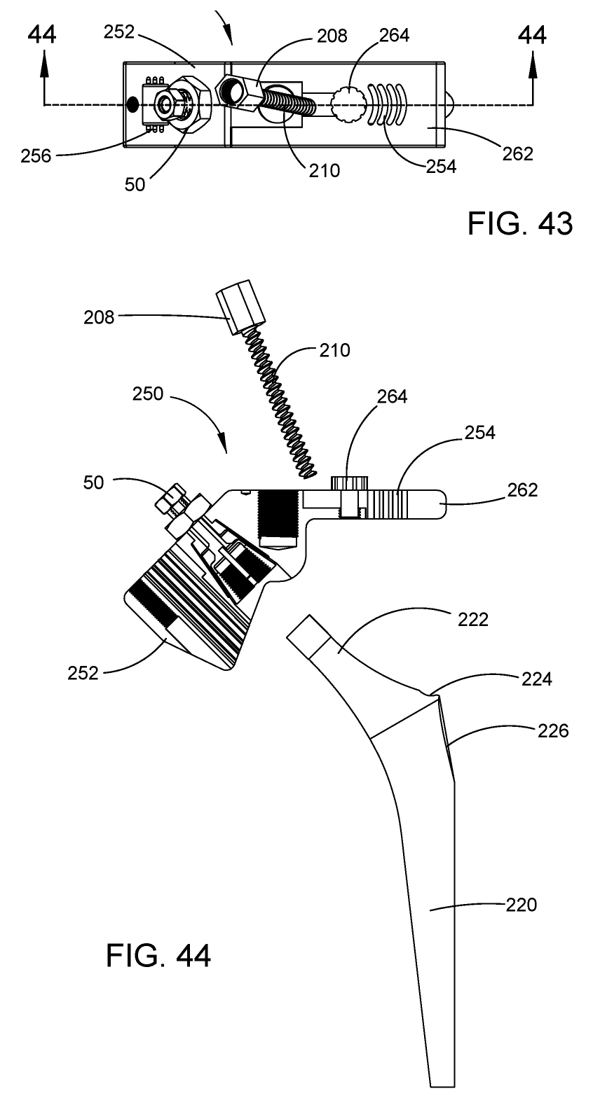

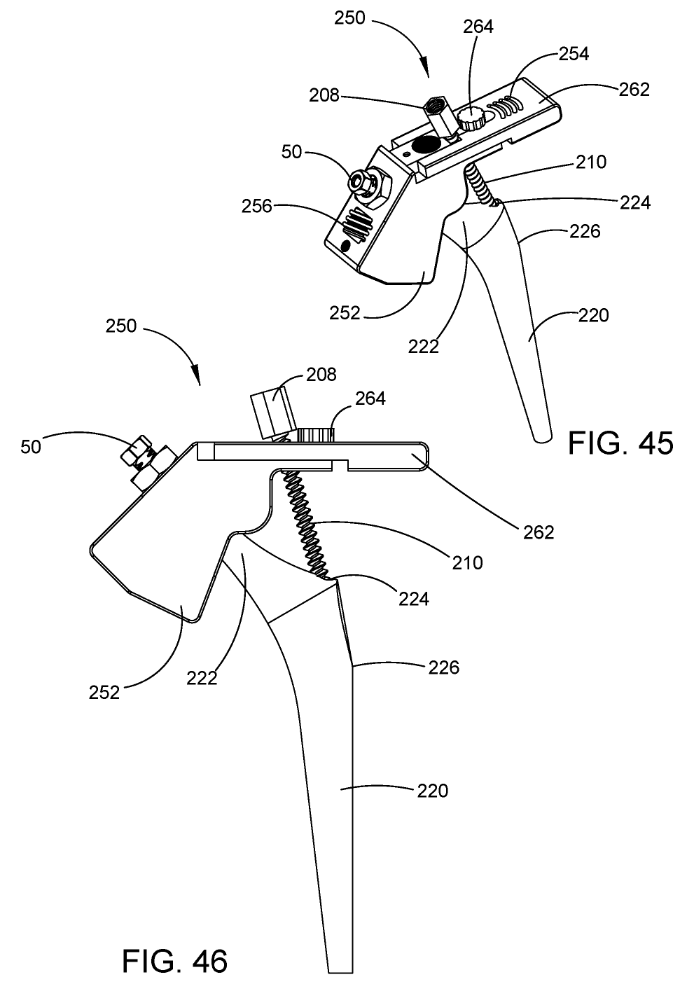

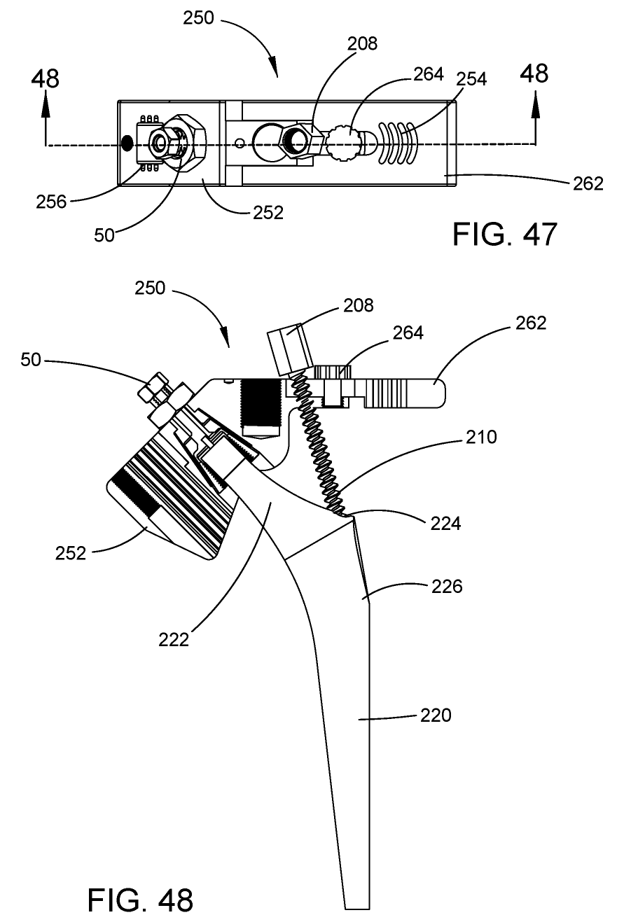

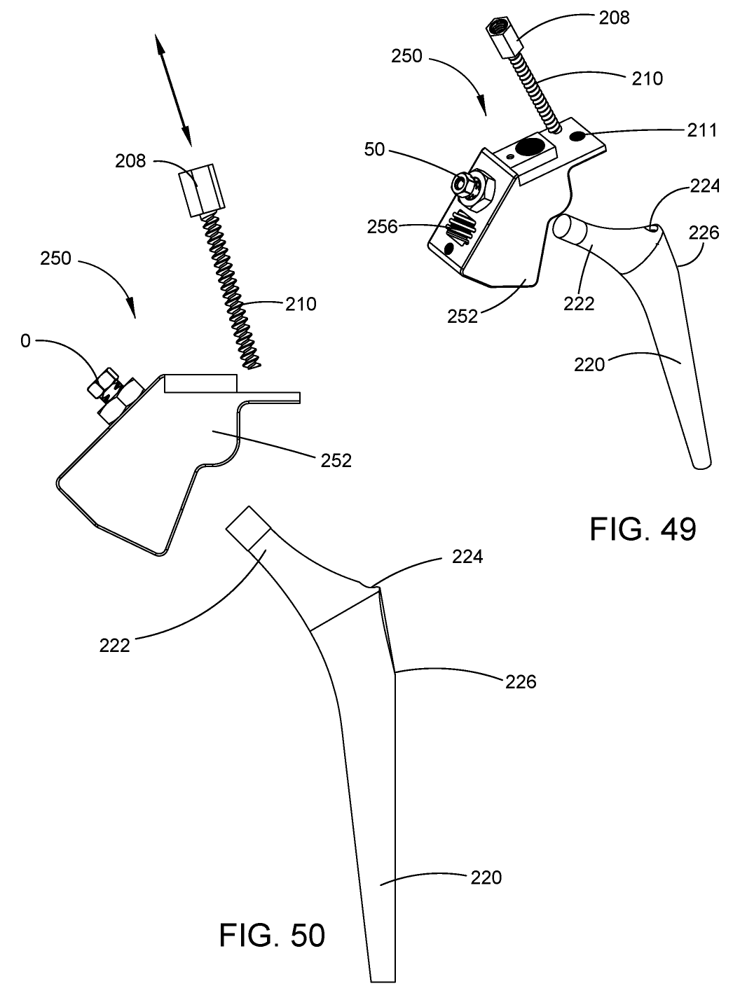

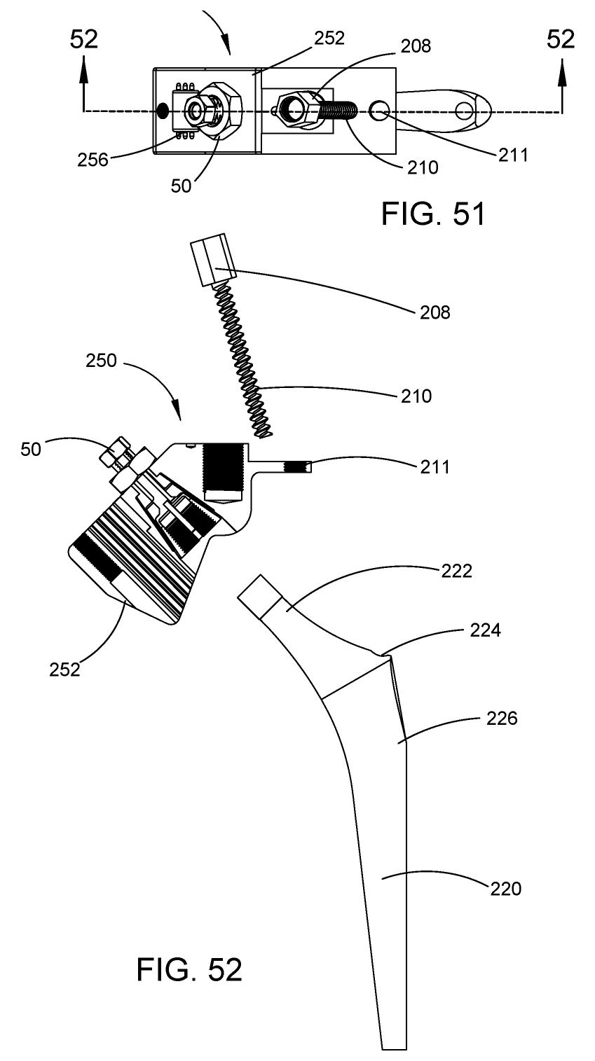

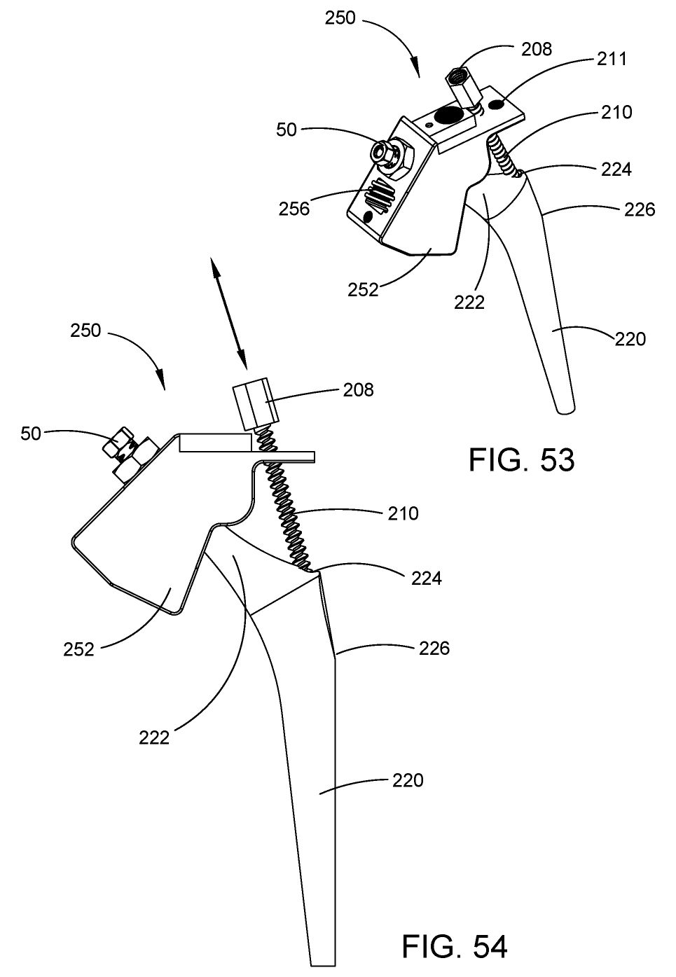

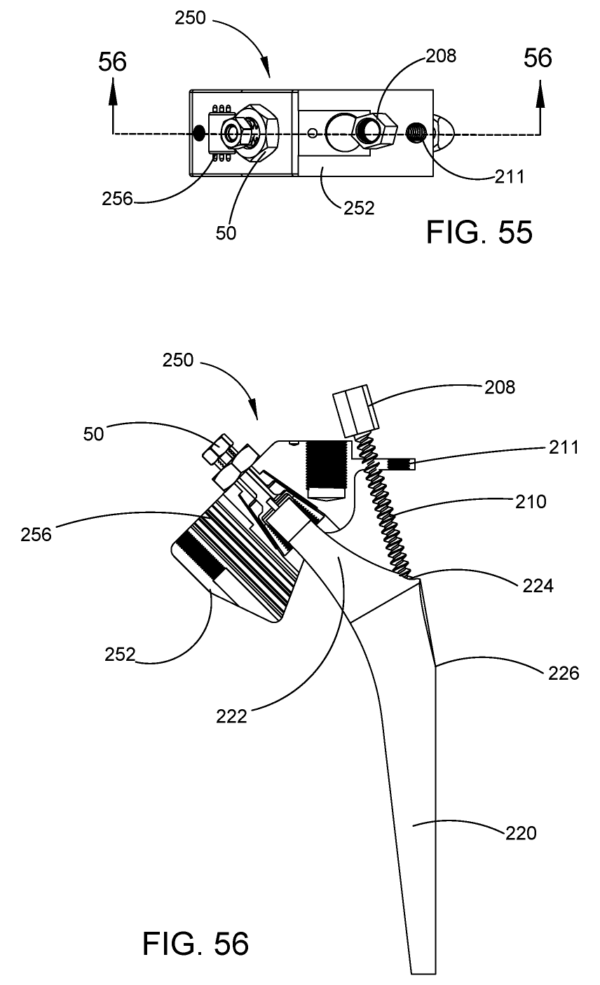

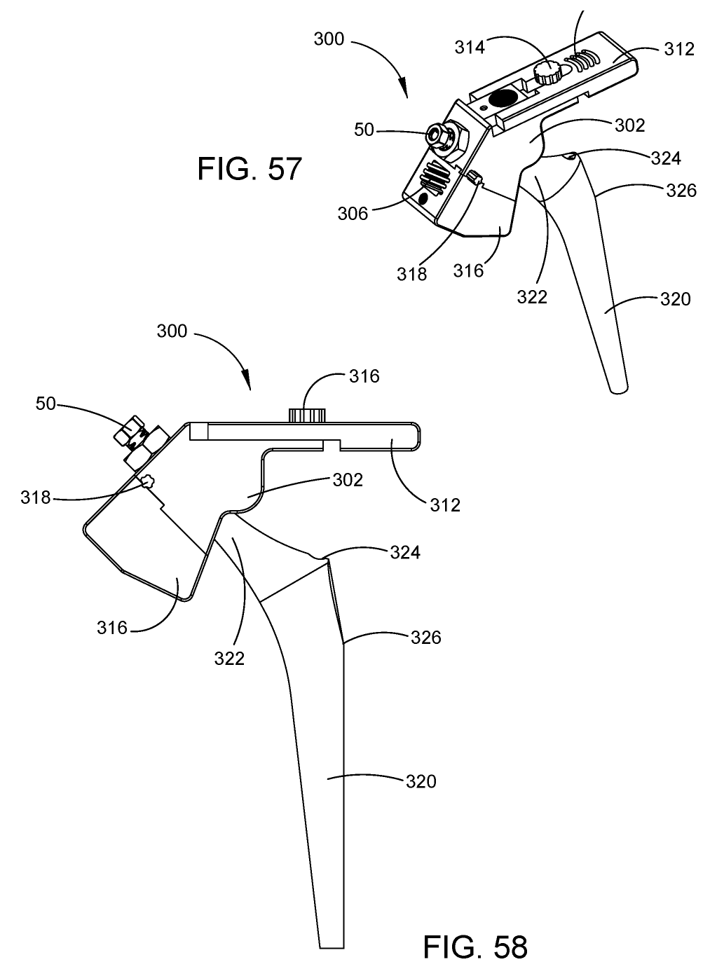

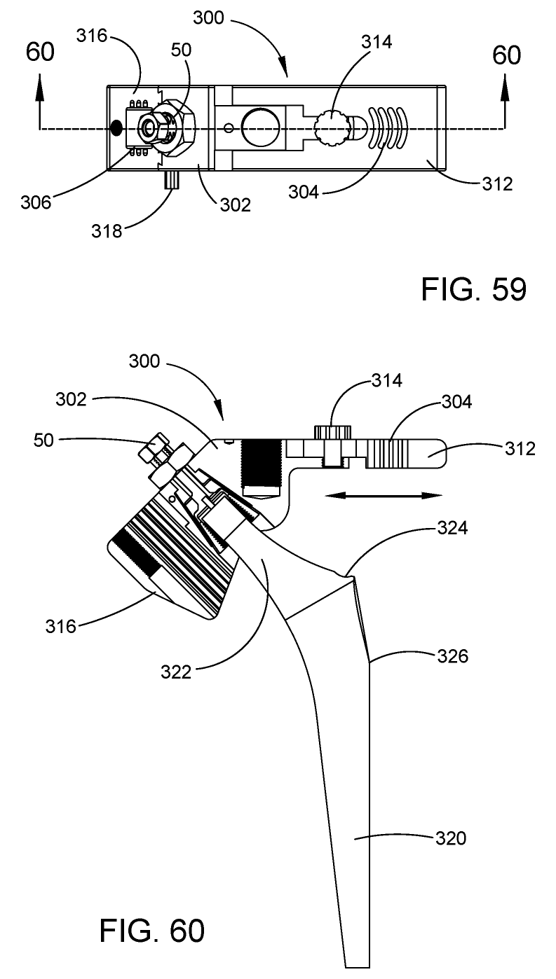

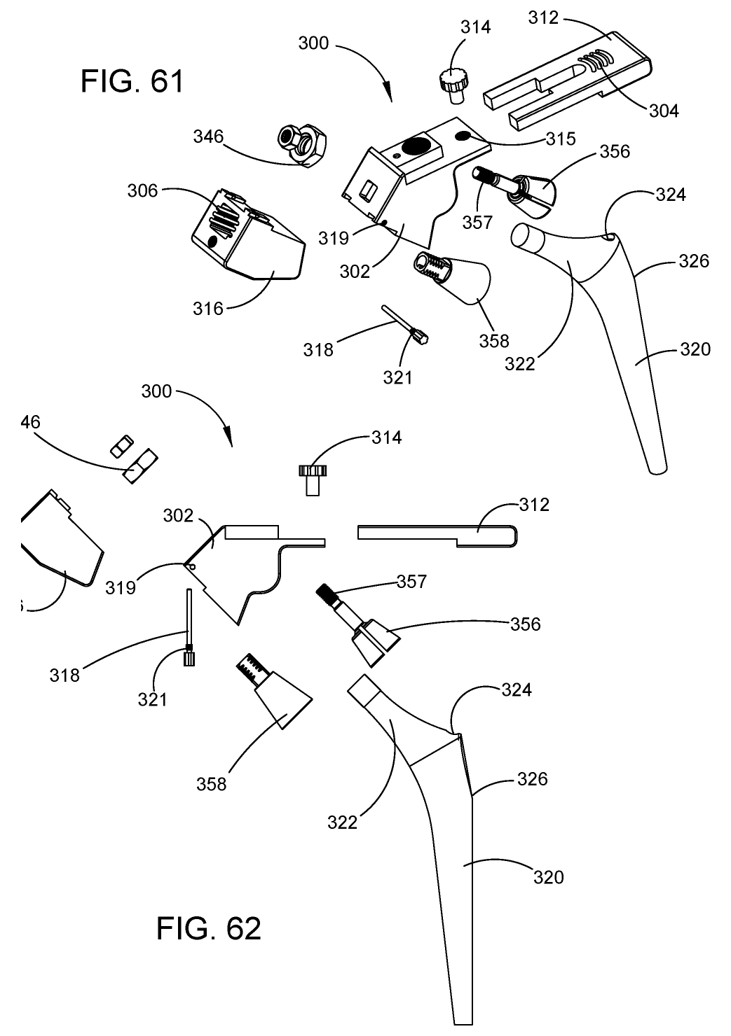

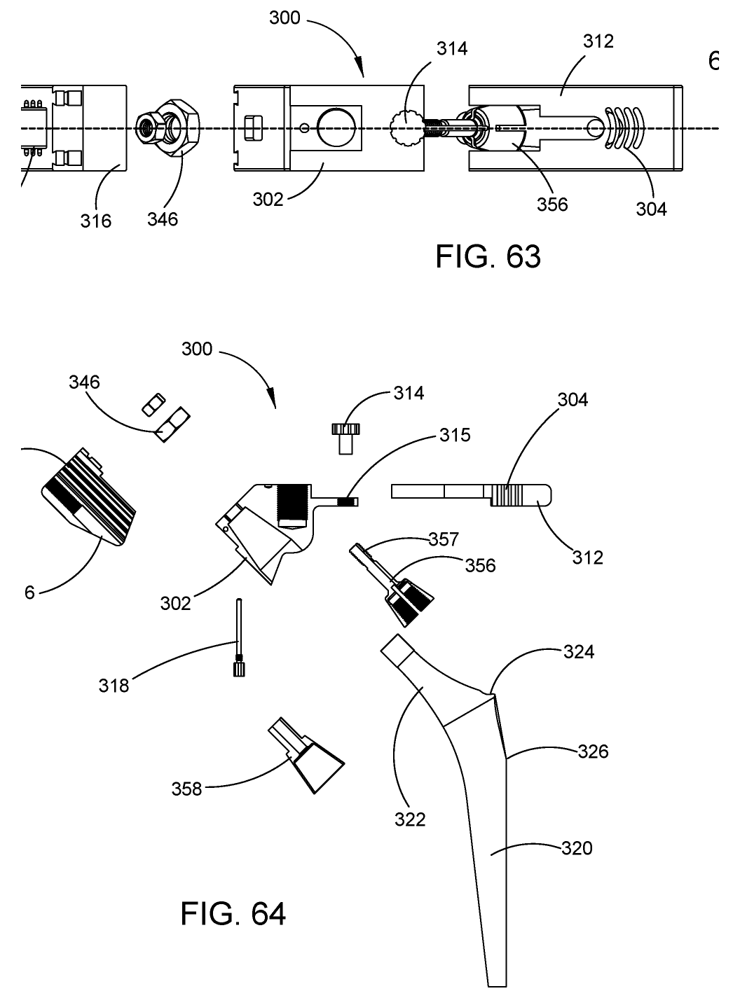

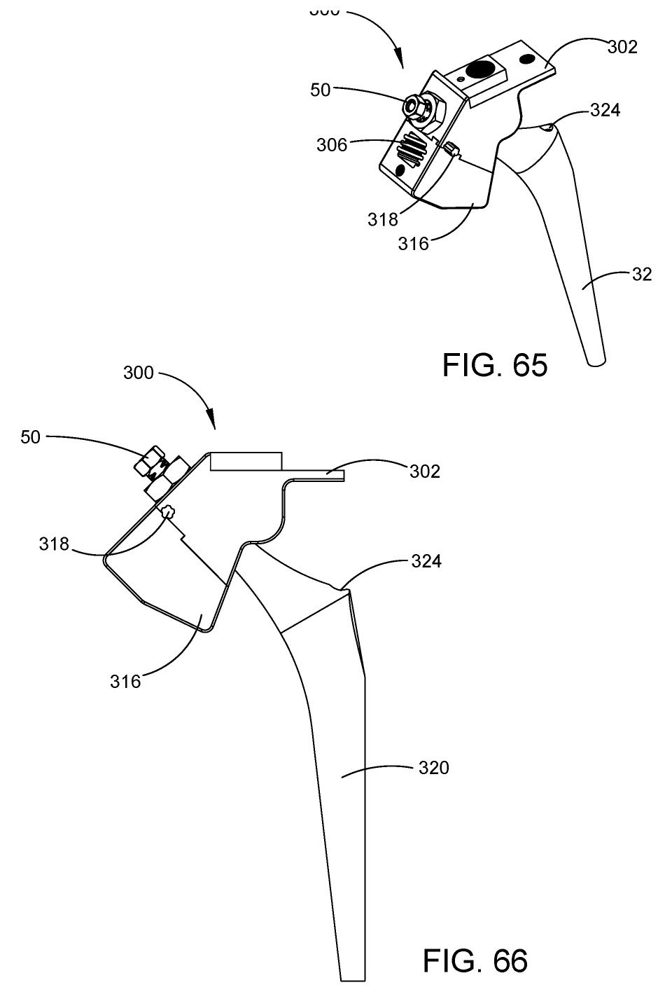

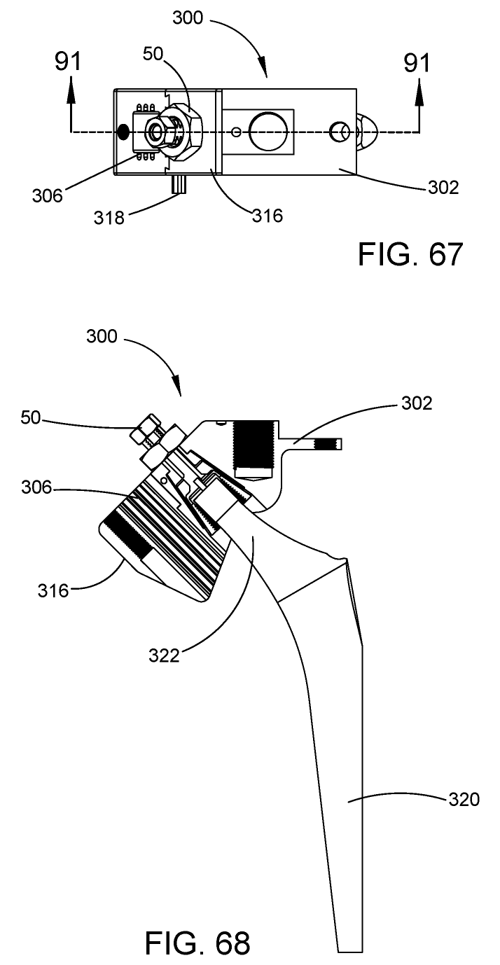

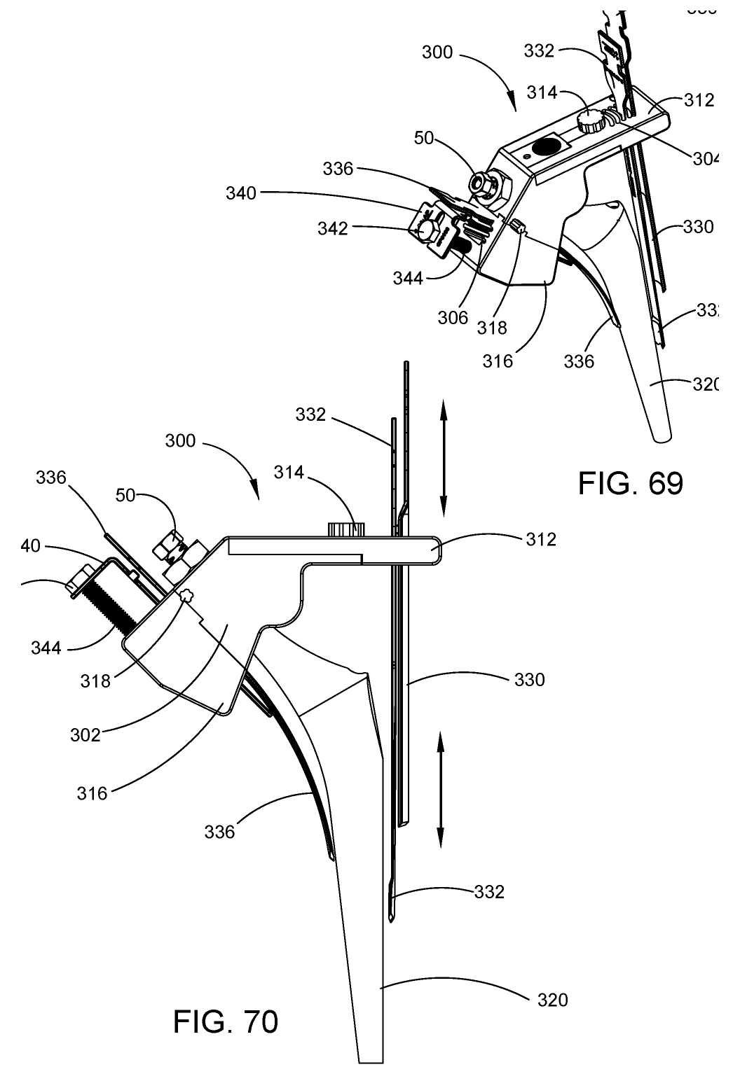

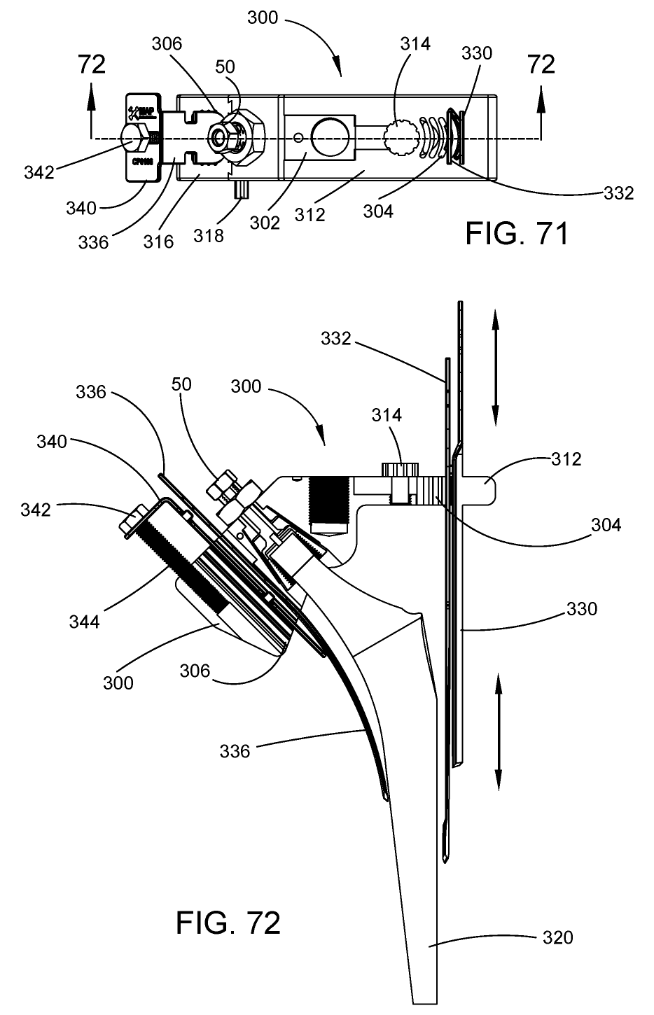

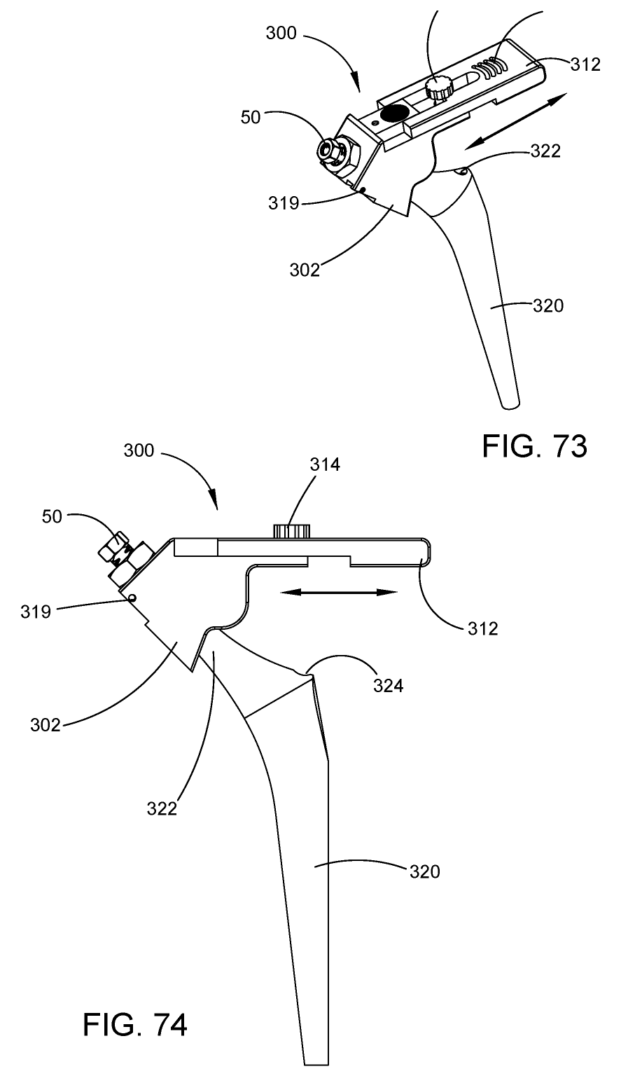

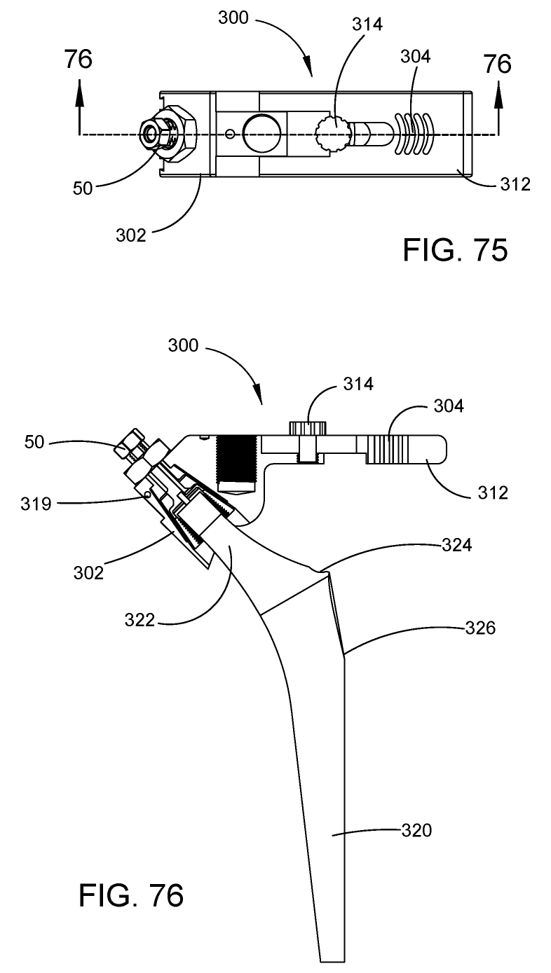

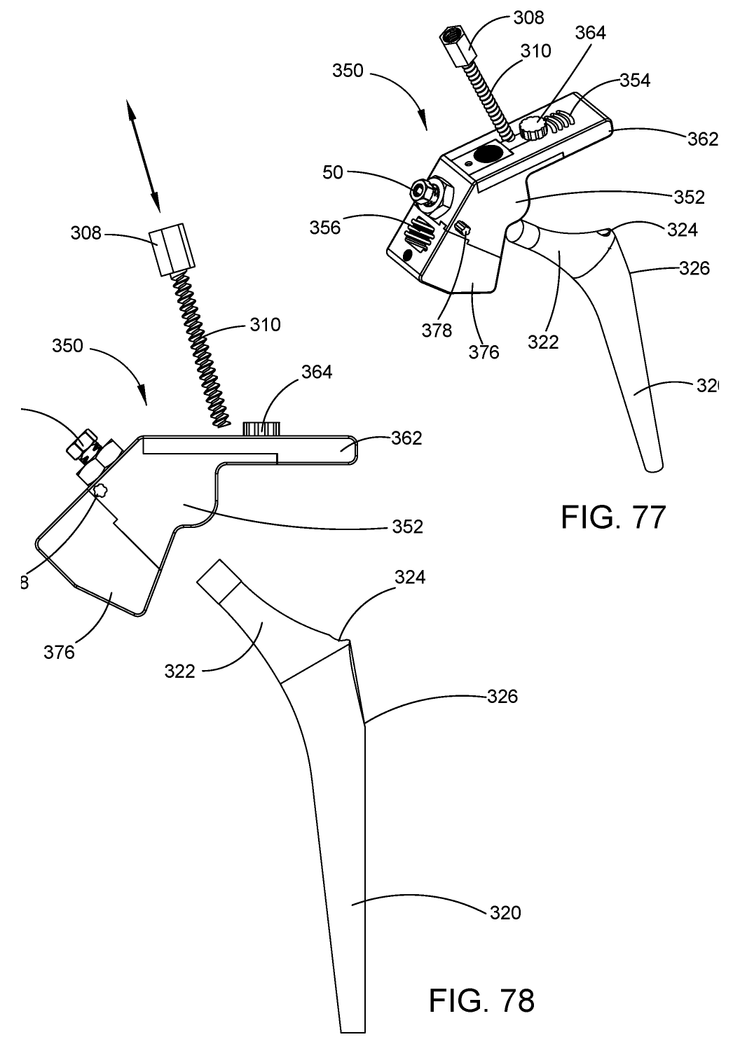

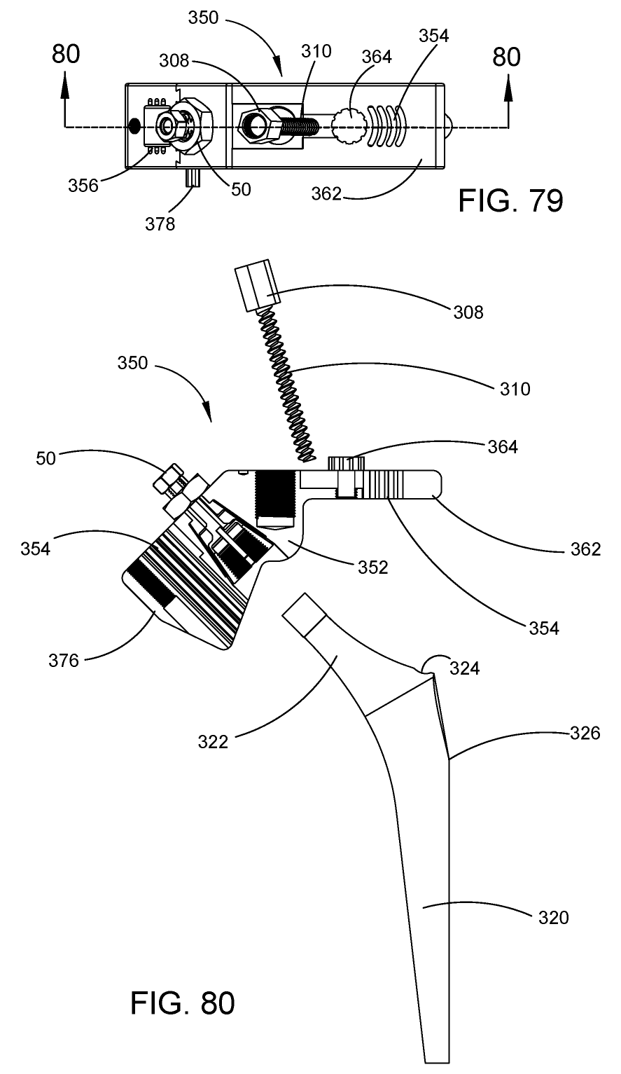

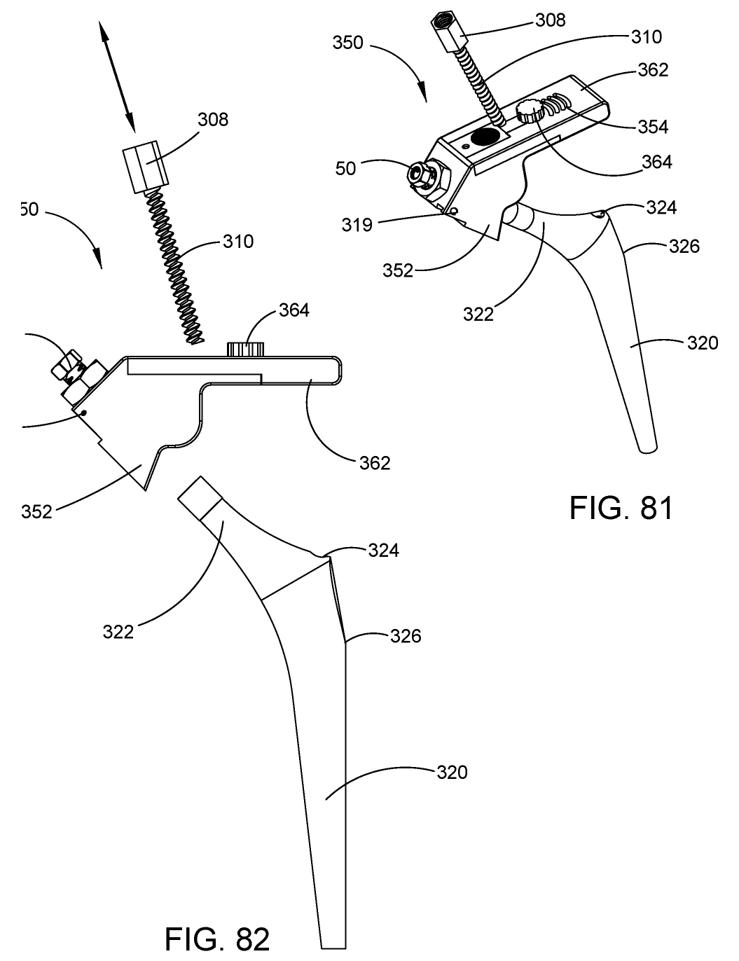

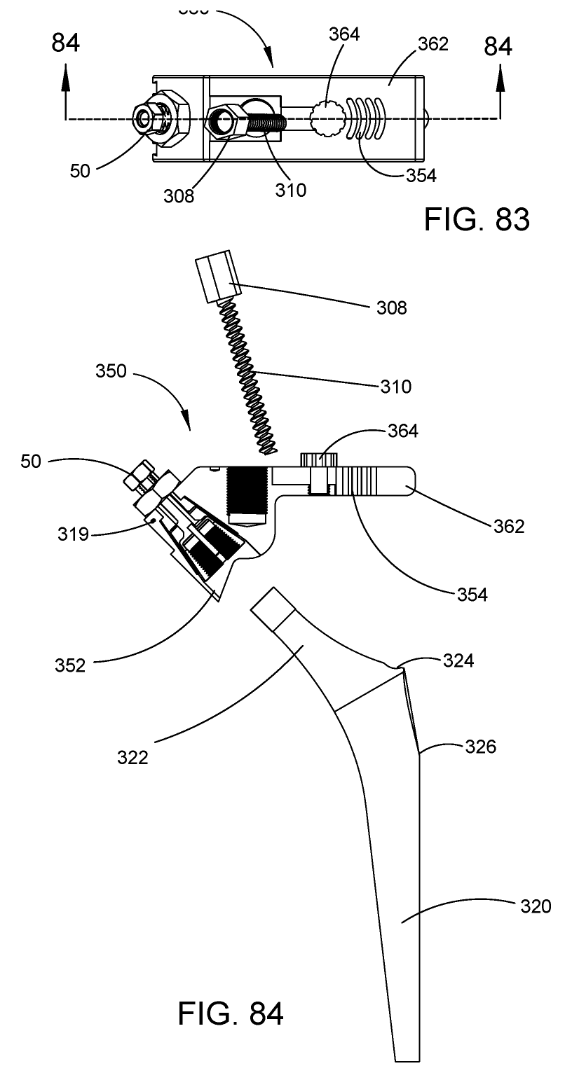

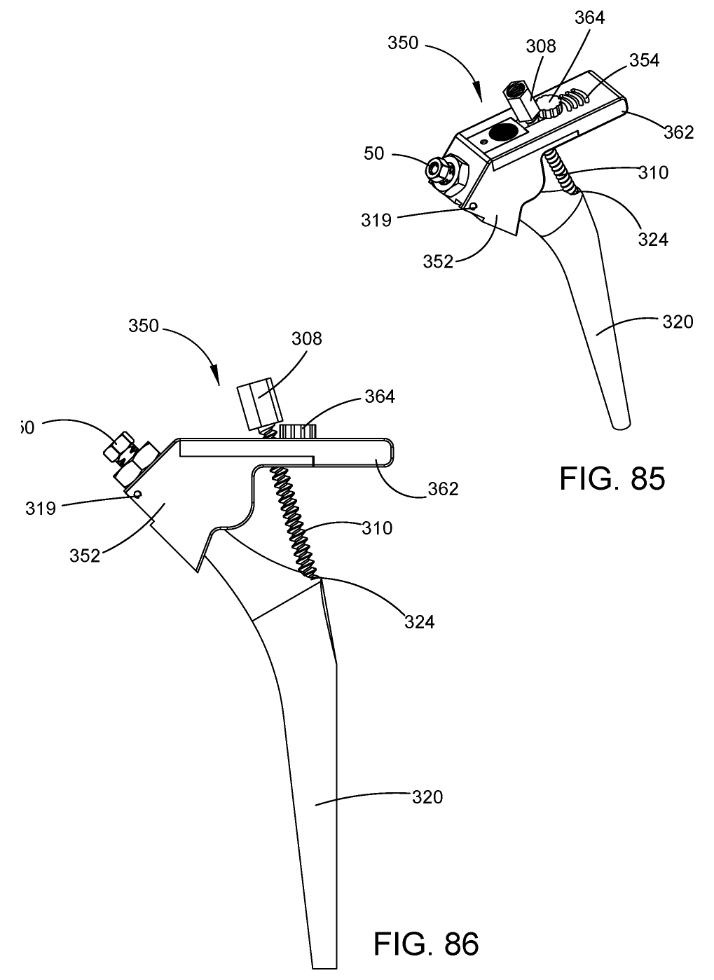

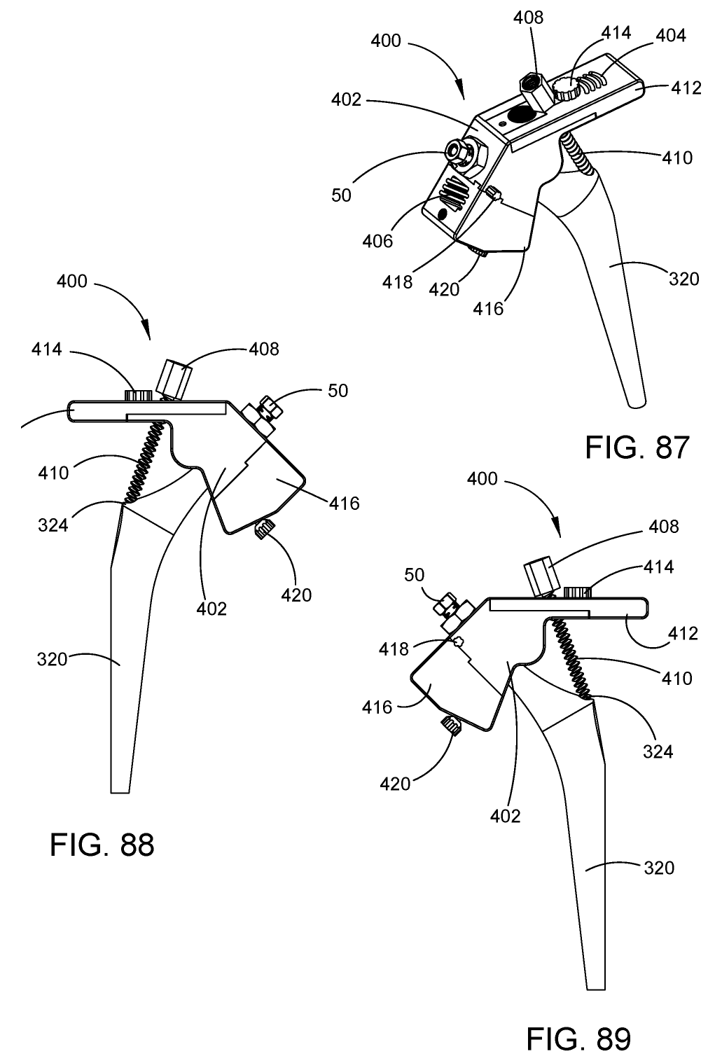

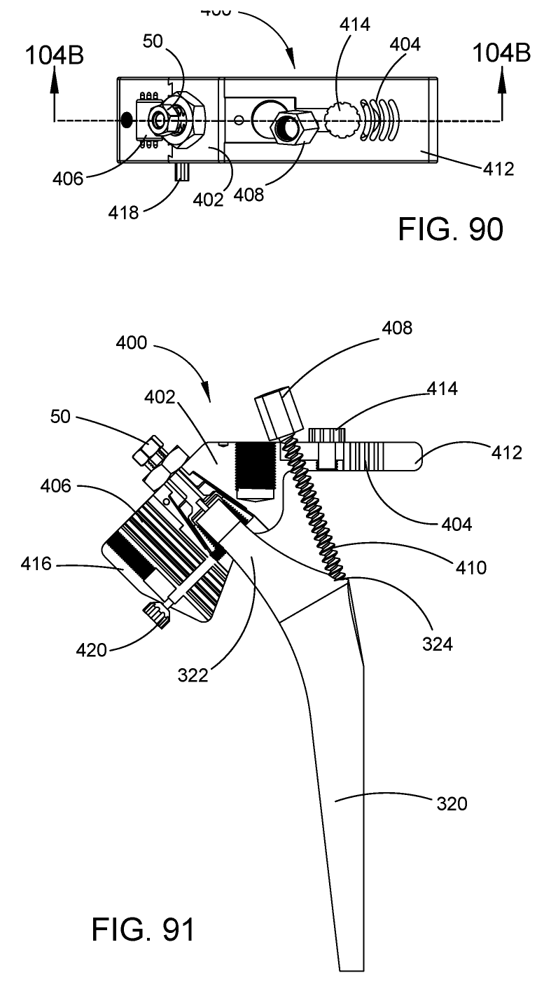

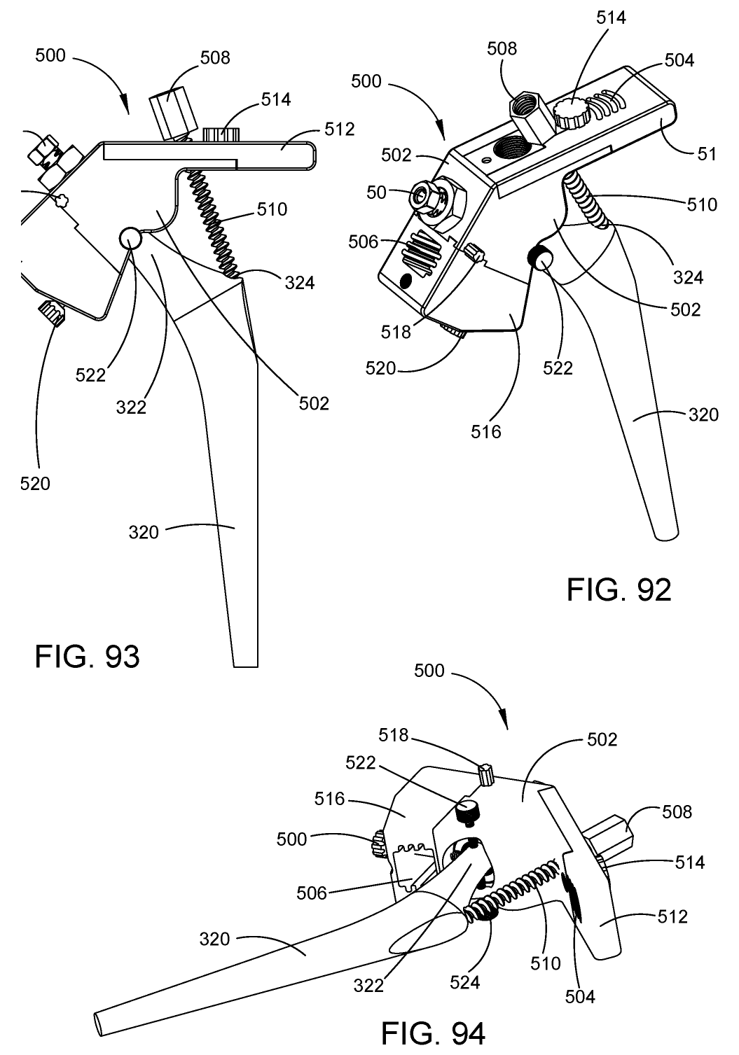

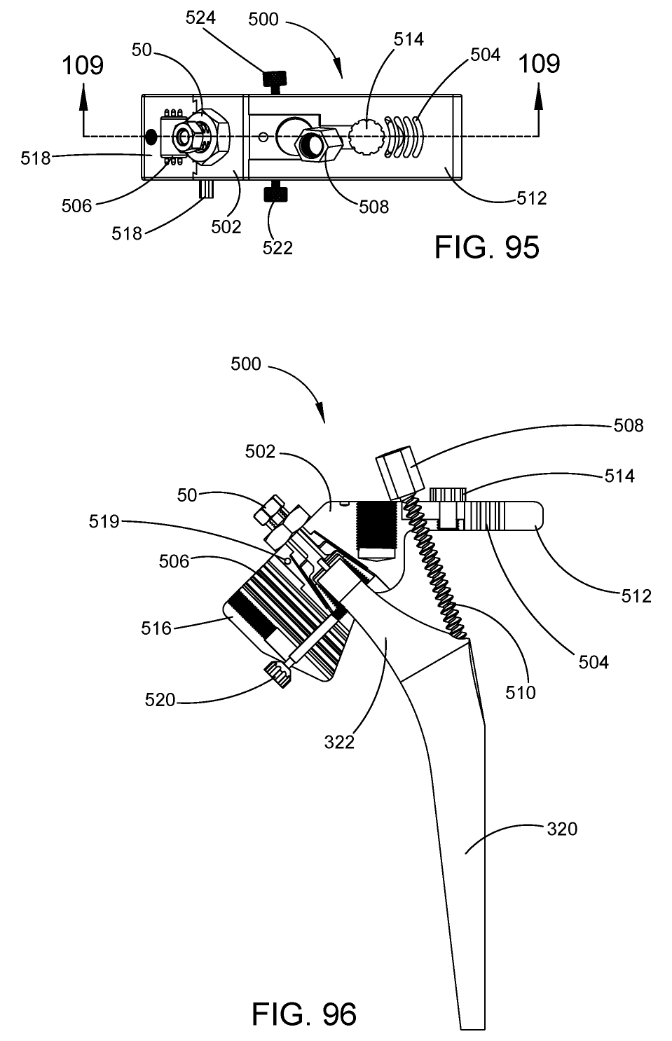

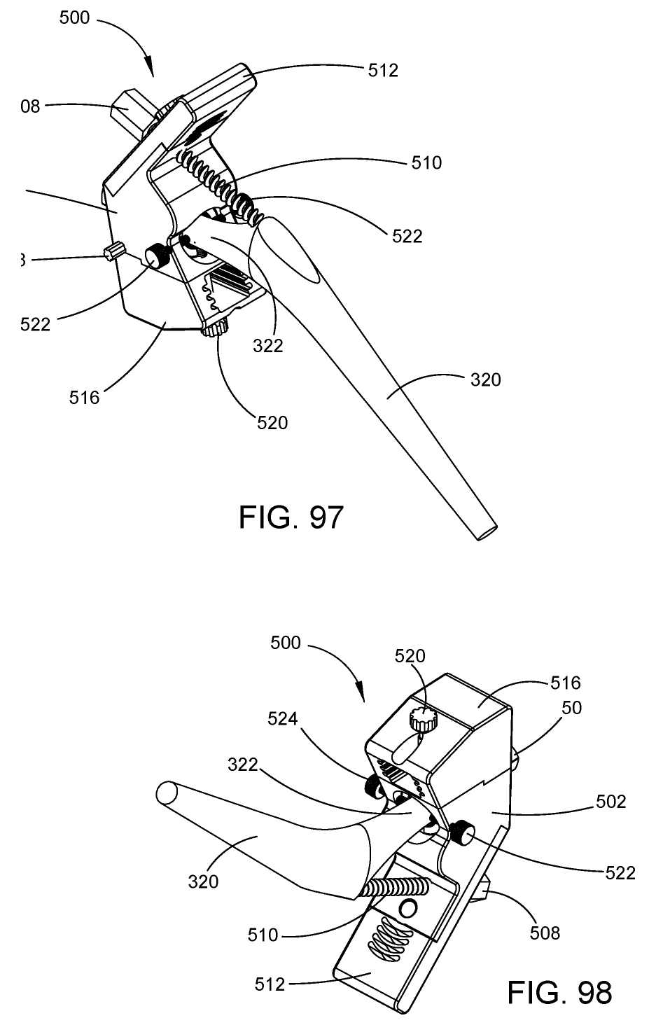

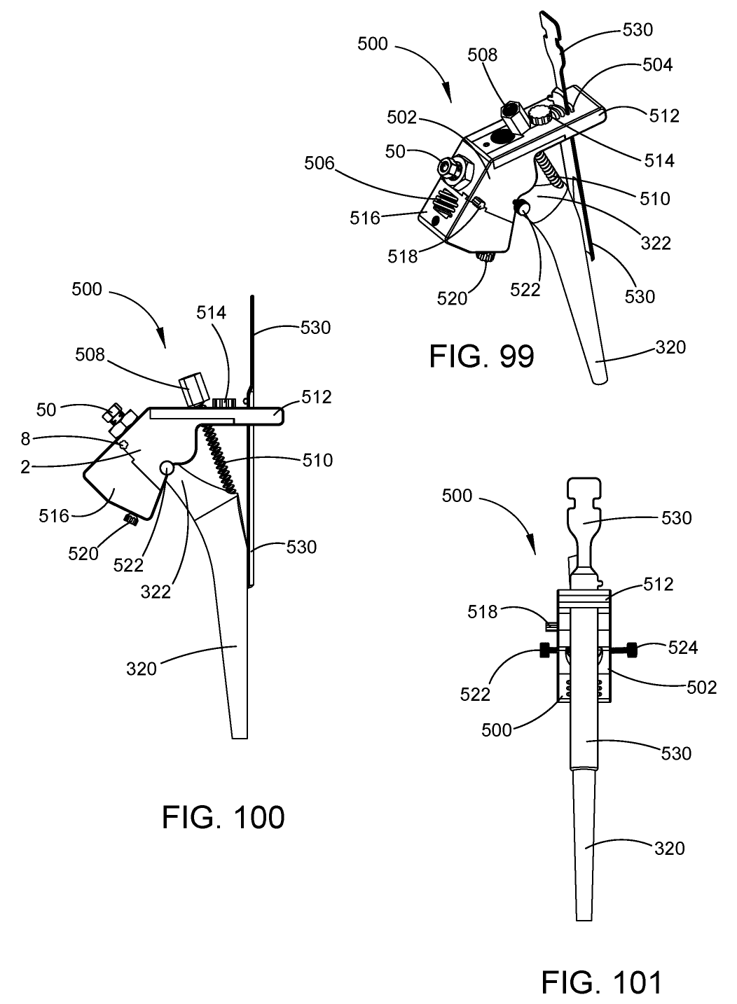

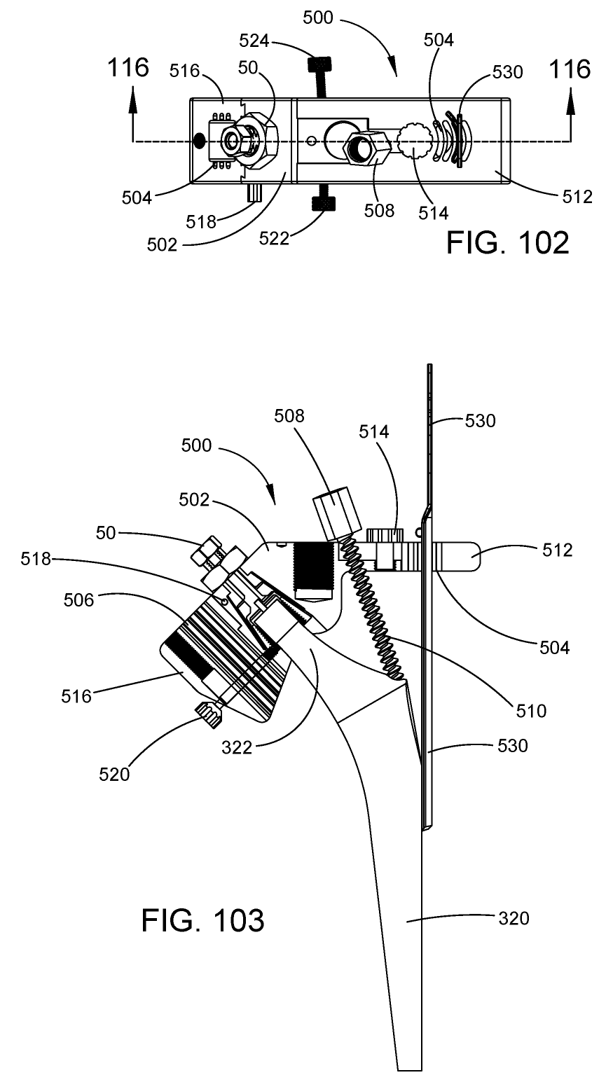

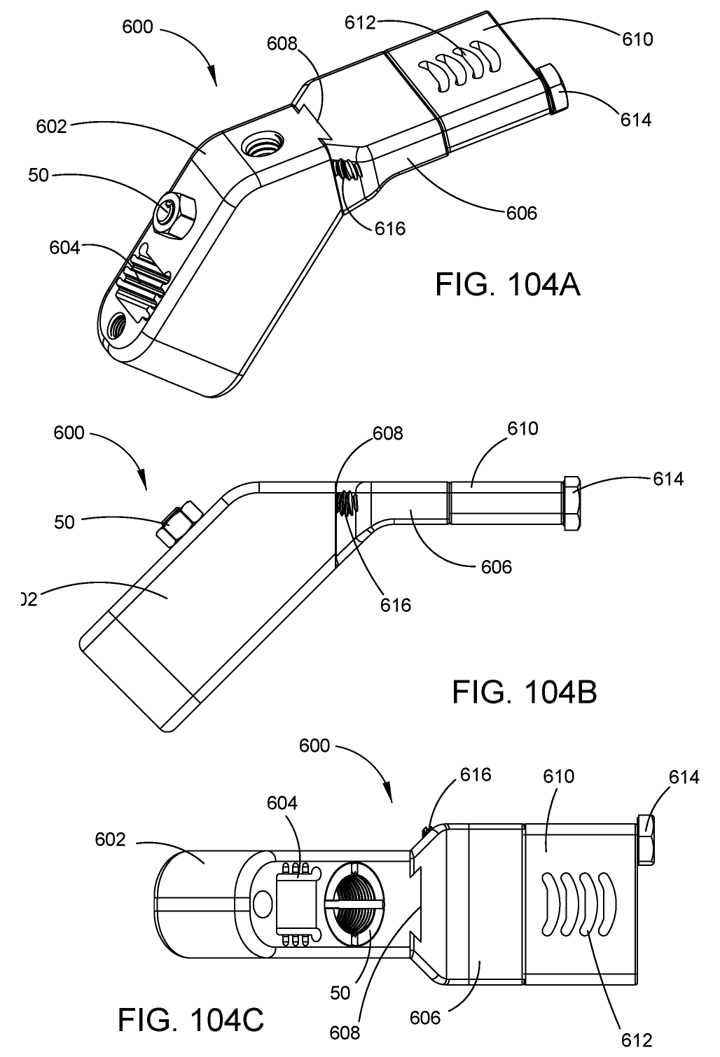

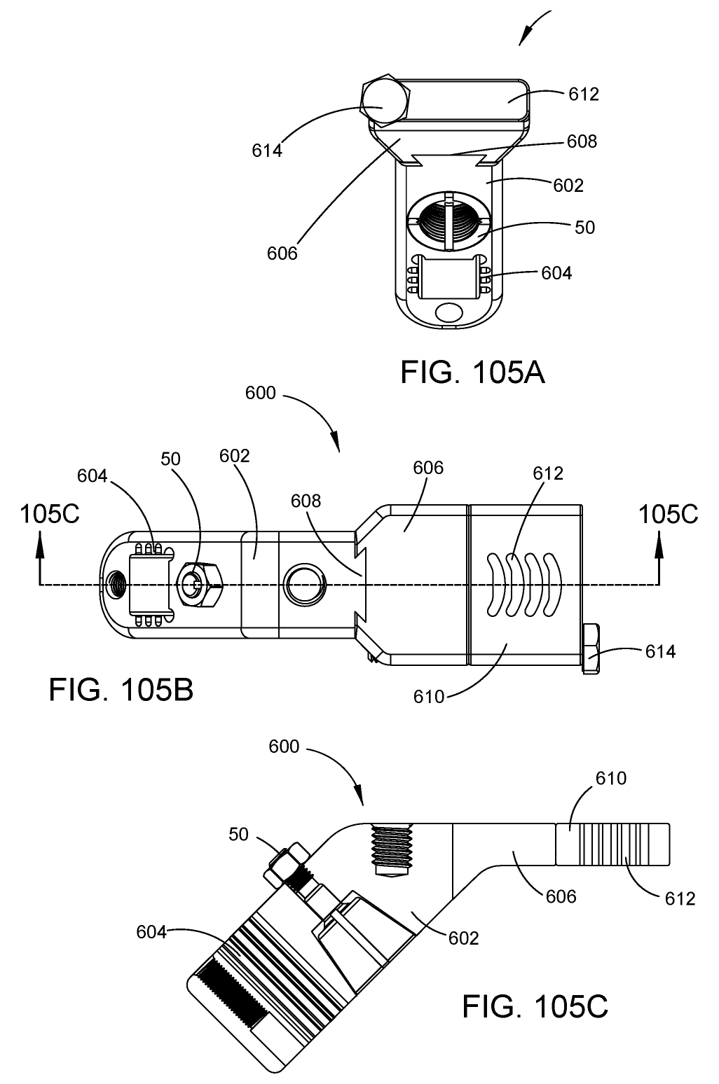

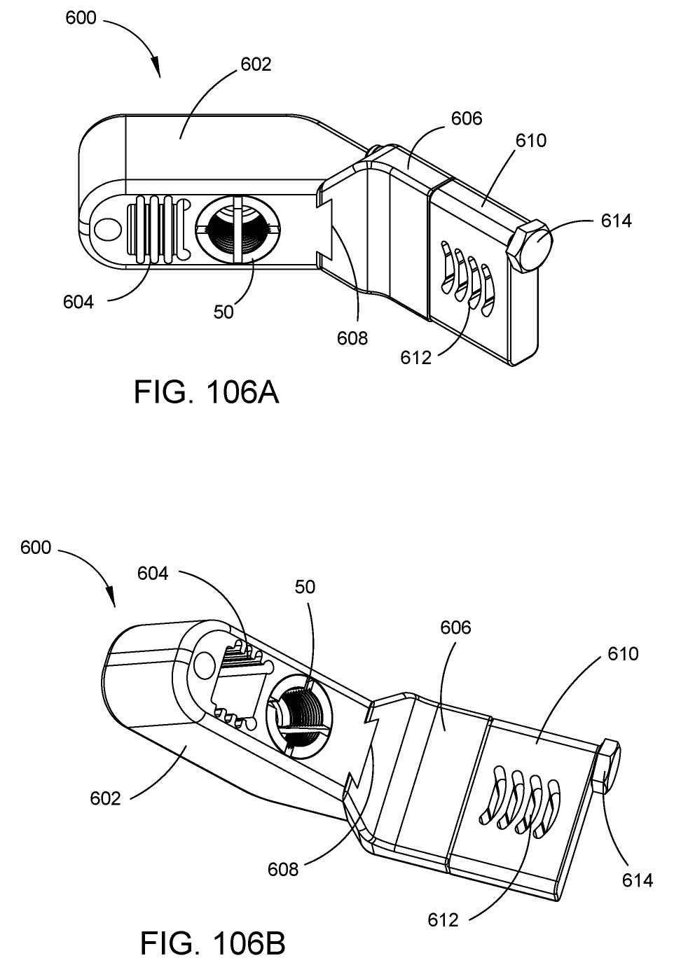

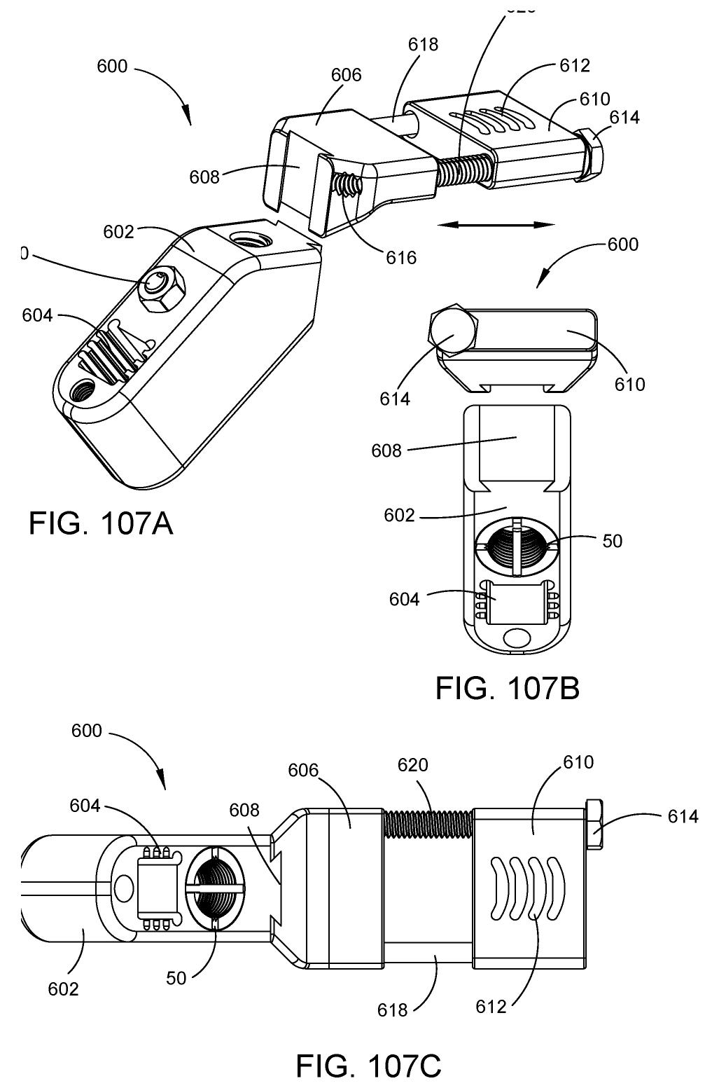

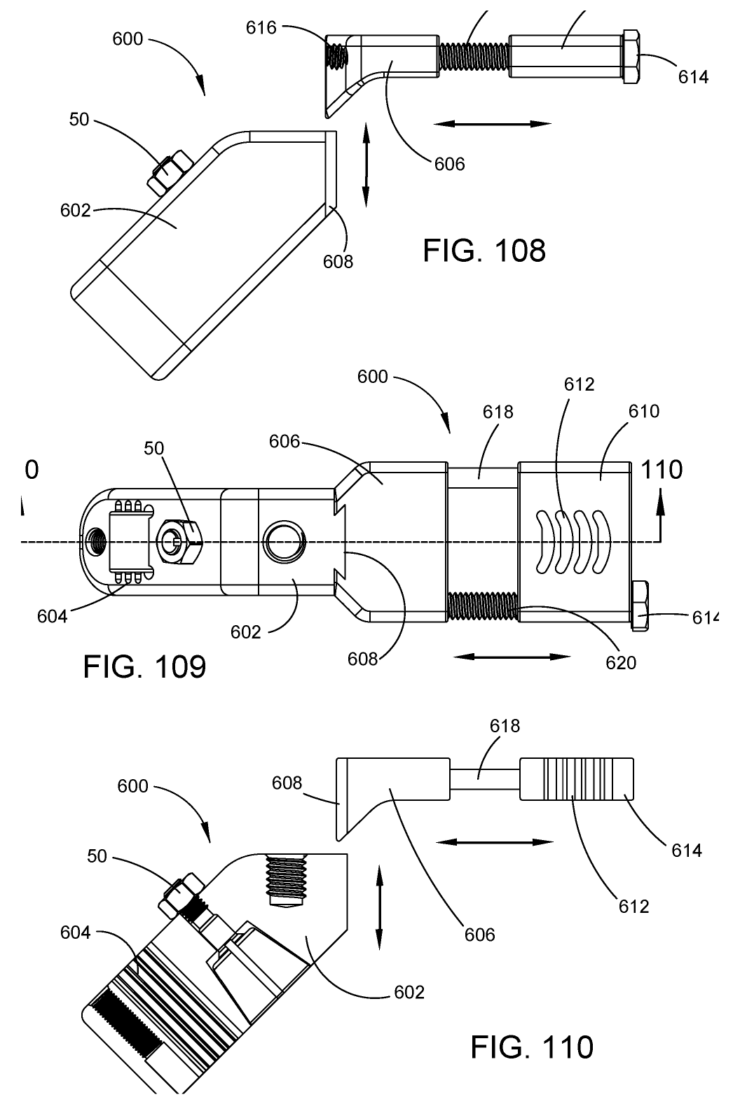

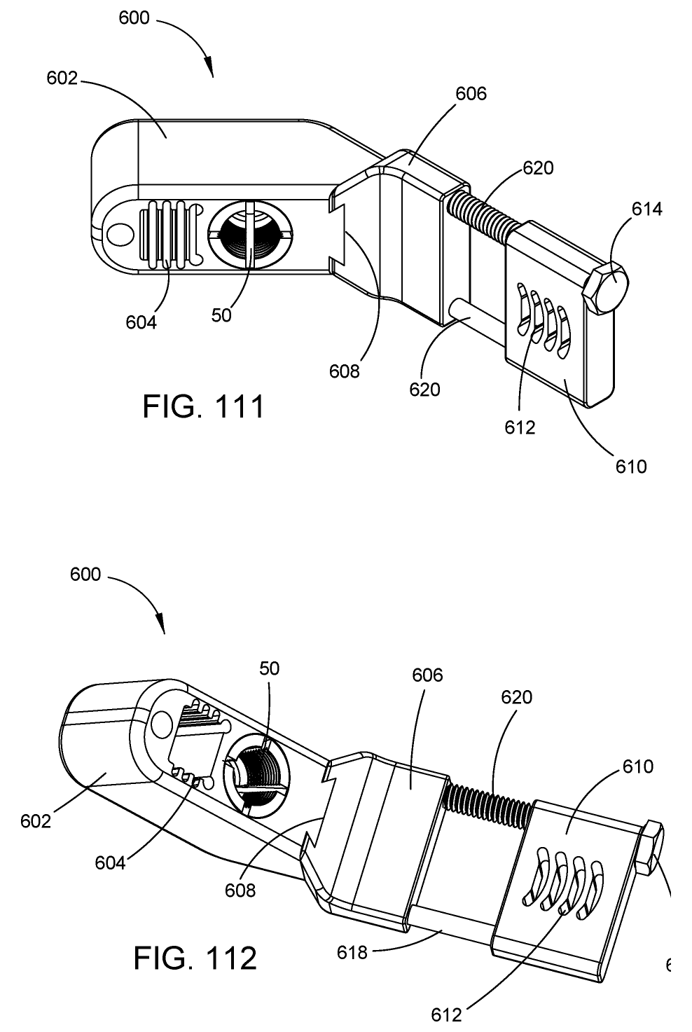

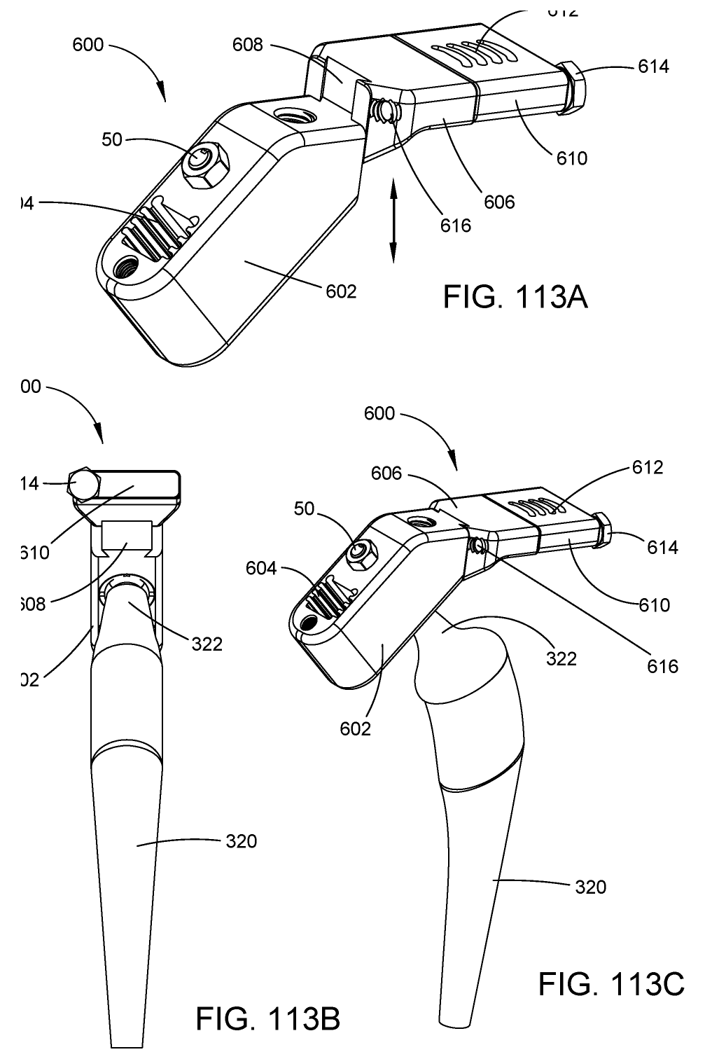

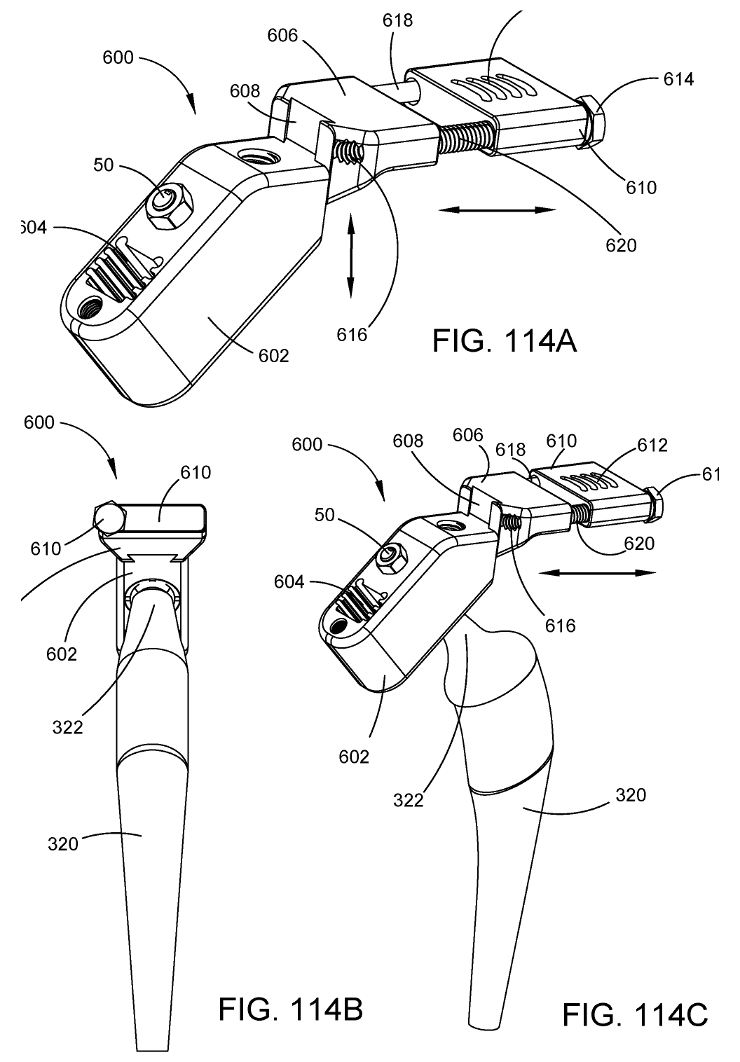

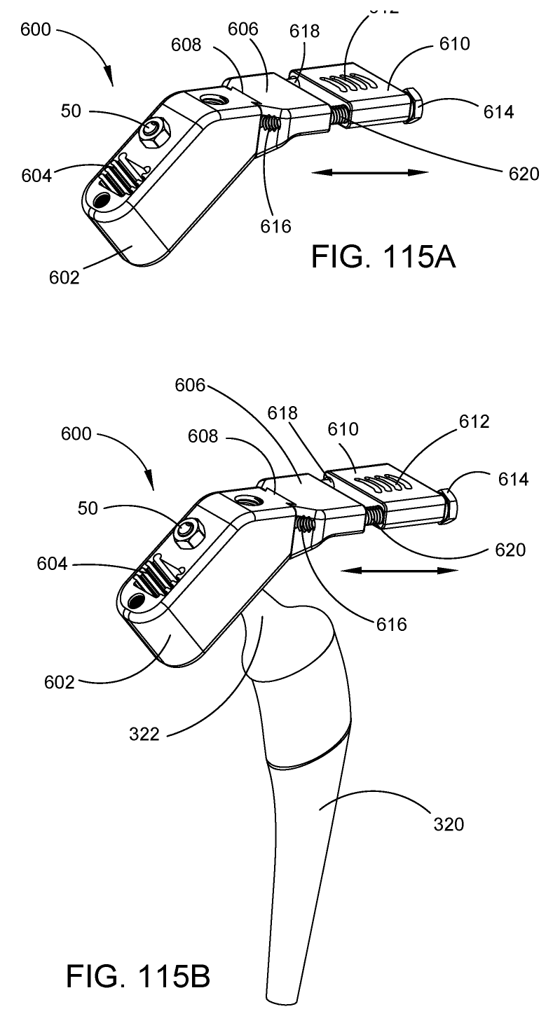

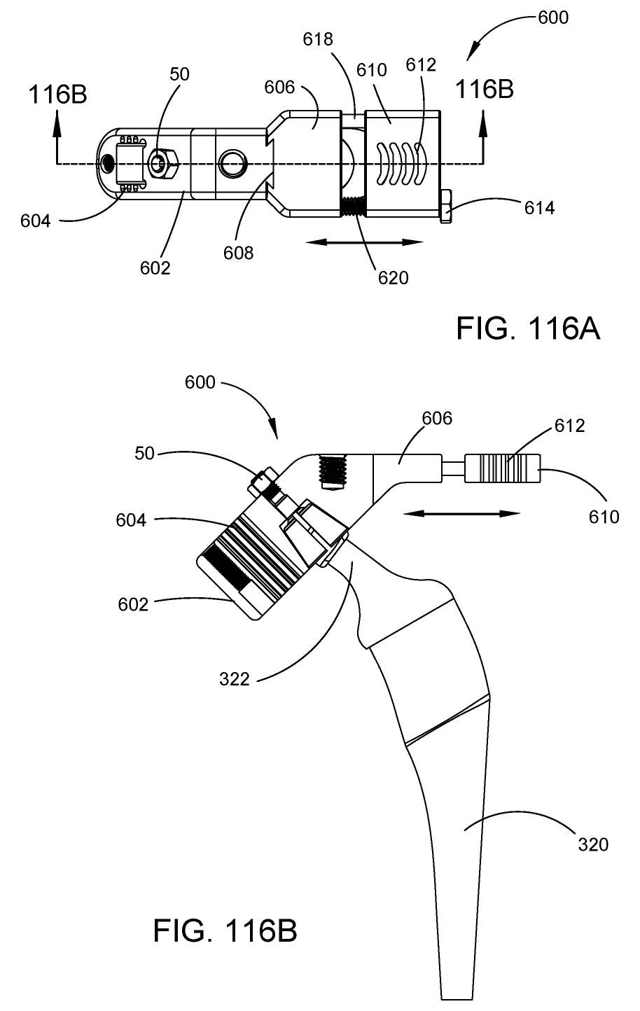

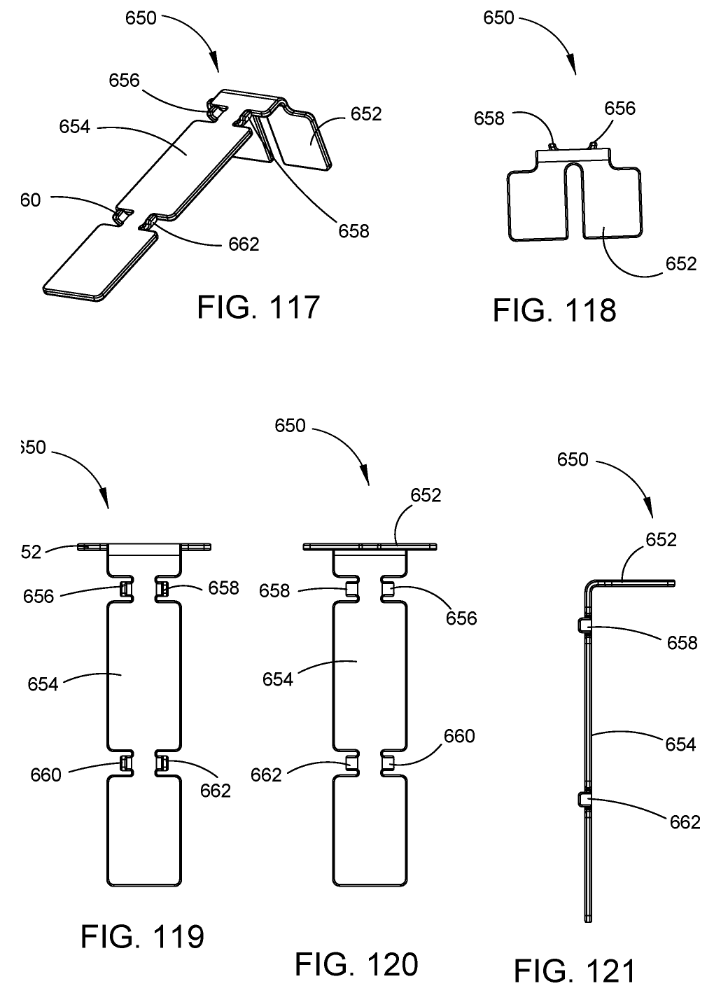

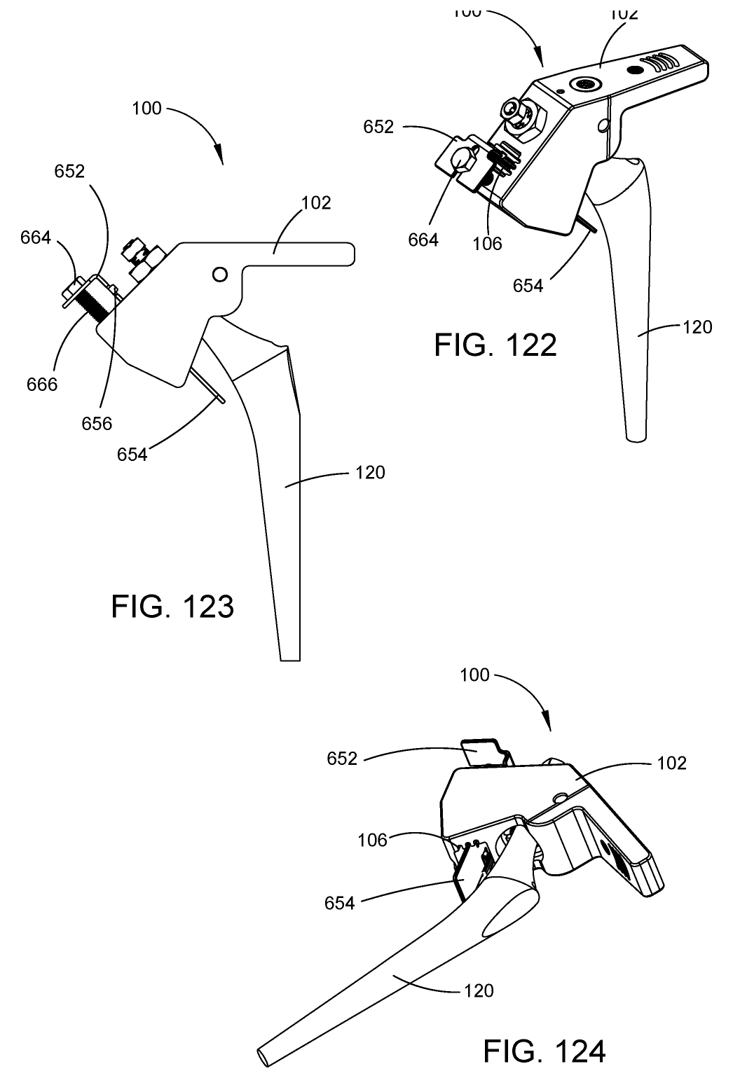

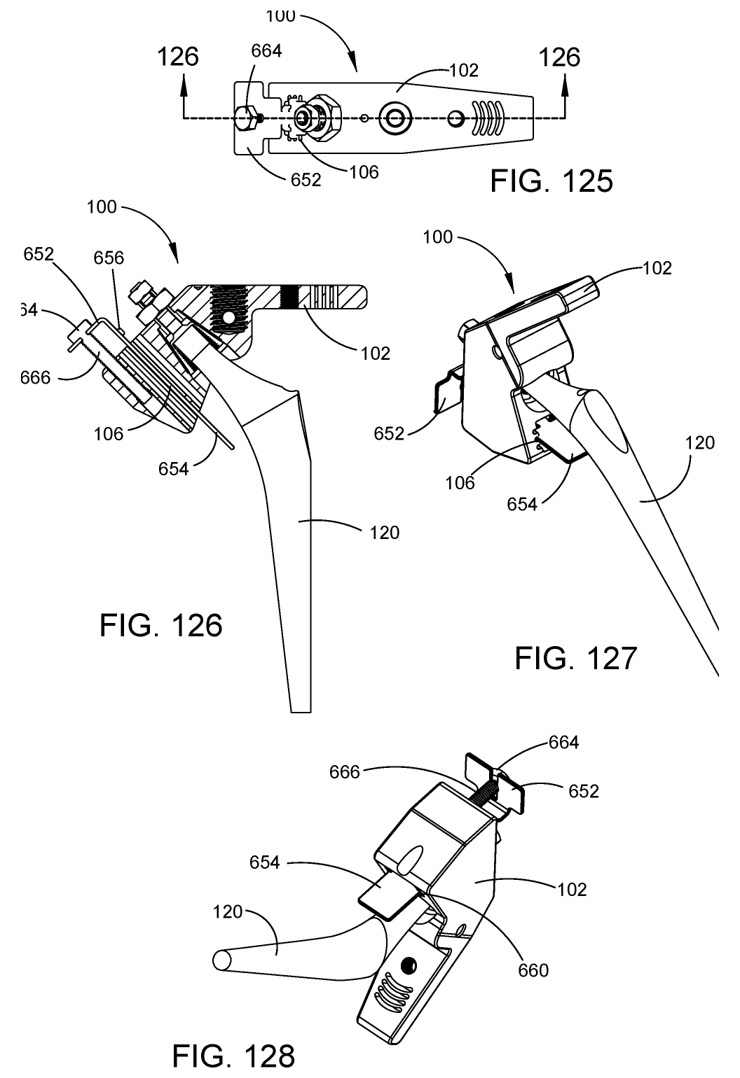

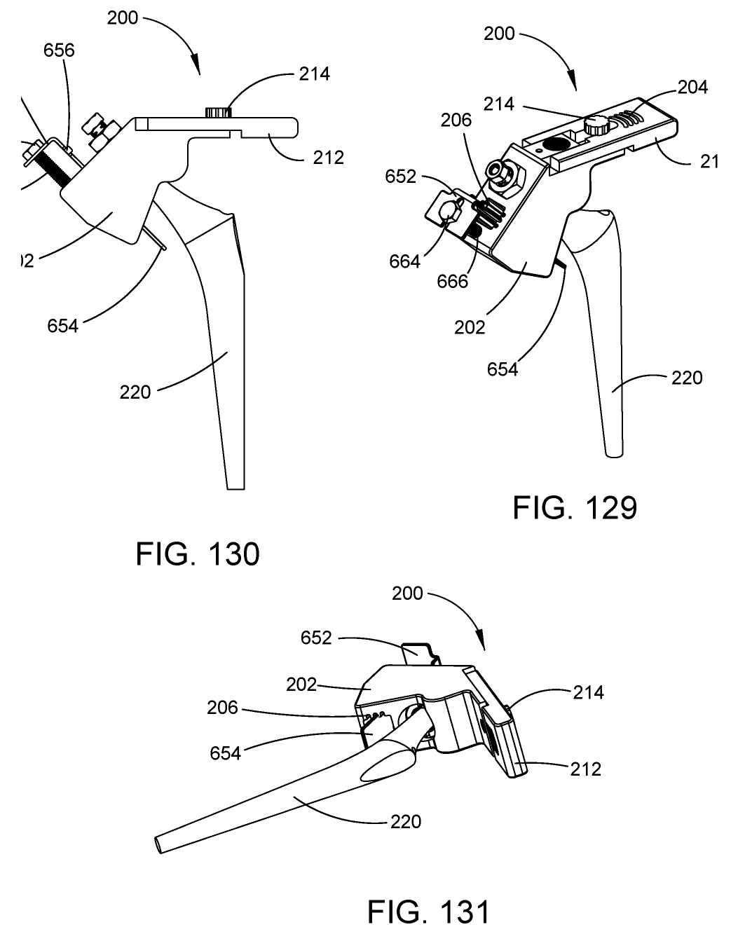

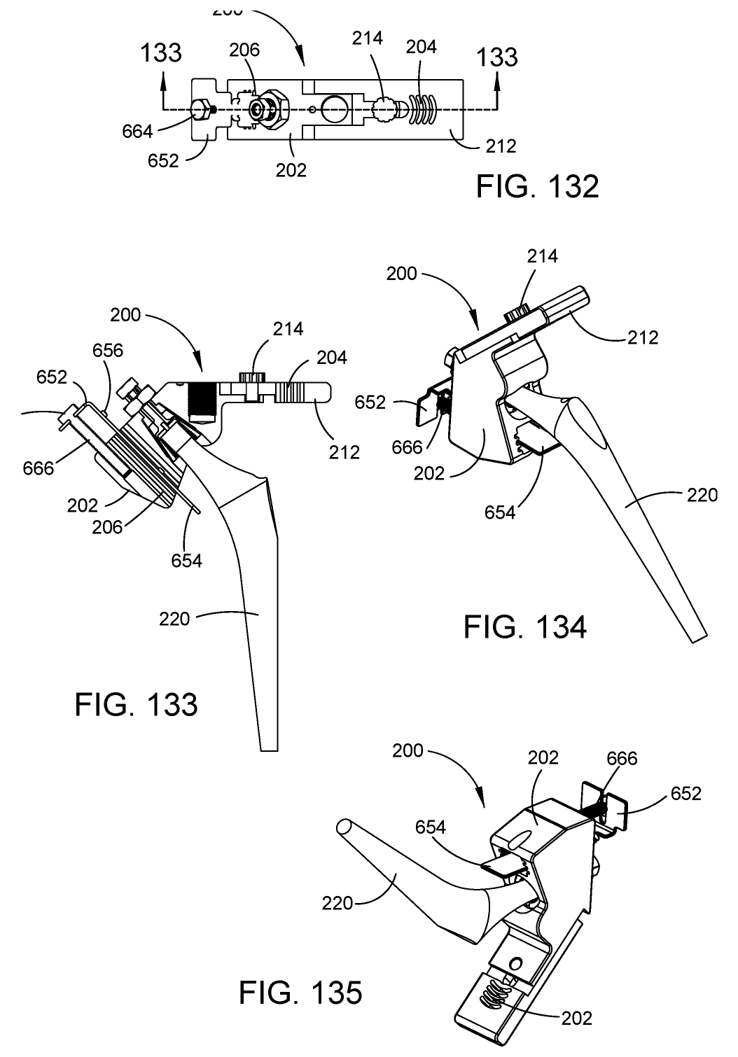

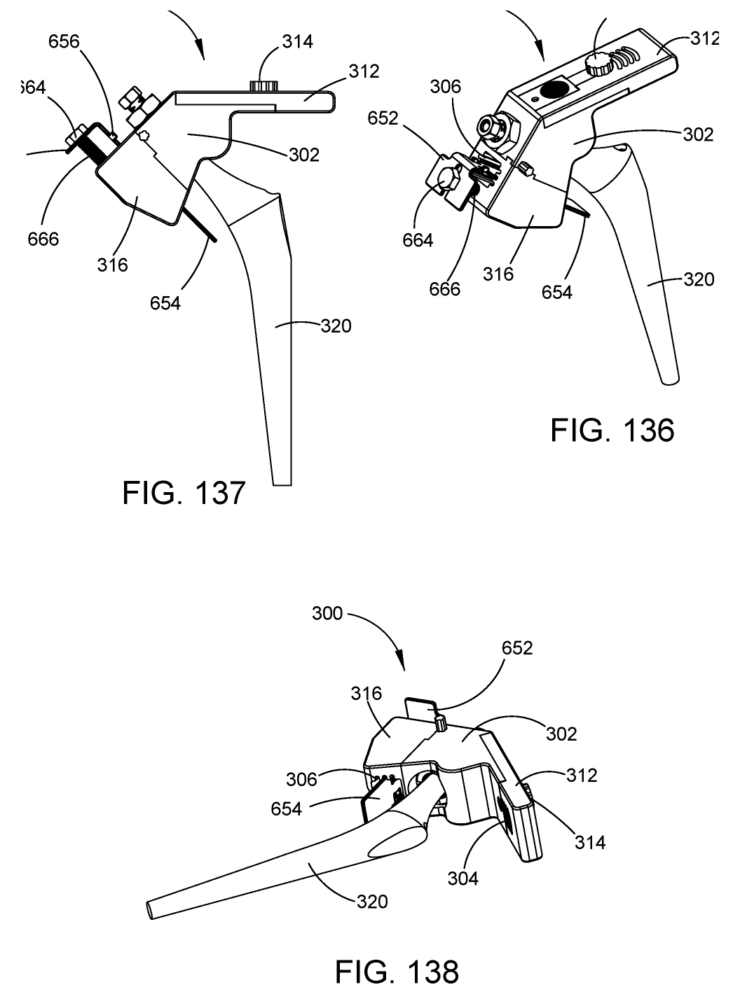

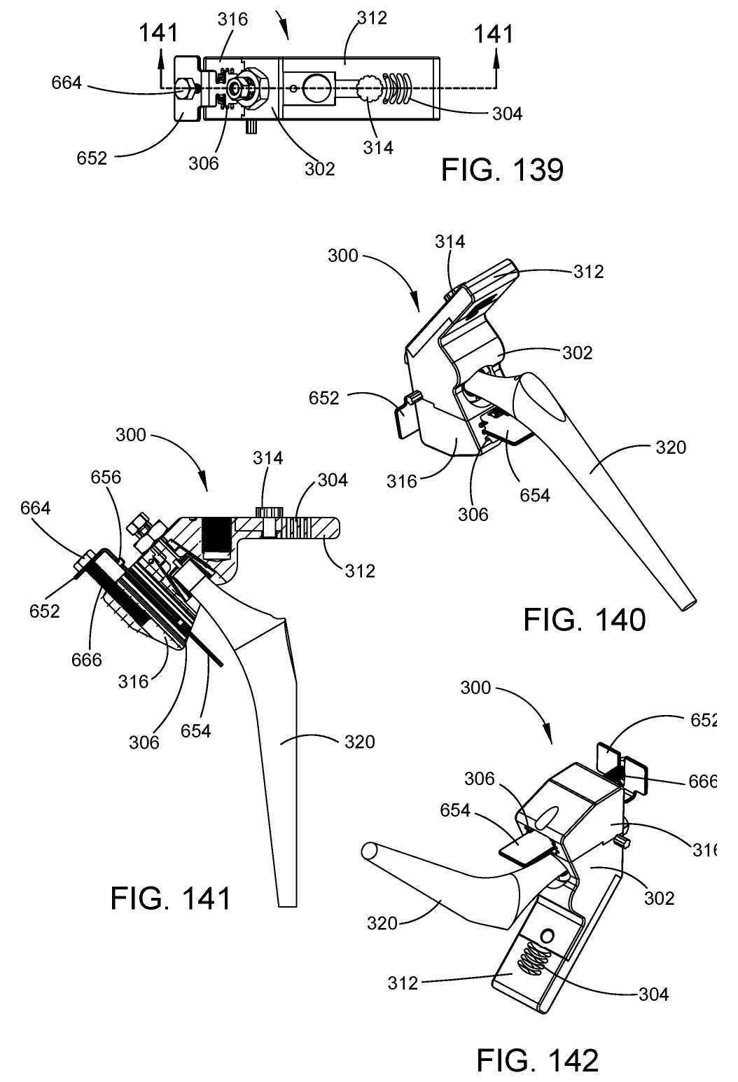

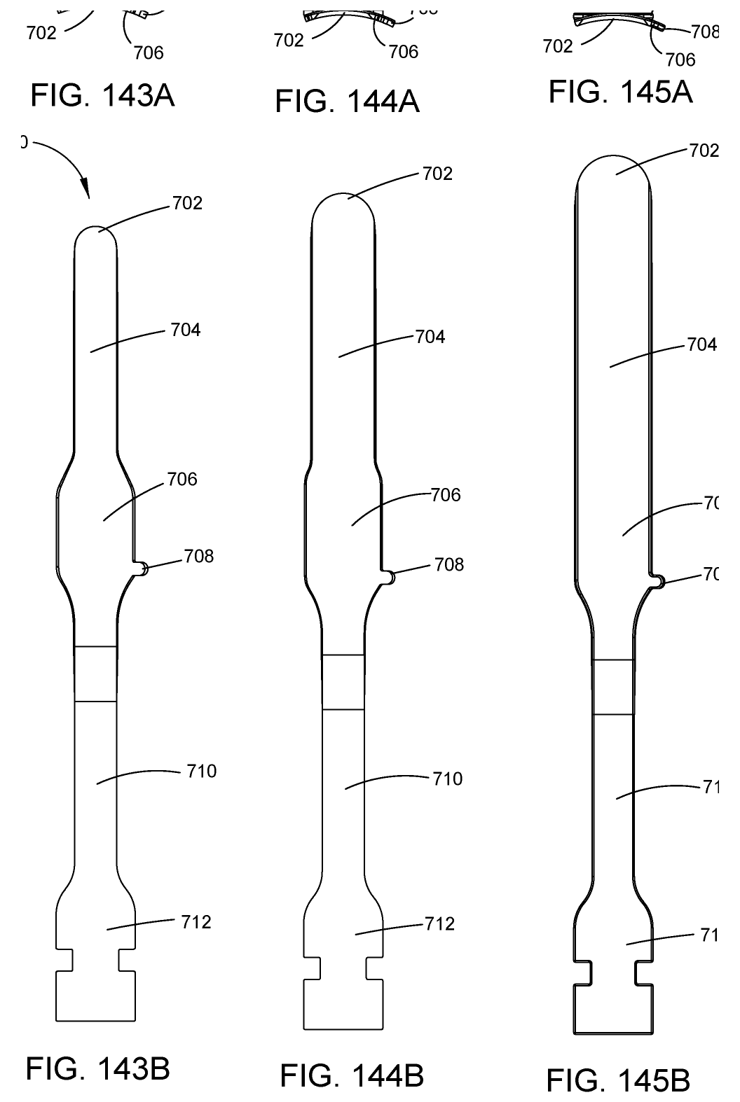

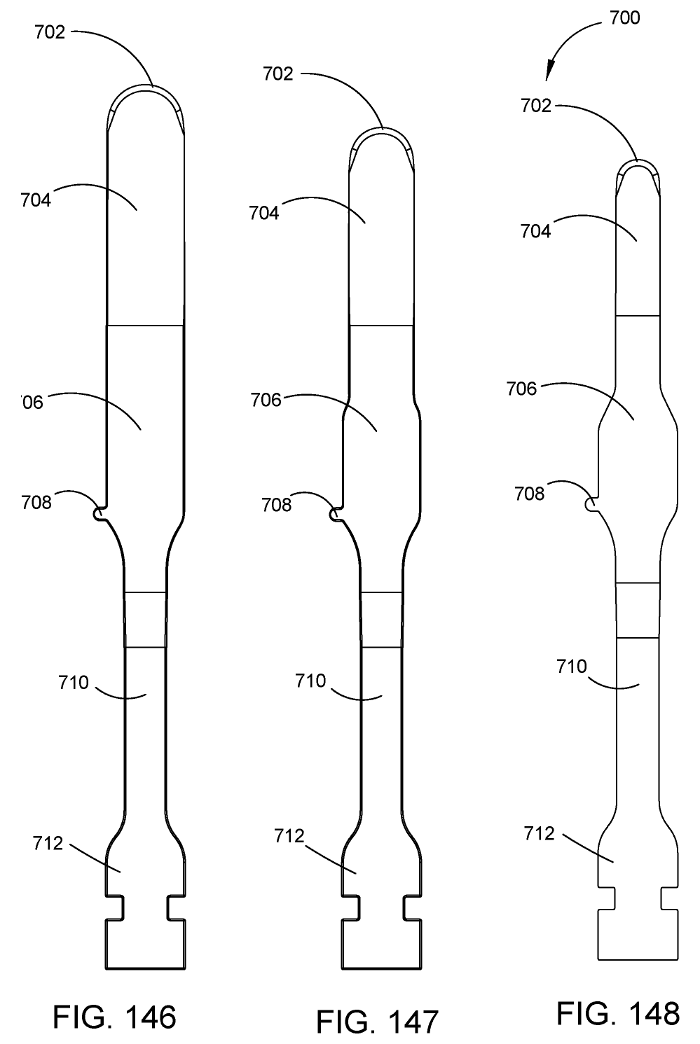

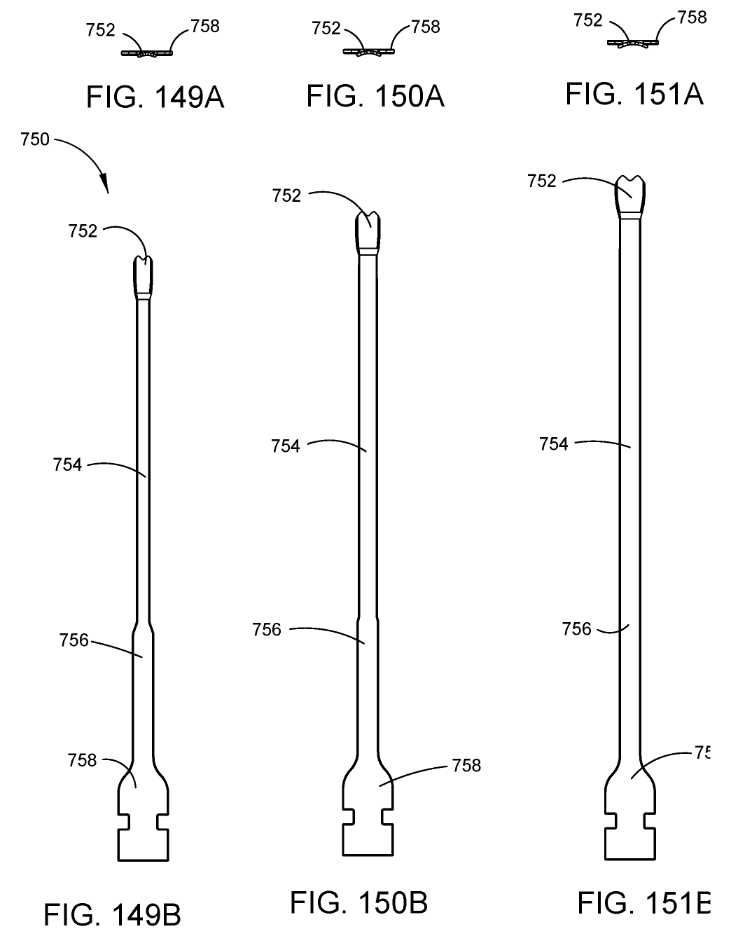

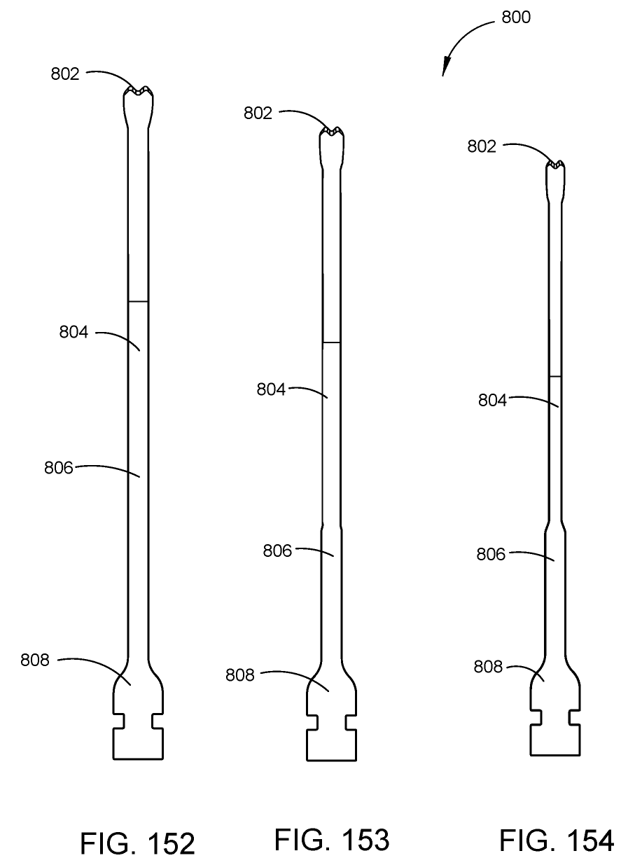

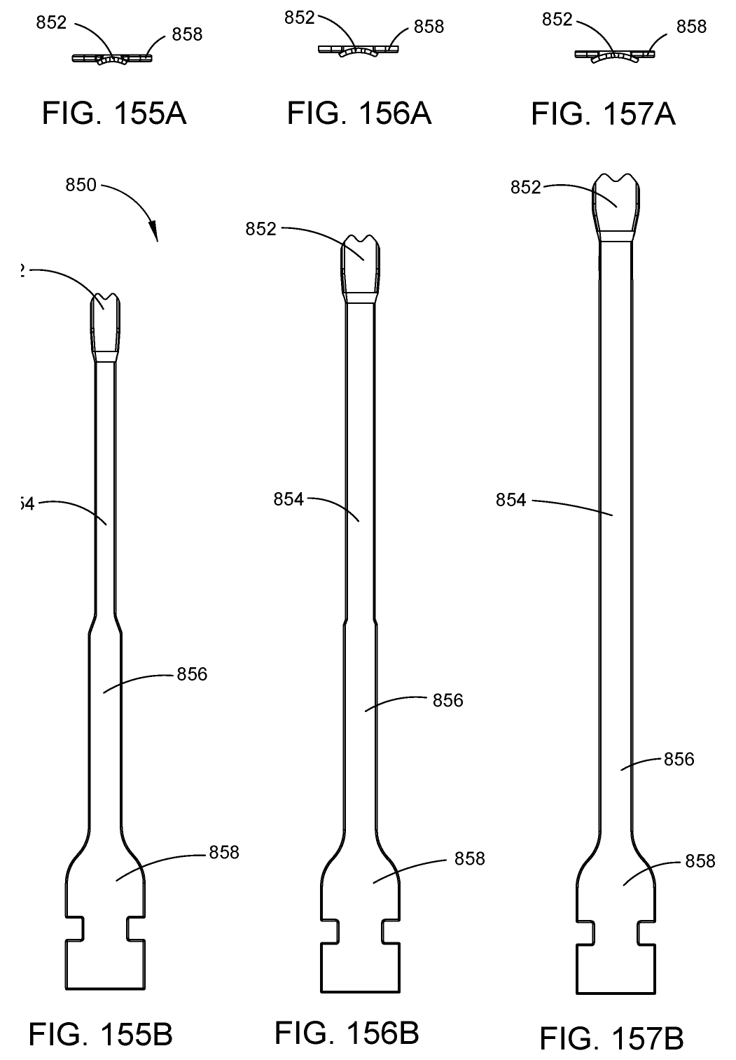

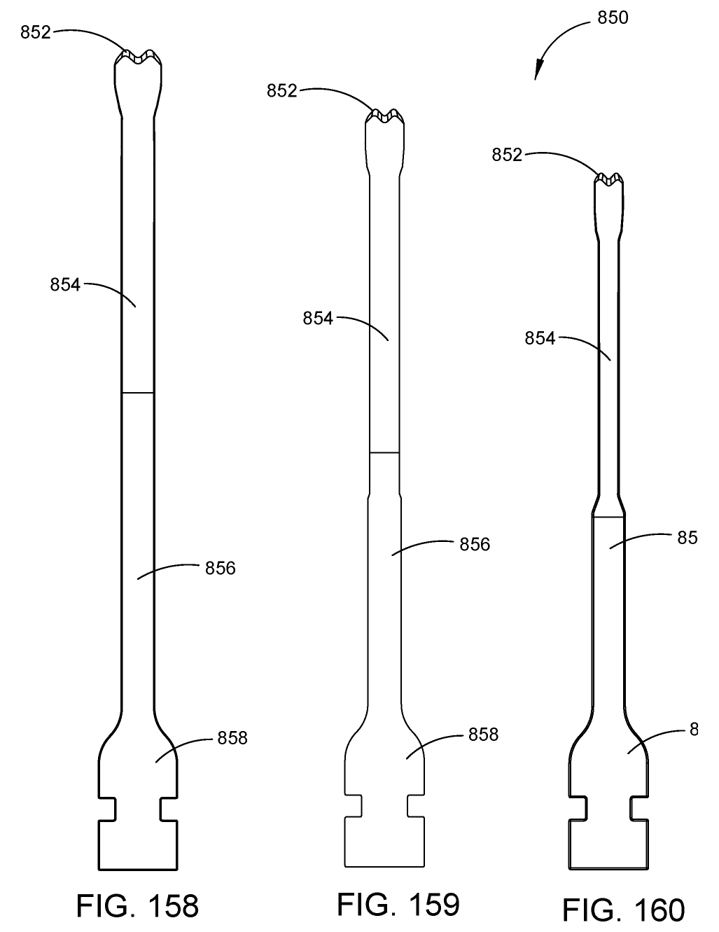

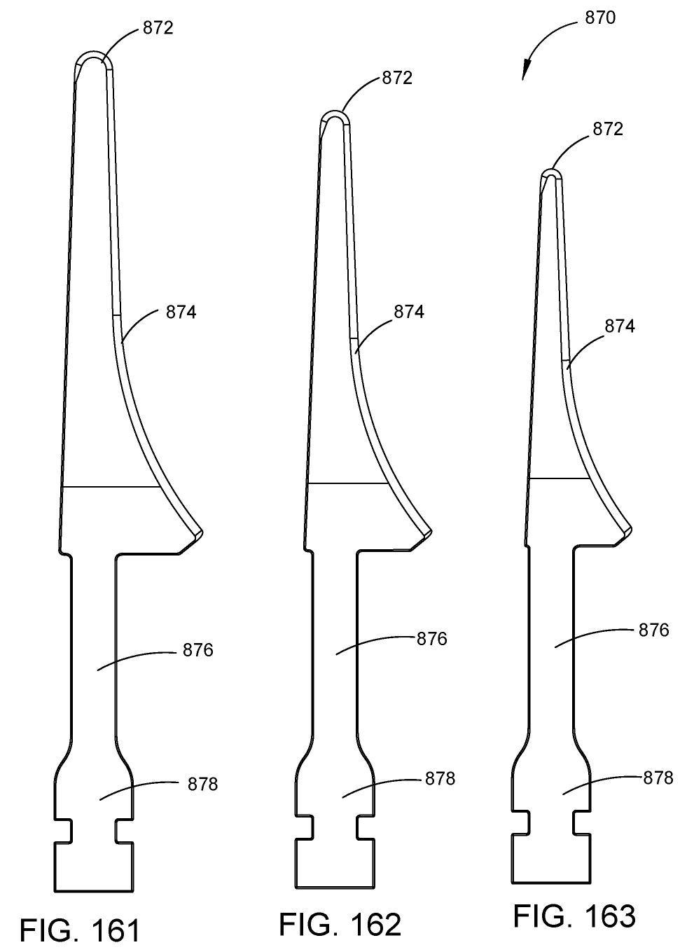

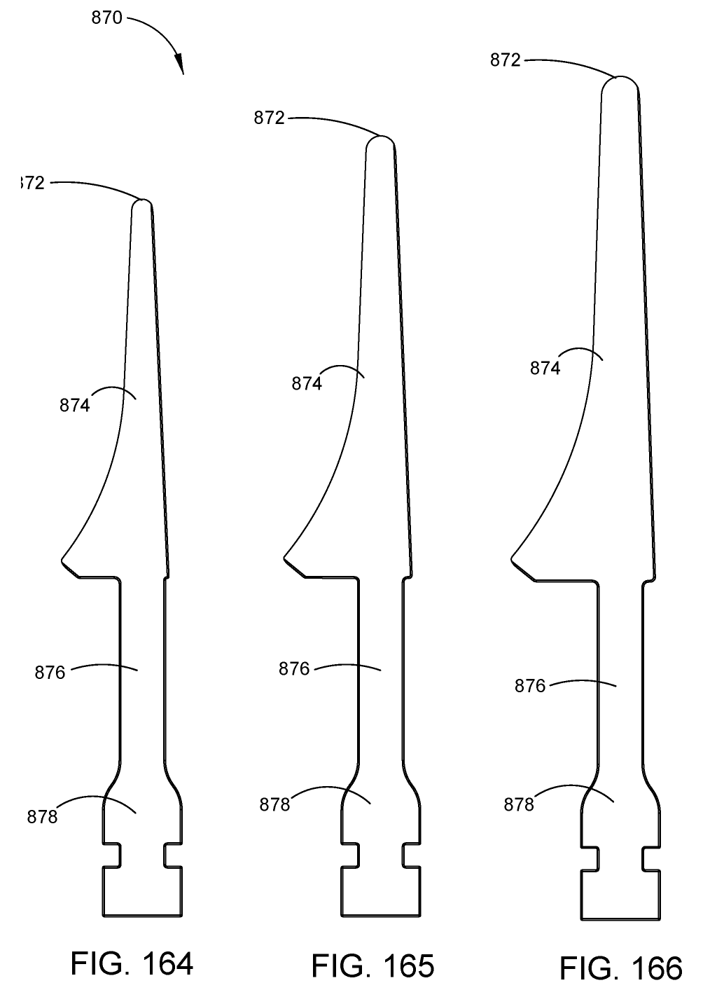

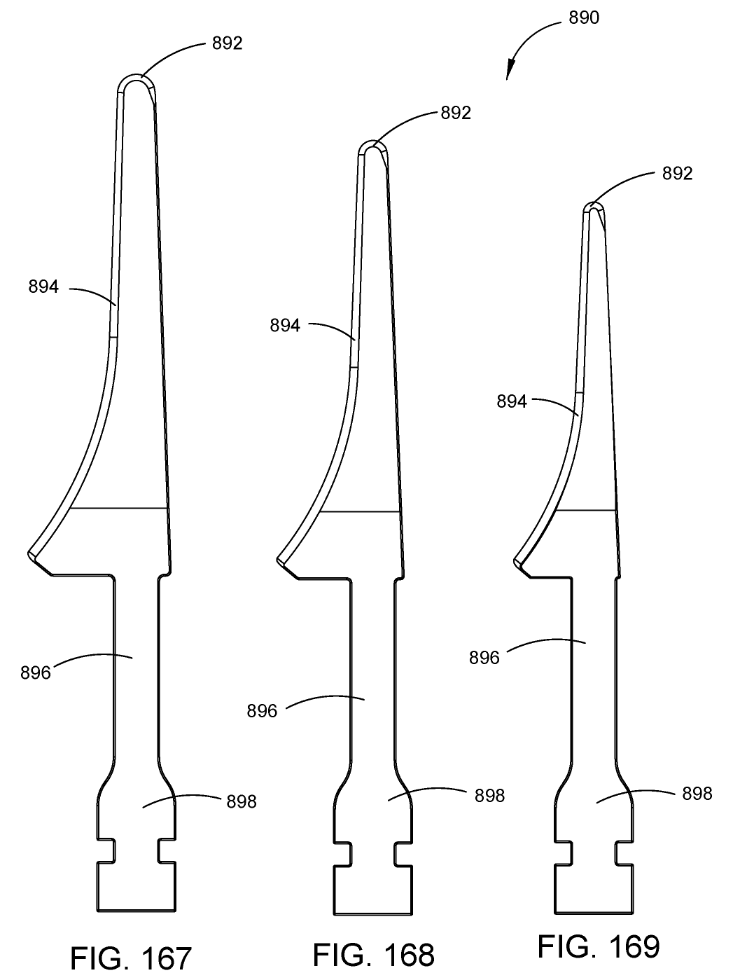

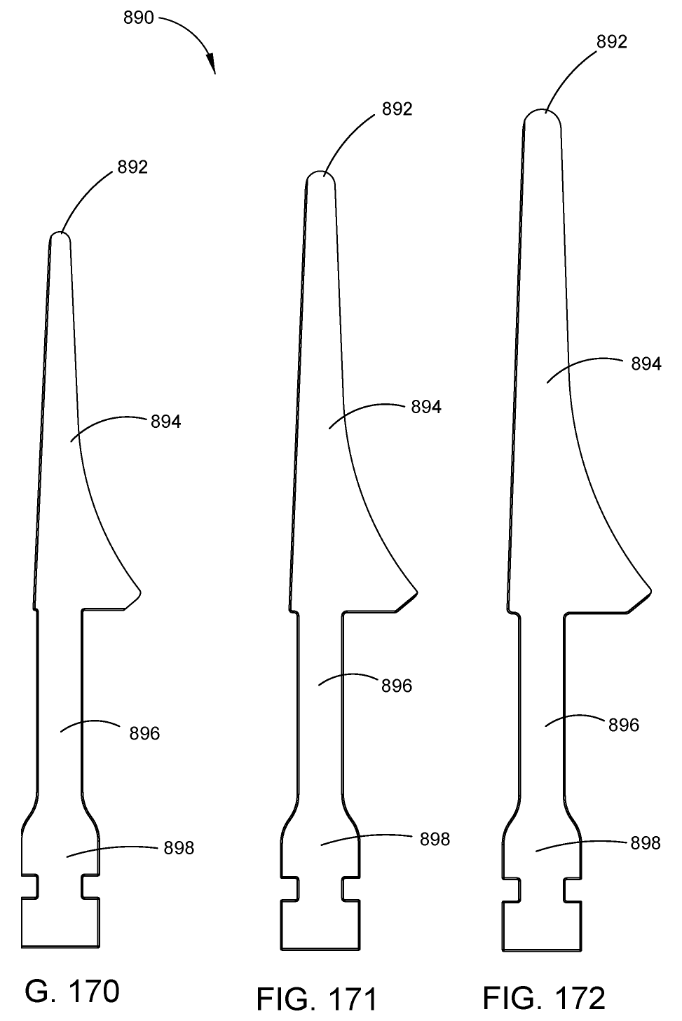

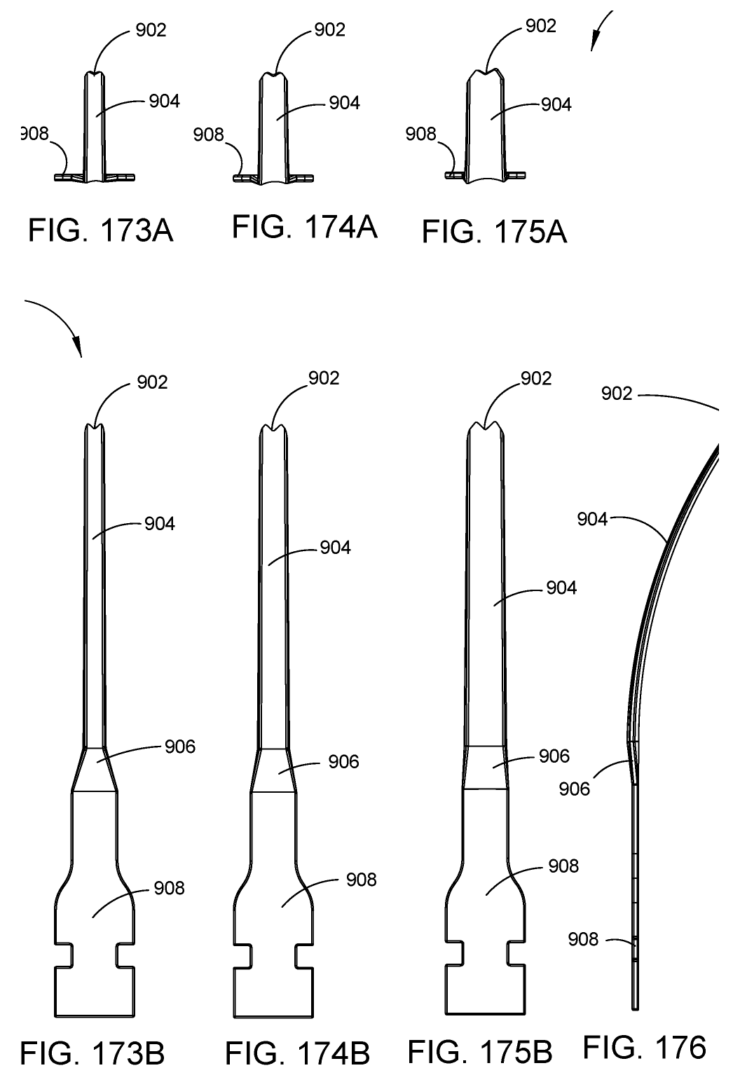

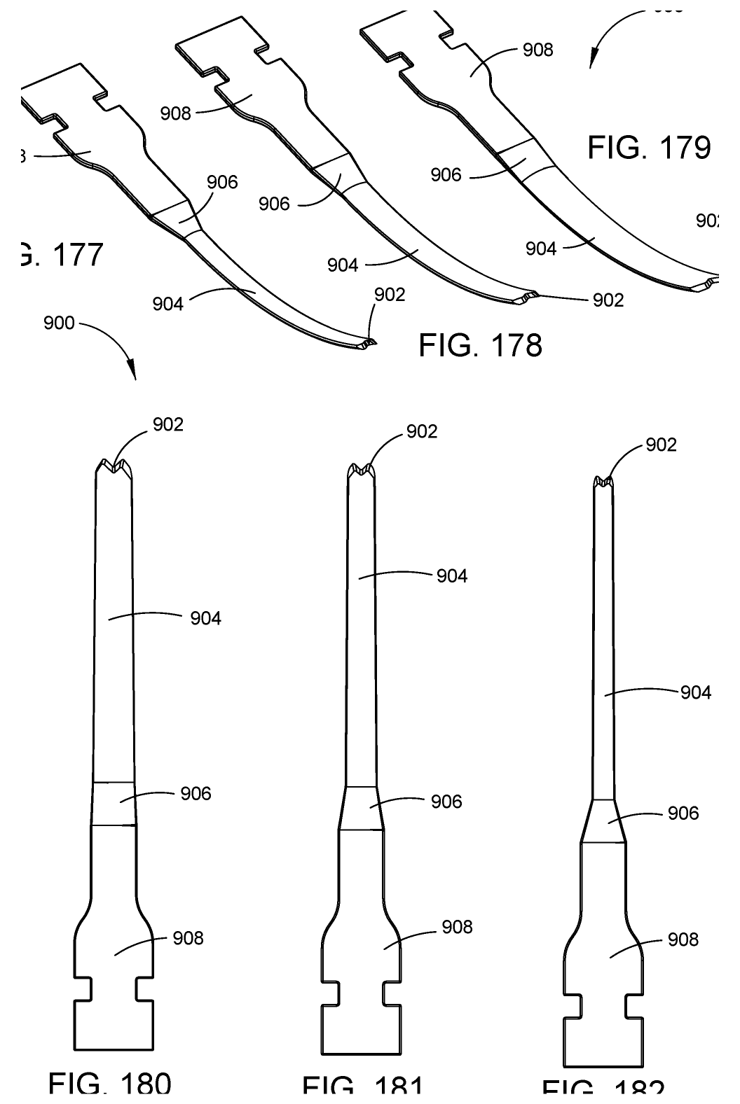

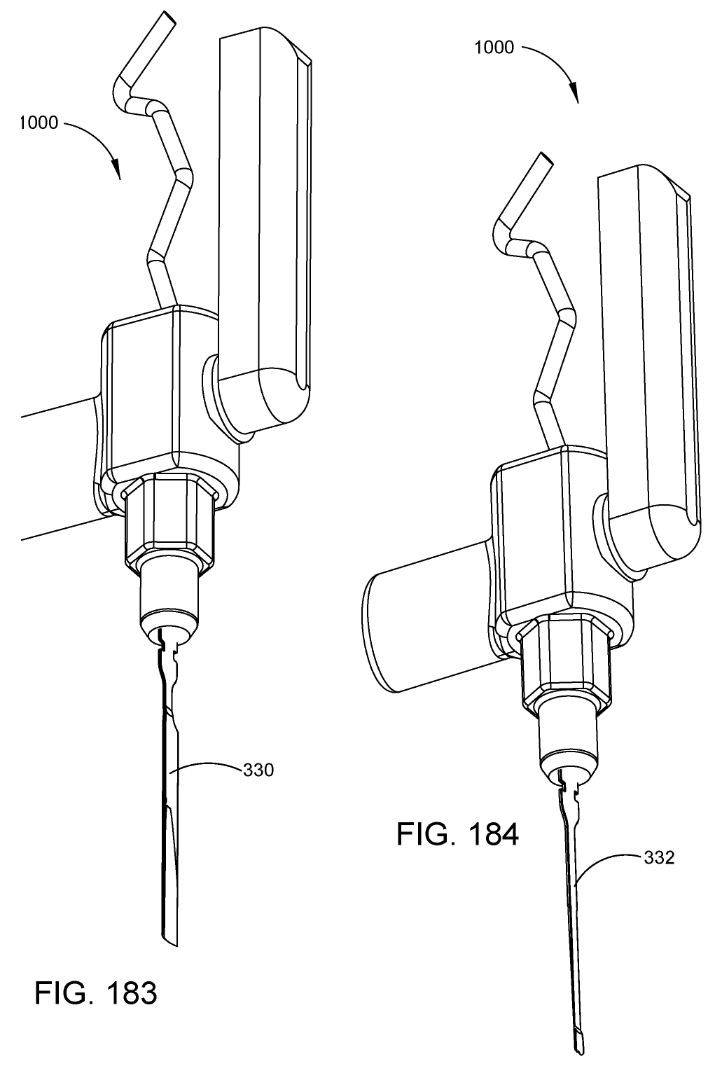

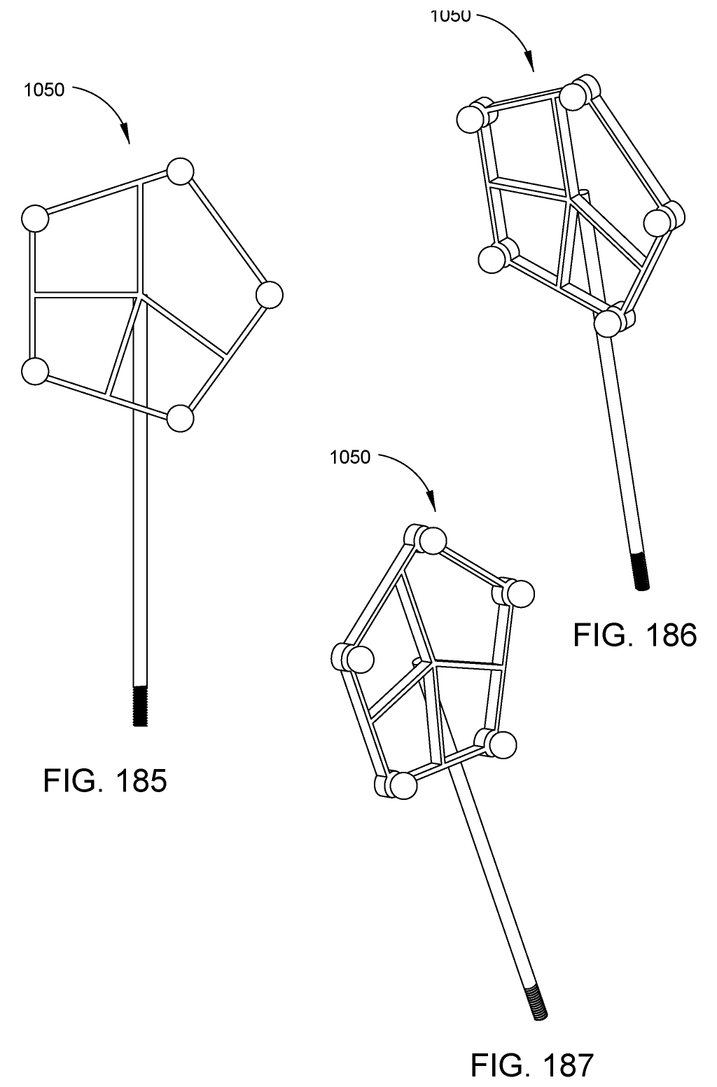

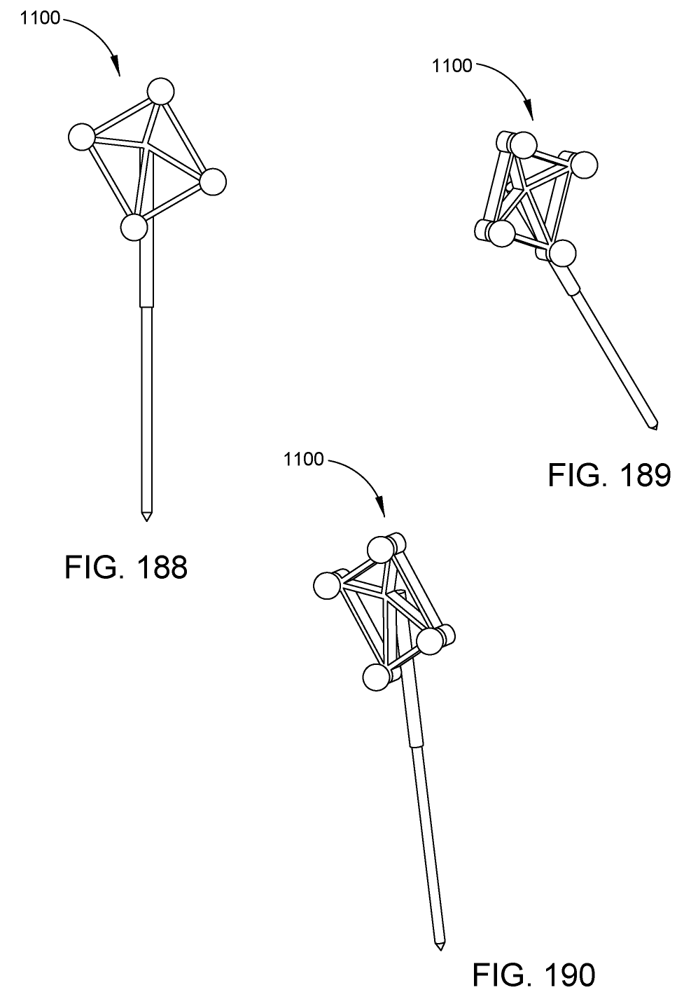

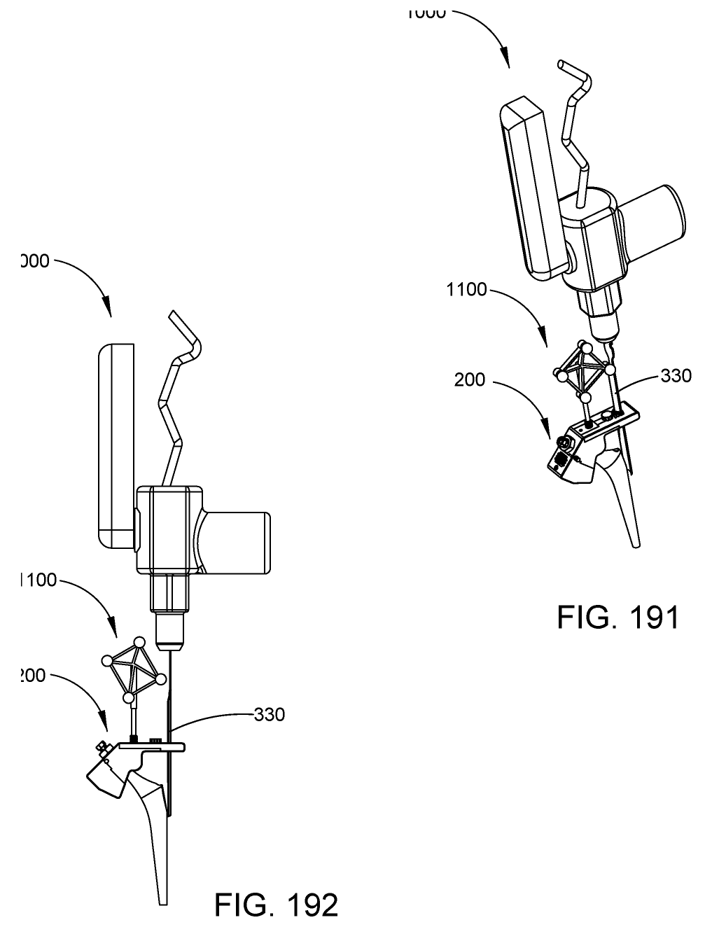

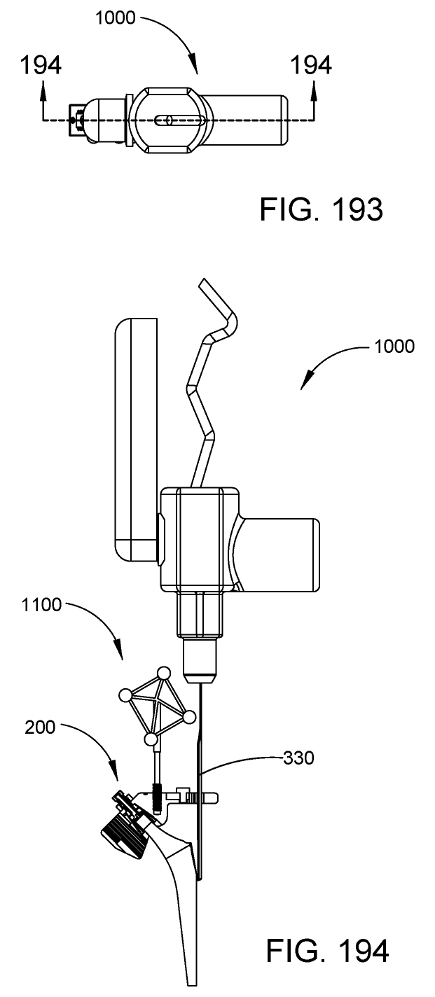

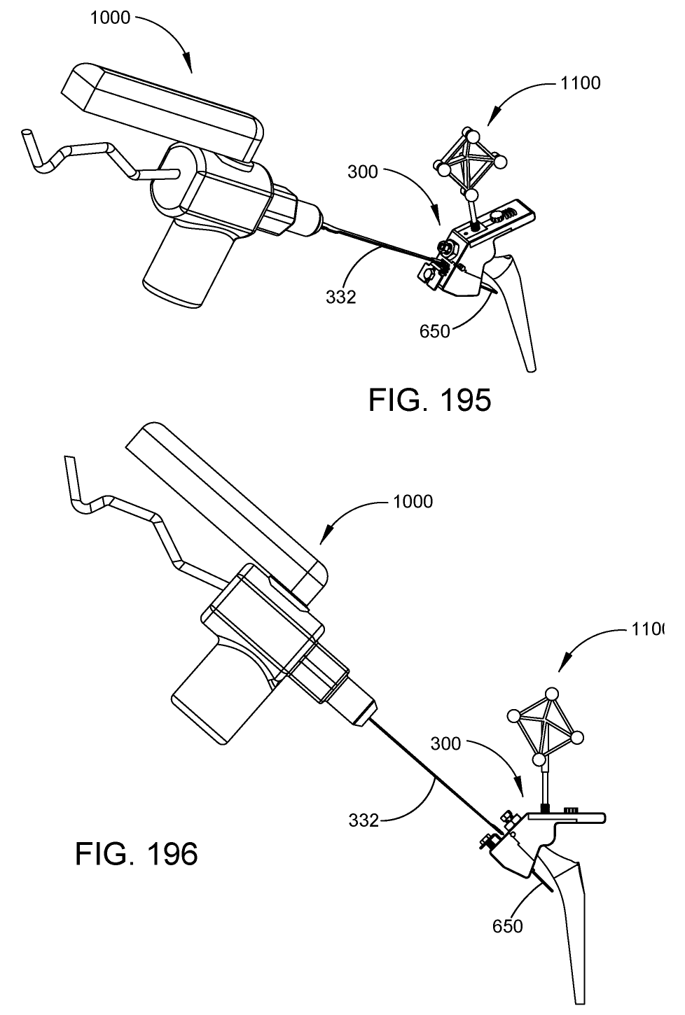

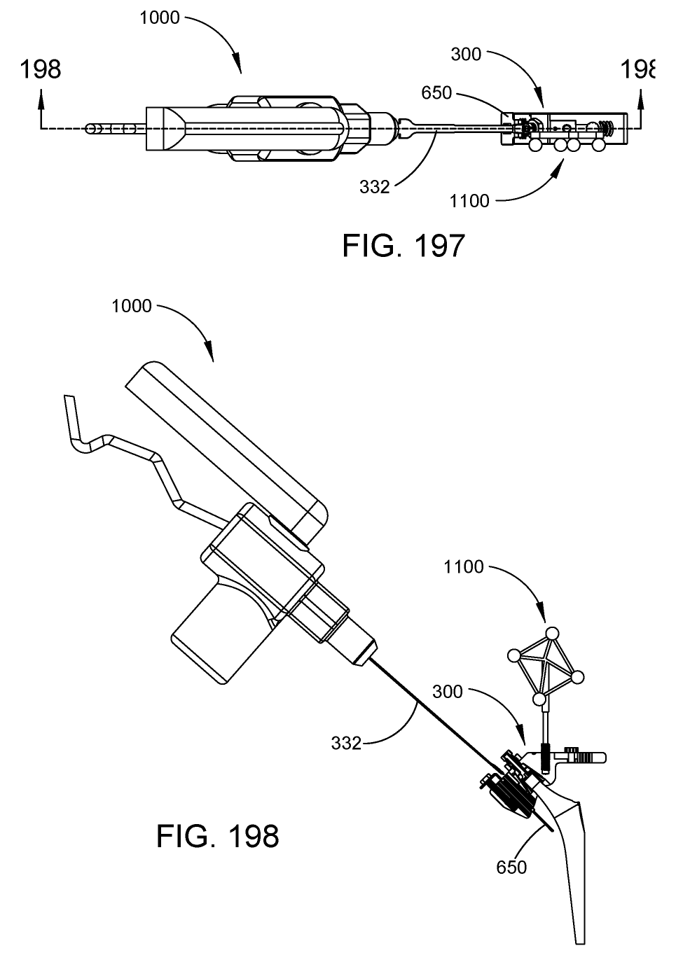

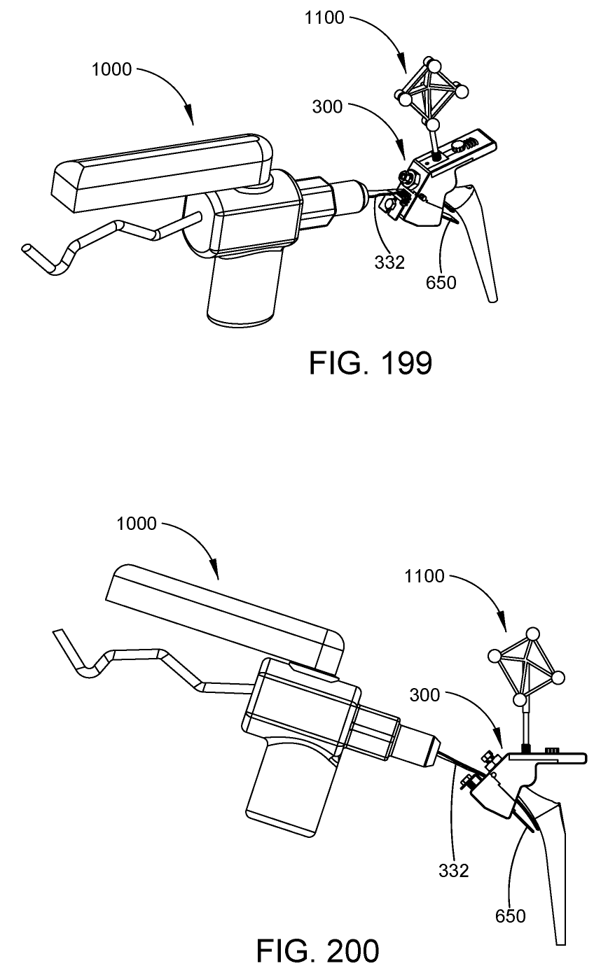

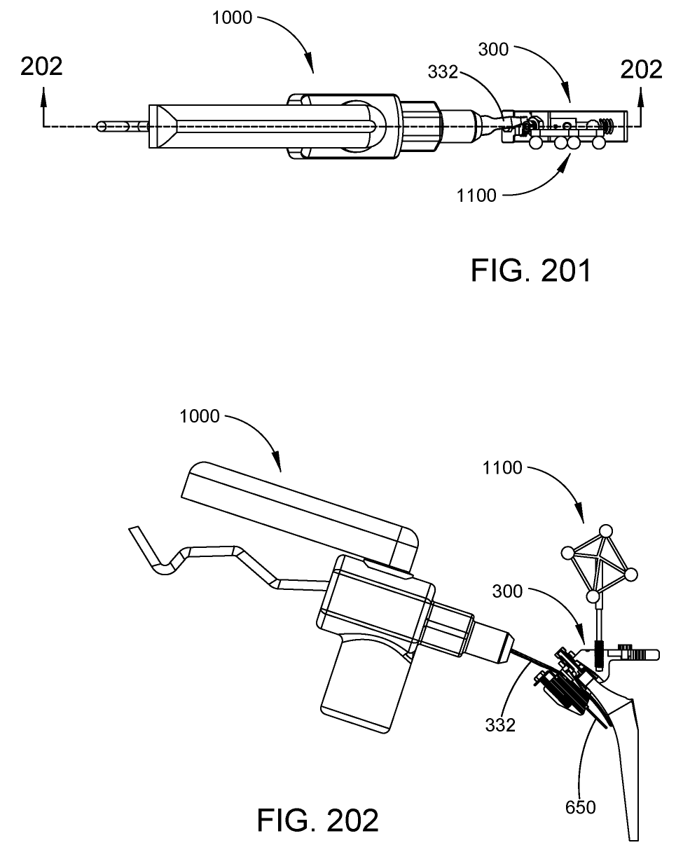

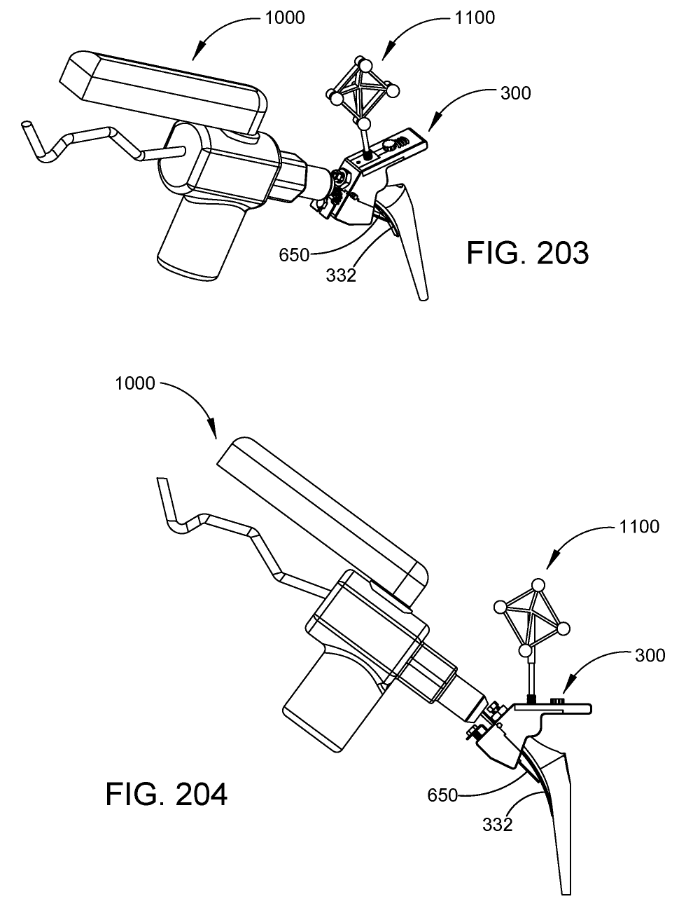

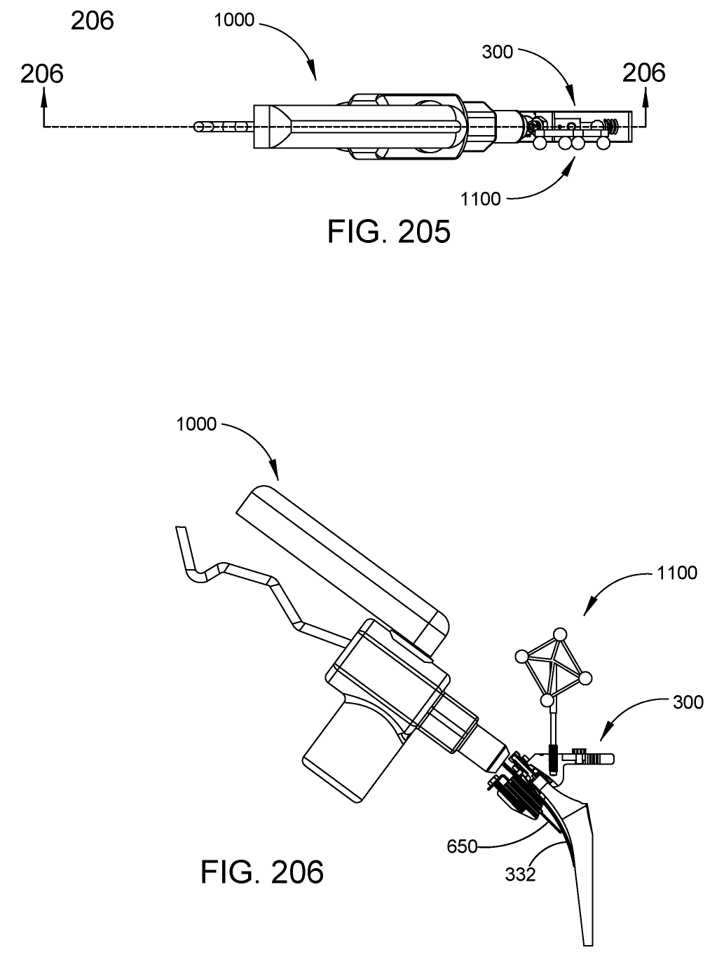

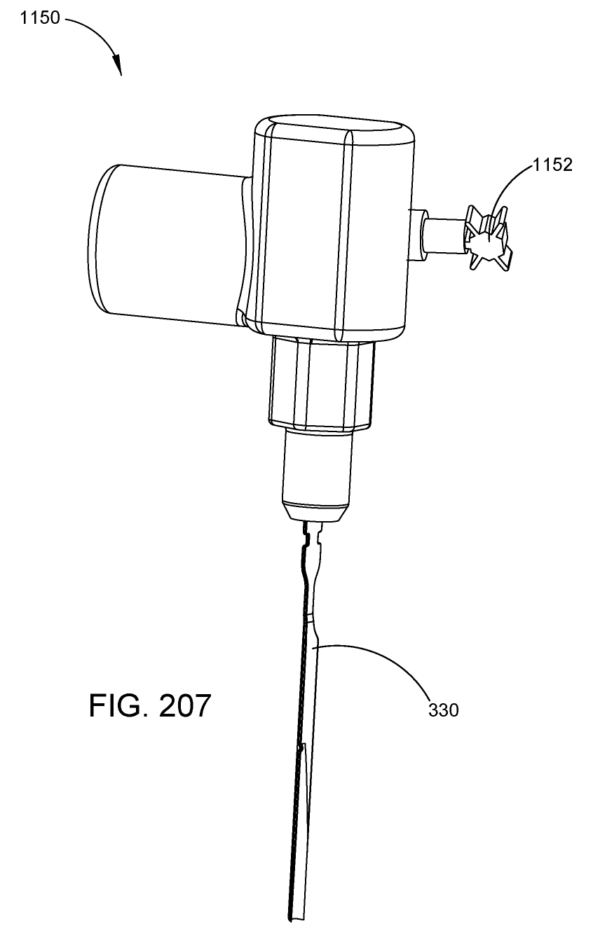

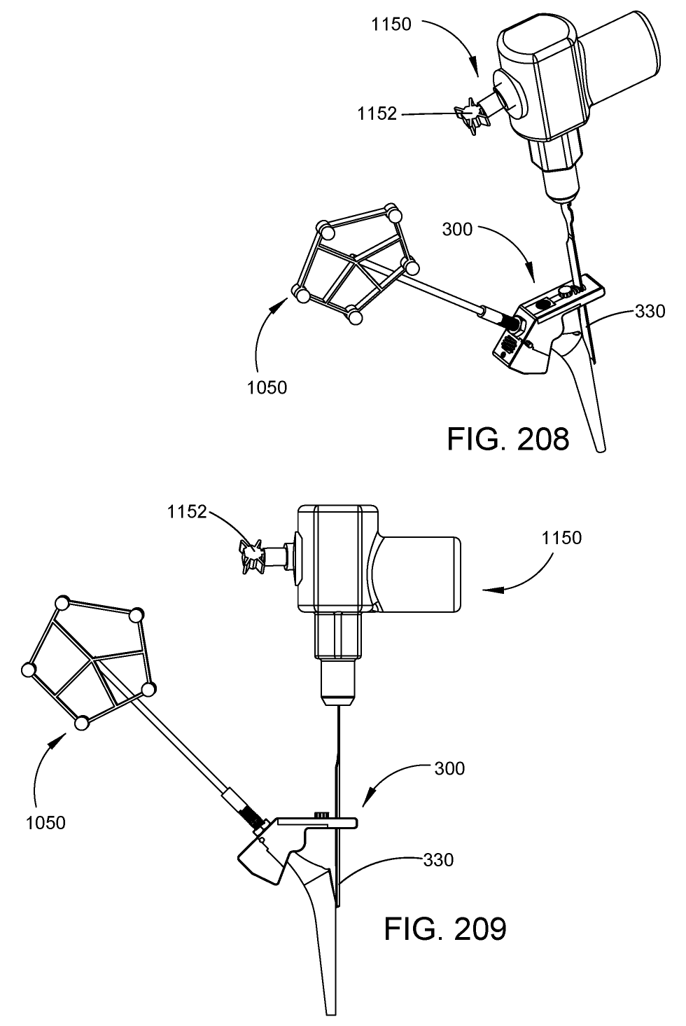

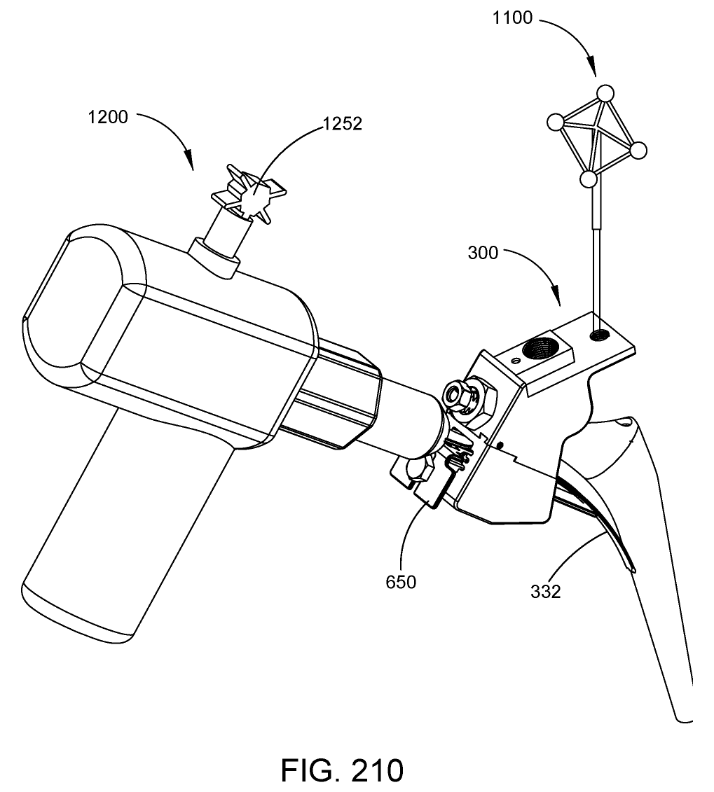

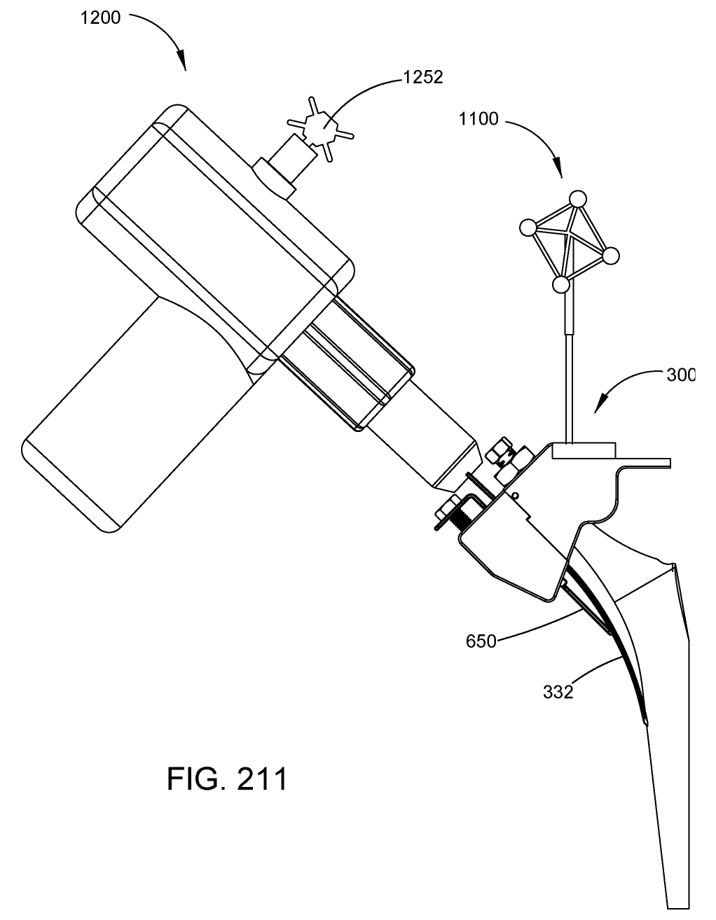

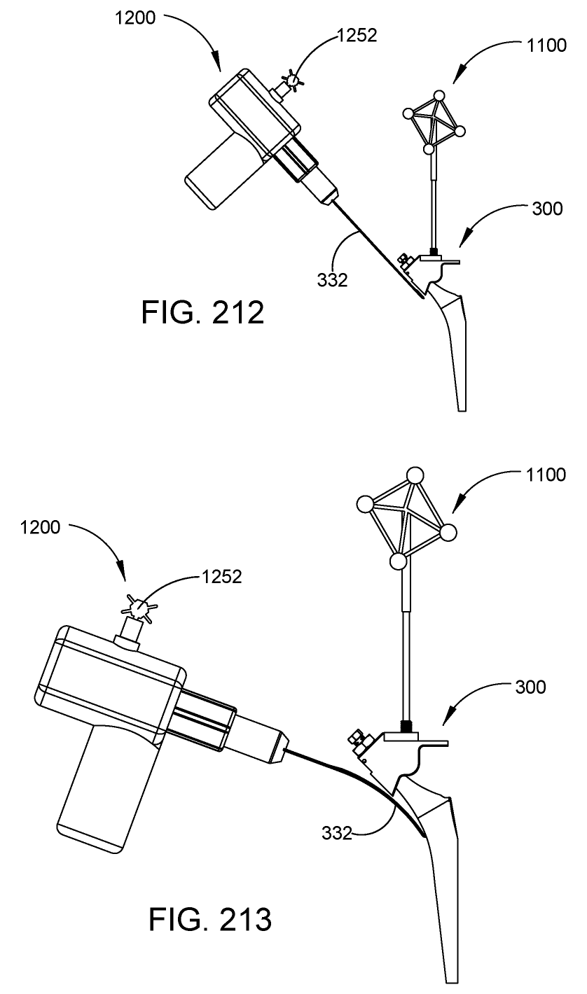

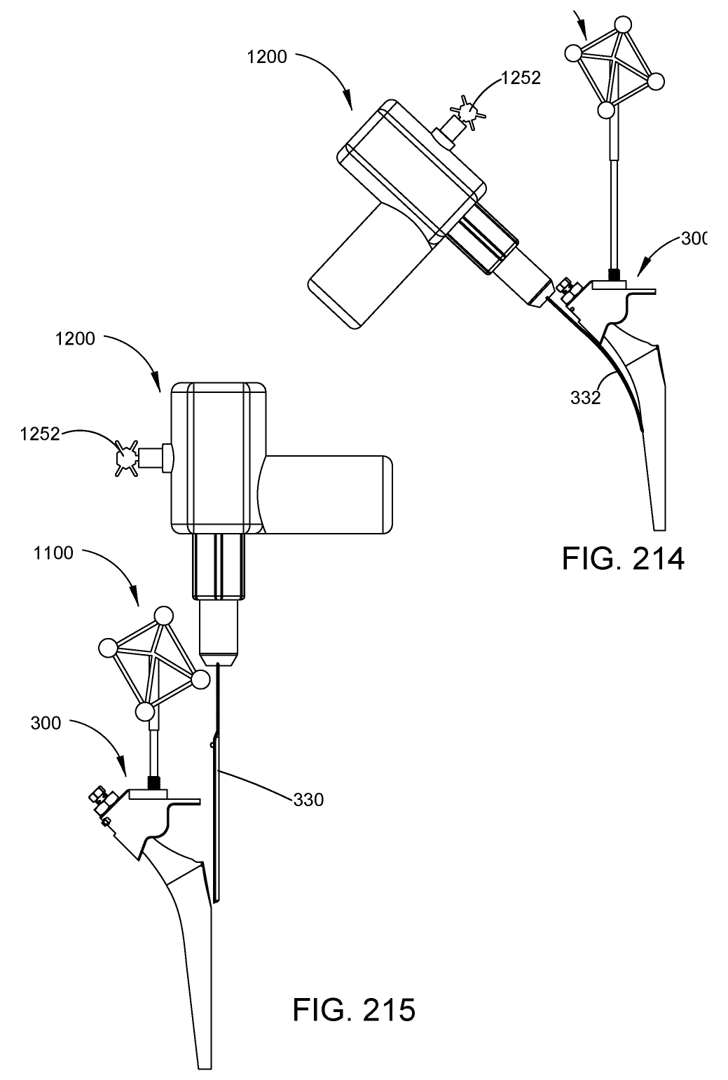

FIG. 12 depicts a typical femoral implant configuration and describes the various parts and portions thereof, illustrating in particular the metaphyseal/diaphyseal junction (MDJ) which will be discussed in detail in relation to the guide block apparatus system improvements described below. The parts of the conventional femoral stem from top to bottom include a trunnion, a neck, an insertion/extraction point, a shoulder, a body, a medial surface and a lateral surface, a metaphyseal/diaphyseal junction (MDJ) located on the lateral surface, and a lower stem portion. Depending on the orientation within the left or right hip, the anterior/posterior sides are delineated. Femoral implants can be cemented into place or cementless where bone grows into the porous or rough surface of the implant. The surface of the body may be rough for cementless implants, as is shown here. And the implant may or may not have a collar, as shown this implant does not have a collar. These femoral stem implant parts will be referred to throughout this specification.

### 1-PIECE

FIG. 13 depicts a top, side elevational and perspective view of a monolithic 1-piece blade guide block having an additional fixation point feature. The monolithic 1-piece blade guide block 100 is comprised of the following common parts: a guide block body 102, a trunnion securing member assembly 50, a plurality of lateral blade slots 104, a plurality of medial blade slots 106, a fixation screw knob 108, and a fixation screw threaded portion 110. The monolithic 1-piece blade guide block 100 is shown affixed to a femoral implant 120 by both the trunnion securing member assembly 50 and the fixation screw 108 and 110 portions which affix to the implant insertion/extraction point 122. The purpose of this FIG. 13 is to show that the blade guide block 100 can be constructed of 1 monolithic piece body 102, and it may have at least one additional fixation points over and above the trunnion fixation, here in the form of a fixation screw 108 and 110 which affixes to the implant insertion/extraction point 124 located at the base of the implant neck 122. Firm fixation to the implant to be extracted is essential in revision surgery. Having two fixation points significantly stabilizes the blade guide 100 block to the implant trunnion and implant insertion/extraction point 122, preventing unwanted movement of the blade guide block 100 during revision surgery. Additional stabilization of the blade guide block 100 also makes it possible to employ robotics and navigation technology to accomplish the revision surgery.

FIG. 14 depicts a side elevational view of the monolithic 1-piece blade guide block having an additional fixation point feature as shown in FIG. 13. Again, as in FIG. 13, the monolithic 1-piece blade guide block 100 is comprised of the following common parts: a guide block body 102, a trunnion securing member assembly 50, a plurality of lateral blade slots 104, a plurality of medial blade slots 106, a fixation screw knob 108, and a fixation screw threaded portion 110. The monolithic 1-piece blade guide block 100 is shown affixed to a femoral implant 120 by both the trunnion securing member assembly 50 and the fixation screw 108 and 110 portions which affix to the implant insertion/extraction point 124 located at the base of the implant neck 122. The purpose of this FIG. 14 is again to show that the blade guide block 100 can be constructed of 1 monolithic piece body 102, and it may have at least one additional fixation points over and above the trunnion fixation, here in the form of a fixation screw 108 and 110 which affixes to the implant insertion/extraction point 122. Firm fixation to the implant to be extracted is essential in revision surgery. Having two fixation points significantly stabilizes the blade guide 100 block to the implant trunnion and implant insertion/extraction point 122, preventing unwanted movement of the blade guide block 100 during revision surgery. Additional stabilization of the blade guide block 100 also makes it possible to employ robotics and navigation technology to accomplish the revision surgery.

FIG. 15 depicts a top plan view of the monolithic 1-piece blade guide block having an additional fixation point feature as shown in FIG. 13. Again, as in FIG. 14, the monolithic 1-piece blade guide block 100 is comprised of the following common parts: a guide block body 102, a trunnion securing member assembly 50, a plurality of lateral blade slots 104, a plurality of medial blade slots 106, a fixation screw knob 108, and a fixation screw threaded portion 110. The monolithic 1-piece blade guide block 100 is shown affixed to a femoral implant 120 by both the trunnion securing member assembly 50 and the fixation screw 108 and 110 portions which affix to the implant insertion/extraction point 124 located at the base of the implant neck 122. The purpose of this FIG. 15 is again to show that the blade guide block 100 can be constructed of 1 monolithic piece body 102, and it may have at least one additional fixation points over and above the trunnion fixation, here in the form of a fixation screw 108 and 110 which affixes to the implant insertion/extraction point 122. Firm fixation to the implant to be extracted is essential in revision surgery. Having two fixation points significantly stabilizes the blade guide 100 block to the implant trunnion and implant insertion/extraction point 122, preventing unwanted movement of the blade guide block 100 during revision surgery. Additional stabilization of the blade guide block 100 also makes it possible to employ robotics and navigation technology to accomplish the revision surgery.

FIG. 16 depicts a cross-sectional view of the monolithic 1-piece blade guide block 100 having an additional fixation point feature as shown in FIG. 15. The purpose of this FIG. 16 is to show how the threaded portion 110 of the fixation screw 108 and 110 can be tightened down to the implant insertion/extraction point 124 by passing through female threads located in the blade guide block 100 body 102.

FIG. 17 depicts a top, side elevational and perspective view of a monolithic 1-piece blade guide block 100 having a fixation point screw illustrating a rigid lateral guide blade guiding a flexible lateral cutting blade toward the lateral shoulder surface of the femoral stem to be extracted. The purpose of this FIG. 17 is to show how the rigid lateral guide blade 130 is inserted into one of the plurality of guide slots 104 and moved downward to contact the MDJ of the implant before acting to guide the flexible lateral cutting blade 132 down the lateral surface hugging the MDJ and effectively releasing the stem 120.

FIG. 18 depicts a side elevational view of the monolithic 1-piece blade guide block 100 having a fixation point screw shown in FIG. 17, illustrating a rigid lateral blade guiding a flexible lateral cutting blade toward the lateral shoulder surface of the femoral stem to be extracted. Again, as with FIG. 17, the purpose of this FIG. 18 is to show how the rigid lateral guide blade 130 is inserted into one of the plurality of guide slots 104 and moved downward to contact the MDJ of the implant before acting to guide the flexible lateral cutting blade 132 down the lateral surface hugging the MDJ and effectively releasing the stem 120.

FIG. 19 depicts a top plan view of the monolithic 1-piece blade guide block 100 having a fixation point screw 108 shown in FIG. 17, illustrating a rigid lateral blade 130 guiding a flexible lateral cutting blade 132 toward the lateral shoulder surface of the femoral stem to be extracted.

FIG. 20 depicts a cross-sectional view of the monolithic 1-piece blade guide block 100 having a fixation point screw 108 and 110 shown in FIG. 19, illustrating a rigid lateral blade 130 guiding a flexible lateral cutting blade 132 toward the lateral shoulder surface of the femoral stem 120 to be extracted. Here the rigid lateral blade 130 is all the way down in place to guide the flexible lateral cutting blade 132 which has advanced only part way toward the lateral shoulder surface of the femoral stem 120 to be extracted.

FIG. 21 depicts a top, side elevational and perspective view of a monolithic 1-piece blade guide block 100 having a fixation point screw 108 and 110 shown in FIG. 19, illustrating a rigid lateral blade 130 guiding a flexible lateral cutting blade 132 toward the lateral shoulder surface of the femoral stem 120 to be extracted. Here the rigid lateral blade 130 is all the way down in place to guide the flexible lateral cutting blade 132 which has advanced further down the lateral shoulder surface of the femoral stem 120 to be extracted. In this way, the rigid lateral blade 130 guides the flexible lateral cutting blade 132 to cut close to the lateral surface of the stem 120, past the MDJ 124 thereby preventing unwanted cortical bone penetration by accurate fine adjustment location guidance of the flexible blades, resulting in significant preservation of critical bone mass during the revision surgery.

FIG. 22 depicts a side elevational view of the monolithic 1-piece blade guide block 100 having a fixation point screw 108 and 110 shown in FIG. 21, illustrating a rigid lateral blade 130 guiding a flexible lateral cutting blade 132 toward the lateral shoulder surface of the femoral stem 120 to be extracted. Here again the rigid lateral blade 130 is all the way down in place to guide the flexible lateral cutting blade 132 which has advanced further down the lateral shoulder surface of the femoral stem 120 to be extracted. The purpose of this FIG. 22 is to show that the rigid lateral blade 130 guides the flexible lateral cutting blade 132 to cut close to the lateral surface of the stem 120, past the MDJ 124 thereby preserving critical bone mass during the revision surgery.

FIG. 23 depicts a top plan view of the monolithic 1-piece blade guide block 100 having a fixation point screw 108 and 110 shown in FIG. 21, illustrating a rigid lateral blade 130 guiding a flexible lateral cutting blade 132 toward the lateral shoulder surface of the femoral stem 120 to be extracted. Here again the rigid lateral blade 130 is all the way down in place to guide the flexible lateral cutting blade 132 which has advanced further down the lateral shoulder surface of the femoral stem 120 to be extracted. The purpose of this FIG. 23 is to show that the rigid lateral blade 130 guides the flexible lateral cutting blade 132 to cut close to the lateral surface of the stem 120, past the MDJ 124 thereby preventing unwanted cortical bone penetration by accurate fine adjustment location guidance of the flexible blades, resulting in significant preservation of critical bone mass during the revision surgery.

FIG. 24 depicts a cross-sectional view of the monolithic 1-piece blade guide block 100 having a fixation point screw 108 and 110 shown in FIG. 21, illustrating a rigid lateral blade 130 guiding a flexible lateral cutting blade 132 toward the lateral shoulder surface of the femoral stem 120 to be extracted. Here again the rigid lateral blade 130 is all the way down in place to guide the flexible lateral cutting blade 132 which has advanced further down the lateral shoulder surface of the femoral stem 120 to be extracted. The purpose of this FIG. 24 is to show that the rigid lateral blade 130 guides the flexible lateral cutting blade 132 to cut close to the lateral surface of the stem 120, past the MDJ 124 thereby preventing unwanted cortical bone penetration by accurate fine adjustment location guidance of the flexible blades, resulting in significant preservation of critical bone mass during the revision surgery.

### 2-PIECE

FIG. 25 depicts a top, side elevational and perspective view of a 2-piece modular blade guide block 200 illustrating a laterally movably adjustable piece 212 housing the lateral blade guide slots 204. The 2-piece modular blade guide block 200 is comprised of the following common parts: a universal central guide block body 202, a trunnion securing member assembly 50, a plurality of lateral blade slots 204, a plurality of medial blade slots 206, an adjustable lateral modular portion 212 and an adjustment screw knob 214. Here, the modular 2-piece blade guide block 200 is shown affixed to a femoral implant 220. The purpose of this FIG. 25 is to show that the 2-piece modular blade guide block 200 can be constructed of a universal central guide block body 202 as well as a laterally adjustable second piece 212, which acts to change the distance between the guide slots 204 and the lateral surface of the stem 220 to allow for the guidance of flexible lateral cutting blades by rigid lateral blades (see below).

FIG. 26 depicts a side elevational view of the 2-piece modular blade guide block 200 illustrating a laterally movably adjustable piece 212 housing the lateral blade guide slots 204 as shown in FIG. 25. Again as in FIG. 25, the 2-piece modular blade guide block 200 is comprised of the following common parts: a universal central guide block body 202, a trunnion securing member assembly 50, a plurality of lateral blade slots 204, a plurality of medial blade slots 206, an adjustable lateral modular portion 212 and an adjustment screw knob 214. Here, the modular 2-piece blade guide block 200 is shown affixed to a femoral implant 220. The purpose of this FIG. 25 is to show that the 2-piece modular blade guide block 200 can be constructed of a universal central guide block body 202 as well as a laterally adjustable second piece 212, which acts to change the distance between the guide slots 204 and the lateral surface of the stem 220 to allow for the guidance of flexible lateral cutting blades by rigid lateral blades (see figures below).

FIG. 27 depicts a top plan view of the 2-piece modular blade guide block 200 illustrating a laterally movably adjustable piece 212 housing the lateral blade guide slots 204 as shown in FIG. 25.

FIG. 28 depicts a cross-sectional view of the 2-piece modular blade guide block 200 illustrating a laterally movably adjustable piece 212 housing the lateral blade guide slots 204 as shown in FIG. 27. The purpose of this FIG. 28 is to show that the 2-piece modular blade guide block 200 illustrating a laterally movably adjustable piece 212 housing the lateral blade guide slots 204 is to show that this configuration has one fixation point, namely, the trunnion securing assembly 50. For a description of optional additional fixation points, please see those related figures and descriptions below.

FIG. 29 depicts a side elevational view of the 2-piece modular blade guide block 200 illustrating a laterally movably adjustable piece 212 housing the lateral blade guide slots 204, showing the laterally movably adjustable piece 212 moving outwardly laterally, see direction arrows, here shown moved moderately outward laterally. In operation, the adjustment screw 214 would be loosened, then the laterally movably adjustable piece 212 would slide along the universal central body piece 202 to a desired position before being tightened down and secured in the desired lateral position. Differing sized implants will require that the laterally movably adjustable piece 212 be adjusted for optimal guiding by rigid lateral blades and cutting by flexible guided lateral cutting blades (see below) for the differing sizes of the femoral implant such that the lateral surface of the stem 220 is closely cut to both prevent unwanted cortical bone penetration by accurate fine adjustment location guidance of the flexible blades and to preserve critical bone mass during revision surgery.

FIG. 30 depicts a side elevational view of the 2-piece modular blade guide block 200 illustrating a laterally movably adjustable piece 212 housing the lateral blade guide slots 204, showing the laterally movably adjustable piece 212 moving outwardly laterally, see direction arrows, here shown moved outward laterally to the full extent of motion.

FIG. 31 depicts a top plan view of the 2-piece modular blade guide block 200 illustrating a laterally movably adjustable piece 212 housing the lateral blade guide slots 204, showing the laterally movably adjustable piece 212 moving outwardly laterally, see direction arrows, here shown moved outward laterally to the full extent of motion.

FIG. 32 depicts a cross-sectional view of the 2-piece modular blade guide block 200 illustrating a laterally movably adjustable piece 212 housing the lateral blade guide slots 204, showing the laterally movably adjustable piece 212 moving outwardly laterally, see direction arrows, here shown moved outward laterally to the full extent of motion as shown in FIG. 31.

FIG. 33 depicts an exploded perspective view of the 2-piece modular blade guide block 200 illustrating a laterally movably adjustable piece 212 housing the lateral blade guide slots 204. Again as in FIG. 32, the 2-piece modular blade guide block 200 is comprised of the following common parts: a universal central guide block body 202, a trunnion securing member assembly 50, a plurality of lateral blade slots 204, a plurality of medial blade slots 206, an adjustable lateral modular portion 212 and an adjustment screw knob 214. Additionally seen in this exploded view in FIG. 33 are the parts of the trunnion securing assembly 50 comprising the securing nut 246, the stem trunnion securing member 50 has a partially hollow body 258, having a ribbed inner portion 256 running roughly half way from the large diameter to the small diameter proximal to the threaded portion 248. It was previously disclosed that trunnion securing members come in varying sized trunnion adapters so as to fit any size implant trunnion as the original and first fixation point.

FIG. 34 depicts an exploded side elevational view of the 2-piece modular blade guide block 200 illustrating a laterally movably adjustable piece 212 housing the lateral blade guide slots 204. Again as in FIG. 33, the 2-piece modular blade guide block 200 is comprised of the following common parts: a universal central guide block body 202, a trunnion securing member assembly 50, a plurality of lateral blade slots 204, a plurality of medial blade slots 206, an adjustable lateral modular portion 212 and an adjustment screw knob 214.

Additionally seen in this exploded view in FIG. 33 are the parts of the trunnion securing assembly 50 comprising the securing nut 246, the stem trunnion securing member 50 has a partially hollow body 258, having a ribbed inner portion 256 running roughly half way from the large diameter to the small diameter proximal to the threaded portion 248.

FIG. 35 depicts a partially exploded top plan view of the 2-piece modular blade guide block 200 illustrating a laterally movably adjustable piece 212 housing the lateral blade guide slots 204. The 2-piece modular blade guide block 200 is comprised of the following common parts: a universal central guide block body 202, a trunnion securing member assembly 50, a plurality of lateral blade slots 204, a plurality of medial blade slots 206, an adjustable lateral modular portion 212 and an adjustment screw knob 214.

FIG. 36 depicts a fully exploded cross-sectional view of the 2-piece modular blade guide block illustrating a laterally movably adjustable piece housing the lateral blade guide slots as shown in FIG. 35. 200 illustrating a laterally movably adjustable piece 212 housing the lateral blade guide slots 204. Again, as in FIG. 34, the 2-piece modular blade guide block 200 is comprised of the following common parts: a universal central guide block body 202, a trunnion securing member assembly 50, a plurality of lateral blade slots 204, a plurality of medial blade slots 206, an adjustable lateral modular portion 212 and an adjustment screw knob 214. Additionally seen in this exploded view in FIG. 33 are the parts of the trunnion securing assembly 50 comprising the securing nut 246, the stem trunnion securing member 50 has a partially hollow body 258, having a ribbed inner portion 256 running roughly halfway from the large diameter to the small diameter proximal to the threaded portion 248. It was previously disclosed that trunnion securing members come in varying sized trunnion adapters so as to fit any size implant trunnion as the first and original fixation point.

FIG. 37 depicts a top, side elevational and perspective view of a 2-piece modular blade guide block 200 illustrating a laterally movably adjustable piece housing 214 the lateral blade guide slots 204, here shown with the rigid lateral guide blade 230 guiding the flexible lateral cutting blade 232 down the lateral surface of the femoral stem 220 to be extracted. The purpose of this FIG. 37 is to show that the laterally movably adjustable piece housing 214 the lateral blade guide slots 204 can be adjusted for differing sized femoral implants such that the rigid lateral guide blade 230 will accurately guide the flexible lateral cutting blade 232 down the lateral surface of the femoral stem 220 to be extracted, thereby enabling the preservation of critical bone mass during joint revision surgery.

FIG. 38 depicts a side elevational view of the 2-piece modular blade guide block 200 illustrating a laterally movably adjustable piece housing 214 the lateral blade guide slots 204, here shown with the rigid lateral guide blade 230 guiding the flexible lateral cutting blade 232 down the lateral surface of the femoral stem 220 to be extracted, as shown in FIG. 37. Here, the rigid lateral guide blade 230 guiding the flexible lateral cutting blade 232 down the lateral surface is extended far down the lateral surface of the femoral implant to cut the stem away from the bone and facilitate is removal.

FIG. 39 depicts a top plan view of the 2-piece modular blade guide block 200 illustrating a laterally movably adjustable piece housing 214 the lateral blade guide slots 204, here shown with the rigid lateral guide blade 230 guiding the flexible lateral cutting blade 232 down the lateral surface of the femoral stem 220 to be extracted, as shown in FIG. 37. The laterally movably adjustable piece 212 housing the lateral blade guide slots 204 is here shown fully contracted inward to accommodate a small sized femoral implant and accurately cut the lateral surface of the implant 220 from the bone it is adhered to, during revision surgery.

FIG. 40 depicts a cross-sectional view of the 2-piece modular blade guide block 200 illustrating a laterally movably adjustable piece housing 214 the lateral blade guide slots 204, here shown with the rigid lateral guide blade 230 guiding the flexible lateral cutting blade 232 down the lateral surface of the femoral stem 220 to be extracted, as shown in FIG. 37. The laterally movably adjustable piece 212 housing the lateral blade guide slots 204 is here shown fully contracted inward to accommodate a small sized femoral implant and accurately cut the lateral surface of the implant 220 from the bone it is adhered to, during revision surgery.

FIG. 41 depicts a partially exploded top, side elevational and perspective of a 2-piece modular blade guide block 250 illustrating a laterally movably adjustable piece 262 housing the lateral blade guide slots 254, here shown with an additional fixation point feature. The additional fixation point feature comprises a fixation screw having a fixation screw knob 208 and a threaded portion 210. Additionally, part as shown include a universal central guide block body 252, a trunnion securing assembly 50, a plurality of medial guide slots 256, and the aforementioned plurality of lateral guide slots 254. The parts of the femoral implant 220 include the neck 222, the insertion/extraction point 224, the MDJ 226 and the lower portion of the stem 220.

FIG. 42 depicts a partially exploded side elevational view of the 2-piece modular blade guide block 250 illustrating a laterally movably adjustable piece 262 housing the lateral blade guide slots 254, here shown with an additional fixation point feature as shown in FIG. 41. The additional fixation point feature comprises a fixation screw having a fixation screw knob 208 and a threaded portion 210. Additionally, part as shown include a universal central guide block body 252, a trunnion securing assembly 50, a plurality of medial guide slots 256, and the aforementioned plurality of lateral guide slots 254. The parts of the femoral implant 220 include the neck 222, the insertion/extraction point 224, the MDJ 226 and the lower portion of the stem 220.

illustrating a laterally movably adjustable piece housing the lateral blade guide slots, with an additional fixation point screw as shown in FIG. 41.

FIG. 43 depicts a partially exploded side elevational view of the 2-piece modular blade guide block 250 illustrating a laterally movably adjustable piece 262 housing the lateral blade guide slots 254, here shown with an additional fixation point feature as shown in FIG. 41.

FIG. 44 depicts a partially exploded cross-sectional view of the 2-piece modular blade guide block 250 illustrating a laterally movably adjustable piece 262 housing the lateral blade guide slots 254, here shown with an additional fixation point feature as shown in FIG. 43.

FIG. 45 depicts a top, side elevational and perspective of a 2-piece modular blade guide block 250 illustrating a laterally movably adjustable piece 262 housing the lateral blade guide slots 254, here shown with an additional fixation point screw 208 and 210 threaded all the way down and affixed to the femoral stem to be extracted. This view also shows the laterally movably adjustable piece 262 extended out a moderate distance. The purpose of this FIG. 45 is to show that an effective second fixation point is accomplished by threading the screw threads 210 down through the central universal body 252 to make solid contact with the insertion/extraction point 224 on the femoral implant 220. This additional fixation screw stabilizes the guide block and strongly affixes it to the femoral implant to be extracted. This is essential for accurate guidance and cutting of the lateral surface and medial calcar of the implant to be extracted. It also significantly stabilizes the guide block for use with robotics and navigation technologies. This ultimately adds to the efficiency of the revision surgery process and leads to better outcomes and quicker recoveries.

FIG. 46 depicts a side elevational view of the 2-piece modular blade guide block 250 illustrating a laterally movably adjustable piece 262 housing the lateral blade guide slots 254, with an additional fixation point screw 208 and 210 threaded all the way down and affixed to the insertion/extraction point 224 of the femoral stem 220 to be extracted as shown in FIG. 45.

FIG. 47 depicts a top plan view of the 2-piece modular blade guide block 250 illustrating a laterally movably adjustable piece 262 housing the lateral blade guide slots 254, with an additional fixation point screw 208 and 210 threaded all the way down and affixed to the insertion/extraction point 224 of the femoral stem 220 to be extracted as shown in FIG. 45.

FIG. 48 depicts a cross-sectional view of the 2-piece modular blade guide block 250 illustrating a laterally movably adjustable piece 262 housing the lateral blade guide slots 254, with an additional fixation point screw 208 and 210 threaded all the way down and affixed to the insertion/extraction point 224 of the femoral stem 220 to be extracted as shown in FIG. 47.

FIG. 49 depicts a partially exploded top, side elevational and perspective of a 2-piece modular blade guide block 250 illustrating a removed laterally movably adjustable piece (not shown), here shown with an additional fixation point screw knob 208 and threads 210. The purpose of this FIG. 49 is to show that the universal central body piece 252 can be used without the laterally movably adjustable piece 262 housing the lateral blade guide slots 254 installed on the modular guide block 250.

FIG. 50 depicts a partially exploded side elevational view of the 2-piece modular blade guide block 250 illustrating a removed laterally movably adjustable piece (not shown), here shown with an additional fixation point screw knob 208 and threads 210 as shown in FIG. 49. The purpose of this FIG. 50 is to show that the universal central body piece 252 can be used without the laterally movably adjustable piece 262 housing the lateral blade guide slots 254 installed on the modular guide block 250.

FIG. 51 depicts a partially exploded top plan view of the 2-piece modular blade guide block 250 illustrating a removed laterally movably adjustable piece (not shown), here shown with an additional fixation point screw knob 208 and threads 210 as shown in FIG. 49. With the laterally movably adjustable piece remove the threaded orifice 211 is revealed where the adjustment screw threads is located and when knob 208 is turned tightens down the laterally movably adjustable piece.

FIG. 52 depicts a partially exploded cross-sectional view of the 2-piece modular blade guide block 250 illustrating a removed laterally movably adjustable piece (not shown), here shown with an additional fixation point screw knob 208 and threads 210 as shown in FIG. 51. In this cross-sectional view, the threaded orifice 211 is also seen where the adjustment screw threads is located and when knob 208 is turned tightens down the laterally movably adjustable piece.

FIG. 53 depicts a top, side elevational and perspective of a 2-piece modular blade guide block 250 illustrating a removed laterally movably adjustable piece (not shown), here shown with an additional fixation point screw knob 208 and threads 210 having the screw all the way down and affixed to the insertion/extraction point 224 of the femoral stem 220 to be extracted. The purpose of this FIG. 53 is to show that the fixation screw knob 208 and threads 210 can be used without the lateral adjustment module piece in place on the universal central body 252 of the 2-piece modular blade guide block 250.

FIG. 54 depicts a side elevational view of the 2-piece modular blade guide block 250 illustrating a removed laterally movably adjustable piece (not shown), here shown with an additional fixation point screw knob 208 and threads 210 having the screw all the way down and affixed to the insertion/extraction point 224 of the femoral stem 220 to be extracted. Again, the purpose of this FIG. 53 is to show that the fixation screw knob 208 and threads 210 can be used without the lateral adjustment module piece in place on the universal central body 252 of the 2-piece modular blade guide block 250.

FIG. 55 depicts a top plan view of the 2-piece modular blade guide block 250 illustrating a removed laterally movably adjustable piece (not shown), here shown with an additional fixation point screw knob 208 and threads 210 having the screw all the way down and affixed to the insertion/extraction point 224 of the femoral stem 220 to be extracted as shown in FIG. 53.

FIG. 56 depicts a cross-sectional view of the 2-piece modular blade guide block 250 illustrating a removed laterally movably adjustable piece (not shown), here shown with an additional fixation point screw knob 208 and threads 210 having the screw all the way down and affixed to the insertion/extraction point 224 of the femoral stem 220 to be extracted as shown in FIG. 55.

### 3-PIECE

FIG. 57 depicts a top, side elevational and perspective view of a 3-piece modular blade guide block 300 illustrating a laterally movably adjustable piece modular piece 312 and a medial modular piece 316 attached to the central universal piece 302, also showing a medial modular piece retaining pin 318 feature. The parts of the 3-piece modular blade guide block 300 are: the universal central body 302, the trunnion securing member assembly 50, the laterally moveably adjustable modular piece 312 having a plurality of guide slots 304 and being secured by the adjustment screw 314, the medial piece module 316 having a plurality of guide slots 306 and being secured by medial module securing pin 318. Also shown here is the 3-piece modular blade guide block 300 secured to the femoral implant 320 at its neck portion 322 and having an insertion/extraction point 324 and a MDJ 326. In this FIG. 57 view, the laterally moveably adjustable modular piece 312 having a plurality of guide slots 304 and being secured by the adjustment screw 314 is extended outward in a moderately outward position to adjust for the differing sizes of femoral implants 320.

FIG. 58 depicts a side elevational view of the 3-piece modular blade guide block 300 illustrating a laterally movably adjustable piece modular piece 312 and a medial modular piece 316 attached to the central universal piece 302, also showing a medial modular piece retaining pin 318 feature as shown in FIG. 57. Again, the parts of the 3-piece modular blade guide block 300 are: the universal central body 302, the trunnion securing member assembly 50, the laterally moveably adjustable modular piece 312 having a plurality of guide slots 304 and being secured by the adjustment screw 314, the medial piece module 316 having a plurality of guide slots 306 and being secured by medial module securing pin 318. Also shown here is the 3-piece modular blade guide block 300 secured to the femoral implant 320 at its neck portion 322 and having an insertion/extraction point 324 and a MDJ 326. In this FIG. 57 view, the laterally moveably adjustable modular piece 312 having a plurality of guide slots 304 and being secured by the adjustment screw 314 is extended outward in a moderately outward position to adjust for the differing sizes of femoral implants 320.

FIG. 59 depicts a top plan view of the 3-piece modular blade guide block 300 illustrating a laterally movably adjustable piece modular piece 312 and a medial modular piece 316 attached to the central universal piece 302, also showing a medial modular piece retaining pin 318 feature as shown in FIG. 57. Again, the parts of the 3-piece modular blade guide block 300 are: the universal central body 302, the trunnion securing member assembly 50, the laterally moveably adjustable modular piece 312 having a plurality of guide slots 304 and being secured by the adjustment screw 314, the medial piece module 316 having a plurality of guide slots 306 and being secured by medial module securing pin 318 as shown in FIG. 57. Again, in this FIG. 57 view, the laterally moveably adjustable modular piece 312 having a plurality of guide slots 304 and being secured by the adjustment screw 314 is extended outward in a moderately outward position to adjust for the differing sizes of femoral implants 320.

FIG. 60 depicts a cross-sectional view of the 3-piece modular blade guide block 300 illustrating a laterally movably adjustable piece modular piece 312 and a medial modular piece 316 attached to the central universal piece 302, also showing a medial modular piece retaining pin 318 feature as shown in FIG. 59. Again, the parts of the 3-piece modular blade guide block 300 are: the universal central body 302, the trunnion securing member assembly 50, the laterally moveably adjustable modular piece 312 having a plurality of guide slots 304 and being secured by the adjustment screw 314, the medial piece module 316 having a plurality of guide slots 306 and being secured by medial module securing pin 318. Also shown here is the 3-piece modular blade guide block 300 secured to the femoral implant 320 at its neck portion 322 and having an insertion/extraction point 324 and a MDJ 326. In this FIG. 57 view, the laterally moveably adjustable modular piece 312 having a plurality of guide slots 304 and being secured by the adjustment screw 314 is extended outward in a moderately outward position to adjust for the differing sizes of femoral implants 320.

FIG. 61 depicts an exploded top, side elevational and perspective view of a 3-piece modular blade guide block 300 illustrating the laterally movably adjustable modular piece 312 and the medial modular piece 316 detached from the central universal piece 302 and also showing a medial piece retaining pin 318. In addition to the parts of the 3-piece modular blade guide block 300 previously shown, namely, the universal central body 302, the trunnion securing member assembly 50, the laterally moveably adjustable modular piece 312 having a plurality of guide slots 304 and being secured by the adjustment screw 314, the medial piece module 316 having a plurality of guide slots 306 and being secured by medial module securing pin 318. Also seen is the threaded orifice 315 where the laterally moveably adjustable modular piece 312 is secured by securing screw 314 and the threaded orifice 319 where the medial modular piece 316 attached to the central universal piece 302.

by securing pin 318 which has threads 321 on its proximal end near the screw knob. Additionally seen in this exploded view in FIG. 61 are the parts of the trunnion securing assembly 50 comprising the securing nut 346, the stem trunnion securing member 50 has a partially hollow body 358, having a ribbed inner portion 356 running roughly half way from the large diameter to the small diameter proximal to the threaded portion 357. It was previously disclosed that the conical trunnion securing members come in varying sized conical trunnion adapters so as to fit any size implant trunnion as the first and original fixation point.

FIG. 62 depicts an exploded side elevational view of the 3-piece modular blade guide block 300 illustrating the laterally movably adjustable modular piece 312 and the medial modular piece 316 detached from the central universal piece, also showing a medial piece retaining pin 318 as shown in FIG. 61. Again, as seen in FIG. 61 above the parts shown include the universal central body 302, the trunnion securing member assembly 50, the laterally moveably adjustable modular piece 312 having a plurality of guide slots 304 and being secured by the adjustment screw 314, the medial piece module 316 having a plurality of guide slots 306 and being secured by medial module securing pin 318. Also seen is the threaded orifice 315 where the laterally moveably adjustable modular piece 312 is secured by securing screw 314 and the threaded orifice 319 where the medial modular piece 316 attached to the central universal piece 302 by securing pin 318 which has threads 321 on its proximal end near the screw knob. Additionally seen in this exploded view in FIG. 62 are the parts of the trunnion securing assembly 50 comprising the securing nut 346, the stem trunnion securing member 50 has a partially hollow body 358, having a ribbed inner portion 356 running roughly half way from the large diameter to the small diameter proximal to the threaded portion 357.

FIG. 63 depicts an exploded top plan view of the 3-piece modular blade guide block 300 illustrating the laterally movably adjustable modular piece 312 and the medial modular piece 316 detached from the central universal piece 302, also showing a medial piece retaining pin 318 as shown in FIG. 62. Additionally, the trunnion securing assembly securing nut 346, the stem conical trunnion securing member 50 partially hollow conical body 358 can be seen.

FIG. 64 depicts an exploded cross-sectional view of the 3-piece modular blade guide block 300 illustrating the laterally movably adjustable modular piece 312 and the medial modular piece 316 detached from the central universal piece, also showing a medial piece retaining pin 318 as shown in FIG. 63. Again, as seen in FIG. 62 above the parts shown include the universal central body 302, the trunnion securing member assembly 50, the laterally moveably adjustable modular piece 312 having a plurality of guide slots 304 and being secured by the adjustment screw 314, the medial piece module 316 having a plurality of guide slots 306 and being secured by medial module securing pin 318. Also seen is the threaded orifice 315 where the laterally moveably adjustable modular piece 312 is secured by securing screw 314 and the threaded orifice 319 where the medial modular piece 316 attached to the central universal piece 302 by securing pin 318 which has threads 321 on its proximal end near the screw knob. Additionally seen in this exploded view in FIG. 62 are the parts of the trunnion securing assembly 50 comprising the securing nut 346, the stem trunnion securing member 50 has a partially hollow body 358, having a ribbed inner portion 356 running roughly half way from the large diameter to the small diameter proximal to the threaded portion 357. Moreover, the parts of the femoral implant are seen as the neck 322, the insertion/extraction point 324 the MDJ 326 and the lower stem portion 320.

FIG. 65 depicts a top, side elevational and perspective view of a 3-piece modular blade guide block 300 illustrating a laterally movably adjustable modular piece removed (not shown) and the medial modular piece 316 attached to the central universal piece 302, having a plurality of blade guide slots 306 therein, and showing a medial piece retaining pin 318. The purpose of this FIG. 65 is to show that a surgeon can use the 3-piece modular blade guide block 300 with the laterally movably adjustable modular piece removed and the medial modular piece 316 attached during revision surgery as needed in this configuration.

FIG. 66 depicts a side elevational view of the 3-piece modular blade guide block 300 illustrating a laterally movably adjustable modular piece removed and the medial modular piece 316 attached to the central universal piece 302, having a plurality of blade guide slots 306 therein, and showing a medial piece retaining pin 318 as shown in FIG. 65. Again, the purpose of this FIG. 66 is to show that a surgeon can use the 3-piece modular blade guide block 300 with the laterally movably adjustable modular piece removed and the medial modular piece 316 attached during revision surgery as needed in this configuration.

FIG. 67 depicts a top plan view of the 3-piece modular blade guide block 300 illustrating a laterally movably adjustable modular piece removed and the medial modular piece 316 attached to the central universal piece 302, having a plurality of blade guide slots 306 therein, and showing a medial piece retaining pin 318 as shown in FIG. 65. Again, the purpose of this FIG. 67 is to show that a surgeon can use the 3-piece modular blade guide block 300 with the laterally movably adjustable modular piece removed and the medial modular piece 316 attached during revision surgery as needed in this configuration.

FIG. 68 depicts a cross-sectional view of the 3-piece modular blade guide block 300 illustrating a laterally movably adjustable modular piece removed and the medial modular piece 316 attached to the central universal piece 302, having a plurality of blade guide slots 306 therein, and showing a medial piece retaining pin 318 as shown in FIG. 65. Again, the purpose of this FIG. 68 is to show that a surgeon can use the 3-piece modular blade guide block 300 with the laterally movably adjustable modular piece removed and the medial modular piece 316 attached during revision surgery as needed in this configuration.

FIG. 69 depicts a top, side elevational and perspective view of a 3-piece modular blade guide block 300 illustrating the laterally movably adjustable modular piece 312 and the medial modular piece 316 attached to the central universal piece 302, also showing a medial piece retaining pin 318, and showing a backpressure plate 340 guiding a flexible medial calcar cutting blade 336 through the plurality of guide slots 306 down the medial calcar of a femoral stem 320 to be removed, and also showing a rigid lateral guide blade 330 extending down through the plurality of guide slots 304 guiding a flexible lateral cutting blade 332 down the lateral surface of a femoral stem 320 to be extracted. Also seen is the laterally movably adjustable modular piece 312 fully contracted inward toward the central universal piece 302 and secured by the adjustment screw 314. In operation, the backpressure plate 340 would act to guide the flexible medial calcar cutting blade 336 down the medial calcar of a femoral stem 320 to be removed, and the rigid lateral guide blade 330 would act to guide the flexible lateral cutting blade 332 down the lateral surface of a femoral stem 320 to be extracted. In this way, the cutting blades stay close to the medial and lateral surfaces as they cut the cement away from the stem 320 and thereby preserving critical bone mass during a revision surgical procedure.

FIG. 70 depicts a side elevational view of the 3-piece modular blade guide block 300 illustrating the laterally movably adjustable modular piece 312 and the medial modular piece 316 attached to the central universal piece 302, also showing a medial piece retaining pin 318, and showing a backpressure plate 340 guiding a flexible medial calcar cutting blade 336 through the plurality of guide slots 306 down the medial calcar of a femoral stem 320 to be removed, and also showing a rigid lateral guide blade 330 extending down through the plurality of guide slots 304 guiding a flexible lateral cutting blade 332 down the lateral surface of a femoral stem 320 to be extracted as shown in FIG. 69. Also seen is the laterally movably adjustable modular piece 312 fully contracted inward toward the central universal piece 302 and secured by the adjustment screw 314. In operation, the backpressure plate 340 would act to guide the flexible medial calcar cutting blade 336 down the medial calcar of a femoral stem 320 to be removed, and the rigid lateral guide blade 330 would act to guide the flexible lateral cutting blade 332 down the lateral surface of a femoral stem 320 to be extracted. In this way, the cutting blades stay close to the medial and lateral surfaces as they cut the cement and or bone away from the stem 320 and thereby preserving critical bone mass during a revision surgical procedure.

FIG. 71 depicts a top plan view of the 3-piece modular blade guide block 300 illustrating the laterally movably adjustable modular piece 312 and the medial modular piece 316 attached to the central universal piece 302, also showing a medial piece retaining pin 318, and showing a backpressure plate 340 guiding a flexible medial calcar cutting blade 336 through the plurality of guide slots 306 down the medial calcar of a femoral stem 320 to be removed, and also showing a rigid lateral guide blade 330 extending down through the plurality of guide slots 304 guiding a flexible lateral cutting blade 332 down the lateral surface of a femoral stem 320 to be extracted as shown in FIG. 69. Not seen in this top plan view of FIG. 71 is the laterally movably adjustable modular piece 312 fully contracted inward toward the central universal piece 302 and secured by the adjustment screw 314. In operation, the backpressure plate 340 would act to guide the flexible medial calcar cutting blade 336 down the medial calcar of a femoral stem 320 to be removed, and the rigid lateral guide blade 330 would act to guide the flexible lateral cutting blade 332 down the lateral surface of a femoral stem 320 to be extracted. In this way, the cutting blades stay close to the medial and lateral surfaces as they cut the cement away from the stem 320 and thereby preserving critical bone mass during a revision surgical procedure.

FIG. 72 depicts a cross-sectional view of the 3-piece modular blade guide block 300 illustrating the laterally movably adjustable modular piece 312 and the medial modular piece 316 attached to the central universal piece 302, also showing a medial piece retaining pin 318, and showing a backpressure plate 340 guiding a flexible medial calcar cutting blade 336 through the plurality of guide slots 306 down the medial calcar of a femoral stem 320 to be removed, and also showing a rigid lateral guide blade 330 extending down through the plurality of guide slots 304 guiding a flexible lateral cutting blade 332 down the lateral surface of a femoral stem 320 to be extracted as shown in FIG. 71. Also seen is the laterally movably adjustable modular piece 312 fully contracted inward toward the central universal piece 302 and secured by the adjustment screw 314. In operation, the backpressure plate 340 would act to guide the flexible medial calcar cutting blade 336 down the medial calcar of a femoral stem 320 to be removed, and the rigid lateral guide blade 330 would act to guide the flexible lateral cutting blade 332 down the lateral surface of a femoral stem 320 to be extracted. In this way, the cutting blades stay close to the medial and lateral surfaces as they cut the cement away from the stem 320 and thereby preserving critical bone mass during a revision surgical procedure.

FIG. 73 depicts a top, side elevational and perspective view of a 3-piece modular blade guide block 300 illustrating a laterally movably adjustable modular piece 312 attached to the central universal piece 302 and a medial modular piece removed (not shown), also showing the laterally movably adjustable piece 312 having a plurality of blade guide slots 304 extended laterally outward from the central universal piece 302 and secured by the adjustment screw 314. With the medial modular piece removed, there is seen a threaded orifice 319 in the central universal piece 302. This threaded orifice accepts the medial modular piece (not shown) retaining pin (also not shown). The purpose of this FIG. 73 is to show that a surgeon can use the 3-piece modular blade guide block 300 with the laterally movably adjustable modular piece attached and the medial modular piece 316 removed during revision surgery as needed in this configuration.

FIG. 74 depicts a side elevational view of the 3-piece modular blade guide block 300 illustrating a laterally movably adjustable modular piece 312 attached to the central universal piece 302 and a medial modular piece removed (not shown), also showing the laterally movably adjustable piece 312 having a plurality of blade guide slots 304 extended laterally outward from the central universal piece 302 and secured by the adjustment screw 314. With the medial modular piece removed, there is seen a threaded orifice 319 in the central universal piece 302. This threaded orifice accepts the medial modular piece (not shown) retaining pin (also not shown) as shown in FIG. 73.

FIG. 75 depicts a top plan view of the 3-piece modular blade guide block 300 illustrating a laterally movably adjustable modular piece 312 attached to the central universal piece 302 and the medial modular piece removed, also showing the laterally movably adjustable piece 312 extended laterally fully outward from the central universal piece 302 and secured by the adjustment screw 314 as shown in FIG. 73.

FIG. 76 depicts a cross-sectional view of the 3-piece modular blade guide block 300 illustrating a laterally movably adjustable modular piece 312 attached to the central universal piece 302 and a medial modular piece removed (not shown), also showing the laterally movably adjustable piece 312 having a plurality of blade guide slots 304 extended laterally outward from the central universal piece 302 and secured by the adjustment screw 314. With the medial modular piece removed, there is seen a threaded orifice 319 in the central universal piece 302. This threaded orifice accepts the medial modular piece (not shown) retaining pin (also not shown) as shown in FIG. 75.

FIG. 77 depicts a partially exploded top, side elevational and perspective view of a 3-piece modular blade guide block 350 illustrating a laterally movably adjustable modular piece 362 and a medial modular piece 376 attached to the central universal piece 352, also showing an additional fixation point feature comprising a screw knob 308 and a screw threaded portion 310. Here, the laterally movably adjustable modular piece 362 is contracted inward fully. The screw knob 308 and screw threads 310 are configured to thread into a threaded orifice in the central universal piece 352 and make solid contact with the insertion/extraction point 324 on the femoral implant 320. In this way, the additional fixation to the stem is achieved and enables significantly greater stability for the connection of the 3-piece modular blade guide block 350. This prevents unwanted motion during revision surgery and enables the use of robotics and navigation technologies to perform the surgical procedure.

FIG. 78 depicts a partially exploded side elevational view of the 3-piece modular blade guide block 350 illustrating a laterally movably adjustable modular piece 362 and a medial modular piece 376 attached to the central universal piece 352, also showing an additional fixation point feature comprising a screw knob 308 and a screw threaded portion 310 as shown in FIG. 77. Again, the laterally movably adjustable modular piece 362 is contracted inward fully. The screw knob 308 and screw threads 310 are configured to thread into a threaded orifice in the central universal piece 352 and make solid contact with the insertion/extraction point 324 on the femoral implant 320. In this way, the additional fixation to the stem is achieved and enables significantly greater stability for the connection of the 3-piece modular blade guide block 350. This prevents unwanted motion during revision surgery and enables the use of robotics and navigation technologies to perform the surgical procedure.

FIG. 79 depicts a partially exploded top plan view of the 3-piece modular blade guide block 350 illustrating a laterally movably adjustable modular piece 362 and a medial modular piece 376 attached to the central universal piece 352, also showing an additional fixation point feature comprising a screw knob 308 and a screw threaded portion 310 as shown in FIG. 77. Also seen in this FIG. 79 is the lateral plurality of blade guide slots 354 and the medial plurality of blade guide slots 356.

FIG. 80 depicts a partially exploded cross-sectional view of the 3-piece modular blade guide block 350 illustrating a laterally movably adjustable modular piece 362 and a medial modular piece 376 attached to the central universal piece 352, also showing an additional fixation point feature comprising a screw knob 308 and a screw threaded portion 310 as shown in FIG. 79. Again, the laterally movably adjustable modular piece 362 is contracted inward fully. The screw knob 308 and screw threads 310 are configured to thread into a threaded orifice in the central universal piece 352 and make solid contact with the insertion/extraction point 324 on the femoral implant 320. In this way, the additional fixation to the stem is achieved and enables significantly greater stability for the connection of the 3-piece modular blade guide block 350. This prevents unwanted motion during revision surgery and enables the use of robotics and navigation technologies to perform the surgical procedure.

FIG. 81 depicts a partially exploded top, side elevational and perspective view of a 3-piece modular blade guide block 350 illustrating a laterally movably adjustable modular piece 362 attached to the central universal piece and the medial modular piece removed (not shown), also showing an additional fixation point screw knob 308 and screw threads 310. The medial modular piece is removed by removing the securing pin (not shown) by unthreading it out from the threaded securing pin accepting orifice 319. The purpose of this FIG. 81 is to show that a surgeon could use the 3-piece modular blade guide block 350 with the laterally movably adjustable modular piece 362 attached to the central universal piece 352 and the medial modular piece removed when required by the surgical procedure.

FIG. 82 depicts a partially exploded side elevational view of a 3-piece modular blade guide block 350 illustrating a laterally movably adjustable modular piece 362 attached to the central universal piece and the medial modular piece removed (not shown), also showing an additional fixation point screw knob 308 and screw threads 310 as shown in FIG. 81.

FIG. 83 depicts a top plan view of a 3-piece modular blade guide block 350 illustrating a laterally movably adjustable modular piece 362 attached to the central universal piece and the medial modular piece removed (not shown), also showing an additional fixation point screw knob 308 and screw threads 310 as shown in FIG. 81.

FIG. 84 depicts a cross-sectional view of a 3-piece modular blade guide block 350 illustrating a laterally movably adjustable modular piece 362 attached to the central universal piece and the medial modular piece removed (not shown), also showing an additional fixation point screw knob 308 and screw threads 310 as shown in FIG. 83.

FIG. 85 depicts a top, side elevational and perspective view of a 3-piece modular blade guide block 350 illustrating the laterally movably adjustable modular piece 362 attached to the central universal piece 352 and the medial modular piece removed, also showing an additional fixation point screw knob 308 and screw threads 310 which has been extended all the way down to make solid contact with the insertion/extraction point 324 of the femoral stem 320 to be extracted.

FIG. 86 depicts a side elevational view of the 3-piece modular blade guide block 350 illustrating the laterally movably adjustable modular piece 362 attached to the central universal piece 302 and the medial modular piece removed, also showing an additional fixation point screw extended all the way down to make contact with the femoral stem 320 to be extracted, as shown in FIG. 85. When the fixation point screw 308 and 310 are in solid contact with the insertion/extraction point 324 of the femoral stem 320 to be extracted a significantly greater stability is achieved. This enables the surgeon to conduct the revision surgical procedure in a more efficient and effective manner. Greater stability prevents unwanted motion and enables the use of robotics and navigation technologies to perform the revision surgery with greater efficiency and effectiveness, leading to improved outcomes.

FIG. 87 depicts a top, side elevational and perspective view of a 3-piece modular blade guide block 400 illustrating the laterally movably adjustable modular piece 412 and the medial modular piece 416 attached to the central universal piece 402, also showing a medial piece retaining pin 418, and showing two additional fixation point features, one extending downward from the laterally movably adjustable modular piece 412 comprising a screw knob 408 and screw threads 410, and one a fixation screw 420 extending inward from the medial modular piece. When you include the trunnion securing member assembly 50 and the screw knob 408 and screw threads 410 and add the fixation screw 420 extending inward from the medial modular piece, the total number of fixation points is three. This confers significantly greater stability on the connection between the 3-piece modular blade guide block 400 and the femoral implant 320. This enables the surgeon to conduct the revision surgical procedure in an even more efficient and effective manner. Moreover, greater stability prevents unwanted motion and enables the use of robotics and navigation technologies to perform the revision surgery with greater efficiency and effectiveness, leading to improved outcomes.

FIG. 88 depicts a left side elevational view of the 3-piece modular blade guide block 400 illustrating the laterally movably adjustable modular piece 412 and the medial modular piece 416 attached to the central universal piece 402, also showing a medial piece retaining pin 418, and showing two additional fixation point features, one extending downward from the laterally movably adjustable modular piece 412 comprising a screw knob 408 and screw threads 410, and one additional fixation screw 420 extending inward from the medial modular piece. as shown in FIG. 87.

FIG. 89 depicts a right side elevational view of the 3-piece modular blade guide block 400 illustrating the laterally movably adjustable modular piece 412 and the medial modular piece 416 attached to the central universal piece 402, also showing a medial piece retaining pin 418, and showing two additional fixation point features, one extending downward from the laterally movably adjustable modular piece 412 comprising a screw knob 408 and screw threads 410, and one additional fixation screw 420 extending inward from the medial modular piece. as shown in FIG. 87.

FIG. 90 depicts a top plan view of the 3-piece modular blade guide block 400 illustrating the laterally movably adjustable modular piece 412 and the medial modular piece 416 attached to the central universal piece 402, also showing a medial piece retaining pin 418, and showing two additional fixation point features, one extending downward from the laterally movably adjustable modular piece 412 comprising a screw knob 408 and screw threads 410, and one additional fixation screw 420 extending inward from the medial modular piece, as shown in FIG. 89.

FIG. 91 depicts a cross-sectional view of the 3-piece modular blade guide block 400 illustrating the laterally movably adjustable modular piece 412 and the medial modular piece 416 attached to the central universal piece 402, also showing a medial piece retaining pin 418, and showing two additional fixation point features, one extending downward from the laterally movably adjustable modular piece 412 comprising a screw knob 408 and screw threads 410, and one a fixation screw 420 extending inward from the medial modular piece, as shown in FIG. 90.

FIG. 92 depicts a top, side elevational and perspective view of a 3-piece modular blade guide block 500 illustrating the laterally movably adjustable modular piece 512 and the medial modular piece 516 attached to the central universal piece 502, also showing a medial piece retaining pin 518, and showing four additional fixation point features over and above the trunnion securing member assembly 50, one extending downward from the laterally movably adjustable modular piece comprising a screw knob 508 and screw threads 510, one extending inward from the medial modular piece 520, one fixation screw 522 extending inward from the right side of the central universal piece 502 and one fixation screw 524 extending inward from the left side of the central universal piece 502 (not shown, see FIG. 95 and FIG. 98). When you include the trunnion securing member assembly 50 and the screw knob 508 and screw threads 510 and add the fixation screw 520 extending inward from the medial modular piece, and one fixation screw 522 extending inward from the right side of the central universal piece 502 and one fixation screw 524 extending inward from the left side of the central universal piece 502 the total number of fixation points is five. This confers significantly greater stability on the connection between the 3-piece modular blade guide block 500 and the femoral implant 320. This enables the surgeon to conduct the revision surgical procedure in an even more efficient and effective manner. Moreover, greater stability prevents unwanted motion and enables the use of robotics and navigation technologies to perform the revision surgery with greater efficiency and effectiveness, leading to improved outcomes.

FIG. 93 depicts a side elevational view of the 3-piece modular blade guide block 500 illustrating the laterally movably adjustable modular piece 512 and the medial modular piece 516 attached to the central universal piece 502, also showing a medial piece retaining pin 518, and showing four additional fixation point features over and above the trunnion securing member assembly 50, one extending downward from the laterally movably adjustable modular piece comprising a screw knob 508 and screw threads 510, one extending inward from the medial modular piece 520, one fixation screw 522 extending inward from the right side of the central universal piece 502 and one fixation screw 524 extending inward from the left side of the central universal piece 502 (left side not seen, see FIG. 95 and FIG. 98) as shown in FIG. 92.

FIG. 94 depicts a bottom, side elevational and perspective view of the 3-piece modular blade guide block 500 illustrating the laterally movably adjustable modular piece 512 and the medial modular piece 516 attached to the central universal piece 502, also showing a medial piece retaining pin 518, and showing four additional fixation point features over and above the trunnion securing member assembly 50, one extending downward from the laterally movably adjustable modular piece comprising a screw knob 508 and screw threads 510, one extending inward from the medial modular piece 520, one fixation screw 522 extending inward from the right side of the central universal piece 502 and one fixation screw 524 extending inward from the left side of the central universal piece 502 as shown in FIG. 92.

FIG. 95 depicts a top plan view of the 3-piece modular blade guide block 500 illustrating the laterally movably adjustable modular piece 512 and the medial modular piece 516 attached to the central universal piece 502, also showing a medial piece retaining pin 518, and showing four additional fixation point features over and above the trunnion securing member assembly 50, one extending downward from the laterally movably adjustable modular piece comprising a screw knob 508 and screw threads 510, one extending inward from the medial modular piece 520, one fixation screw 522 extending inward from the right side of the central universal piece 502 and one fixation screw 524 extending inward from the left side of the central universal piece 502 as shown in FIG. 92.

FIG. 96 depicts a cross-sectional view of the 3-piece modular blade guide block 500 illustrating the laterally movably adjustable modular piece 512 and the medial modular piece 516 attached to the central universal piece 502, also showing a medial piece retaining pin 518, and showing four additional fixation point features over and above the trunnion securing member assembly 50, one extending downward from the laterally movably adjustable modular piece comprising a screw knob 508 and screw threads 510, one extending inward from the medial modular piece 520, one fixation screw 522 extending inward from the right side of the central universal piece 502 and one fixation screw 524 extending inward from the left side of the central universal piece 502 as shown in FIG. 95. Having up to five fixation points on the 3-piece modular blade guide block 500 confers maximum stability on the guide block-to-stem connection.

FIG. 97 depicts a bottom and right side perspective view of the 3-piece modular blade guide block 500 illustrating the laterally movably adjustable modular piece 512 and the medial modular piece 516 attached to the central universal piece 502, also showing a medial piece retaining pin 518, and showing four additional fixation point features over and above the trunnion securing member assembly 50, one extending downward from the laterally movably adjustable modular piece comprising a screw knob 508 and screw threads 510, one extending inward from the medial modular piece 520, one fixation screw 522 extending inward from the right side of the central universal piece 502 and one fixation screw 524 extending inward from the left side of the central universal piece 502 as shown in FIG. 92.

FIG. 98 depicts a bottom and left side perspective view of the 3-piece modular blade guide block 500 illustrating the laterally movably adjustable modular piece 512 and the medial modular piece 516 attached to the central universal piece 502, also showing a medial piece retaining pin 518, and showing four additional fixation point features over and above the trunnion securing member assembly 50, one extending downward from the laterally movably adjustable modular piece comprising a screw knob 508 and screw threads 510, one extending inward from the medial modular piece 520, one fixation screw 522 extending inward from the right side of the central universal piece 502 and one fixation screw 524 extending inward from the left side of the central universal piece 502 as shown in FIG. 97.

FIG. 99 depicts a top, side elevational and perspective view of a 3-piece modular blade guide block 500 illustrating the laterally movably adjustable modular piece 512 and the medial modular piece 516 attached to the central universal piece 502, also showing a medial piece retaining pin 518, and showing four additional fixation point features over and above the trunnion securing member assembly 50, one extending downward from the laterally movably adjustable modular piece comprising a screw knob 508 and screw threads 510, one extending inward from the medial modular piece 520, one fixation screw 522 extending inward from the right side of the central universal piece 502 and one fixation screw 524 (not seen in this view) extending inward from the left side of the central universal piece 502 and also showing a rigid guide blade 530 extending downward to contact the lateral shoulder of a femoral stem 320 to be extracted. Here the laterally movably adjustable modular piece 512 is fully contracted inward, but could be adjusted outward as required to extract differing sizes of femoral implants.

FIG. 100 depicts a side elevational view of the 3-piece modular blade guide block 500 illustrating the laterally movably adjustable modular piece 512 and the medial modular piece 516 attached to the central universal piece 502, also showing a medial piece retaining pin 518, and showing four additional fixation point features over and above the trunnion securing member assembly 50, one extending downward from the laterally movably adjustable modular piece comprising a screw knob 508 and screw threads 510, one extending inward from the medial modular piece 520, one fixation screw 522 extending inward from the right side of the central universal piece 502 and one fixation screw 524 (not seen in this view) extending inward from the left side of the central universal piece 502 and also showing a rigid guide blade 530 extending downward to contact the lateral shoulder of a femoral stem 320 to be extracted as shown in FIG. 99.

FIG. 101 depicts a rear view of the 3-piece modular blade guide block 500 illustrating the laterally movably adjustable modular piece 512 and the medial modular piece 516 attached to the central universal piece 502, also showing a medial piece retaining pin 518, and showing four additional fixation point features over and above the trunnion securing member assembly 50, one extending downward from the laterally movably adjustable modular piece comprising a screw knob 508 and screw threads 510, one extending inward from the medial modular piece 520, one fixation screw 522 extending inward from the right side of the central universal piece 502 and one fixation screw 524 extending inward from the left side of the central universal piece 502 and also showing a rigid guide blade 530 extending downward to contact the lateral shoulder of a femoral stem 320 to be extracted as shown in FIG. 99.

FIG. 102 depicts a top plan view of the 3-piece modular blade guide block 500 illustrating the laterally movably adjustable modular piece 512 and the medial modular piece 516 attached to the central universal piece 502, also showing a medial piece retaining pin 518, and showing four additional fixation point features over and above the trunnion securing member assembly 50, one extending downward from the laterally movably adjustable modular piece comprising a screw knob 508 and screw threads 510, one extending inward from the medial modular piece 520, one fixation screw 522 extending inward from the right side of the central universal piece 502 and one fixation screw 524 extending inward from the left side of the central universal piece 502 and also showing a rigid guide blade 530 extending downward to contact the lateral shoulder of a femoral stem 320 to be extracted as shown in FIG. 99.

FIG. 103 depicts a cross-sectional view of the 3-piece modular blade guide block 500 illustrating the laterally movably adjustable modular piece 512 and the medial modular piece 516 attached to the central universal piece 502, also showing a medial piece retaining pin 518, and showing four additional fixation point features over and above the trunnion securing member assembly 50, one extending downward from the laterally movably adjustable modular piece comprising a screw knob 508 and screw threads 510, one extending inward from the medial modular piece 520, one fixation screw 522 extending inward from the right side of the central universal piece 502 and one fixation screw 524 extending inward from the left side of the central universal piece 502 and also showing a rigid guide blade 530 extending downward to contact the lateral shoulder of a femoral stem 320 to be extracted as shown in FIG. 102.

FIG. 104A depicts a top, side elevational and perspective view of an alternate embodiment of a 2-piece modular blade guide block 600 illustrating a 2- piece laterally movably adjustable modular piece 606 and 610 removably attached to a universal central body 602 by a dovetail connection point 608 and having a screw adjustment feature 614 capable of very fine lateral extension adjustment. The 2- piece laterally movably adjustable modular piece comprises a first stationary piece 606 having a female dovetail connection feature 608 and a second laterally movable piece 610 having a male dovetail connection feature 608 interface and a plurality of blade guide slots 612 therein. Lateral movement of the second laterally movable piece 610 having a plurality of blade guide slots 612 therein is accomplished by rotation of a threaded bolt 614 within a threaded channel 616 located in the first stationary piece 606.

FIG. 104B depicts a side elevational view of an alternate embodiment of a 2-piece modular blade guide block 600 illustrating a 2- piece laterally movably adjustable modular piece 606 and 610 removably attached to a universal central body 602, including a plurality of blade guide slots 604, by a dovetail connection point 608 and having a screw adjustment feature 614 capable of very fine lateral extension adjustment as shown in FIG. 104A. The 2- piece laterally movably adjustable modular piece comprises a first stationary piece 606 having a female dovetail connection feature 608 and a second laterally movable piece 610 having a male dovetail connection feature 608 interface and a plurality of blade guide slots 612 therein. Lateral movement of the second laterally movable piece 610 having a plurality of blade guide slots 612 therein is accomplished by rotation of a threaded bolt 614 within a threaded channel 616 located in the first stationary piece 606. as shown in FIG. 104A.

FIG. 104C depicts a bottom view of an alternate embodiment of a 2-piece modular blade guide block 600 illustrating a 2- piece laterally movably adjustable modular piece 606 and 610 removably attached to a universal central body 602, including a plurality of blade guide slots 604, by a dovetail connection point 608 and having a screw adjustment feature 614 capable of very fine lateral extension adjustment as shown in FIG. 104B. Also seen in this view is the trunnion securing member assembly 50.

FIG. 105A depicts a rear view of an alternate embodiment of a 2-piece modular blade guide block 600 illustrating a 2- piece laterally movably adjustable modular piece 606 and 610 removably attached to a universal central body 602, including a plurality of blade guide slots 604, by a dovetail connection point 608 and having a screw adjustment feature 614 capable of very fine lateral extension adjustment as shown in FIG. 104C. Also seen in this view is the previously described trunnion securing member assembly 50.

FIG. 105B depicts a top plan view of an alternate embodiment of a 2-piece modular blade guide block 600 illustrating a 2- piece laterally movably adjustable modular piece 606 and 610 removably attached to a universal central body 602, including a plurality of blade guide slots 604, by a dovetail connection point 608 and having a screw adjustment feature 614 capable of very fine lateral extension adjustment as shown in FIG. 105A. Also seen in this view is the trunnion securing member assembly 50.

FIG. 105C depicts a cross-sectional view of an alternate embodiment of a 2-piece modular blade guide block 600 illustrating a 2- piece laterally movably adjustable modular piece 606 and 610 removably attached to a universal central body 602, including a plurality of blade guide slots 604, by a dovetail connection point 608 and having a screw adjustment feature 614 capable of very fine lateral extension adjustment as shown in FIG. 105B. Also seen in this view is the trunnion securing member assembly 50.

FIG. 106A depicts a bottom, side and perspective view of an alternate embodiment of a 2-piece modular blade guide block 600 illustrating a 2- piece laterally movably adjustable modular piece 606 and 610 removably attached to a universal central body 602, including a plurality of blade guide slots 604, by a dovetail connection point 608 and having a screw adjustment feature 614 capable of very fine lateral extension adjustment as shown in FIG. 104. Also seen in this view is the trunnion securing member assembly 50.

FIG. 106B depicts a bottom, side and perspective view of an alternate embodiment of a 2-piece modular blade guide block 600 illustrating a 2- piece laterally movably adjustable modular piece 606 and 610 removably attached to a universal central body 602, including a plurality of blade guide slots 604, by a dovetail connection point 608 and having a screw adjustment feature 614 capable of very fine lateral extension adjustment as shown in FIG. 106A. Also seen in this view is the trunnion securing member assembly 50. Note that in all of the views of the alternate embodiment of a 2-piece modular blade guide block 600 illustrating a 2- piece laterally movably adjustable modular piece 606 and 610 removably attached to a universal central body 602 FIG.’s 104 through 106B the second laterally movable piece 610 is contracted fully inward toward the first stationary piece 606.

FIG. 107A depicts a partially exploded top, side elevational and perspective view of an alternate embodiment of a 2-piece modular blade guide block 600 illustrating a 2- piece laterally movably adjustable modular piece 606 and 610 removably attached to a universal central body 602 by a dovetail connection point 608 and having a screw adjustment feature 614 capable of very fine lateral extension adjustment. The 2- piece laterally movably adjustable modular piece comprises a first stationary piece 606 having a female dovetail connection feature 608 and a second laterally movable piece 610 having a male dovetail connection feature 608 interface and a plurality of blade guide slots 612 therein. Lateral movement of the second laterally movable piece 610 having a plurality of blade guide slots 612 therein is accomplished by rotation of a threaded bolt 614 within a threaded channel 616 located in the first stationary piece 606. This FIG. 107A shows the laterally movably adjustable modular piece 606 and 610 removed, lifted off of the universal central body 602 thereby exposing the dovetail connection point 608. Additionally, the laterally movably adjustable modular piece 606 and 610 having a screw adjustment feature capable of very fine lateral extension adjustment, is shown with the first laterally movable modular piece 610 extended fully outward exposing both the non-threaded guide rail 618 and the threaded bolt 620.

FIG. 107B depicts a partially exploded rear view of an alternate embodiment of a 2-piece modular blade guide block 600 illustrating a 2- piece laterally movably adjustable modular piece 606 and 610 removably attached to a universal central body 602 by a dovetail connection point 608 and having a screw adjustment feature 614 capable of very fine lateral extension adjustment as shown in FIG. 107A. Also seen in this view is the trunnion securing member assembly 50.

FIG. 107C depicts a partially exploded rear view of an alternate embodiment of a 2-piece modular blade guide block 600 illustrating a 2- piece laterally movably adjustable modular piece 606 and 610 removably attached to a universal central body 602 by a dovetail connection point 608 and having a screw adjustment feature 614 capable of very fine lateral extension adjustment shown in FIG. 107A. Again, the 2- piece laterally movably adjustable modular piece comprises a first stationary piece 606 having a female dovetail connection feature 608 and a second laterally movable piece 610 having a male dovetail connection feature 608 interface and a plurality of blade guide slots 612 therein. Lateral movement of the second laterally movable piece 610 having a plurality of blade guide slots 612 therein is accomplished by rotation of a threaded bolt 614 within a threaded channel 616 located in the first stationary piece 606. This FIG. 107A shows the laterally movably adjustable modular piece 606 and 610 removed, lifted off of the universal central body 602 thereby exposing the dovetail connection point 608. Additionally, the laterally movably adjustable modular piece 606 and 610 having a screw adjustment feature capable of very fine lateral extension adjustment, is shown with the first laterally movable modular piece 610 extended fully outward exposing both the non-threaded guide rail 618 and the threaded bolt 620. Also seen in this view is the trunnion securing member assembly 50.

FIG. 108 depicts an exploded side elevational view of an alternate embodiment 2-piece modular blade guide block 600 illustrating a 2- piece laterally movably adjustable modular piece 606 and 610 removably attached to a universal central body 602 by a dovetail connection point 608 and having a screw adjustment feature 614 capable of very fine lateral extension adjustment. The 2- piece laterally movably adjustable modular piece comprises a first stationary piece 606 having a female dovetail connection feature 608 and a second laterally movable piece 610 having a plurality of blade guide slots 612 therein. Lateral movement of the second laterally movable piece 610 having a plurality of blade guide slots 612 therein is accomplished by rotation of a threaded bolt 614 within a threaded channel 616 located in the first stationary piece 606. This FIG. 107A shows the laterally movably adjustable modular piece 606 and 610 removed, lifted off of the universal central body 602 thereby exposing the dovetail connection point 608. In this way, the first stationary piece 606 can be translated in the superior direction and the inferior direction, up and down, to accommodate differing sizes and shapes of the greater trochanter found in differing patients. Additionally, the laterally movably adjustable modular piece 606 and 610 having a screw adjustment feature capable of very fine lateral extension adjustment, is shown with the first laterally movable modular piece 610 extended fully outward exposing both the non-threaded guide rail 618 (not seen) and the threaded bolt 620.

FIG. 109 depicts an exploded top plan view of an alternate embodiment 2-piece modular blade guide block 600 illustrating a 2- piece laterally movably adjustable modular piece 606 and 610 removably attached to a universal central body 602 by a dovetail connection point 608 and having a screw adjustment feature 614 capable of very fine lateral extension adjustment, here shown with the lateral moveable piece 610 fully extended outward, as shown in FIG. 108.

FIG. 110 depicts an exploded cross-sectional view of an alternate embodiment 2-piece modular blade guide block 600 illustrating a 2- piece laterally movably adjustable modular piece 606 and 610 removably attached to a universal central body 602 by a dovetail connection point 608 and having a screw adjustment feature 614 capable of very fine lateral extension adjustment, here shown with the lateral moveable piece 610 fully extended outward, as shown in FIG. 109. In this cross-sectional view of FIG. 110, the non-threaded guide rail 618 is clearly seen on the opposite side of the threaded bolt (not seen). In this way, the laterally movably adjustable modular piece 606 and 610 can be translated in the superior direction and the inferior direction, up and down, to accommodate differing sizes and shapes of the greater trochanter found in differing patients. The purpose of these figures in FIG. 107A through FIG. 110 is to show that the laterally movably adjustable modular piece 606 and 610 can be removed from the universal central body 602 by the surgeon as required during revision surgery procedures.

FIG. 111 depicts a bottom, side and perspective view of an alternate embodiment of a 2-piece modular blade guide block 600 illustrating a 2- piece laterally movably adjustable modular piece 606 and 610 removably attached to a universal central body 602 by a dovetail connection point 608 and having a screw adjustment feature 614 capable of very fine lateral extension adjustment, here shown with the lateral moveable piece 610 fully extended outward, as shown in FIG. 109.

FIG. 112 depicts a bottom, side and perspective view of an alternate embodiment of a 2-piece modular blade guide block 600 illustrating a 2- piece laterally movably adjustable modular piece 606 and 610 removably attached to a universal central body 602 by a dovetail connection point 608 and having a screw adjustment feature 614 capable of very fine lateral extension adjustment, here shown with the lateral moveable piece 610 fully extended outward, as shown in FIG. 111.

FIG. 113A depicts a top, side elevation and perspective view of an alternate embodiment 2-piece modular blade guide block 600 illustrating a 2- piece laterally movably adjustable modular piece 606 and 610 removably attached to a universal central body 602 by a dovetail connection point 608 and having a screw adjustment feature 614 capable of very fine lateral extension adjustment, here shown with the lateral moveable piece 610 fully retracted. Here in this FIG. 113A it is shown that the 2- piece laterally movably adjustable modular piece 606 and 610 removably attached to a universal central body 602 by a dovetail connection point 608 is partially disengaged from the universal central body 602. The purpose of this FIG. 113A is to show that the surgeon may use the alternate embodiment 2-piece modular blade guide block 600 illustrating a 2- piece laterally movably adjustable modular piece 606 and 610 while that modular piece is only partially engaged with the universal central body 602 by the dovetail connection point 608, if required during joint revision surgical procedures. In this way, laterally movably adjustable modular piece 606 and 610 can be translated in the superior direction and the inferior direction, up and down, to accommodate differing sizes and shapes of the greater trochanter found in differing patients.

FIG. 113B depicts a rear view of an alternate embodiment 2-piece modular blade guide block 600 illustrating a 2- piece laterally movably adjustable modular piece 606 and 610 removably attached to a universal central body 602 by a dovetail connection point 608 and having a screw adjustment feature 614 capable of very fine lateral extension adjustment, here shown with the lateral moveable piece 610 fully retracted as shown in FIG. 113A. In this view, the alternate embodiment 2-piece modular blade guide block 600 is secured to a femoral implant by the trunnion securing member assembly (not shown).

FIG. 113C depicts a top, side elevation and perspective view of an alternate embodiment 2-piece modular blade guide block 600 illustrating a 2- piece laterally movably adjustable modular piece 606 and 610 removably attached to a universal central body 602 by a dovetail connection point 608 and having a screw adjustment feature 614 capable of very fine lateral extension adjustment, here shown with the lateral moveable piece 610 fully retracted as shown in FIG. 113B. In this view, the alternate embodiment 2-piece modular blade guide block 600 is secured to a femoral implant by the trunnion securing member assembly 50.

FIG. 114A depicts a top, side elevation and perspective view of an alternate embodiment 2-piece modular blade guide block 600 illustrating a 2- piece laterally movably adjustable modular piece 606 and 610 removably attached to a universal central body 602 by a dovetail connection point 608 and having a screw adjustment feature 614 capable of very fine lateral extension adjustment, here shown with the lateral moveable piece 610 fully extended outward. Here in this FIG. 113A it is shown that the 2- piece laterally movably adjustable modular piece 606 and 610 removably attached to a universal central body 602 by a dovetail connection point 608 is partially disengaged from the universal central body 602. The purpose of this FIG. 113A is to show that the surgeon may use the alternate embodiment 2-piece modular blade guide block 600 illustrating a 2- piece laterally movably adjustable modular piece 606 and 610 while that modular piece is only partially engaged with the universal central body 602 by the dovetail connection point 608, if required during joint revision surgical procedures. In this way, laterally movably adjustable modular piece 606 and 610 can be translated in the superior direction and the inferior direction, up and down, to accommodate differing sizes and shapes of the greater trochanter found in differing patients.

FIG. 114B depicts a rear of an alternate embodiment 2-piece modular blade guide block 600 illustrating a 2- piece laterally movably adjustable modular piece 606 and 610 removably attached to a universal central body 602 by a dovetail connection point 608 and having a screw adjustment feature 614 capable of very fine lateral extension adjustment, here shown with the lateral moveable piece 610 fully extended outward as shown in FIG. 114A. In this view, the alternate embodiment 2-piece modular blade guide block 600 is secured to a femoral implant by the trunnion securing member assembly (not shown).

FIG. 114C depicts a top, side elevation and perspective view of an alternate embodiment 2-piece modular blade guide block 600 illustrating a 2- piece laterally movably adjustable modular piece 606 and 610 removably attached to a universal central body 602 by a dovetail connection point 608 and having a screw adjustment feature 614 capable of very fine lateral extension adjustment, here shown with the lateral moveable piece 610 fully extended outward as shown in FIG. 114B. In this view, the alternate embodiment 2-piece modular blade guide block 600 is secured to a femoral implant by the trunnion securing member assembly 50.

FIG. 115A depicts a top, side elevation and perspective view of an alternate embodiment 2-piece modular blade guide block 600 illustrating a 2- piece laterally movably adjustable modular piece 606 and 610 removably attached to a universal central body 602 by a dovetail connection point 608 and having a screw adjustment feature 614 capable of very fine lateral extension adjustment, here shown with the lateral moveable piece 610 partially extended outward.

FIG. 115B depicts a top, side elevation and perspective view of the alternate embodiment 2-piece modular blade guide block 600 illustrating a 2- piece laterally movably adjustable modular piece 606 and 610 removably attached to a universal central body 602 by a dovetail connection point 608 and having a screw adjustment feature 614 capable of very fine lateral extension adjustment, here shown with the lateral moveable piece 610 partially extended outward as shown in FIG. 115A. In this view, the alternate embodiment 2-piece modular blade guide block 600 is secured to a femoral implant by the trunnion securing member assembly 50.

FIG. 116A depicts a top plan view of the alternate embodiment 2-piece modular blade guide block 600 illustrating a 2- piece laterally movably adjustable modular piece 606 and 610 removably attached to a universal central body 602 by a dovetail connection point 608 and having a screw adjustment feature 614 capable of very fine lateral extension adjustment, here shown with the lateral moveable piece 610 partially extended outward.

FIG. 116B depicts a cross-sectional view of the alternate embodiment 2-piece modular blade guide block 600 illustrating a 2- piece laterally movably adjustable modular piece 606 and 610 removably attached to a universal central body 602 by a dovetail connection point 608 and having a screw adjustment feature 614 capable of very fine lateral extension adjustment, here shown with the lateral moveable piece 610 partially extended outward, as shown in FIG. 116A.

FIG. 117 depicts a rear, top and side elevational perspective view of the improved blade centering backpressure plate 650 having blade centering guide tabs thereon. The L-shaped blade centering backpressure plate 650 comprised an upper portion 652, a lower portion 654. Located on both sides of the upper section of the blade centering backpressure plate 650 lower portion 654 are upper centering tabs 656 and 658 and located on the lower section of the blade centering backpressure plate 650 lower portion 654 are lower centering tabs 660 and 662. In operation, when a blade is inserted into one of the plurality of medial blade guide slots, the upper centering tabs 656 and 658 as well as the lower centering tabs 660 and 662 guide the blade shaft to be centered as it extends down to make contact with the femoral implant to be extracted.

FIG. 118 depicts a front view of an improved blade cantering backpressure plate 650 having blade centering guide tabs 656 and 658 seen thereon.

FIG. 119 depicts a top plan view of the improved blade centering backpressure plate 650 having blade centering guide tabs thereon. The L-shaped blade centering backpressure plate 650 comprised an upper portion 652, a lower portion 654. Located on both sides of the upper section of the blade centering backpressure plate 650 lower portion 654 are upper centering tabs 656 and 658 and located on the lower section of the blade centering backpressure plate 650 lower portion 654 are lower centering tabs 660 and 662. In operation, when a blade is inserted into one of the plurality of medial blade guide slots, the upper centering tabs 656 and 658 as well as the lower centering tabs 660 and 662 guide the blade shaft to be centered as it extends down to make contact with the femoral implant to be extracted.

FIG. 120 depicts a bottom view of the improved backpressure plate having blade centering guide tabs thereon. 650 having blade centering guide tabs thereon. The L-shaped blade centering backpressure plate 650 comprised an upper portion 652, a lower portion 654. Located on both sides of the upper section of the blade centering backpressure plate 652 lower portion 654 are upper centering tabs 656 and 658 and located on the lower section of the blade centering backpressure plate 650 lower portion 654 are lower centering tabs 660 and 662, as seen in FIG. 119.

FIG. 121 depicts a side elevational view of the improved backpressure plate 650 having blade centering guide tabs 658 and 662 seen thereon.

FIG. 122 depicts a top, side elevational and perspective view of a monolithic 1-piece modular blade guide block 100 having a 1-piece body 102 (previously described) illustrating an improved blade centering backpressure plate 650 extending down through the 1-piece body 102 medial blade guide slots 106. The blade centering backpressure plate 650 comprises an upper portion 652 and a lower portion 654 and multiple blade centering guide tabs 652 as seen here. The blade centering backpressure plate 650 is adjustably mounted within the monolithic 1-piece modular blade guide block 100 body 102 using the adjustment bolt 664 with threads 666 and rotating the adjustment bolt to achieve the desired adjustment as described in FIG. 1 through FIG. 5.

FIG. 123 depicts a side elevational view of the monolithic 1-piece modular blade guide block 100 having a 1-piece body 102 illustrating an improved blade centering backpressure plate 650 extending down through the 1-piece body 102 medial blade guide slots (not seen) as shown in FIG. 122. The blade centering backpressure plate 650 comprises an upper portion 652 and a lower portion 654 and multiple blade centering guide tabs 652 as seen here. The blade centering backpressure plate 650 is adjustably mounted within the monolithic 1-piece modular blade guide block 100 body 102 using the adjustment bolt 664 with threads 666 and rotating the adjustment bolt to achieve the desired adjustment as described in FIG. 1 through FIG. 5.

FIG. 124 depicts a bottom, side elevational and perspective view of the monolithic 1-piece modular blade guide block 100 having a 1-piece body 102 illustrating an improved blade centering backpressure plate 650 extending down through the 1-piece body 102 medial blade guide slots 106 as shown in FIG. 123. The blade centering backpressure plate 650 comprises an upper portion 652 and a lower portion 654 and multiple blade centering guide tabs not seen here.

FIG. 125 depicts a top plan view of the monolithic 1-piece modular blade guide block 100 having a 1-piece body 102 illustrating an improved blade centering backpressure plate 650 extending down through the 1-piece body 102 medial blade guide slots 106 as shown in FIG. 122. The blade centering backpressure plate 650 comprises an upper portion 652 and a lower portion 654 and multiple blade centering guide tabs 656 as seen here.

FIG. 126 depicts a cross-sectional view of the monolithic 1-piece modular blade guide block 100 having a 1-piece body 102 illustrating an improved blade centering backpressure plate 650 extending down through the 1-piece body 102 medial blade guide slots 106 as shown in FIG. 125. The blade centering backpressure plate 650 comprises an upper portion 652 and a lower portion 654 and multiple blade centering guide tabs 656 as seen here.

FIG. 127 depicts a bottom, rear, right side elevational and perspective view of the monolithic 1-piece modular blade guide block illustrating an improved backpressure plate having blade centering guide tabs adjustably mounted within the monolithic 1-piece modular blade guide block as shown in FIG. 126. The blade centering backpressure plate 650 comprises an upper portion 652 and a lower portion 654 and multiple blade centering guide tabs not seen here.

FIG. 128 depicts a bottom, rear, left side elevational and perspective view of the 1-piece modular blade guide block illustrating an improved backpressure plate having blade centering guide tabs adjustably mounted within the 1-piece modular blade guide block as shown in FIG. 127. The blade centering backpressure plate 650 comprises an upper portion 652 and a lower portion 654 and multiple blade centering guide tabs not seen here.

FIG. 129 depicts a top, side elevational and perspective view of a 2-piece modular blade guide block 200 having a laterally adjustable modular lateral piece 212 illustrating an improved blade centering backpressure plate 650 having blade centering guide tabs 206 seen here. The blade centering backpressure plate 650 is adjustably mounted within the 2-piece modular blade guide block 200 by inserting it into the plurality of blade guide slots 206. The blade centering backpressure plate 650 is adjusted upwardly and downwardly using the adjustment bolt 664 and threads 666 by rotating it to the desired height.

FIG. 130 depicts a side elevational view of the 2-piece modular blade guide block 200 having a laterally adjustable modular lateral piece 212 illustrating an improved blade centering backpressure plate 650 having blade centering guide tabs 656 seen here. The blade centering backpressure plate 650 is adjustably mounted within the 2-piece modular blade guide block 200 by inserting it into the plurality of blade guide slots 206 as shown in FIG. 129.

FIG. 131 depicts a bottom, right side elevational and perspective view of the 2-piece modular blade guide block 200 having a laterally adjustable modular lateral piece 212 illustrating an improved blade centering backpressure plate 650 having blade centering guide tabs not seen here. The blade centering backpressure plate 650 is adjustably mounted within the 2-piece modular blade guide block 200 by inserting it into the plurality of blade guide slots 206, as shown in FIG. 130.

FIG. 132 depicts a top plan view of the 2-piece modular blade guide block 200 having a laterally adjustable modular lateral piece 212 illustrating an improved blade centering backpressure plate 650 having blade centering guide tabs not seen here. The blade centering backpressure plate 650 is adjustably mounted within the 2-piece modular blade guide block 200 by inserting it into the plurality of blade guide slots 206, as shown in FIG. 130. Here the laterally moveable adjustment module 212 has been extended moderately outward.

FIG. 133 depicts a cross-sectional view of the 2-piece modular blade guide block 200 having a laterally adjustable modular lateral piece 212 illustrating an improved blade centering backpressure plate 650 having blade centering guide tabs 656 seen here. The blade centering backpressure plate 650 is adjustably mounted within the 2-piece modular blade guide block 200 by inserting it into the plurality of blade guide slots 206 as shown in FIG. 132. Again, the laterally moveable adjustment module 212 has been extended moderately outward.

FIG. 134 depicts a bottom, rear, right side elevational and perspective view of the 2-piece modular blade guide block 200 having a laterally adjustable modular lateral piece 212 illustrating an improved blade centering backpressure plate 650 having blade centering guide tabs 656 seen here. The blade centering backpressure plate 650 is adjustably mounted within the 2-piece modular blade guide block 200 by inserting it into the plurality of blade guide slots 206 as shown in FIG. 129. Here the laterally moveable adjustment module 212 has been extended moderately outward.

FIG. 135 depicts a bottom, rear, left side elevational and perspective view of the 2-piece modular blade guide block 200 having a laterally adjustable modular lateral piece 212 illustrating an improved blade centering backpressure plate 650 having blade centering guide tabs 656 seen here. The blade centering backpressure plate 650 is adjustably mounted within the 2-piece modular blade guide block 200 by inserting it into the plurality of blade guide slots 206 as shown in FIG. 134. Again, the laterally moveable adjustment module 212 has been extended moderately outward.

FIG. 136 depicts a top, side elevational and perspective view of a 3-piece modular blade guide block 300 having a laterally adjustable modular lateral piece 312 and a medial modular piece 316 illustrating a blade centering backpressure plate 650 having blade centering guide tabs (not seen) adjustably mounted within the 3-piece modular blade guide block by insertion into the medial plurality of blade guide slots 306. The blade centering backpressure plate 650 is adjusted upwardly and downwardly using the adjustment bolt 664 and threads 666 thereon. Here the laterally adjustable modular lateral piece 312 has been retracted fully inward.

FIG. 137 depicts a top, side elevational and perspective view of the 3-piece modular blade guide block 300 having a laterally adjustable modular lateral piece 312 and a medial modular piece 316 illustrating a blade centering backpressure plate 650 having blade centering guide tabs (not seen) adjustably mounted within the 3-piece modular blade guide block by insertion into the medial plurality of blade guide slots (not seen) as shown in FIG. 136. Here the laterally adjustable modular lateral piece 312 has been retracted fully inward.

FIG. 138 depicts a bottom, right side elevational and perspective view of the 3-piece modular blade guide block 300 having a laterally adjustable modular lateral piece 312 and a medial modular piece 316 illustrating a blade centering backpressure plate 650 having blade centering guide tabs (not seen) adjustably mounted within the 3-piece modular blade guide block by insertion into the medial plurality of blade guide slots 306 as shown in FIG. 136. Here the laterally adjustable modular lateral piece 312 has been retracted fully inward.

FIG. 139 depicts a top plan view of the 3-piece modular blade guide block 300 having a laterally adjustable modular lateral piece 312 and a medial modular piece 316 illustrating a blade centering backpressure plate 650 having blade centering guide tabs (not seen) adjustably mounted within the 3-piece modular blade guide block by insertion into the medial plurality of blade guide slots 306 as shown in FIG. 136. Again, the laterally adjustable modular lateral piece 312 has been retracted fully inward.

FIG. 140 depicts a bottom, right side elevational and perspective view of the 3-piece modular blade guide block 300 having a laterally adjustable modular lateral piece 312 and a medial modular piece 316 illustrating a blade centering backpressure plate 650 having blade centering guide tabs (not seen) adjustably mounted within the 3-piece modular blade guide block by insertion into the medial plurality of blade guide slots 306 as shown in FIG. 136.

FIG. 141 depicts a cross-sectional view of the 3-piece modular blade guide block 300 having a laterally adjustable modular lateral piece 312 and a medial modular piece 316 illustrating a blade centering backpressure plate 650 having blade centering guide tabs (not seen) adjustably mounted within the 3-piece modular blade guide block by insertion into the medial plurality of blade guide slots 306 as shown in FIG. 139. Here the laterally adjustable modular lateral piece 312 has been retracted fully inward.

FIG. 142 depicts a bottom, left side elevational view of the 3-piece modular blade guide block 300 having a laterally adjustable modular lateral piece 312 and a medial modular piece 316 illustrating a blade centering backpressure plate 650 having blade centering guide tabs (not seen) adjustably mounted within the 3-piece modular blade guide block by insertion into the medial plurality of blade guide slots 306 as shown in FIG. 140. Here the laterally adjustable modular lateral piece 312 has been retracted fully inward.

FIG. 143A depicts a top plan view of a small width sized curved rigid lateral guide blade 700 having a blade stop 708 thereon. The curved rigid lateral guide blade 700 comprises a cutting edge 702, a cutting blade shaft 704, a widened blade shaft portion 706, a blade stop 708, a blade handle shaft 710 and a slotted blade handle accepting portion 712. This top plan view shows the cutting edge 702, the widened blade shaft portion 706, a blade stop 708 and the slotted blade handle accepting portion 712.

FIG. 143B depicts a front view of the small width sized curved rigid lateral guide blade 700 having a blade stop 708 thereon. The curved rigid lateral guide blade 700 comprises a cutting edge 702, a cutting blade shaft 704, a widened blade shaft portion 706, a blade stop 708, a blade handle shaft 710 and a slotted blade handle accepting portion 712. The widened blade shaft portion 706 is specifically configured in size to work with the blade slots 612 (see FIG. 104A through FIG. 104C) and the blade stop 708 limits the distance each blade can be inserted.

FIG. 144A depicts a top plan view of a medium width sized curved rigid lateral guide blade 700 having a blade stop 708 thereon. The curved rigid lateral guide blade 700 comprises a cutting edge 702, a cutting blade shaft 704, a widened blade shaft portion 706, a blade stop 708, a blade handle shaft 710 and a slotted blade handle accepting portion 712. This top plan view shows the cutting edge 702, the widened blade shaft portion 706, a blade stop 708 and the slotted blade handle accepting portion 712.

FIG. 144B depicts a front view of the medium width sized curved rigid lateral guide blade 700 having a blade stop 708 thereon. The curved rigid lateral guide blade 700 comprises a cutting edge 702, a cutting blade shaft 704, a widened blade shaft portion 706, a blade stop 708, a blade handle shaft 710 and a slotted blade handle accepting portion 712. The widened blade shaft portion 706 is specifically configured in size to work with the blade centering backpressure plate 650 to fit snuggly within the blade centering tabs on the backpressure plate 650 and force the inserted blade to be centered as it passes down through the blade guide slots, as shown in FIG. 144A.

FIG. 145A depicts a top plan view of a large width sized curved rigid lateral guide blade 700 having a blade stop 708 thereon. The curved rigid lateral guide blade 700 comprises a cutting edge 702, a cutting blade shaft 704, a widened blade shaft portion 706, a blade stop 708, a blade handle shaft 710 and a slotted blade handle accepting portion 712. This top plan view shows the cutting edge 702, the widened blade shaft portion 706, a blade stop 708 and the slotted blade handle accepting portion 712.

FIG. 145B depicts a front view of the large width sized curved rigid lateral guide blade 700 having a blade stop 708 thereon. The curved rigid lateral guide blade 700 comprises a cutting edge 702, a cutting blade shaft 704, a widened blade shaft portion 706, a blade stop 708, a blade handle shaft 710 and a slotted blade handle accepting portion 712. The widened blade shaft portion 706 is specifically configured in size to work with the blade centering backpressure plate 650 to fit snuggly within the blade centering tabs on the backpressure plate 650 and force the inserted blade to be centered as it passes down through the blade guide slots, as shown in FIG. 145A.

FIG. 146 depicts a rear view of the large width sized curved rigid lateral guide blade 700 having a blade stop 708 as shown in FIG. 145B, here illustrating the beveled cutting edge. The 700 comprises a cutting edge 702 beveled on one side, a cutting blade shaft 704, a widened blade shaft portion 706, a blade stop 708, a blade handle shaft 710 and a slotted blade handle accepting portion 712. The widened blade shaft portion 706 is specifically configured in size to work with the blade centering backpressure plate 650 to fit snuggly within the blade centering tabs on the backpressure plate 650 and force the inserted blade to be centered as it passes down through the blade guide slots, as shown in FIG. 145B.

FIG. 147 depicts a rear view of the medium width sized curved rigid lateral guide blade 700 having a blade stop 708 shown in FIG. 144B, illustrating the beveled edge. The 700 comprises a cutting edge 702 beveled on one side, a cutting blade shaft 704, a widened blade shaft portion 706, a blade stop 708, a blade handle shaft 710 and a slotted blade handle accepting portion 712. The widened blade shaft portion 706 is specifically configured in size to work with the blade centering backpressure plate 650 to fit snuggly within the blade centering tabs on the backpressure plate 650 and force the inserted blade to be centered as it passes down through the blade guide slots, as shown in FIG. 144B.

FIG. 148 depicts a rear view of the small width sized curved rigid lateral guide blade 700 having a blade stop 708 shown in FIG. 143B, illustrating the beveled edge. The 700 comprises a cutting edge 702 beveled on one side, a cutting blade shaft 704, a widened blade shaft portion 706, a blade stop 708, a blade handle shaft 710 and a slotted blade handle accepting portion 712. The widened blade shaft portion 706 is specifically configured in size to work with the blade centering backpressure plate 650 to fit snuggly within the blade centering tabs on the backpressure plate 650 and force the inserted blade to be centered as it passes down through the blade guide slots, as shown in FIG. 143B.

FIG. 149A depicts a top plan view of a small width sized curved elongated shaft flexible medial calcar and/or lateral cutting blade 750 having a cutting edge 752 and a slotted blade handle accepting portion 758 seen in this figure.

FIG. 149B depicts a front view of the small width sized curved flexible lateral cutting blade having a cutting edge and an elongated blade shaft as shown in FIG. 149A. The small width sized curved elongated flexible medial calcar and/or lateral cutting blade 750 comprises the following parts: a cutting edge 752 beveled on one side, an elongated cutting blade shaft 754, a widened blade shaft portion 756, and a slotted blade handle accepting portion 758. The blade shaft portion 754 is specifically configured in size to work with the blade centering backpressure plate 650 to fit snuggly within the blade centering tabs on the backpressure plate 650 and force the inserted blade to be centered as it passes down through the blade guide slots located both medially and laterally.

FIG. 150A depicts a top plan view of a medium width sized curved elongated flexible medial calcar and/or lateral cutting blade 750 having a cutting edge 752 and a slotted blade handle accepting portion 758 seen in this figure.

FIG. 150B depicts a front view of the medium width sized curved elongated flexible medial calcar and/or lateral cutting blade 750 having a cutting edge 752 and an elongated blade shaft 754 as shown in FIG. 150A. The small width sized curved elongated flexible medial calcar and/or lateral cutting blade 750 comprises the following parts: a cutting edge 752 beveled on one side, an elongated cutting blade shaft 754, a widened blade shaft portion 756, and a slotted blade handle accepting portion 758. The blade shaft portion 754 is specifically configured in size to work with the blade centering backpressure plate 650 to fit snuggly within the blade centering tabs on the backpressure plate 650 and force the inserted blade to be centered as it passes down through the blade guide slots.

FIG. 151A depicts a top plan view of a large width sized curved elongated flexible medial calcar and/or lateral cutting blade 750 having a cutting edge 752 and a slotted blade handle accepting portion 758 seen in this figure.

FIG. 151B depicts a front view of the large width sized curved elongated flexible medial calcar and/or lateral cutting blade 750 having a cutting edge 752 and an elongated blade shaft 754 as shown in FIG. 151A. The small width sized curved elongated flexible medial calcar and/or lateral cutting blade 750 comprises the following parts: a cutting edge 752 beveled on one side, an elongated cutting blade shaft 754, a widened blade shaft portion 756, and a slotted blade handle accepting portion 758. The blade shaft portion 754 is specifically configured in size to work with the blade centering backpressure plate 650 to fit snuggly within the blade centering tabs on the backpressure plate 650 and force the inserted blade to be centered as it passes down through the blade guide slots

FIG. 152 depicts a rear view of the large width sized curved flexible elongated blade shaft medial calcar and/or lateral cutting blade 800 having a cutting edge 802 and an elongated blade shaft 804 as shown in FIG. 151B, illustrating the beveled cutting-edge side on cutting edge 802. The large width sized curved elongated flexible medial calcar and/or lateral cutting blade 800 comprises the following parts: a cutting edge 802 beveled on one side, an elongated cutting blade shaft 804, a widened blade shaft portion 806, and a slotted blade handle accepting portion 808. The blade shaft portion 804 is specifically configured in size to work with the blade centering backpressure plate 650 to fit snuggly within the blade centering tabs on the backpressure plate 650 and force the inserted blade to be centered as it passes down through the blade guide slots

FIG. 153 depicts a rear view of the medium width sized curved elongated blade shaft flexible medial calcar and/or lateral cutting blade 800 having a cutting edge 802 and an elongated blade shaft 804 as shown in FIG. 150A. The medium width sized curved elongated flexible medial calcar and/or lateral cutting blade 800 comprises the following parts: a cutting edge 802 beveled on one side, an elongated cutting blade shaft 804, a widened blade shaft portion 806, and a slotted blade handle accepting portion 808. The blade shaft portion 804 is specifically configured in size to work with the blade centering backpressure plate 650 to fit snuggly within the blade centering tabs on the backpressure plate 650 and force the inserted blade to be centered as it passes down through the blade guide slots

FIG. 154 depicts a rear view of the small width sized curved elongated blade shaft flexible medial calcar and/or lateral cutting blade 800 having a cutting edge 802 and an elongated blade shaft 804 as shown in FIG. 150A. The small width sized curved elongated flexible medial calcar and/or lateral cutting blade 800 comprises the following parts: a cutting edge 802 beveled on one side, an elongated cutting blade shaft 804, a widened blade shaft portion 806, and a slotted blade handle accepting portion 808. The blade shaft portion 804 is specifically configured in size to work with the blade centering backpressure plate 650 to fit snuggly within the blade centering tabs on the backpressure plate 650 and force the inserted blade to be centered as it passes down through the blade guide slots

FIG. 155A depicts a top plan view of a small width sized curved short blade shaft flexible medial calcar and/or lateral cutting blade 850 having a cutting edge 852 and a shortened blade shaft (not seen). The slotted blade handle accepting portion 858 can be seen in this view as well as the curvature of the cutting edge 852 and a shortened blade shaft 854.

FIG. 155B depicts a front view of the small width sized curved short blade shaft flexible medial calcar and/or lateral cutting blade 850 having a cutting edge 852 and a shortened blade shaft 854 as shown in FIG. 155A. The small width sized curved elongated flexible medial calcar and/or lateral cutting blade 850 comprises the following parts: a cutting edge 852 beveled on one side, an elongated cutting blade shaft 854, a widened blade shaft portion 856, and a slotted blade handle accepting portion 858. The blade shaft portion 854 is specifically configured in size to work with the blade centering backpressure plate 650 to fit snuggly within the blade centering tabs on the backpressure plate 650 and force the inserted blade to be centered as it passes down through the blade guide slots.

FIG. 156A depicts a top plan view of a medium width sized curved short blade shaft flexible medial calcar and/or lateral cutting blade 850 having a cutting edge 852 and a shortened blade shaft (not seen). The slotted blade handle accepting portion 858 can be seen in this view as well as the curvature of the cutting edge 852 and a shortened blade shaft 854.

FIG. 156B depicts a front view of the medium width sized curved short blade shaft flexible medial calcar and/or lateral cutting blade 850 having a cutting edge 852 and a shortened blade shaft 854 as shown in FIG. 156A. The medium width sized curved elongated flexible medial calcar and/or lateral cutting blade 850 comprises the following parts: a cutting edge 852 beveled on one side, an elongated cutting blade shaft 854, a widened blade shaft portion 856, and a slotted blade handle accepting portion 858. The blade shaft portion 854 is specifically configured in size to work with the blade centering backpressure plate 650 to fit snuggly within the blade centering tabs on the backpressure plate 650 and force the inserted blade to be centered as it passes down through the blade guide slots.

FIG. 157A depicts a top plan view of a large width sized curved short blade shaft flexible medial calcar and/or lateral cutting blade 850 having a cutting edge 852 and a shortened blade shaft (not seen). The slotted blade handle accepting portion 858 can be seen in this view as well as the curvature of the cutting edge 852 and a shortened blade shaft 854.

FIG. 157B depicts a front view of the large width sized short blade shaft flexible medial calcar and/or lateral cutting blade 850 having a cutting edge 852 and a shortened blade shaft 854

as shown in FIG. 157A. The large width sized curved elongated flexible medial calcar and/or lateral cutting blade 850 comprises the following parts: a cutting edge 852 beveled on one side, an elongated cutting blade shaft 854, a widened blade shaft portion 856, and a slotted blade handle accepting portion 858. The blade shaft portion 854 is specifically configured in size to work with the blade centering backpressure plate 650 to fit snuggly within the blade centering tabs on the backpressure plate 650 and force the inserted blade to be centered as it passes down through the blade guide slots.

FIG. 158 depicts a rear view of the large width sized short blade shaft flexible medial calcar and/or lateral cutting blade 850 having a cutting edge 852 and a shortened blade shaft 854 as shown in FIG. 157B, illustrating the beveled cutting edge 852.

FIG. 159 depicts a rear view of the medium width sized short blade shaft flexible medial calcar and/or lateral cutting blade 850 having a cutting edge 852 and a shortened blade shaft 854 as shown in FIG. 156B, illustrating the beveled cutting edge 852.

FIG. 160 depicts a rear view of the small width sized short blade shaft flexible medial calcar and/or lateral cutting blade 850 having a cutting edge 852 and a shortened blade shaft 854 as shown in FIG. 155B, illustrating the beveled cutting edge 852.

FIG. 161 depicts a front view of a large sized anterior/posterior (A/P) osteotome 870, which blade is right-left (R-L) oriented and illustrates the beveled cutting edge 872. The various parts are as follows: a one-sided beveled cutting edge 872, an elongated curved one-sided beveled cutting edge 874, a blade handle shaft 876 and a slotted blade handle accepting portion 878. The elongated curved one-sided beveled cutting edge 874 is configured to follow the curved contour of the femoral stem to be extracted. In operation, the anterior/posterior (A/P) osteotome 870 is inserted along the anterior or posterior of the femoral stem, with the beveled side out away from the stem to cut the stem away from the bone during revision surgery procedures.

FIG. 162 depicts a front view of a medium sized anterior/posterior (A/P) osteotome 870, which blade is right-left (R-L) oriented and illustrates the beveled cutting edge 872. The various parts are as follows: a one-sided beveled cutting edge 872, an elongated curved one-sided beveled cutting edge 874, a blade handle shaft 876 and a slotted blade handle accepting portion 878. The elongated curved one-sided beveled cutting edge 874 is configured to follow the curved contour of the femoral stem to be extracted. In operation, the anterior/posterior (A/P) osteotome 870 is inserted along the anterior or posterior of the femoral stem, with the beveled side out away from the stem to cut the stem away from the bone during revision surgery procedures.

FIG. 163 depicts a front view of a small sized anterior/posterior (A/P) osteotome 870, which blade is right-left (R-L) oriented and illustrates the beveled cutting edge 872. The various parts are as follows: a one-sided beveled cutting edge 872, an elongated curved one-sided beveled cutting edge 874, a blade handle shaft 876 and a slotted blade handle accepting portion 878. The elongated curved one-sided beveled cutting edge 874 is configured to follow the curved contour of the femoral stem to be extracted. In operation, the anterior/posterior (A/P) osteotome 870 is inserted along the anterior or posterior of the femoral stem, with the beveled side out away from the stem to cut the stem away from the bone during revision surgery procedures.

FIG. 164 depicts a rear view of a small sized anterior/posterior (A/P) osteotome 870, which blade is right-left (R-L) oriented as shown in FIG. 163. The rear side of the anterior/posterior (A/P) osteotome 870 is not beveled, and during the revision surgery makes contact with the outer surface of the femoral stem to be extracted.

FIG. 165 depicts a rear view of a medium sized anterior/posterior (A/P) osteotome 870, which blade is right-left (R-L) oriented as shown in FIG. 162. The rear side of the anterior/posterior (A/P) osteotome 870 is not beveled, and during the revision surgery makes contact with the outer surface of the femoral stem to be extracted.

FIG. 166 depicts a rear view of a large sized anterior/posterior (A/P) osteotome 870, which blade is right-left (R-L) oriented as shown in FIG. 161. The rear side of the anterior/posterior (A/P) osteotome 870 is not beveled, and during the revision surgery makes contact with the outer surface of the femoral stem to be extracted.

FIG. 167 depicts a front view of a large sized anterior/posterior (A/P) osteotome 890, which blade is right-left (L-R) oriented and illustrates the beveled cutting edge 892. The various parts of the anterior/posterior (A/P) osteotome 890 are as follows: a one-sided beveled cutting edge 892, an elongated curved one-sided beveled cutting edge 894, a blade handle shaft 896 and a slotted blade handle accepting portion 898. The elongated curved one-sided beveled cutting edge 894 is configured to follow the curved contour of the femoral stem to be extracted. In operation, the anterior/posterior (A/P) osteotome 890 is inserted along the anterior or posterior of the femoral stem, with the beveled side out away from the stem to cut the stem away from the bone during revision surgery procedures.

FIG. 168 depicts a front view of a medium sized anterior/posterior (A/P) osteotome 890, which blade is left-right (L-R) oriented and illustrates the beveled cutting edge 892. The various parts of the anterior/posterior (A/P) osteotome 890 are as follows: a one-sided beveled cutting edge 892, an elongated curved one-sided beveled cutting edge 894, a blade handle shaft 896 and a slotted blade handle accepting portion 898. The elongated curved one-sided beveled cutting edge 894 is configured to follow the curved contour of the femoral stem to be extracted. In operation, the anterior/posterior (A/P) osteotome 890 is inserted along the anterior or posterior of the femoral stem, with the beveled side out away from the stem to cut the stem away from the bone during revision surgery procedures.

FIG. 169 depicts a front view of a small sized anterior/posterior (A/P) osteotome 890, which blade is left-right (L-R) oriented and illustrates the beveled cutting edge 892. The various parts of the anterior/posterior (A/P) osteotome 890 are as follows: a one-sided beveled cutting edge 892, an elongated curved one-sided beveled cutting edge 894, a blade handle shaft 896 and a slotted blade handle accepting portion 898. The elongated curved one-sided beveled cutting edge 894 is configured to follow the curved contour of the femoral stem to be extracted. In operation, the anterior/posterior (A/P) osteotome 890 is inserted along the anterior or posterior of the femoral stem, with the beveled side out away from the stem to cut the stem away from the bone during revision surgery procedures.

FIG. 170 depicts a rear view of a small sized anterior/posterior (A/P) osteotome 890, which blade is left-right (L-R) oriented as shown in FIG. 169. This side is not beveled.

FIG. 171 depicts a rear view of a medium sized anterior/posterior (A/P) osteotome 890, which blade is left-right (L-R) oriented as shown in FIG. 168. This side is not beveled.

FIG. 172 depicts a rear view of a large sized anterior/posterior (A/P) osteotome 890, which blade is left-right (L-R) oriented as shown in FIG. 167. This side is not beveled.

FIG. 173A depicts a top plan view of a small sized compound curved flexible medial calcar blade 900, illustrating that the cutting blade is curved in two planes. The various parts seen in this view include: a cutting edge 902, a curved cutting shaft 904 and a slotted blade handle accepting portion 908. The cutting edge 902 and cutting shaft 904 are compound curved, that is they are curved in both the latitudinal aspect and the longitudinal aspect. This compound curved medial calcar blade is configured to hug the curved medial calcar surface of the femoral stem to be extracted.

FIG. 173B depicts a rear view of a small sized compound curved flexible medial calcar blade 900, illustrating that the cutting blade is curved in two planes as shown in FIG. 173A. The various parts seen in this view include: a cutting edge 902, a curved cutting shaft 904, a widened-out blade shaft 906 and a slotted blade handle accepting portion 908. The cutting edge 902 and cutting shaft 904 are compound curved, that is they are curved in both the latitudinal aspect and the longitudinal aspect. This compound curved medial calcar blade is configured to hug the curved medial calcar surface of the femoral stem to be extracted.

FIG. 174A depicts a top plan view of a medium sized compound curved flexible medial calcar blade 900, illustrating that the cutting blade is curved in two planes. The various parts seen in this view include: a cutting edge 902, a curved cutting shaft 904 and a slotted blade handle accepting portion 908.

FIG. 174B depicts a rear view of a medium sized compound curved flexible medial calcar blade 900, illustrating that the cutting blade is curved in two planes as shown in FIG. 174A. The various parts seen in this view include: a cutting edge 902, a curved cutting shaft 904, a widened-out blade shaft 906 and a slotted blade handle accepting portion 908. The cutting edge 902 and cutting shaft 904 are compound curved, that is they are curved in both the latitudinal aspect and the longitudinal aspect. This compound curved medial calcar blade is configured to hug the curved medial calcar surface of the femoral stem to be extracted.

FIG. 175A depicts a top plan view of a large width sized compound curved flexible medial calcar blade 900, illustrating that the cutting blade is curved in two planes. The various parts seen in this view include: a cutting edge 902, a curved cutting shaft 904 and a slotted blade handle accepting portion 908.

FIG. 175B depicts a rear view of a large width sized compound curved flexible medial calcar blade 900, illustrating that the cutting blade is curved in two planes as shown in FIG. 175A. The various parts seen in this view include: a cutting edge 902, a curved cutting shaft 904, a widened-out blade shaft 906 and a slotted blade handle accepting portion 908. The cutting edge 902 and cutting shaft 904 are compound curved, that is they are curved in both the latitudinal aspect plane and the longitudinal aspect plane. This compound curved medial calcar blade is configured to hug the curved medial calcar surface of the femoral stem to be extracted.

FIG. 176 depicts a side elevational view of the large width sized compound curved flexible medial calcar blade 900, illustrating that the cutting blade is curved in two planes, the latitudinal plane and the longitudinal plane, as shown in FIG. 175A and 175B. The curvature is specifically configured to follow the curved contour of the medial calcar of the femoral stem to be extracted during revision surgery procedures.

FIG. 177 depicts a top, side and perspective view of the small width sized compound curved flexible medial calcar blade 900, illustrating that the cutting blade is curved in two planes as shown in FIG. 173B. Again, the parts include: a cutting edge 902, a curved cutting shaft 904, a widened-out blade shaft 906 and a slotted blade handle accepting portion 908.

FIG. 178 depicts a top, side and perspective view of the medium width sized compound curved flexible medial calcar blade 900, illustrating that the cutting blade is curved in two planes as shown in FIG. 174B.

FIG. 179 depicts a top, side and perspective view of the large width sized compound curved flexible medial calcar blade 900, illustrating that the cutting blade is curved in two planes as shown in FIG. 175B.

FIG. 180 depicts a front view of a large width sized compound curved flexible medial calcar blade 900, illustrating that the cutting blade is curved in two planes as shown in FIG. 175B, here showing the beveled cutting edge. a cutting edge 902, a curved cutting shaft 904, a widened-out blade shaft 906 and a slotted blade handle accepting portion 908.

FIG. 181 depicts a front view of a medium width sized compound curved flexible medial calcar blade 900, illustrating that the cutting blade is curved in two planes as shown in FIG. 174B, here showing the beveled cutting edge.

FIG. 182 depicts a front view of a small width sized compound curved flexible medial calcar blade 900, illustrating that the cutting blade is curved in two planes as shown in FIG. 173B, here showing the beveled cutting edge.

### ROBOTICS/NAVIGATION

FIG. 183 depicts a top, side elevational and perspective view of an embodiment of the joint revision surgery apparatus illustrating a rigid osteotome blade/surgical chisel 330 attached to a powered impaction device 1000 that is part of/attached to a robotic arm. The powered impaction device 1000 may be powered by electricity, air or any other means.

FIG. 184 depicts a top, side elevational and perspective view of an embodiment of the joint revision surgery apparatus illustrating a flexible osteotome blade/surgical chisel 332 attached to a powered impaction device 1000 that is part of/attached to a robotic arm. The powered impaction device 1000 may be powered by electricity, air or any other means.

FIG. 185, 186 and 187 depict a direct head on as well as both oblique views as an example of the navigation, computer navigation or robotic navigation array or tracker 1050 that may be attached to the universal block and or the femoral stem. The array or tracker 1050 is visualized by camera(s) that are part of the navigation and or computer navigation system. The arrays or trackers 1050 may be passive or active (powered). While these example figures show 5 fixed detection points on each array, the arrays or trackers 1050 many have any number of detection points per array or tracker. The arrays or trackers 1050 may also be attached to the handheld impaction device or the robotic arm impaction device or anywhere on the robot.

FIG. 188, 189 and 190 depicts a direct head on as well as both oblique views of an array as used with navigation, computer navigation or robotic navigation The array or tracker 1100 may be attached to the modular adjustable guide block and or the femoral stem or both. The arrays or trackers 1100 are visualized by cameras that are part of the navigation and or computer navigation system. The arrays or trackers 1100 may be passive or active (powered). While these example pictures show 4 fixed detection points on each array or tracker 1100, the arrays or trackers many have any number of detection points per array or tracker. The arrays or trackers 1100 may also be attached to the hand-held impaction device or the robotic arm impaction device or anywhere on the robot.

FIG. 191 and 192 depicts a perspective and side elevational view of an embodiment of the joint revision surgery apparatus illustrating a rigid osteotome blade/surgical chisel 330 attached to a powered impaction device 1000 that is part of/attached to a robotic arm. The powered impaction device 1000 may be powered by electricity, air or any other means. The rigid or flexible blade 330 is attached to the powered impaction device 1000 on a robotic arm which is helping, along with the 2-piece modular adjustable guide block 200, to guide the rigid lateral blade 330 down the stem both for directional and depth assistance. The rigid or flexible blade 330 is shown having been advanced through the 2-piece modular adjustable guide block 200 to the metaphyseal diaphyseal junction (MDJ) of the femoral stem.

FIG. 193 depicts a top elevational view of an embodiment of the joint revision surgery apparatus illustrating a rigid osteotome blade/surgical chisel 330 attached to a powered impaction device 1000 that is part of/attached to a robotic arm. The powered impaction device 1000 may be powered by electricity, air or any other means. The rigid or flexible blade 330 is attached to the powered impaction device 1000 on a robotic arm which is helping, along with the 2-piece modular adjustable guide block 200, to guide the rigid or flexible blade 330 down the stem. The rigid or flexible blade 330 is shown having been advanced through the 2-piece modular adjustable guide block 200 to the metaphyseal diaphyseal junction (MDJ) of the femoral stem.

FIG. 194 depicts a side elevational view of an embodiment of the joint revision surgery apparatus illustrating a rigid or flexible osteotome blade/surgical chisel 330 attached to a powered impaction device 1000 that is part of/attached to a robotic arm. The powered impaction device 1000 may be powered by electricity, air or any other means. The rigid or flexible blade 330 is attached to the powered impaction device 1000 on a robotic arm which is helping, along with the 2-piece modular adjustable guide block 200, to guide the rigid or flexible blade 330 down the stem both for directional and depth assistance. The rigid or flexible blade 330 is shown having been advanced through the 2-piece modular adjustable guide block 200 to the metaphyseal diaphyseal junction (MDJ) of the femoral stem. The 2-piece modular adjustable guide block 200 is shown here in cross-sectional view as shown in FIG. 193.

FIG. 195, 196 and 197 depicts a perspective, side and top view of an embodiment of the joint revision surgery apparatus illustrating the 3-piece modular adjustable guide block 300 attached to the femoral stem with an array or tracker 1100 attached to the modular adjustable guide block 300. The powered impaction device 1000 is attached to a robotic arm which will help, along with the 3-piece modular adjustable guide block 300, to guide a flexible blade 332 down the medial side of the stem both for directional and depth assistance. The blade 332 has been introduced to the 3-piece modular adjustable guide block 300 but not advanced. The adjustable backpressure plate 650 is seen protruding through the modular adjustable guide block.

FIG. 198 depicts a side view of an embodiment of the joint revision surgery apparatus illustrating the 3-piece modular adjustable guide block 300 attached to the femoral stem with an array or tracker 1100 attached to the 3-piece modular adjustable guide block 300. The powered impaction device 1000 is attached to a robotic arm which will help, along with the 3-piece modular adjustable guide block 300, to guide a flexible blade 332 down the medial side of the stem both for directional and depth assistance. The blade 332 has been introduced to the 3-piece modular adjustable guide block 300 but not advanced. The adjustable backpressure plate 650 is seen protruding through the modular adjustable guide block. The modular adjustable guide block is shown in cross-sectional view as shown in FIG. 197.

FIG. 199, 200 and 201 depicts a perspective, side and top view of an embodiment of the joint revision surgery apparatus illustrating the 3-piece modular adjustable guide block 300 attached to the femoral stem with an array or tracker 1100 attached to the 3-piece modular adjustable guide block 300. The powered impaction device 1000 is attached to a robotic arm which will help, along with the 3-piece modular adjustable guide block 300, to guide a flexible blade 332 down the medial side of the stem both for directional and depth assistance. The flexible blade 332 is shown advancing through the 3-piece modular adjustable guide block 300 and down the medial femoral stem. The adjustable backpressure plate 650 is seen protruding through the 3-piece modular adjustable guide block 300.

FIG. 202 depicts a side view of an embodiment of the joint revision surgery apparatus illustrating the 3-piece modular adjustable guide block 300 attached to the femoral stem with an array or tracker 1100 attached to the 3-piece modular adjustable guide block 300. The powered impaction device 1000 is attached to a robotic arm which will help, along with the 3-piece modular adjustable guide block 300, to guide a flexible blade 332 down the medial side of the stem both for directional and depth assistance. The flexible blade 332 is shown advancing through the 3-piece modular adjustable guide block 300 and down the medial femoral stem. The adjustable backpressure plate 650 is seen protruding through the 3-piece modular adjustable guide block 300. The 3-piece modular adjustable guide block 300 is shown in a cross-sectional view as shown in FIG. 201.

FIG. 203, 204 and 205 depicts a perspective, side and top view of an embodiment of the joint revision surgery apparatus illustrating the 3-piece modular adjustable guide block 300 attached to the femoral stem with an array or tracker 1100 attached to the 3-piece modular adjustable guide block 300. The powered impaction device 1000 is attached to a robotic arm which will help, along with the 3-piece modular adjustable guide block 300, to guide a flexible blade 332 down the medial side of the stem both for directional and depth assistance. The flexible blade 332 is shown advancing through the 3-piece modular adjustable guide block 300 well down the medial femoral stem. The adjustable backpressure plate 650 is seen protruding through the 3-piece modular adjustable guide block 300 and deforming the flexible blade 332 to conform it to the medial femoral component.

FIG. 206 depicts a side view of an embodiment of the joint revision surgery apparatus illustrating the 3-piece modular adjustable guide block 300 attached to the femoral stem with an array or tracker 1100 attached to the 3-piece modular adjustable guide block 300. The powered impaction device 1000 is attached to a robotic arm which will help, along with the 3-piece modular adjustable guide block 300, to guide a flexible blade 332 down the medial side of the stem both for directional and depth assistance. The flexible blade 332 is shown advancing through the 3-piece modular adjustable guide block 300 and well down the medial femoral stem. The adjustable backpressure plate 650 is seen protruding through the 3-piece modular adjustable guide block 300 and deforming the flexible blade to conform it to the medial femoral component. The 3-piece modular adjustable guide block 300 is shown in cross-sectional view as shown in FIG. 205.

FIG. 207 depicts a side view of an embodiment of the joint revision surgery apparatus illustrating a rigid osteotome blade/surgical chisel 330 attached to a handheld powered impaction device that is guided by computer navigation 1150. The powered impaction device 1150 is guided by computer navigation through the use of the array or tracker 1152 mounted thereon and the powered impaction device 1150 may be powered by electricity, air or any other means.

FIG. 208 and 209 depicts a perspective and side elevational view of an embodiment of the joint revision surgery apparatus illustrating a rigid osteotome blade/surgical chisel 330 attached to a handheld powered impaction device 1150 that is guided by computer navigation. The powered impaction device 1150 may be powered by electricity, air or any other means. The rigid or flexible blade 330 is attached to the handheld navigated powered impaction device 1150, which is helping, along with the 3-piece modular adjustable guide block 300, to guide the rigid lateral blade 330 down the stem both for directional and depth assistance. The rigid or flexible blade 330 is shown having been advanced through the 3-piece modular adjustable guide block 300 to the metaphyseal diaphyseal junction (MDJ) of the femoral stem. In this way, the guide blade 330 enables the cutting blade (not shown) to go past the MDJ and cut down (release all the way down) to the lower section and tip of the stem.

FIG. 210 depicts a perspective view of an embodiment of the joint revision surgery apparatus illustrating the 3-piece modular adjustable guide block 300 attached to the femoral stem with an array or tracker 1100 attached to the 3-piece modular adjustable guide block 300. The navigated handheld powered impaction device 1200 will help, along with the 3-piece modular adjustable guide block 300, to guide a flexible blade 332 down the medial side of the stem both for directional and depth assistance. The flexible blade 332 is shown advancing through the 3-piece modular adjustable guide block 300 and well down the medial femoral stem. The adjustable backpressure plate 650 is seen protruding through the 3-piece modular adjustable guide block 300 and deforming the flexible blade to conform it to the medial femoral component.

FIG. 211 depicts a side view of an embodiment of the joint revision surgery apparatus illustrating the 3-piece modular adjustable guide block 300 attached to the femoral stem with an array or tracker 1100 attached to the 3-piece modular adjustable guide block 300. The navigated handheld powered impaction device 1200 will help, along with the 3-piece modular adjustable guide block 300, to guide a flexible blade down the medial side of the stem both for directional and depth assistance. The flexible blade 332 is shown advancing through the 3-piece modular adjustable guide block 300 and well down the medial femoral stem. The adjustable backpressure plate 650 is seen protruding through the 3-piece modular adjustable guide block 300 and deforming the flexible blade 332 to conform it to the medial femoral component.

FIG. 212 and 213 depicts a side view of an embodiment of the joint revision surgery apparatus illustrating the 3-piece modular adjustable guide block 300 attached to the femoral stem with an array or tracker 1100 attached to the 3-piece modular adjustable guide block 300. The 3-piece modular medial and lateral parts of the 3-piece modular adjustable guide block 300 have been removed. The navigated handheld powered impaction device 1200 will guide a flexible blade 332 down the medial side of the stem. The flexible blade 332 is shown advancing past the 3-piece modular adjustable guide block 300 in FIG. 212 and extended down the medial femoral stem in FIG. 213.

FIG. 214 depicts a side view of an embodiment of the joint revision surgery apparatus illustrating the 3-piece modular adjustable guide block 300 attached to the femoral stem with an array or tracker 1100 attached to the 3-piece modular adjustable guide block 300. The modular medial and lateral parts of the 3-piece modular adjustable guide block 300 have been removed. The navigated handheld powered impaction device 1200 will guide a flexible blade 332 down the medial side of the stem. The flexible blade 332 is shown advancing well down the medial femoral stem.

FIG. 215 depicts a side elevational view of an embodiment of the joint revision surgery apparatus illustrating the 3-piece modular adjustable guide block 300 attached to the femoral stem with an array or tracker 1100 attached to the 3-piece modular adjustable guide block 300. The modular medial and lateral parts of the 3-piece modular adjustable guide block 300 have been removed. A rigid osteotome blade/surgical chisel 330 attached to a handheld powered impaction device 1200 that is guided by computer navigation through the array tracker 1252 thereon. The powered impaction device may be powered by electricity, air or any other means. The rigid or flexible blade 330 is attached to the handheld navigated powered impaction device 1200 which is guiding the rigid or flexible blade 330 down the stem both for directional and depth assistance. The rigid or flexible blade 330 is shown having been advanced to the metaphyseal diaphyseal junction (MDJ) of the femoral stem.

In summary, a one-piece modular adjustable blade guide block is disclosed, comprising (a) a one piece body including a central portion, a medial portion and a lateral portion and having a centrally located cavity; (b) a trunnion securing member assembly mounted within the centrally located cavity; (c) a plurality of blade guide slots located in the one piece body medial portion; and (d) a plurality of blade guide slots located in the one piece body lateral portion. The one-piece modular adjustable blade guide block wherein said one piece body includes a threaded orifice located in the central portion. The one-piece modular adjustable blade guide block further including a fixation screw having a screw knob and screw threads wherein said screw threads mate with said threaded orifice located in the central portion of said one piece body. The one-piece modular adjustable blade guide block wherein said plurality of blade guide slots located in the one piece body medial portion are configured to accept an upwardly and downwardly adjustable backpressure plate. And the one-piece modular adjustable blade guide block wherein said upwardly and downwardly adjustable backpressure plate further includes blade centering tabs.

Additionally, a two-piece modular adjustable blade guide block is disclosed, comprising: (a) a two piece body including a central piece having a central portion and a medial portion and a removable adjustable lateral piece and having a centrally located cavity within said central portion; (b) a trunnion securing member assembly mounted within the centrally located cavity; (c) a plurality of blade guide slots located in the removable adjustable lateral piece; and (d) a plurality of blade guide slots located in the one piece body medial portion. The two-piece modular adjustable blade guide block wherein said removable adjustable lateral piece is slidably secured to the central portion using an adjustment screw. The two-piece modular adjustable blade guide block further wherein said central piece includes a threaded orifice located in the central portion. The two-piece modular adjustable blade guide block further including a fixation screw having a screw knob and screw threads wherein said screw threads mate with said threaded orifice located in the central portion. The two-piece modular adjustable blade guide block further including a robotics navigation tracker array having screw threads wherein said screw threads mate with said threaded orifice located in the central portion. The two-piece modular adjustable blade guide block wherein said plurality of blade guide slots located in the medial portion are configured to accept an upwardly and downwardly adjustable backpressure plate. And the two-piece modular adjustable blade guide block wherein said upwardly and downwardly adjustable backpressure plate further includes blade centering tabs.

Furthermore, a three-piece modular adjustable blade guide block, comprising: (a) a three piece body including a central piece having a central portion and having a centrally located cavity within said central portion, a removeable medial piece and a removable adjustable lateral; (b) a trunnion securing member assembly mounted within the centrally located cavity; (c) a plurality of blade guide slots located in the removable adjustable lateral piece; and (d) a plurality of blade guide slots located in the medial piece. The three-piece modular adjustable blade guide block wherein said removable adjustable lateral piece is slidably secured to the central portion using an adjustment screw. The three-piece modular adjustable blade guide block further wherein said central piece includes a threaded orifice located in the central portion. The three-piece modular adjustable blade guide block further including a fixation screw having a screw knob and screw threads wherein said screw threads mate with said threaded orifice located in the central portion. The three-piece modular adjustable blade guide block further including a robotics navigation tracker array having screw threads wherein said screw threads mate with said threaded orifice located in the central portion. The three-piece modular adjustable blade guide block wherein said plurality of blade guide slots located in the medial portion are configured to accept an upwardly and downwardly adjustable backpressure plate. The three-piece modular adjustable blade guide block wherein said upwardly and downwardly adjustable backpressure plate further includes blade centering tabs. The three-piece modular adjustable blade guide block wherein said medial piece further includes a threaded orifice configured to accept a fixation point screw. The three-piece modular adjustable blade guide block further including a fixation point screw having a screw knob and screw threads and configured to thread into said threaded orifice located in said medial piece. The three-piece modular adjustable blade guide block wherein said central piece includes one or more threaded orifices. The three-piece modular adjustable blade guide block further including one or more fixation point screws having a screw knob and screw threads and wherein said one or more fixation point screws are configured to be threaded into said threaded orifices located in said central piece.

Moreover, the one-piece, two-piece and three-piece modular adjustable blade guide blocks all include a plurality of blade guide slots located in the lateral portion or piece which are configured to accept rigid lateral guide blades and flexible lateral cutting blades. And the one-piece, two-piece and three-piece modular adjustable blade guide blocks all include a plurality of blade guide slots located in the medial portion or piece which are configured to accept rigid compound curved medial calcar guide blades and flexible medial calcar cutting blades.

The Modular Adjustable Blade Guide Block for Joint Revision Surgery 10A, 100, 200, 300, 350, 400, 450, 500 and 600 shown in the drawings and described in detail herein disclose arrangements of elements of particular construction and configuration for illustrating preferred embodiments of structure and method of operation of the present design. It is to be understood, however, that elements of different construction and configuration and other arrangements thereof, other than those illustrated and described may be employed for providing a Modular Adjustable Blade Guide Block for Joint Revision Surgery 10A, 100, 200, 300, 350, 400, 450, 500 and 600 in accordance with the spirit of this application, and such changes, alternations and modifications as would occur to those skilled in the art are considered to be within the scope of this application as broadly defined in the appended claims.

While certain embodiments of the inventions have been described, these embodiments have been presented by way of example only, and are not intended to limit the scope of the disclosure. Indeed, the novel methods and systems described herein may be embodied in a variety of other forms. Furthermore, various omissions, substitutions and changes in the systems and methods described herein may be made without departing from the spirit of the disclosure. For example, one portion of one of the embodiments described herein can be substituted for another portion in another embodiment described herein. The accompanying claims and their equivalents are intended to cover such forms or modifications as would fall within the scope and spirit of the disclosure. Accordingly, the scope of the present inventions is defined only by reference to the appended claims.

Features, materials, characteristics, or groups described in conjunction with a particular aspect, embodiment, or example are to be understood to be applicable to any other aspect, embodiment or example described in this section or elsewhere in this specification unless incompatible therewith. All of the features disclosed in this specification (including any accompanying claims, abstract and drawings), and/or all of the steps of any method or process so disclosed, may be combined in any combination, except combinations where at least some of such features and/or steps are mutually exclusive. The protection is not restricted to the details of any foregoing embodiments. The protection extends to any novel one, or any novel combination, of the features disclosed in this specification (including any accompanying claims, abstract and drawings), or to any novel one, or any novel combination, of the steps of any method or process so disclosed.

Furthermore, certain features that are described in this disclosure in the context of separate implementations can also be implemented in combination in a single implementation. Conversely, various features that are described in the context of a single implementation can also be implemented in multiple implementations separately or in any suitable subcombination. Moreover, although features may be described above as acting in certain combinations, one or more features from a claimed combination can, in some cases, be excised from the combination, and the combination may be claimed as a subcombination or variation of a subcombination.

Moreover, while operations may be depicted in the drawings or described in the specification in a particular order, such operations need not be performed in the particular order shown or in sequential order, or that all operations be performed, to achieve desirable results. Other operations that are not depicted or described can be incorporated in the example methods and processes. For example, one or more additional operations can be performed before, after, simultaneously, or between any of the described operations. Further, the operations may be rearranged or reordered in other implementations. Those skilled in the art will appreciate that in some embodiments, the actual steps taken in the processes illustrated and/or disclosed may differ from those shown in the figures. Depending on the embodiment, certain of the steps described above may be removed, others may be added. Furthermore, the features and attributes of the specific embodiments disclosed above may be combined in different ways to form additional embodiments, all of which fall within the scope of the present disclosure. Also, the separation of various system components in the implementations described above should not be understood as requiring such separation in all implementations, and it should be understood that the described components and systems can generally be integrated together in a single product or packaged into multiple products.

For purposes of this disclosure, certain aspects, advantages, and novel features are described herein. Not necessarily all such advantages may be achieved in accordance with any particular embodiment. Thus, for example, those skilled in the art will recognize that the disclosure may be embodied or carried out in a manner that achieves one advantage or a group of advantages as taught herein without necessarily achieving other advantages as may be taught or suggested herein.

Conditional language, such as “can,” “could,” “might,” or “may,” unless specifically stated otherwise, or otherwise understood within the context as used, is generally intended to convey that certain embodiments include, while other embodiments do not include, certain features, elements, and/or steps. Thus, such conditional language is not generally intended to imply that features, elements, and/or steps are in any way required for one or more embodiments or that one or more embodiments necessarily include logic for deciding, with or without user input or prompting, whether these features, elements, and/or steps are included or are to be performed in any particular embodiment.

Conjunctive language such as the phrase “at least one of X, Y, and Z,” unless specifically stated otherwise, is otherwise understood with the context as used in general to convey that an item, term, etc. may be either X, Y, or Z. Thus, such conjunctive language is not generally intended to imply that certain embodiments require the presence of at least one of X, at least one of Y, and at least one of Z.

Language of degree used herein, such as the terms “approximately,” “about,” “generally,” and “substantially” as used herein represent a value, amount, or characteristic close to the stated value, amount, or characteristic that still performs a desired function or achieves a desired result. For example, the terms “approximately”, “about”, “generally,” and “substantially” may refer to an amount that is within less than 10% of, within less than 5% of, within less than 1% of, within less than 0.1% of, and within less than 0.01% of the stated amount. As another example, in certain embodiments, the terms “generally parallel” and “substantially parallel” refer to a value, amount, or characteristic that departs from exactly parallel by less than or equal to 15 degrees, 10 degrees, 5 degrees, 3 degrees, 1 degree, or 0.1 degree.

The scope of the present disclosure is not intended to be limited by the specific disclosures of preferred embodiments in this section or elsewhere in this specification, and may be defined by claims as presented in this section or elsewhere in this specification or as presented in the future. The language of the claims is to be interpreted broadly based on the language employed in the claims and not limited to the examples described in the present specification or during the prosecution of the application, which examples are to be construed as non-exclusive.

Further, the purpose of the foregoing abstract is to enable the U.S. Patent Office, foreign patent offices worldwide and the public generally, and especially the scientists, engineers and practitioners in the art who are familiar with patent or legal terms or phraseology, to determine quickly from a cursory inspection the nature and essence of the technical disclosure of the application. The abstract is neither intended to define the invention of the application, which is measured by the claims, nor is it intended to be limiting as to the scope of the invention in any way.

I claim:

1. A modular adjustable blade guide block, comprising:
(a) a one piece body including a central portion, a medial portion and a lateral portion and having a centrally located cavity;
(b) a trunnion securing member assembly mounted within the centrally located cavity;
(c) a plurality of blade guide slots located in the one piece body medial portion; and
(d) a plurality of blade guide slots located in the one piece body lateral portion.

2. The modular adjustable blade guide block according to claim 1, further wherein said one piece body includes a threaded orifice located in the central portion.

3. The modular adjustable blade guide block according to claim 2, further including a fixation screw having a fixation screw knob and fixation screw threads wherein said fixation screw threads mate with said threaded orifice located in the central portion of said one piece body.

4. The modular adjustable blade guide block according to claim 1, wherein said plurality of blade guide slots located in the one piece body medial portion are configured to accept an upwardly and downwardly adjustable backpressure plate.

5. The modular adjustable blade guide block according to claim 4, wherein said upwardly and downwardly adjustable backpressure plate further includes blade centering tabs.

6. A modular adjustable blade guide block, comprising:
(a) a two piece body including a central piece having a central portion and a medial portion and a removable adjustable lateral piece and having a centrally located cavity within said central portion;
(b) a trunnion securing member assembly mounted within the centrally located cavity;
(c) a plurality of blade guide slots located in the removable adjustable lateral piece; and
(d) a plurality of blade guide slots located in the one piece body medial portion.

7. The modular adjustable blade guide block according to claim 6, wherein said removable adjustable lateral piece is slidably secured to the central portion using an adjustment screw.

8. The modular adjustable blade guide block according to claim 6, further wherein said central piece includes a threaded orifice located in the central portion.

9. The modular adjustable blade guide block according to claim 8, further including a fixation screw having a fixation screw knob and fixation screw threads wherein said fixation screw threads mate with said threaded orifice located in the central portion.

10. The modular adjustable blade guide block according to claim 8, further including a robotics navigation tracker array having tracker array screw threads wherein said tracker array screw threads mate with said threaded orifice located in the central portion.

11. The modular adjustable blade guide block according to claim 6, wherein said plurality of blade guide slots located in the medial portion are configured to accept an upwardly and downwardly adjustable backpressure plate.

12. The modular adjustable blade guide block according to claim 11, wherein said upwardly and downwardly adjustable backpressure plate further includes blade centering tabs.

13. A modular adjustable blade guide block, comprising:
(a) a three piece body including a central piece having a central portion and having a centrally located cavity within said central portion, a removeable medial piece and a removable adjustable lateral;
(b) a trunnion securing member assembly mounted within the centrally located cavity;
(c) a plurality of blade guide slots located in the removable adjustable lateral piece; and
(d) a plurality of blade guide slots located in the medial piece.

14. The modular adjustable blade guide block according to claim 13, wherein said removable adjustable lateral piece is slidably secured to the central portion using an adjustment screw.

15. The modular adjustable blade guide block according to claim 13, further wherein said central piece includes a threaded orifice located in the central portion.

16. The modular adjustable blade guide block according to claim 15, further including a fixation screw having a fixation screw knob and fixation screw threads wherein said fixation screw threads mate with said threaded orifice located in the central portion.

17. The modular adjustable blade guide block according to claim 15, further including a robotics navigation tracker array having tracker array screw threads wherein said tracker array screw threads mate with said threaded orifice located in the central portion.

18. The modular adjustable blade guide block according to claim 13, wherein said plurality of blade guide slots located in the medial portion are configured to accept an upwardly and downwardly adjustable backpressure plate.

19. The modular adjustable blade guide block according to claim 18, wherein said upwardly and downwardly adjustable backpressure plate further includes blade centering tabs.

20. The modular adjustable blade guide block according to claim 12, wherein said medial piece further includes a medial piece threaded orifice configured to accept a fixation point screw.

21. The modular adjustable blade guide block according to claim 12, further including a medial piece fixation point screw having a fixation screw knob and fixation screw threads and wherein said medial piece fixation screw threads are configured to thread into said medial piece threaded orifice located in said medial piece.

22. The modular adjustable blade guide block according to claim 12, wherein said central piece includes one or more central piece threaded orifices.

23. The modular adjustable blade guide block according to claim 22, further including one or more fixation point screws having a fixation screw knob and fixation screw threads and wherein said one or more fixation point screw threads are configured to be threaded into said central piece threaded orifices located in said central piece.

24. The modular adjustable blade guide block according to claim 12, wherein said plurality of blade guide slots located in the removable adjustable lateral piece are configured to accept rigid lateral guide blades and flexible lateral cutting blades.

25. The modular adjustable blade guide block according to claim 12, wherein said plurality of blade guide slots located in the medial piece are configured to accept rigid compound curved medial calcar guide blades and flexible medial calcar cutting blades.
